(19) United States
(12) Patent Application Publication
(10) Pub. No.: US 2025/0352472 A1
(43) Pub. Date: Nov. 20, 2025

(54) LIPID NANOPARTICLE (LNP) COMPOSITIONS FOR PLACENTA-SELECTIVE CARGO DELIVERY, AND METHODS OF USE THEREOF

(71) Applicant: The Trustees of the University of Pennsylvania, Philadelphia, PA (US)

(72) Inventors: Michael J. Mitchell, Philadelphia, PA (US); Kelsey L. Swingle, Philadelphia, PA (US); Hannah Safford, Philadelphia, PA (US); Hannah Geisler, Philadelphia, PA (US)

(21) Appl. No.: 19/288,484

(22) Filed: Aug. 1, 2025

Related U.S. Application Data
(63) Continuation-in-part of application No. 19/120,621, filed on Jan. 1, 0001.
(60) Provisional application No. 63/379,107, filed on Oct. 11, 2022.
(60) Provisional application No. 63/496,862, filed on Apr. 18, 2023.
(60) Provisional application No. 63/496,825, filed on Apr. 18, 2023.
(60) Provisional application No. 63/678,940, filed on Aug. 2, 2024.

(51) Int. Cl. A61K 9/1271; A61K 45/06

(52) U.S. Cl. CPC A61K 9/1271; A61K 45/06

(57) ABSTRACT

In one aspect, the present disclosure relates to lipid nanoparticles (LNPs) comprising at least one ionizable lipid, at least one helper lipid, cholesterol, and at least one polymer conjugated lipid. In certain embodiments, the LNP further comprises at least one cargo molecule. In certain embodiments, the LNP comprises an epidermal growth factor (EGFR) targeting domain. In another aspect, the present disclosure provides a method of delivering a cargo to the placenta of a subject. In another aspect, the present disclosure provides a method of treating, preventing, and/or ameliorating a placental disease and/or disorder in a subject. In certain embodiments, the placental disease and/or disorder is pre-eclampsia.

### CROSS-REFERENCE TO RELATED APPLICATIONS

This application is a Continuation-In-Part of, and claims priority to, U.S. Provisional Patent Application No. 63/678,940 filed Aug. 2, 2024 and U.S. patent application Ser. No. 19/120,621 filed Apr. 11, 2025, which is a 35 U.S.C. § 371 national phase application from, and claims priority to, PCT International Patent Application No. PCT/US2023/076572 filed

Oct. 11, 2023, which claims priority under 35 U.S.C. § 119(e) to U.S. Provisional Patent Application No. 63/379,107 filed Oct. 11, 2022, U.S. Provisional Patent Application No. 63/496,825 filed Apr. 18, 2023, and U.S. Provisional Patent Application No. 63/496,862 filed Apr. 18, 2023, all of which applications are incorporated herein by reference in their entireties.

### STATEMENT REGARDING FEDERALLY SPONSORED RESEARCH OR DEVELOPMENT

This invention was made with government support under TR002776 awarded by the National Institutes of Health. The government has certain rights in the invention.

### SEQUENCE LISTING

The XML file named “046483-7401WO1—Sequence Listing.xml” created on Oct. 4, 2023, comprising 2,200 Bytes, is hereby incorporated by reference in its entirety.

### BACKGROUND

Viral and non-viral nucleic acid delivery approaches have been explored for a variety of clinical applications including vaccines, protein and enzyme replacement therapies, and gene editing technologies. Viral platforms for nucleic acid delivery require genomic integration and therefore result in permanent gene expression. However, these platforms pose risks associated with immunogenicity and ectopic genomic integration which can be particularly harmful in gene editing applications. Non-viral approaches include the delivery of therapeutic messenger RNA (mRNA), which does not require nuclear transport and initiates transient protein expression in the cytosol. mRNA faces several delivery challenges in vivo including rapid degradation by nucleases and poor cellular uptake due to its large size and negative charge. Drug delivery platforms such as lipid nanoparticles (LNPs) can address these challenges as they have demonstrated efficient cellular uptake and potent mRNA delivery in vivo. Currently, LNPs are the most clinically advanced non-viral drug delivery platform for nucleic acid therapeutics such as mRNA. Specifically, LNPs are utilized by Moderna and Pfizer/BioNTech's COVID-19 mRNA vaccines and Intellia's gene editing therapies for congenital disorders. For these reasons, much attention has been devoted to exploring LNP-mediated mRNA delivery for novel applications.

LNP-mediated nucleic acid therapy has been relatively unexplored for applications including placental disorders during pregnancy. The placenta is an organ that is fetal in origin and develops rapidly during gestation to supply nutrients and oxygen to the fetus. Insufficient vasodilation in the placenta can result in disorders such as pre-eclampsia which affects 3-8% of all pregnancies. During pre-eclampsia, placental vasodilation is compromised and maternal blood pressure rises in an effort to continue providing nutrients and oxygen to the fetus. In severe cases, fetal growth restriction (FGR) develops, which is characterized by abnormally low fetal growth rates. FGR is the leading cause of stillbirth and prematurity worldwide, as the only curative treatment option for pre-eclampsia and FGR involves delivering the fetus regardless of viability and gestational age.

To address pre-eclampsia and FGR, attempts have been made to developed gene therapies to improve placental vasodilation and angiogenesis. This has been done by either upregulating vascular endothelial growth factor (VEGF) or downregulating soluble fms-like tyrosine kinase-1 (sFlt-1, the soluble version of VEGF receptor 1) which is overexpressed in pre-eclampsia. Most of these therapies have used viral approaches, however due to the challenges associated with permanent, off-target VEGF expression, these therapies have been administered locally to the placenta via an invasive intra-uterine artery injection.

Alternatively, non-viral platforms such as mRNA LNPs offer the opportunity for transient VEGF expression via a simpler injection route such as intravenous administration. However, LNP-mediated mRNA delivery to the placenta has been minimally evaluated during pregnancy.

Thus, there is a need in the art for LNPs suitable for delivery of cargo to the placenta, and methods of use thereof. The present disclosure addresses this need.

### BRIEF SUMMARY

In one aspect, the present disclosure provides a lipid nanoparticle (LNP). In certain embodiments, the LNP comprises at least one ionizable lipid. In certain embodiments, the LNP comprises at least one helper lipid. In certain embodiments, the LNP comprises cholesterol and/or a derivative thereof (e.g., sterols, such as cholesterol, 24-α-methyl-cholesterol (campesterol), 24-α-ethyl-cholestanol (stigmastanol), and 24-α-ethyl-cholesterol (β-sitosterol)). In certain embodiments, the LNP comprises at least one polymer conjugated lipid.

In certain embodiments, the at least one ionizable lipid comprises a compound of Formula (I), or a salt, solvate, stereoisomer, or isotopologue thereof, wherein R^{1a}, R^{1b}, R^{1a}, R^{2b}, R^{2c}, R^{2d}, R^{2e}, R^{2f}, R^{2g}, and R^{2h} are defined elsewhere herein:

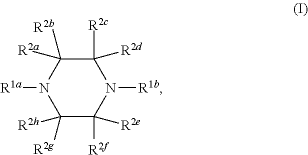

In certain embodiments, the LNP comprises at least one cargo molecule. In certain embodiments, the cargo molecule is one or more of a nucleic acid, small molecule, protein, therapeutic agent, antibody, and any combinations thereof. In certain embodiments, the LNP mediates placenta-selective cargo delivery.

In certain embodiments, the LNP comprises an epidermal growth factor (EGFR) targeting domain. In certain embodiments, the EGFR targeting domain is covalently conjugated to at least one component of the LNP.

In another aspect, the present disclosure provides a method of delivering cargo to the placenta of a pregnant subject. In certain embodiments, the method comprises administering to the subject a therapeutically effective amount of at least one lipid nanoparticle (LNP) of the present disclosure comprising at least one cargo molecule. In certain embodiments, the cargo is mRNA. In certain embodiments, the mRNA encodes VEGF.

In another aspect, the present disclosure provides a method of treating, preventing, and/or ameliorating a placental disease and/or disorder in a subject. In certain embodiments, the method comprises administering to the subject a therapeutically effective amount of at least one lipid nanoparticle (LNP) of the present disclosure comprising at least one cargo molecule. In certain embodiments, the cargo is mRNA. In certain embodiments, the mRNA encodes VEGF.

In certain embodiments, the placental disease or disorder is selected from the group consisting of pre-eclampsia, fetal growth restriction (FGR), intrauterine growth restriction (IUGR), placenta previa, placenta accreta, placenta increta, and placenta percreta.

### BRIEF DESCRIPTION OF THE FIGURES

The drawings illustrate generally, by way of example, but not by way of limitation, various embodiments of the present application.

FIG. 1 depicts engineering lipid nanoparticles (LNPs) for in vivo mRNA delivery to the placenta in pregnant mice. Engineering placenta-specific mRNA LNPs with applications in mediating vasodilation for placental insufficiency disorders. First, an LNP library was prepared wherein each LNP was formulated with a unique ionizable lipid to screen in vitro luciferase mRNA delivery in placental cells. After identifying a lead candidate, LNP-mediated luciferase mRNA delivery LNP was evaluated in the maternal organs, fetuses, and placentas of non-pregnant and pregnant mice. Finally, VEGF mRNA LNPs demonstrated increased vasodilation in the placenta with potential applications in treating placental insufficiency disorders such as pre-eclampsia.

FIGS. 2A-2D depict an overview of the ionizable lipid library, LNP formulation, and characterization. FIG. 2A depicts the chemical structures of the three epoxides (e.g., C12 (A), C14 (B), and C16 (C)) and five polyamine cores (e.g., polyamine cores 1, 2, 3, 4, and 5) used in the preparation of the ionizable lipids disclosed herein and utilized in the LNP library screen. FIG. 2B provides a schematic showing the synthesis of ionizable lipid A4 via nucleophilic addition (e.g., epoxide opening reaction). FIG. 2C depicts formulation of LNPs via microfluidic mixing of an ethanol phase containing ionizable lipid, phospholipid, cholesterol, and lipid-PEG and an aqueous phase containing mRNA. FIG. 2D provides exemplary size, polydispersity index (PDI), mRNA encapsulation efficiency, and pKₐ characterization data for the LNP library. Data are reported as mean±standard deviation (n=3 observations).

FIGS. 3A-3E depict in vitro LNP-mediated luciferase mRNA delivery to placental cells. FIG. 3A depicts regions of the mouse placenta from the maternal to fetal side (left) and cell types separating the maternal and fetal blood spaces in the labyrinth region (right). FIGS. 3B-3C: JEG-3 trophoblast cells were treated with LNPs or Lipofectamine MessengerMAX at a dose of 50 ng of luciferase mRNA per 25,000 cells for 24 h. Normalized luciferase expression was quantified by subtracting bioluminescence values from untreated cells and normalizing to the Lipofectamine group. Normalized luciferase expression is reported as mean±SEM of n=6 biological replicates (averaged from n=5 technical replicates each). Percent cell viability for each treatment condition was normalized to untreated cells and is reported as mean±SEM of n=4 biological replicates (averaged from n=4 technical replicates each). Nested one-way ANOVAs with post hoc Student's t tests using the Holm-Šidák correction for multiple comparisons were used to compare normalized luciferase expression or cell viability across treatment groups to Lipofectamine. FIGS. 3D-3E: luciferase expression and cell viability were evaluated in a dose dependent manner at 25, 50, 75, 100, and 150 ng of luciferase mRNA per 25,000 cells. Normalized luciferase expression is reported as mean±SEM of n=6 biological replicates (averaged from n=5 technical replicates each) and percent cell viability is reported as mean±SEM of n=4 biological replicates (averaged from n=3 technical replicates each). Nested one-way ANOVAs with post hoc Student's t tests using the Holm-Šidák correction for multiple comparisons were used to compare normalized luciferase expression or cell viability across treatment groups to Lipofectamine at each dose. *:p≤0.05, **:p≤0.01, ***:p≤0.001, ****:p≤0.0001.

FIGS. 4A-4G depict in vivo LNP-mediated luciferase mRNA delivery to certain organs of non-pregnant and pregnant mice. FIGS. 4A-4B depict IVIS images and quantification of luciferase mRNA LNP delivery (0.6 mg/kg) to the heart, lung, liver, kidneys, and spleen in non-pregnant (FIG. 4A) and pregnant mice (FIG. 4B). FIG. 4C provides the spleen to liver ratio for each LNP treatment group in non-pregnant and pregnant mice. FIGS. 4D-4G provide IVIS images (FIGS. 4D-4E) and quantification (FIGS. 4F-4G) of luciferase mRNA LNP delivery to the uterus in non-pregnant (FIG. 4D and FIG. 4F) and pregnant mice (FIG. 4E and FIG. 4G). All data are reported as mean±SEM (n=5-10 biological replicates). One-way ANOVAs were performed on normalized flux measurements with post hoc Student's t tests. Asterisks without bars denote significant comparisons to the PBS treated group. *:p≤0.05, **:p≤0.01, ***:p≤0.001, ****:p≤0.0001.

FIGS. 5A-5F depict in vivo LNP-mediated luciferase mRNA delivery to certain organs of non-pregnant and pregnant mice. FIGS. 5A-5B provide IVIS images and quantification of luciferase mRNA LNP delivery (0.6 mg/kg) to the placentas (FIG. 5A) and fetuses (FIG. 5B) of pregnant mice. One-way ANOVAs were performed on normalized flux measurements with post hoc Student's t tests. Asterisks without bars denote significant comparisons to the PBS-treated group. **:p≤0.01, ***:p≤0.001. FIG. 5C depicts organ specificity of each LNP treatment group calculated as a percent of total luminescent flux for the spleen, placenta, and liver. FIGS. 5D-5F depict mCherry fluorescence intensity histograms and percent mCherry positive CD31+/CD45− endothelial cells (FIG. 5D) or CK7+/CD31−/CD45− trophoblasts (FIG. 5E), and CD45+ immune cells (FIG. 5F) isolated from PBS or mCherry LNP-treated pregnant mice at 1 mg/kg. Data are reported as mean±SEM (n=4-8 placentas from 5 biological replicates; M1-M5=mouse 1-mouse 5).

FIGS. 6A-6E depict evaluation of VEGF expression and toxicity in pregnant mice treated with VEGF mRNA LNPs. FIGS. 6A-6C depict VEGF expression in serum (FIG. 6A), livers (FIG. 6B), and placentas (FIG. 6C) both 6 h and 48 h following treatment with PBS or VEGF mRNA LNPs as a dose of 1 mg mRNA/kg. VEGF concentration in livers and placentas was normalized to mass of total protein in the tissue homogenate. One-way ANOVAs were performed on VEGF concentration measurements with post hoc Student's t tests. Data are reported as mean±SEM (for serum: n=6 observations from 3 biological replicates, for livers: n=18 observations from 3 biological replicates, for placentas: n=4-8 placentas and n=3 biological replicates). FIG. 6D depicts ALT and AST enzyme levels in serum 48 h after treatment with PBS or VEGF mRNA LNPs. A two-way ANOVA was performed on serum enzyme levels with post hoc Student's t tests. Data are reported as mean±SEM (n=6 observations from 3 biological replicates). FIG. 6E depicts cytokine levels in placental tissue homogenates 6 h and 48 h after treatment with PBS or VEGF mRNA LNPs. For each cytokine, data are normalized to the average of the optical density measurements for the PBS-treated mice. Data are reported as mean±SEM (n=3 observations). ***:p≤0.0002, ****:p≤0.0001.

FIGS. 7A-7D: assessing local VEGF expression and vasodilation in the placenta after treating pregnant mice with VEGF mRNA LNPs. FIG. 7A: 4× and 40× images from VEGFR1 stained placentas. In the 4× images, the junctional zone and labyrinth are divided by a dashed black line. In 40× images, the brown VEGFR1 staining is darker in the LNP A4 group, particularly in the regions surrounding the white blood spaces. FIG. 7B: 4× and 20× images from CD31 stained placentas. Regions positive for CD31 are stained brown and denote fetal blood spaces. For VEGFR1 and CD31 staining, representative images are shown for each treatment group that had percent positive VEGFR1 positive area or mean fetal blood vessel area measurements closest to the mean. FIG. 7C: quantification of percent VEGFR1 positive area from VEGFR1 stained placentas using ImageJ. FIG. 7D: quantification of fetal blood vessel area from CD31 stained placentas using ImageJ. Nested one-way ANOVAs with post hoc Student's t test using the Holm-Šidák correction for multiple comparisons were used to compare percent VEGFR1 positive area or mean fetal blood vessel area across treatment groups. Data are reported as median with 25th and 75th quartiles (n=3 biological replicates with n=3 placentas per mouse, n=2 sections per placenta, and n=3 distinct images per section for a total of n=54 images per treatment group). M: mouse, *:p≤0.05, **:p≤0.01.

FIG. 8 depicts the chemical structures of industry standard ionizable lipids C12-200 and DLin-MC3-DMA.

FIG. 9 depicts a representative gating scheme for mCherry positive endothelial cells (CD31+ CD 45−) and trophoblast cells (CK7+ CD31− CD45−).

FIGS. 10A-10F: in vitro screening of library A′ for LNP-mediated luciferase mRNA delivery to BeWo b30 trophoblast cells. FIG. 10A: schematic of orthogonal DOE process used to generate library A′. FIG. 10B: levels of excipient molar ratios used to generate library A′, comprising 16 LNP formulations. FIGS. 10C-10D: luciferase expression (FIG. 10C) and cell viability (FIG. 10D) of BeWo b30 cells 24 hours after treatment with library A′ LNPs or the initial lead formulation (S1). Cells were treated with 50 ng of luciferase mRNA per 50,000 cells. Relative luminescence was quantified by normalizing to cells treated with S1 (dashed line in FIG. 10C) and cell viability was measured by normalizing to untreated cells (dashed line in FIG. 10D). Results are reported as mean±standard deviation from n=3 biological replicates. Nested one-way ANOVAs with post hoc Student's t tests using the Holm-Ši\acute{d}a\acute{k} correction for multiple comparisons were used to compare the luciferase expression or cell viability across treatment groups to S1, ***p≤0.001, ****p≤0.0001. FIG. 10E: average luminescence values of the 4 LNP formulations at each molar ratio of C12-494, DOPE, cholesterol and PEG. Error bars=standard error of the mean. FIG. 10F: average luminescence values of the 2 LNP formulations at a specific molar ratio of one excipient (C12-494 or cholesterol) and either the lower or higher molar ratios of the second excipient (DOPE, cholesterol or C12-494) were plotted. Error bars=standard error of the mean.

FIGS. 11A-11E: in vitro screening of libraries B′ and C′ for LNP-mediated luciferase mRNA delivery to trophoblast cells. FIG. 11A: schematic of orthogonal DOE process used to generate library B′ and C′ where both library B′ and C′ contain 8 LNP formulations. Levels of excipient molar ratios used to generate (FIG. 11B) library B′ or (FIG. 11C) library C′. FIGS. 11E-11E: Luciferase expression (FIG. 11D) and cell viability (FIG. 11E) of BeWo b30 cells 24 hours after treatment with library B′ and C′ LNPs or S1. Cells were treated with 50 ng of luciferase mRNA per 50,000 cells. Relative luminescence was quantified by normalizing to cells treated with S1 (dashed line) and cell viability was measured by normalizing to untreated cells (dashed line). Results are reported as mean±standard deviation from n=3 biological replicates. Nested one-way ANOVAs with post hoc Student's t tests using the Holm-Ši\acute{d}a\acute{k} correction for multiple comparisons were used to compare the luciferase expression or cell viability across treatment groups to S1. *p≤0.05, **p≤0.01, ****p≤0.0001.

FIGS. 12A-12B: in vitro dose-response of top performing LNPs from library A′, B′ and C′. Luciferase expression (FIG. 12A) and cell viability (FIG. 12B) of BeWo b30 cells 24 hours after treatment with S1, A′1, B′5, and C′5 LNPs. Cells were treated in a dose dependent manner at 10, 25, 50, 100 or 250 ng per 50,000 cells. Luminescence and cell viability were quantified by normalizing to untreated cells (dashed line). Results are reported as mean±standard deviation from n=3 biological replicates. A two-way ANOVA with post hoc Student's t tests using the Holm-Ši\acute{d}a\acute{k} correction for multiple comparisons was used to compare the luciferase expression or cell viability across treatment groups and dosing amounts to S1. *p≤0.05, **p≤0.01, ****p≤0.0001.

FIGS. 13A-13F: in vivo luciferase mRNA LNP delivery in pregnant mice to the maternal organs, placentas and fetuses. FIGS. 13A-13B: IVIS images (FIG. 13A) and quantification (FIG. 13B) of luciferase mRNA LNP delivery to the maternal heart, lung, liver, kidney and spleen of pregnant mice. Representative IVIS images are shown from the mouse with the normalized flux in the spleen closest to the mean. Normalized flux is reported as mean±standard deviation from n=3 biological replicates. A two-way ANOVA with post hoc Student's t tests using the Holm-S̅i\acute{d}a\acute{k} correction for multiple comparisons was used to compare the normalized flux across treatment groups and organs to S1. **p≤0.01, ****p≤0.0001. FIGS. 13C-13D: IVIS images (FIG. 13C) and quantification (FIG. 13D) of luciferase mRNA LNP delivery to the placentas of pregnant mice. FIGS. 13E-13F IVIS images (FIG. 13E) and quantification (FIG. 13F) of luciferase mRNA LNP delivery to the fetuses of pregnant mice. Representative IVIS images of both the placentas and fetuses are shown from the mouse with the normalized flux in the placentas closest to the mean. Normalized flux is reported as mean±standard deviation from n=3 biological replicates (with n=6-9 placentas and fetuses). Nested one-way ANOVAs with post hoc Student's t tests using the Holm-Ši\acute{d}a\acute{k} correction for multiple comparisons were used to compare normalized flux across treatment groups. ****p≤0.0001.

FIGS. 14A-14H: average luminescence values of the 2 LNP formulations at a specific molar ratio of one excipient and either the lower or higher molar ratios of the second excipient. Error bars=standard error of the mean.

FIGS. 15A-15D: IVIS images of maternal organs 6 hours after luciferase mRNA LNP delivery via intravenous injection in pregnant mice.

FIGS. 16A-16C: IVIS images of fetuses and placentas 6 hours after luciferase mRNA LN delivery via intravenous injection in pregnant mice.

FIG. 17: organ specificity of LNPs A′1, C′5, and S1 calculated as a percentage of total luminescent flux of the maternal organs, placentas, and fetuses.

FIGS. 18A-18H: high-throughput in vivo screening using DNA barcode (b-DNAs) to identify extrahepatic LNP formulations. FIG. 18A: each of these 98 LNPs were formulated encapsulating a unique b-DNA enabling high-throughput, in vivo screening. The pooled LNPs were administered to non-pregnant (n 6 biological replicates) and pregnant mice (n 6 biological replicates) via tail vein administration following which organs were collected, processed, and prepared for next generation sequencing (NGS). Demultiplexing and subsequent data analysis identified an extrahepatic, placenta-tropic LNP formulation that was used to encapsulate vascular endothelial growth factor (VEGF) mRNA to treat pre-eclampsia during pregnancy. FIG. 18B-18E: characterization of the LNP library; dashed lines representing the mean. FIGS. 18F-18H: mean normalized delivery heatmaps depicting both enrichment and depletion of particular LNP formulations to non-pregnant (FIG. 18F) and pregnant (FIG. 18G) maternal organs as well as placentas and fetuses (FIG. 18H).

FIGS. 19A-19F: effects of polyamine core, epoxide tail length, and excipient formulation on characterization data of the LNP library. FIGS. 19A-19C: Z-average size (FIG. 19A), zeta potential (FIG. 19B), and encapsulation efficiency (FIG. 19C) across polyamine core and epoxide tail length, only for LNPs with the standard excipient formulation. FIGS. 19D-19F: Z-average size (FIG. 19D), zeta potential (FIG. 19E), and encapsulation efficiency (FIG. 19F) across polyamine core and excipient formulation.

FIG. 20: the fetuses and their respective placentas from pregnant mice can be distinguished based on their location in the uterine horn: proximal fetuses/placentas are located closest to the ovary and distal fetuses/placentas are located closest to the cervix. The ovarian artery first supplies blood to the proximal placentas and the uterine artery first supplies blood to the distal placentas.

FIGS. 21A-21B: normalized delivery results from high-throughput in vivo b-DNA screen across polyamine core and epoxide tail length in the liver, lung, spleen, and uterus (FIG. 21A) of non-pregnant (NP) and pregnant (P) mice as well as the placentas and fetuses (FIG. 21). Data points represent the mean±SEM (n 6 biological replicates). The dashed line denotes the mean normalized delivery of the C12-200 LNP formulation.

FIGS. 22A-22B: Normalized delivery results from high-throughput in vivo b-DNA screen across polyamine core and excipient formulation in the liver, lung, spleen, and uterus (FIG. 22A) of non-pregnant (NP) and pregnant (P) mice as well as the placentas and fetuses (FIG. 22B). Data points represent the mean±SEM (n 6 biological replicates).

FIGS. 23A-23G: exemplary LNP formulations enable extrahepatic LNP delivery to the placenta. FIGS. 23A-23C: volcano plots depicting significantly enriched and significantly depleted LNPs compared to the liver-tropic C12-200 LNP formulation in non-pregnant (NP) (FIG. 23A) and pregnant (P) (FIG. 23B) maternal organs as well as fetuses and placentas (c). For each organ, one-way ANOVAs with post hoc Student's t tests using the Holm-Bonferroni correction for multiple comparisons to the C12-200 LNP were used to compare normalized delivery across LNP formulations and generate p values. FIGS. 23D-23G: the squared Pearson's correlation coefficient for mean normalized delivery (R²) was calculated for each organ pair and is presented as a heatmap in non-pregnant maternal organs (FIG. 23D), pregnant maternal organs (FIG. 23E), between non-pregnant and pregnant maternal organs (FIG. 23F), and placentas and fetuses (FIG. 23G).

FIGS. 24A-24B: normalized delivery scatter plots from high-throughput in vivo b-DNA screen comparing LNP delivery in proximal versus distal placentas (FIG. 24A) and proximal versus distal fetuses (FIG. 24B). Data points represent the mean (n 6 biological replicates) normalized delivery of a particular LNP formulation and plots are labeled with the squared Pearson's correlation coefficient for each pair of organs.

FIGS. 25A-25D: normalized delivery scatter plots from high-throughput in vivo b-DNA screen comparing LNP delivery in the pregnant liver versus lung (FIG. 25A), the pregnant liver versus spleen (FIG. 25B), placentas versus liver (FIG. 25C), and placentas versus fetuses (FIG. 25D). Data points represent the mean (n 6 biological replicates) normalized delivery of a particular LNP formulation. Plots are labeled with the squared Pearson's correlation coefficient for each pair of organs.

FIGS. 26A-26M: in vivo high-throughput screening identifies placenta-tropic mRNA LNP 55. LNP 6 (negative control), LNP 55 (placenta-tropic), LNP 97 (C12-200), and LNP 98 (DLin-MC3-DMA) were formulated with luciferase mRNA and administered to non-pregnant (FIGS. 26A-26E) and pregnant (FIGS. 26F-26M) mice at a dose of 0.6 mg mRNA/kg. 6 h after administration, D-Luciferin was administered, and organs were dissected and imaged using an in vivo imaging system (IVIS). Bioluminescent flux was quantified in the maternal organs, namely the lung (FIG. 26B and FIG. 26G), liver (FIG. 26C and FIG. 26H), and spleen (FIG. 26D and FIG. 26I) which was then used to calculate a spleen to liver ratio (FIG. 26E and FIG. 26J). Luminescent flux measurements in the maternal organs are reported as the mean±SEM (n=4 biological replicates). Bioluminescent flux was also quantified in the placentas (FIG. 26L) and fetuses (FIG. 26M). Representative images (FIG. 26K) are shown from the dam with bioluminescent flux values in the placenta closest to the mean for each treatment group. Luminescent flux measurements in the placentas and fetuses are reported as the mean±SEM for each mouse (n 5-10 placentas/fetuses). Either ordinary or nested one-way ANOVAs with post hoc Student's t tests using the Holm-Šidák correction for multiple comparisons were used to compare luminescent flux across treatment groups. ns: non-significant, *:p≤0.05, **:p≤0.01, ***:p≤0.001, ****:p≤0.0001.

FIGS. 27A-27D: comparing bioluminescent flux in the lung (FIG. 27A), liver (FIG. 27B), and spleen (FIG. 27C) as well as the spleen to liver ratios (FIG. 27D) between non-pregnant (NP) and pregnant (P) mice following luciferase mRNA LNP administration. Luminescent flux measurements are reported as the mean±SEM (n=4 biological replicates). Two-way ANOVAs with post hoc Student's t tests using the Holm-Šidák correction for multiple comparisons were used to compare luminescent flux across LNP treatment groups and non-pregnant/pregnant mice. *:p 0.05, **:p≤0.01, ****:p≤0.0001.

FIGS. 28A-28B: IVIS images of all fetuses and their respective placentas from pregnant mice (n=4 biological replicates) following either PBS or luciferase mRNA LNP administration.

FIGS. 29A-29L: A potential endogenous, protein adsorption-based targeting mechanism for LNP delivery to the placenta. FIG. 29A: the C14-494 ionizable lipid in the placenta-tropic LNP 55 formulation contains multiple ether bonds that might promote increased electronegativity. FIG. 29B: while the DLin-MC3-DMA ionizable lipid is known to mediate liver targeting through ApoE binding, it is proposed herein that the adsorption of β2-GPI to LNP 55 promotes delivery to the placenta. FIG. 29C: surface zeta potential measurements for LNP 55 and LNP 98 (n=4). LNPs were pre-coated with either ApoE or #β2-GPI and used to treat liver Hep G2 (FIG. 29D-29F), spleen Jurkat (FIG. 29G-29I), or placenta BeWo b30 cells (FIG. 29J-29L). Luciferase expression was used to evaluate mRNA expression (FIGS. 29D-29E, 29G-29H, and 29J-29K), while confocal microscopy and flow cytometry was used to evaluate intracellular uptake of DiD-labeled LNPs (FIGS. 29F, 29I, and 29L). Normalized luciferase expression is reported as the mean±SEM (n 5 biological replicates with 5 technical replicates each). Representative histograms are shown from the technical replicate with a value for the percent of DiD⁺ cells closest to the mean for each treatment group. Nested one-way ANOVAs with post hoc Student's t tests using the Holm-Šidák correction for multiple comparisons were used to compare normalized luciferase expression across increasing protein concentrations to the uncoated LNP treatment groups. ns: non-significant, *:p≤0.05, **:p≤0.01, ***:p≤0.001, ****:p≤0.0001.

FIGS. 30A-30C: quantifying in vitro intracellular uptake of DiD-labeled LNPs in Hep G2 (FIG. 30A), Jurkat (FIG. 30B), and BeWo b30 cells (FIG. 30C). Cells were treated with 50 ng of LNP 55, either uncoated or pre-incubated in 0.75 μg of ApoE or #β2-GPI per μg of lipid for 30 min. The percent of DiD⁺ cells is reported as the mean±SEM (n=4 biological replicates with n=6 technical replicates each). Nested one-way ANOVAs with post hoc Student's t tests using the Holm-Šidák correction for multiple comparisons were used to compare the percent of DiD⁺ cells across treatment groups. *:p≤0.05, **:p≤0.01, ***:p≤0.001, ****:p≤0.0001.

FIGS. 31A-31L: pre-eclampsia increases LNP delivery to placental cells while decreasing off-target delivery to splenic immune cells. To evaluate differences in biodistribution and mRNA expression between healthy and pre-eclamptic pregnant mice, an early-onset model of pre-eclampsia was induced via injection of an ultra-low dose of lipopolysaccharide (LPS). LNP 55 was formulated with luciferase mRNA and labeled with DiD fluorescent dye and administered at a dose of 1 mg of mRNA/kg. 12 h later, fluorescent and bioluminescent IVIS imaging was performed on the maternal organs, placentas, and fetuses (FIGS. 31A-31D). Representative images are shown from the dam with fluorescent or bioluminescent flux values in the placenta closest to the mean for each treatment group. Cellular LNP delivery was evaluated in the spleen (FIGS. 31E-31G) and placentas (FIGS. 31H-31J) via flow cytometry. The percent of DiD⁺ cells is reported as the mean±SEM (n=4 biological replicates). Representative histograms for splenic CD3⁺ T cells (FIG. 31G) and placental CD45⁺ immune cells (FIG. 31J) are shown from samples with the percent of DiD⁺ cells closest to the mean for each treatment group. Immunofluorescent staining on placental sections for pan-trophoblast marker cytokeratin 7 (CK7) (FIG. 31K) and an endothelial cell marker (endomucin) (FIG. 31L) demonstrates colocalization of placental cells and DiD-labeled LNPs.) One-way ANOVAs with post hoc Student's t tests using the Holm-Šidák correction for multiple comparisons were used to compare the percent of DiD⁺ cells across treatment groups. ns: non-significant, *:p≤0.05, **:p≤0.01, ***:p 0.001, ****:p 0.0001.

FIGS. 32A-32B: DiD fluorescence intensity (FIG. 32A) and luciferase luminescence intensity (FIG. 32B) IVIS images of all fetuses and their respective placentas from pregnant mice (n=4 biological replicates) following either PBS or DiD-labeled luciferase mRNA LNP administration.

FIGS. 33A-33D: DiD fluorescent flux and bioluminescent flux in the lung, liver, spleen, placentas, and fetuses following administration of DiD-labeled luciferase mRNA LNP 55 to either healthy pregnant mice or an LPS-induced model of pre-eclampsia. Fluorescent and bioluminescent flux are reported as the mean±SEM (n=4 biological replicates). Either ordinary or nested one-way ANOVAs with post hoc Student's t tests using the Holm-Šidák correction for multiple comparisons were used to compare fluorescent or luminescent flux across treatment groups. *:p≤0.05, **:p≤0.01, ***:p≤0.001, ****:p≤0.0001.

FIGS. 34A-34B: Quantifying the percent of DiD⁺ CD45⁺ immune cells (FIG. 34A) and CD19⁺ B cells (FIG. 34B) in the spleen following administration of DiD-labeled luciferase mRNA LNP 55 to either healthy pregnant mice or an LPS-induced model of pre-eclampsia. Data are reported as the mean±SEM (n=4 biological replicates). Ordinary one-way ANOVAs with post hoc Student's t tests using the Holm-Šidák correction for multiple comparisons were used to compare the percent of DiD⁺ cells across treatment groups. ****:p≤0.0001.

FIGS. 35A-35H: VEGF mRNA LNP rescues maternal blood pressure and fetal weight while modulating the immunophenotype in a model of early-onset pre-eclampsia. FIG. 35A: pre-eclampsia was induced via intraperitoneal (i.p.) injection of 1 μg/kg lipopolysaccharide (LPS) on gestational day E7.5. 1 mg/kg VEGF mRNA LNP 55 was then administered via tail vein (i.v.) injection on gestational day E11. Maternal weight (FIG. 35B) and mean blood pressure (BP) (FIG. 35C) were recorded daily through the model endpoint on E17 before parturition. On E17, fetal and placental weight (FIG. 35D) were recorded and normalized by litter size. Representative images of the first four fetuses are shown from the dam with fetal weight measurements closest to the mean for each treatment group. FIG. 35E: placental vasculature in the labyrinth was visualized with hematoxylin and eosin staining and used to quantify mean blood vessel area. Immunophenotyping was performed to evaluate differences in the immune cell populations in the blood (FIG. 35F), spleen (FIG. 35G), and placenta (FIG. 35H) during pre-eclampsia and upon administration of VEGF mRNA LNPs. Maternal weight, blood pressure, and immune cell population frequencies in the blood and spleen are reported as mean±SEM (n=8 biological replicates). Fetal and placental weight are reported as median with the 25^{th} and 75^{th} percentiles (n=8 biological replicates with 6-9 fetuses or placentas per mouse). Mean blood vessel area is reported as mean±SEM (n=8 biological replicates with 3 placentas per mouse, 2 sections per placenta, and 3 images per section). Immune cell population frequencies in the placenta are reported as mean±SEM (n=8 biological replicates with 2-3 placentas per mouse). Either ordinary or nested (one-way ANOVAs with post hoc Student's t tests using the Holm-Šidák correction for multiple comparisons were used to compare responses across treatment groups. +: PBS vs. LPS, *: LPS vs. LPS+VEGF mRNA LNP 55, ns: non-significant, *:p≤0.05, **:p≤0.01, ***:p≤0.001, ****:p≤0.0001.

FIGS. 36A-36G: Serum levels of vascular endothelial growth factor (VEGF) (FIG. 36A), soluble fms-like tyrosine kinase-1 (sFlt-1) (FIG. 36B), alanine transaminase (ALT) (FIG. 36C), aspartate aminotransferase (AST) (FIG. 36D), tumor necrosis factor-alpha (TNF-α) (FIG. 36E), interleukin-6 (IL-6) (FIG. 36F), and interferon-gamma (IFN-γ) (FIG. 36G) on gestational days E11.5 and E17 in an LPS-induced model of pre-eclampsia during pregnancy. Data are presented as the mean±SEM (n=8 biological replicates). Two-way ANOVAs with post hoc Student's t tests using the Holm-Šidák correction for multiple comparisons were used to compare serum levels across treatment groups and gestational days. *:p≤0.05, **:p≤0.01, ****:p≤0.0001.

FIG. 37 provides a schematic depicting the design, screening and evaluation of EGFR-LNPs of the present disclosure and the placenta selectivity thereof in pregnant mice.

FIGS. 38A-38E provide a schematic representation of EGFR-LNPs (FIG. 38A) and exemplary characterization data for certain EGFR-LNP formulations of the present disclosure, including diameter (FIGS. 38B-38C), encapsulation efficiency (FIG. 38D), and pKa (FIG. 38E).

FIGS. 39A-39D provide exemplary in vitro mRNA delivery data for certain EGFR-LNP formulations of the present disclosure.

FIGS. 40A-40G provide certain exemplary biodistribution data in certain organs of pregnant or non-pregnant mice using certain EGFR-LNP formulations of the present disclosure.

FIGS. 41A-41C provide certain exemplary biodistribution data in the placenta and fetus of pregnant or non-pregnant mice using certain EGFR-LNP formulations of the present disclosure.

FIGS. 42A-42I provide certain exemplary cell-specific accumulation data in the spleen and placenta of pregnant or non-pregnant mice using certain EGFR-LNP formulations of the present disclosure.

FIGS. 43A-43B depict the fold-change in luminescence observed in the liver (FIG. 43A) and spleen (FIG. 43B) using certain exemplary EGFR-LNPs of the present disclosure in pregnant (P) and non-pregnant (NP) mice.

FIGS. 44A-44B depict DiR and mCherry fluorescent flux of placentas after administration of certain exemplary EGFR-LNPs, using IVIS imaging.

FIGS. 45A-45C depict mCherry mRNA delivery in murine placentas with EGFR-LNPs as compared to LNP A1.

FIGS. 46A-46E: Atomic force microscopy (AFM) measurements of lipid nanoparticles (LNPs) reveals tunable elasticity of LNPs through sterol structure. FIG. 46A: Structures of cholesterol, campesterol, β-sitosterol, and stigmastanol. Differences in structures between cholesterol and the analogs are highlighted. FIG. 46B: Schematic depicting AFM immobilization technique for LNPs to determine Young's modulus. FIGS. 46C-46E: The Young's modulus as measured by AFM of (FIG. 46C) industry standard LNP formulations, (FIG. 46D) MC3 LNPs incorporating each cholesterol analog, and (FIG. 46D) C12-494 LNPs incorporating each cholesterol analog. Young's moduli are reported as mean±standard deviation. A one-way ANOVA with post hoc Student's t tests using the Holm-Šidák correction for multiple comparisons was used to compare the Young's modulus across different LNP formulations, *p≤0.05, **p≤0.01, ***p≤0.001, ****p≤0.0001.

FIGS. 47A-47I: In vitro screening of C12-494 cholesterol analog library for LNP uptake and mCherry mRNA expression in trophoblast cells. FIGS. 47A-47C: (FIG. 47A) Representative hydrodynamic diameter intensity distributions, (FIG. 47B) polydispersity index, and (FIG. 47C) encapsulation efficiency of C12-494 Chol, Camp, Sito and Stig LNPs. Data reported as mean standard deviation (n=3 observations). FIGS. 47D-47F: LNP uptake in BeWo b30 cells as quantified by normalized DiR median fluorescent intensity (MFI) (FIG. 47D) 1 h, (FIG. 47E) 4 h or (FIG. 47F) 18 h after treatment with Chol, Camp, Sito or Stig LNPs at a dose of 100 ng of mRNA per 100,000 cells. FIGS. 47G-47I: mRNA transfection in BeWo b30 cells as quantified by normalized mCherry MFI (FIG. 47G) 1 h, (FIG. 47H) 4 h or (FIG. 47I) 18 h after treatment with Chol, Camp, Sito or Stig LNPs. Normalized MFI was quantified by normalizing to the cells treated with Chol LNPs at each time point. Results are reported as mean±standard deviation from n=4 biological replicates. A nested one-way ANOVA with post hoc Student's t tests using the Holm-Šidák correction for multiple comparisons was used to compare normalized MFI across treatment groups to the Chol LNP, *p≤0.05, ****p≤0.0001.

FIGS. 48A-48B: Investigating the role of LNP elasticity on pathways required for LNP uptake and endosomal escape. FIG. 48A: Schematic depicting different routes of cellular uptake of LNPs and downstream endosomal escape and mRNA translation events. Amiloride (AMI) is an inhibitor of macropinocytosis; chlorpromazine (CPZ) is an inhibitor of clathrin-mediated endocytosis; genistein (GEN) is an inhibitor of caveolae-mediated endocytosis; methyl-β-cyclodextrin (MβCD) is an inhibitor of lipid-raft mediated endocytosis; Bafilomycin A1 (BAF A1) is an inhibitor of endosomal acidification. FIG. 48B: Relative luciferase expression in BeWo b30 cells 24 h after treatment of LNPs at a dose of 50 ng of mRNA per 50,000 cells in the presence of different endocytosis inhibitors or DMSO. Relative luminescence signal was quantified by normalizing to cells treated with LNPs in the absence of endocytosis inhibitors. Results are reported as mean±standard deviation from n=4 biological replicates. A two-way ANOVA with post hoc Student's t tests using the Holm-Šidák correction for multiple comparisons was used to compare luciferase expression across treatment groups and inhibitors to the Chol LNP, *p≤0.05, **p≤0.01.

FIGS. 49A-49H: LNP biodistribution and luciferase expression in the maternal organs of pregnant mice. FIGS. 49A-49D: (FIG. 49A) Fluorescent IVIS images and quantification of normalized DiR signal in the maternal (FIG. 49B) lung, (FIG. 49C) liver and (FIG. 49D) spleen of pregnant mice. FIGS. 49E-49H: (FIG. 49E) Luminescent IVIS images and quantification of normalized luciferase mRNA expression in the maternal (FIG. 49F) lung, (FIG. 49G) liver and (FIG. 49H) spleen of pregnant mice. Normalized flux is reported as mean±standard deviation from n=5 biological replicates. A one-way ANOVA with post hoc Student's t tests using the Holm-Šidák correction for multiple comparisons was used to compare normalized flux across treatment groups, *p≤0.05, **p≤0.01, ****p≤0.0001.

FIGS. 50A-50G: LNP biodistribution, luciferase mRNA expression and toxicity in placentas and fetuses of pregnant mice. FIGS. 50A-50C: (FIG. 50A) Fluorescent IVIS images and quantification of normalized DiR signal in the (FIG. 50B) placentas and (FIG. 50C) fetuses of pregnant mice. FIGS. 50D-50F: (FIG. 50D) Luminescent IVIS images and quantification of normalized luciferase mRNA expression in the (FIG. 50E) placentas and (FIG. 50F) fetuses of pregnant mice. IVIS images of both the placentas and fetuses are shown from the mouse with the lowest normalized flux in the placentas. Normalized flux is reported as mean±standard deviation from n=5 biological replicates (with n=4-11 placentas and fetuses). Nested one-way ANOVAs with post hoc Student's t tests using the Holm-Šidák correction for multiple comparisons were used to compare normalized flux across treatment groups, **p≤0.01, ***p≤0.001, ****p≤0.0001. FIG. 50G: Relative cytokine levels in serum 6 h after PBS or LNP administration in pregnant mice. Relative cytokine levels were quantified by normalizing to the optical density measurement for PBS-treated mice (dashed line). Serum cytokine data is reported as mean±standard deviation from n=4 biological replicates. A two-way ANOVA with post hoc Student's t tests using the Holm-Šidák correction for multiple comparisons was used to compare relative cytokine concentrations across treatment groups and cytokines, **p≤0.01, ****p≤0.0001.

FIG. 51: Z-average size of C12-494 Chol, Camp, Sito and Stig LNPs over 2 weeks when stored at 4° C.

FIG. 52: Cell viability of BeWo b30 cells 24 h after treatment with C12-494 Chol, Camp, Sito or Stig LNPs at a dose of 50 ng of mRNA per 50,000 cells. Cell viability was measured by normalizing to untreated cells (dashed line).

FIG. 53A: The Young's modulus as measured by AFM of C12-494 low cholesterol

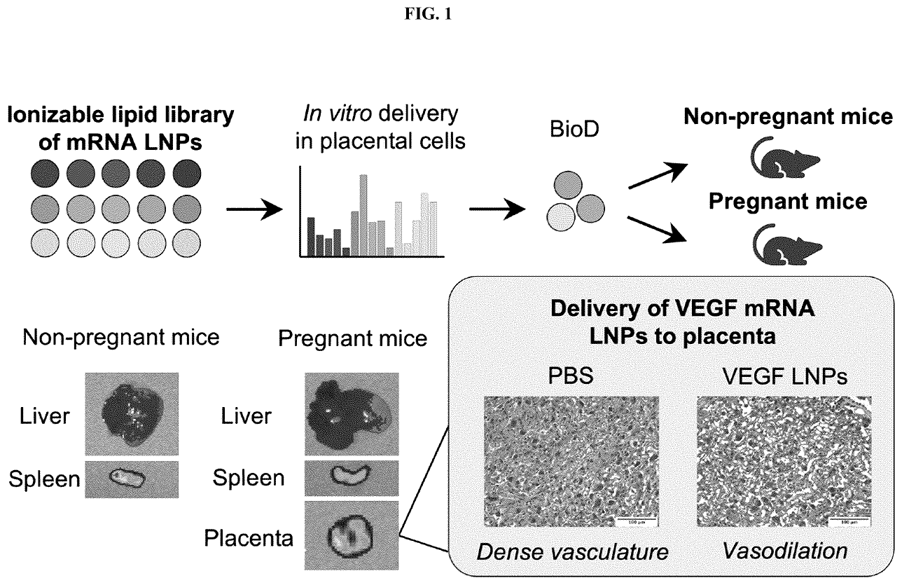

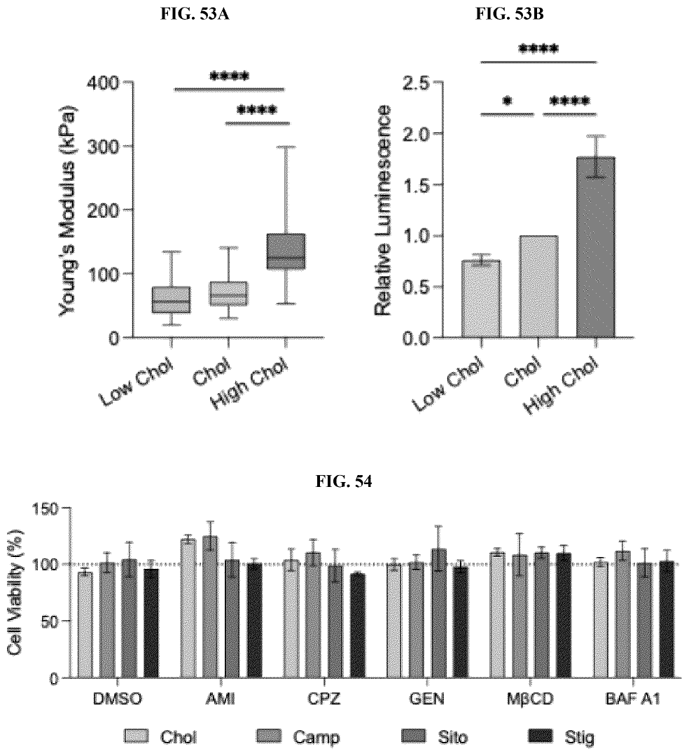

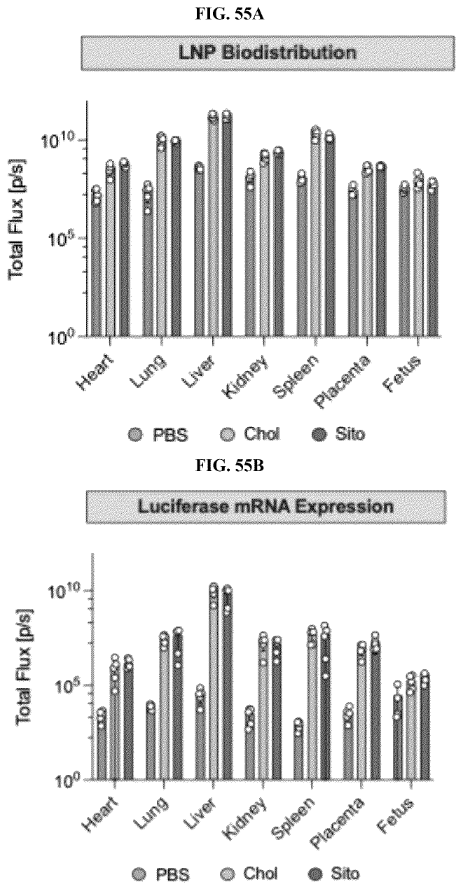

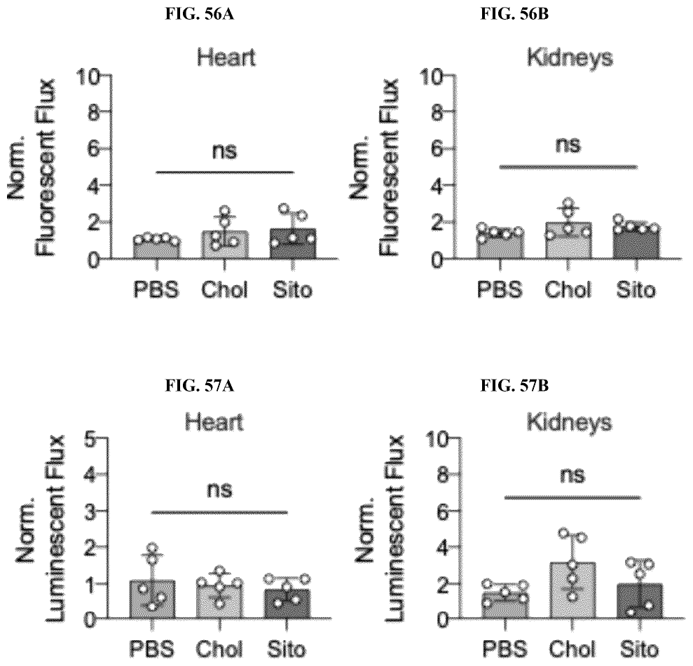

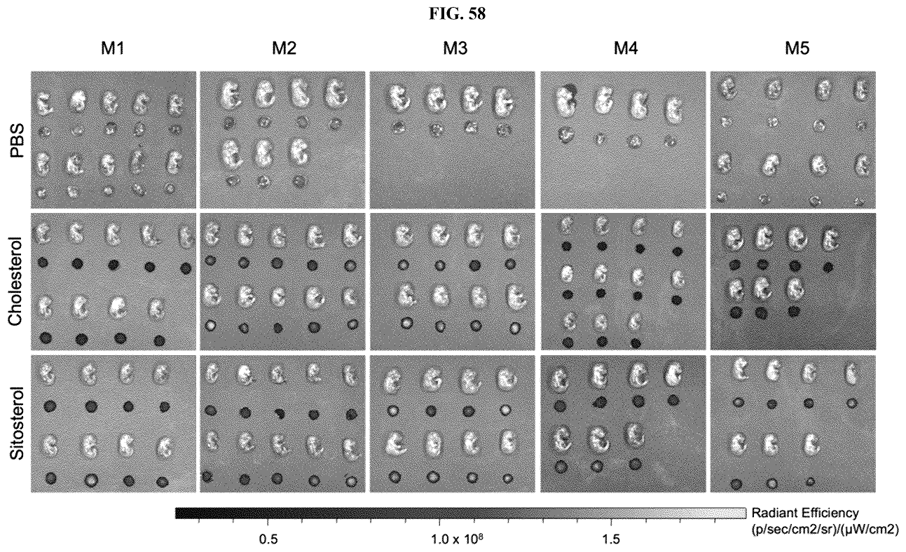

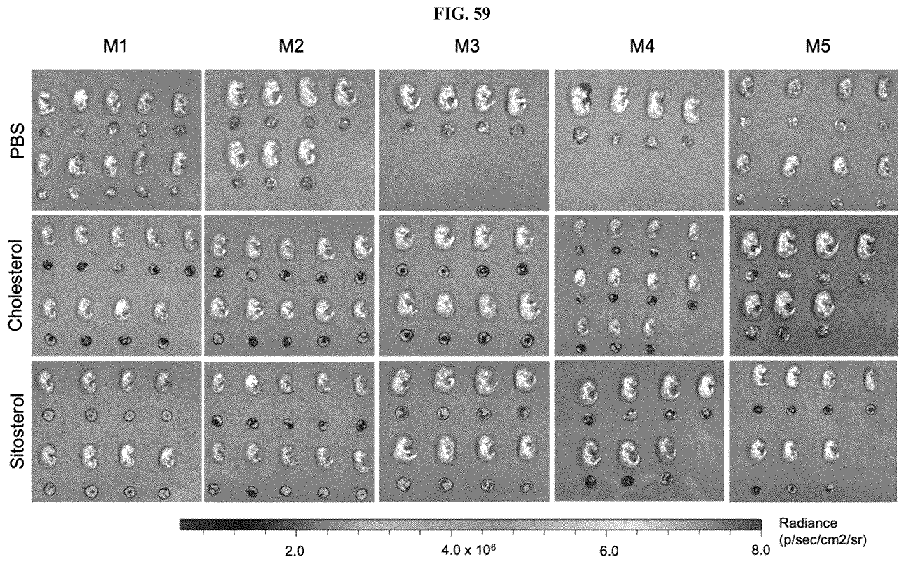

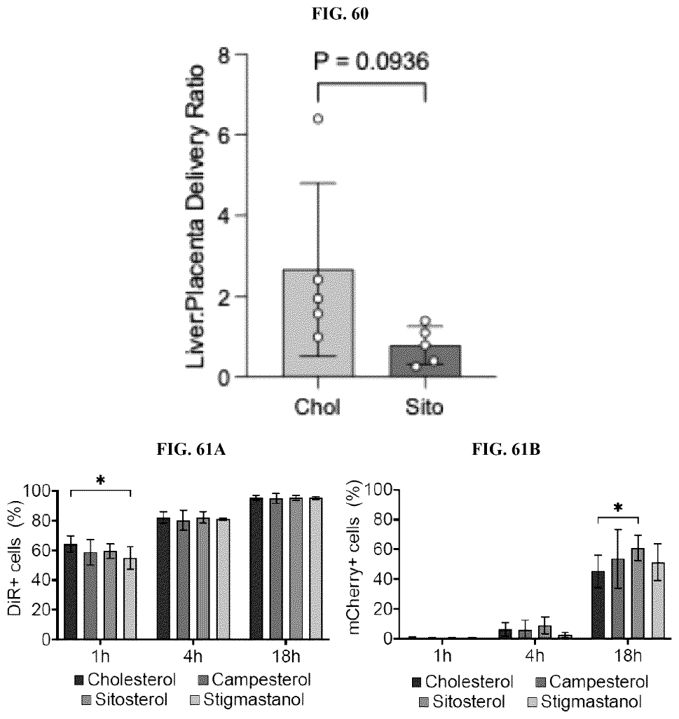

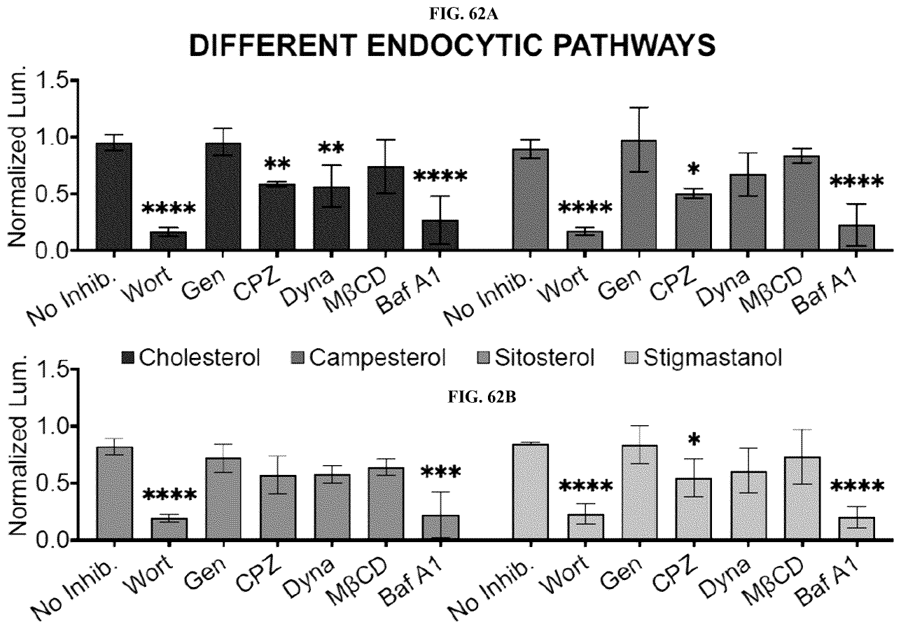

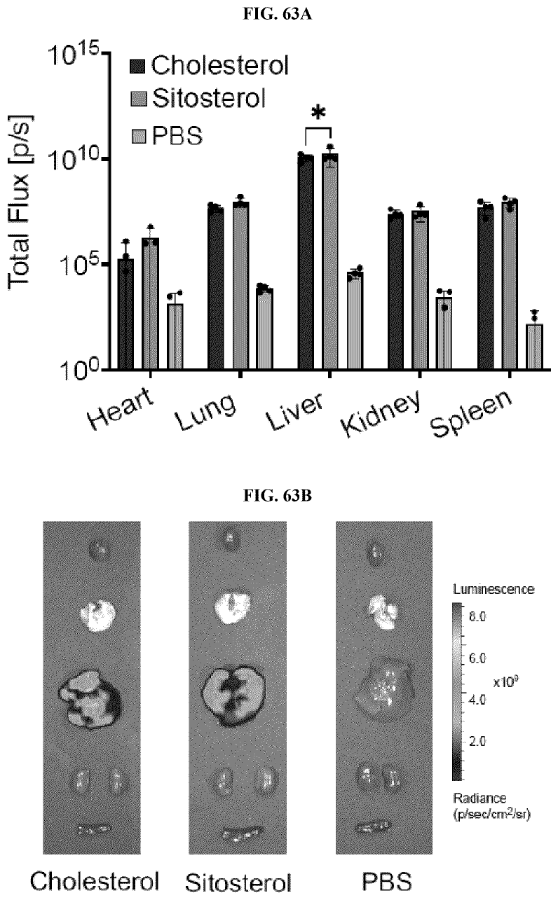

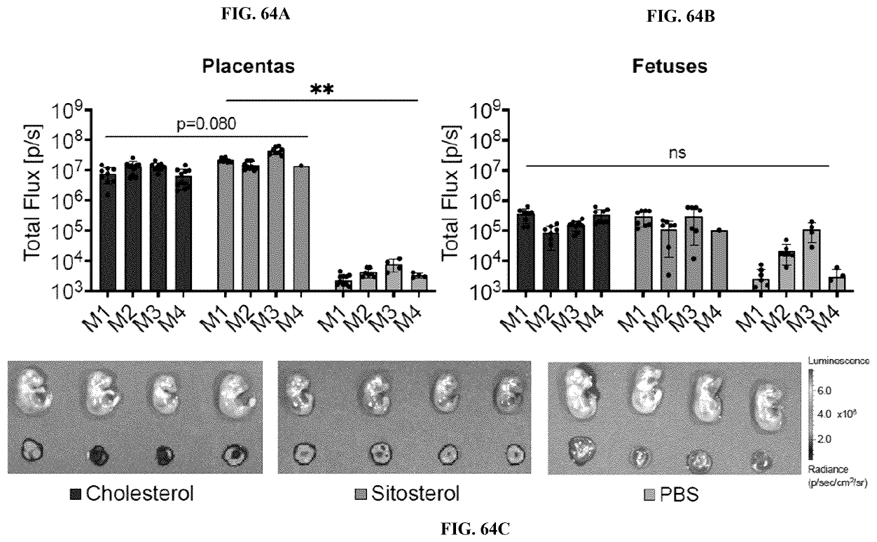

(Low Chol), cholesterol (Chol), or high cholesterol (High Chol) LNPs. A one-way ANOVA with post hoc Student's t tests using the Holm-Šidák correction for multiple comparisons was used to compare the Young's modulus across different LNP formulations. FIG. 53B: Relative luciferase expression in BeWo b30 cells 24 h after treatment of Low Chol, Chol, or High Chol LNPs at a dose of 50 ng of mRNA per 50,000 cells. Relative luminescence was quantified by normalizing to the cells treated with Chol LNPs. A nested one-way ANOVA with post hoc Student's t tests using the Holm-Šidák correction for multiple comparisons was used to compare relative luminescence across treatment groups to the Chol LNP, *p≤0.05, ****p≤0.0001.

FIG. 54: Cell viability of BeWo b30 cells 24 h after treatment of LNPs in the presence of different endocytosis inhibitors or DMSO. Cell viability was measured by normalizing to untreated cells (dashed line).

FIGS. 55A-55B: Quantification of (FIG. 55A) fluorescent flux and (FIG. 55B) luminescent flux in IVIS images of the heart, lung, liver, kidney, spleen, placentas and fetuses of pregnant mice 6 h after intravenous administration of DiR-labeled luciferase mRNA LNPs.

FIGS. 56A-56B: Quantification of normalized DiR signal in the maternal (FIG. 56A) hearts and (FIG. 56B) kidneys of pregnant mice 6 h after intravenous administration of DiR-labeled luciferase mRNA LNPs.

FIGS. 57A-57B: Quantification of normalized luciferase mRNA expression in the maternal (FIG. 57A) hearts and (FIG. 57B) kidneys of pregnant mice 6 h after intravenous administration of DiR-labeled luciferase mRNA LNPs.

FIG. 58: Fluorescent IVIS images of DiR signal in the placentas and fetuses of pregnant mice 6 h after intravenous administration of DiR-labeled luciferase mRNA LNPs.

FIG. 59: Luminescent IVIS images of luciferase mRNA expression in the placentas and fetuses of pregnant mice 6 h after intravenous administration of DiR-labeled luciferase mRNA LNPs.

FIG. 60: Liver to placenta (L:P) delivery ratio for pregnant mice treated with Chol or Sito LNPs.

FIGS. 61A-61B: Percent of trophoblasts that are (FIG. 61A) DiR positive or (FIG. 61B) mCherry positive, 1 h, 4 h, or 18 h after LNP treatment (*p<0.05).

FIGS. 62A-62B: Luciferase expression of (FIG. 62A) cholesterol and campesterol or (FIG. 62B) sitosterol and stigmastanol LNPs in the presence of small molecules inhibiting micropinocytosis (Wortmannin, Wort), caveolae (Genistein, Gen), clathrin (chlorpromazine, CPZ), caveolae and clathrin (Dynasore, Dyna), lipid-raft (Methyl-β-cyclodextrin, MβCD) mediated endocytosis and endosomal acidification (Bafilomycin A1, Baf A1) (*p<0.05, **p<0.005, ***p<0.0005, ****p<0.0001 compared to the no inhibitor treatment group).

FIGS. 63A-63B: Quantification (FIG. 63A) and IVIS images (FIG. 63B) of LNP-mediated luciferase mRNA delivery to the maternal heart, lung, liver, kidney, and spleen in pregnant mice (*p<0.05).

FIGS. 64A-64C: Quantification (FIG. 64A, cholesterol; and FIG. 64B, sitosterol) and (FIG. 64C) IVIS images of LNP-mediated luciferase mRNA delivery to the placentas and fetuses of pregnant mice (**p<0.005).

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Description

### Lipid Nanoparticle (LNP) Formulations and Targeted Delivery of VEGF to the Placenta

Ionizable lipid nanoparticles (LNPs) are the most clinically advanced non-viral platform for mRNA delivery. While they have been explored for applications including vaccines and gene editing, LNPs have not been investigated for placental insufficiency during pregnancy. Placental insufficiency is caused by inadequate blood flow in the placenta that results in increased maternal blood pressure and restricted fetal growth. Therefore, improving vasodilation in the placenta can benefit both maternal and fetal health. Described herein are engineered ionizable LNPs for mRNA delivery to the placenta with applications in mediating placental vasodilation. A library of LNPs were designed for mRNA delivery in placental cells and identified a lead LNP that enables potent and selective in vivo mRNA delivery to trophoblasts and endothelial cells in the placenta. This lead LNP formulation encapsulating VEGF mRNA mediated significant placental vasodilation demonstrating the potential of mRNA LNPs for protein replacement therapy during pregnancy to treat placental disorders.

As indicated elsewhere herein, the present disclosure relates, in part, to an engineered LNP platform for mRNA delivery to the placenta during pregnancy, which may represent one of the first of such works to demonstrate mRNA LNP biodistribution in pregnancy with selectivity to the placenta. The lack of pre-clinical research on the safety and efficacy of drug delivery platforms such as LNPs during pregnancy was revealed during the development of the mRNA LNP COVID-19 vaccines. Fortunately, the COVID-19 mRNA vaccines were found to be safe and effective in humans, with some results suggesting immunity transfer to the fetus. One pre-clinical study performed by a group at Pfizer demonstrated little to no effects of their mRNA LNP vaccine on maternal fertility and fetal development in pregnant rodents.

While it has been shown that benchmark LNPs, such as those containing the C12-200 or DLin-MC3-DMA ionizable lipids, deliver mRNA primarily to the liver, there are several cardiovascular changes that occur during pregnancy that were exploited herein for selective mRNA delivery to the placenta. First, by 24 weeks of gestation in human pregnancy, there is a 45% increase in total cardiac output compared to non-pregnant individuals. Approximately 20-25% of this cardiac output represents blood flow to the uterus and placenta while blood flow to the liver as a function of cardiac output is lower during pregnancy compared to non-pregnant individuals. Due to these effects, it was hypothesized that LNPs capable of delivering mRNA to non-hepatic organs in non-pregnant mice, might be able to deliver mRNA to the placenta in pregnant mice. Interestingly, it was observed that LNP A4 was capable of non-hepatic luciferase mRNA delivery to the spleen in non-pregnant mice which was then partitioned between the spleen and placenta in pregnant mice. With 62% of the total luminescent flux originating from the placenta, LNP A4 demonstrated not only high placental specificity but also the highest magnitude of luciferase expression of the three LNP formulations evaluated.

In a proof-of-concept cell-level evaluation of in vivo mRNA delivery to the placenta, LNP A4 demonstrated mCherry mRNA delivery to both trophoblasts and fetal endothelial cell, which represent the two primary target cell populations of interest for treating placental insufficiency disorders.

After promising results demonstrating both luciferase and mCherry mRNA delivery to the placenta, the delivery of a clinically relevant cargo such as VEGF mRNA to mediate functional vasodilation in the placenta was explored. As expected, the C12-200 LNP mediated higher serum levels of VEGF than the A4 LNP, correlating with the high luciferase mRNA delivery to the liver with the C12-200 LNP and the efficacy of protein production by hepatocytes. In addition to measuring the serum levels of VEGF, a commonly used metric by the field to indicate functional mRNA delivery such as with erythropoietin (EPO) mRNA, liver and placental levels of VEGF were also explored. The trends in liver VEGF levels for the three treatment groups were similar to those observed in the serum. However, in the placenta, there was no measured difference in the VEGF levels between the LNP A4 and PBS groups at 6 h.

Without wishing to be bound by theory, these results can be due to the rapid secretion of VEGF-A by placental cells into the surrounding tissue and serum and also the protein's relatively short half-life (i.e., about 15-30 min). Instead, functional vasodilation in the placentas was used as an indicator of local VEGF mRNA delivery to the placenta. Specifically, staining of placental sections with H&E and CD31 was used to quantify blood vessel area in the labyrinth region. The labyrinth region is the site of nutrient and oxygen transport between the mother and fetus and consists of trophoblasts, which surround maternal blood spaces, and endothelial cells, which surround fetal blood spaces. Both cell types secrete proteins such as VEGF, and vasodilation of both maternal and fetal blood spaces would be essential for treating placental insufficiency disorders that affect both maternal blood pressure and fetal growth.

H&E staining identified both maternal and fetal blood spaces and images taken in the labyrinth region demonstrate clear vasodilation for both the A4 and C12-200 LNP treated groups. However, vasodilation in the A4 LNP group appears more homogenous than the C12-200 LNP group. It was hypothesized that the C12-200 LNPs induced preferential vasodilation of the maternal blood spaces due to the systemic expression of VEGF mRNA by the liver. Instead, it has been hypothesized that LNP A4 mediated local delivery to the placenta via vasodilation of both maternal and fetal blood spaces. To test this hypothesis, placentas with CD31 were stained to quantify fetal blood vessel area only. As hypothesized, LNP A4 mediated significantly higher fetal blood vessel area than the C12-200 LNP. Besides the homogenous vasodilation of both maternal and fetal blood vessels, there are additional benefits of local delivery platforms such as LNP A4 including limited systemic nanoparticle toxicity. C12-200 LNPs increased serum levels of the secreted AST liver enzyme by about 3.5 fold compared to PBS, suggesting some nanoparticle-mediated toxicity due to high accumulation in the liver. These results can limit the clinical translation of C12-200 LNPs for placental insufficiency disorders as repeat dosing would be essential for LNP-mediated protein replacement therapy in the practice of certain aspects of the disclosure of the present disclosure.

The present disclosure further describes optimization of the initial lead formulation (i.e., LNP A4). Orthogonal DOE was used to identify optimized LNP formulations for mRNA delivery to the placenta. Iterative LNP libraries with varied excipient molar ratios were screened in vitro in BeWo b30 cells, placental trophoblasts, for mRNA delivery and cytotoxicity, where LNPs A′1 and C′5 were identified as lead candidates due to their ability to potently deliver mRNA in vitro with minimal cytotoxicity compared to initial lead S1. LNPs A′1 and C′5 were then validated in vivo for mRNA delivery to the placenta following intravenous administration in pregnant mice.

There, LNP C′5 was able to achieve significantly higher mRNA delivery to the placenta compared to S1, while also facilitating extrahepatic mRNA delivery to the spleen. Together, these results confirm that LNP C′5 is a promising delivery vehicle with an optimized formulation for mRNA delivery to the placenta. The optimized C′5 LNP formulation has demonstrated its ability to potently deliver mRNA to the placenta. Additionally, deeper investigations into the mechanisms behind enhanced mRNA delivery as a result of varied excipient composition will inform subsequent LNP design and optimization for both enhanced nucleic acid delivery to the placenta and potentially beyond to other reproductive organs.

The present disclosure further describes the utilization of high-throughput DNA barcoding to screen a library of 98 LNP formulations in vivo for the identification of a placenta-tropic LNP that mediates more than 100-fold higher mRNA delivery to the placenta of pregnant mice than the FDA-approved DLin-MC3-DMA formulation. It is proposed herein that an endogenous, protein adsorption-based targeting mechanism enables placental cell tropism through a non-apolipoprotein E (ApoE) dependent pathway with the four-component LNP. In an early-onset mouse model of pre-eclampsia, the LNP formulations described herein, encapsulating vascular endothelial growth factor (VEGF) mRNA, rescues maternal blood pressure and fetal weight with partial rescue of the pre-eclamptic immunophenotype. Together, these results demonstrate the potential of this modular LNP platform to encapsulate a variety of nucleic acid cargos to treat placental disorders such as pre-eclampsia during pregnancy.

Placenta-Targeted Lipid Nanoparticle (LNP) Formulations Comprising Cholesterol and/or Derivatives or Analogs Thereof

Ionizable lipid nanoparticles (LNPs) have recently emerged as the most clinically advanced non-viral platform for therapeutic delivery of nucleic acids. LNPs are highly advantageous delivery vehicles due to their modular nature, biocompatibility, and ability to enable potent intracellular nucleic acid delivery. As the LNP field continues to grow, new therapeutic applications have emerged beyond the traditional use of LNPs for vaccines and treating liver-centric diseases. In particular, LNP-mediated delivery of messenger RNA (mRNA) to the placenta has been explored for the treatment of placental dysfunction.

The placenta is an organ unique to pregnancy, developing throughout gestation to facilitate nutrient and oxygen exchange between maternal and fetal circulation. Placental disorders, such as pre-eclampsia, can arise during pregnancy as a result of dysfunctional placental development and can lead to immediate and long-term complications for both mother and fetus. Pre-eclampsia affects 5-8% of all pregnancies and is a leading cause of maternal mortality worldwide. Despite the global prevalence of this disorder, no therapeutic has been developed to address the underlying placental dysfunction, inciting the need to develop drug delivery platforms capable of achieving extrahepatic delivery to the placenta.

It has been well established that the physicochemical properties of LNPs, including their size, charge, chemical composition and surface chemistry, can affect their interactions with various cell types and influence biodistibution. To improve extrahepatic mRNA LNP delivery to the placenta, a major research thrust has focused on chemical modifications, altering either the ionizable lipid structure or excipient molar ratios of the LNP formulation.

However, physical cues are also important in influencing nanoparticle-cell interactions. Previously, nanoparticle elasticity has been shown to impact nanoparticle uptake into both immune cells and cancer cells, where stiffer nanoparticles have greater uptake into macrophages and T cells while softer nanoparticles preferentially accumulate in tumors. In the placenta, polymeric microparticle elasticity was shown to impact uptake into placental trophoblasts, the main cell type of the placenta, where uptake was enhanced with rigid microparticles. LNP elasticity has not been well characterized but it has been hypothesized to impact LNP interactions with cellular barriers and subsequent mRNA transfection. Furthermore, no work has been done to study the effects of LNP elasticity on LNP-mediated mRNA delivery to the placenta.

Given the tunable nature of LNPs, exploration was undertaken to see whether LNP elasticity could be modified through changes in excipient composition and if changes in LNP elasticity could impact LNP uptake and mRNA transfection in the placenta. Atomic force microscopy (AFM) is frequently used to quantify nanoparticle elasticity and measure the Young's modulus of nanoparticle formulations. Previously, AFM has been performed on mRNA LNPs to study the structure, polydispersity and surface interactions of LNPs. However, Young's modulus values of mRNA LNPs have not been reported.

To generate LNPs with tunable elasticity, there was interest in modulating the cholesterol component in the formulation. Cholesterol is one of the main excipients used in LNPs and plays an important structural role by modulating membrane rigidity to enhance LNP stability. Recently, a class of naturally occurring cholesterol analogs known as phytosterols have been explored for LNP-mediated mRNA delivery. In particular, formulating LNPs with these analogs demonstrated changes in LNP morphology and lamellarity. These works also investigated the effect of cholesterol analog substitution on membrane rigidity via a fluorescent probe sensitive to lipid bilayer structures, however influence of the phytosterol structure on Young's modulus values were not reported.

Here, methodology was developed to measure LNP elasticity via AFM and it was demonstrated that changes in LNP elasticity can be tuned through sterol structure. Using the placenta-tropic C12-494 ionizable lipid, a library of LNPs was generated which were formulated with cholesterol (Chol) or one of three cholesterol analogs: campesterol (Camp), β-sitosterol (Sito), or stigmastanol (Stig). LNP elasticity was evaluated via AFM where these LNPs had measured Young's moduli ranging from 71.0-411.4 kPa. Hypothesizing that rigid LNPs may lead to improved placental mRNA LNP delivery, this library of LNPs was screened in vitro in trophoblasts to characterize changes in LNP uptake and mRNA transfection. Through these screens, the intermediate stiffness Sito LNP was identified as the lead LNP candidate and was administered to pregnant mice where it mediated reduced liver and increased placental mRNA delivery compared to the standard Chol LNP formulation. Taken together, these results support the potential of the Sito LNP to potently deliver mRNA to the placenta and demonstrate that LNP elasticity is a property that can tuned to improve placental mRNA delivery.

### Definitions

The term “about” as used herein can allow for a degree of variability in a value or range, for example, within 10%, within 5%, or within 1% of a stated value or of a stated limit of a range, and includes the exact stated value or range.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms. Similarly, the terms “heteroalkylenyl”, “cycloalkylenyl”, “heterocycloalkylenyl”, and the like, as used herein, refer to a divalent radical of the moiety corresponding to the base group (e.g., heteroalkyl, cycloalkyl, and/or heterocycloalkyl). A divalent radical possesses two open valencies at any position(s) of the group, wherein each radical may be on a carbon atom or heteroatom. Thus, the divalent radical may form a single bond to two distinct atoms or groups, or may form a double bond with one atom.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequences or a partial nucleotide sequence encoding a protein that elicits an adaptive immune response therefore encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “amine” as used herein refers to primary, secondary, and tertiary amines having, e.g., the formula N (group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “cationic lipid” refers to any of a number of lipid species that carry a net positive charge at a selected pH, such as physiological pH (e.g., pH of about 7.0). It has been found that cationic lipids comprising alkyl chains with multiple sites of unsaturation, e.g., at least two or three sites of unsaturation, are particularly useful for forming lipid particles with increased membrane fluidity. A number of cationic lipids and related analogs, which are also useful in the present disclosure, have been described in U.S. Patent Publication Nos. 20060083780 and 20060240554; U.S. Pat. Nos. 5,208,036; 5,264,618; 5,279,833; 5,283,185; 5,753,613; and 5,785,992; and PCT Publication No. WO 96/10390, the disclosures of which are herein incorporated by reference in their entirety for all purposes. Non-limiting examples of cationic lipids are described in detail herein. In some cases, the cationic lipids comprise a protonatable tertiary amine (e.g., pH titratable) head group, C₁₈ alkyl chains, ether linkages between the head group and alkyl chains, and 0 to 3 double bonds. Such lipids include, e.g., DSDMA, DLinDMA, DLenDMA, and DODMA.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

In particular, in the case of a mRNA, and “effective amount” or “therapeutically effective amount” of a therapeutic nucleic acid as relating to a mRNA is an amount sufficient to produce the desired effect, e.g., mRNA-directed expression of an amount of a protein that causes a desirable biological effect in the organism within which the protein is expressed. For example, in some embodiments, the expressed protein is an active form of a protein that is normally expressed in a cell type within the body, and the therapeutically effective amount of the mRNA is an amount that produces an amount of the encoded protein that is at least 50% (e.g., at least 60%, or at least 70%, or at least 80%, or at least 90%) of the amount of the protein that is normally expressed in the cell type of a healthy individual. For example, in some embodiments, the expressed protein is a protein that is normally expressed in a cell type within the body, and the therapeutically effective amount of the mRNA is an amount that produces a similar level of expression as observed in a healthy individual in an individual with aberrant expression of the protein (i.e., protein deficient individual). Suitable assays for measuring the expression of an mRNA or protein include, but are not limited to dot blots, Northern blots, in situ hybridization, ELISA, immunoprecipitation, enzyme function, as well as phenotypic assays known to those of skill in the art.

The term “encode” as used herein refers to the product specified (e.g., protein and RNA) by a given sequence of nucleotides in a nucleic acid (i.e., DNA and/or RNA), upon transcription or translation of the DNA or RNA, respectively. In certain embodiments, the term “encode” refers to the RNA sequence specified by transcription of a DNA sequence. In certain embodiments, the term “encode” refers to the amino acid sequence (e.g., polypeptide or protein) specified by translation of mRNA. In certain embodiments, the term “encode” refers to the amino acid sequence specified by transcription of DNA to mRNA and subsequent translation of the mRNA encoded by the DNA sequence. In certain embodiments, the encoded product may comprise a direct transcription or translation product. In certain embodiments, the encoded product may comprise post-translational modifications understood or reasonably expected by one skilled in the art.

The term “fully encapsulated” indicates that the active agent or therapeutic agent in the lipid particle is not significantly degraded after exposure to serum or a nuclease or protease assay that would significantly degrade free DNA, RNA, or protein. In a fully encapsulated system, preferably less than about 25% of the active agent or therapeutic agent in the particle is degraded in a treatment that would normally degrade 100% of free active agent or therapeutic agent, more preferably less than about 10%, and most preferably less than about 5% of the active agent or therapeutic agent in the particle is degraded. In the context of nucleic acid therapeutic agents, full encapsulation may be determined by an OLIGREEN® assay. OLIGREEN® is an ultra-sensitive fluorescent nucleic acid stain for quantitating oligonucleotides and single-stranded DNA or RNA in solution (available from Invitrogen Corporation; Carlsbad, Calif.). “Fully encapsulated” also indicates that the lipid particles are serum stable, that is, that they do not rapidly decompose into their component parts upon in vivo administration.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The terms “helper lipid” and “neutral lipid” are used interchangeably herein to refer to a lipid capable of increasing the effectiveness of delivery of lipid-based particles such as cationic lipid-based particles to a target, preferably into a cell. The helper lipid can be neutral, positively charged, or negatively charged. In certain embodiments, the helper lipid is neutral or negatively charged. Non-limiting examples of helper lipids include 1,2-distearoyl-sn-glycero-3-phosphocholine (DSPC), 1,2-di-(9Z-octadecenoyl)-sn-glycero-3-phosphoethanolamine (DOPE), 1-palmitoyl-2-oleoyl-sn-glycero-3phosphocholin (POPC) and 1,2-dioleoyl-sn-glycero-3-phosphocholine (DOPC).

The term “heteroalkyl” as used herein by itself or in combination with another term, means, unless otherwise stated, a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) (e.g., O, N, P, and S) may be placed at any interior position of the heteroalkyl group or at either terminal position at which the group is attached to the remainder of the molecule.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocycloalkyl” as used herein refers to an aliphatic, partially unsaturated or fully saturated, 3- to 14-membered ring system, including single rings of 3 to 8 atoms and bi- and tricyclic ring systems where at least one of the carbon atoms of the ring is replaced with a heteroatom such as, but not limited to, nitrogen, oxygen, sulfur, or phosphorus. A heterocycloalkyl can include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited, to the following exemplary groups: pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C4-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “immune cell,” as used herein refers to any cell involved in the mounting of an immune response. Such cells include, but are not limited to, T cells, B cells, NK cells, antigen-presenting cells (e.g., dendritic cells and macrophages), monocytes, neutrophils, eosinophils, basophils, and the like.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “ionizable lipid” as used herein refers to a lipid (e.g., a cationic lipid) having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or neutral lipid refers to the nature of the predominant species and does not require that all of the lipid be present in the charged or neutral form. Generally, ionizable lipids have a pKₐ of the protonatable group in the range of about 4 to about 7.

The term “local delivery,” as used herein, refers to delivery of an active agent or therapeutic agent such as a messenger RNA directly to a target site within an organism. For example, an agent can be locally delivered by direct injection into a disease site such as a tumor or other target site such as a site of inflammation or a target organ such as the liver, heart, pancreas, kidney, and the like.

The term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

The terms “conjugated lipid” and “polymer conjugated lipid” are used interchangeably herein to refer to a lipid which is conjugated to one or more polymeric groups, which inhibits aggregation of lipid particles. Such lipid conjugates include, but are not limited to, polyamide oligomers (e.g., ATTA-lipid conjugates), PEG-lipid conjugates, such as PEG coupled to dialkyloxypropyls, PEG coupled to diacylglycerols, PEG coupled to cholesterol, PEG coupled to phosphatidylethanolamines, PEG conjugated to ceramides (e.g., U.S. Pat. No. 5,885,613, the disclosure of which is herein incorporated by reference in its entirety for all purposes), cationic PEG lipids, and mixtures thereof. PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In preferred embodiments, non-ester containing linker moieties are used. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG), DSPE-PEG-DBCO, DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid and the like.

As used herein, “lipid encapsulated” can refer to a lipid particle that provides an active agent or therapeutic agent, such as a nucleic acid (e.g., a protein cargo), with full encapsulation, partial encapsulation, or both. In a preferred embodiment, the nucleic acid is fully encapsulated in the lipid particle (e.g., to form an SPLP, pSPLP, SNALP, or other nucleic acid-lipid particle).

The term “lipid nanoparticle” refers to a particle having at least one dimension on the order of nanometers (e.g., 1-1,000 nm) which includes one or more lipids and/or additional agents.

The term “lipid particle” is used herein to refer to a lipid formulation that can be used to deliver an active agent or therapeutic agent, such as a nucleic acid (e.g., mRNA), to a target site of interest. In the lipid particle of the disclosure, which is typically formed from a cationic lipid, a non-cationic lipid, and a conjugated lipid that prevents aggregation of the particle, the active agent or therapeutic agent may be encapsulated in the lipid, thereby protecting the agent from enzymatic degradation.

The term “monovalent” as used herein refers to a substituent connecting via a single bond to a substituted molecule. When a substituent is monovalent, such as, for example, F or Cl, it is bonded to the atom it is substituting by a single bond.

The term “mRNA” or “messenger RNA” as used herein refers to a ribonucleic acid sequences which encodes a peptide or protein. In certain embodiments, the mRNA may comprise a “transcript” that is produced by using a DNA template and encodes a peptide or protein. Typically, mRNA comprises 5′-UTR, protein coding region and 3′-UTR. mRNA can be produced by in vitro transcription from a DNA template. Methods of in vitro transcription are known to those of skill in the art. For example, various in vitro transfer kits are commercially available. According to the present disclosure, mRNA can be modified by further stabilizing modifications and cap formation in addition to the modifications according to the disclosure.

The term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “non-cationic lipid” refers to any amphipathic lipid as well as any other neutral lipid or anionic lipid.

The term “nucleic acid” as used herein refers to a polymer containing at least two deoxyribonucleotides or ribonucleotides in either single- or double-stranded form and includes DNA and RNA. DNA may be in the form of, e.g., antisense molecules, plasmid DNA, pre-condensed DNA, a PCR product, vectors (Pl, PAC, BAC, YAC, artificial chromosomes), expression cassettes, chimeric sequences, chromosomal DNA, or derivatives and combinations of these groups. RNA may be in the form of siRNA, asymmetrical interfering RNA (aiRNA), microRNA (miRNA), mRNA, tRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mal. Cell. Probes, 8:91-98 (1994)).

As used herein, the term “nucleic acid” includes any oligonucleotide or polynucleotide, with fragments containing up to 60 nucleotides generally termed oligonucleotides, and longer fragments termed polynucleotides. In particular embodiments, oligonucleotides of the disclosure are from about 15 to about 60 nucleotides in length. Nucleic acid may be administered alone in the lipid particles of the disclosure, or in combination (e.g., co-administered) with lipid particles of the disclosure comprising peptides, polypeptides, or small molecules such as conventional drugs. In other embodiments, the nucleic acid may be administered in a viral vector.

“Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkyl halides.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

The terms “spacer” or “spacer element” as used herein with reference to a crRNA or sgRNA, refers to the polynucleotide sequence that can specifically hybridize to a target nucleic acid sequence. The spacer element interacts with the target nucleic acid sequence through hydrogen bonding between complementary base pairs (i.e., paired bases). A spacer element binds to a selected DNA target sequence. Accordingly, the spacer element is a DNA target-binding sequence. The spacer element determines the location of Cas protein's site-specific binding and endonucleolytic cleavage. Spacer elements range from −17- to −84 nucleotides in length, depending on the Cas protein with which they are associated, and have an average length of 36 nucleotides. For example, for SpyCas9, the functional length for a spacer to direct specific cleavage is typically about 12-25 nucleotides. Variability of the functional length for a spacer element is known in the art, as indicated in U.S. Published Patent Application No. 2014/0315985, which is incorporated herein by reference in its entirety.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, O-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The terms “peptide,” “polypeptide,” and “protein” are used interchangeably herein, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “placenta” as used herein refers to the maternal organ that connects the developing fetus to the uterine wall. After birth, the placenta is expelled and is referred to as a postpartum placenta.

The term “room temperature” as used herein refers to a temperature of about 15° C. to 28° C.

The term “solvent” as used herein refers to a liquid that can dissolve a solid, liquid, or gas. Non-limiting examples of solvents are silicones, organic compounds, water, alcohols, ionic liquids, and supercritical fluids.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “therapeutic” treatment is a treatment administered to a subject who exhibits signs of pathology, for the purpose of diminishing or eliminating those signs.

The term “therapeutic protein” as used herein refers to a protein or peptide which has a positive or advantageous effect on a condition or disease state of a subject when provided to the subject in a therapeutically effective amount. In certain embodiments, a therapeutic protein or peptide has curative or palliative properties and may be administered to ameliorate, relieve, alleviate, reverse, delay onset of or lessen the severity of one or more symptoms of a disease or disorder. A therapeutic protein or peptide may have prophylactic properties and may be used to delay the onset of a disease or to lessen the severity of such disease or pathological condition. The term “therapeutic protein” includes entire proteins or peptides, and can also refer to therapeutically active fragments thereof. It can also include therapeutically active variants of a protein. Exemplary therapeutic proteins include, but are not limited to, an analgesic protein, an anti-inflammatory protein, an anti-proliferative protein, an proapoptotic protein, an anti-angiogenic protein, a cytotoxic protein, a cytostatic protein, a cytokine, a chemokine, a growth factor, a wound healing protein, a pharmaceutical protein, or a pro-drug activating protein. Therapeutic proteins may include growth factors (EGF, TGF-α, TGF-β, TNF, HGF, IGF, and IL-1-8, inter alia) cytokines, paratopes, Fabs (fragments, antigen binding), and antibodies.

The terms “treat,” “treating” and “treatment,” as used herein, means reducing the frequency or severity with which symptoms of a disease or condition are experienced by a subject by virtue of administering an agent or compound to the subject.

### Lipids

In one aspect, the present disclosure provides an ionizable lipid of Formula (I), or a salt, solvate, stereoisomer, or isotopologue thereof:

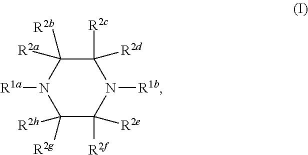

wherein:
R^{1a} and R^{1b} are each independently

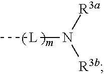

R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₇-C₁₃ aralkyl, optionally substituted C₆-C₁₀ aryl, and optionally substituted C₂-C₁₀ heteroaryl;
each occurrence of R^{3a}, R^{3b}, and R^{3c} is independently selected from the group consisting of H, -(optionally substituted C₁-C₆ alkylenyl)-C(═O)OR⁴, -(optionally substituted C₁-C₆ alkylenyl)-C(═O)N(R⁴)(R⁵), -(optionally substituted C₁-C₆ alkylenyl)-C(═O)R⁴, -(optionally substituted C₁-C₆ alkylenyl)-(R⁴), —C(═O)OR⁴, —C(═O)N(R⁴)(R⁵), —C(═O)R⁴, and R⁴,
   wherein no more than one of each occurrence of R^{3a}, R^{3b}, and R^{3c} is H;
R⁴ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁵ is selected from the group consisting of H and optionally substituted C₁-C₆ alkyl;
each occurrence of L is independently selected from the group consisting of -(optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, —X-(optionally substituted C₁-C₁₂ alkylenyl)-, —X-(optionally substituted C₂-C₁₂ alkenylenyl)-, —X-(optionally substituted C₁-C₁₂ alkynylenyl)-, —X-(optionally substituted C₁-C₁₂ heteroalkylenyl)-, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—; and
each occurrence of m is independently an integer selected from the group consisting of 1, 2, 3, and 4.

In certain embodiments, at least one selected from the group consisting of R^{2a}, R^{2b}, R^{2c}, R^{2d}, R^{2e}, R^{2f}, R^{2g}, and R^{2h} is H. In certain embodiments, at least two selected from the group consisting of R^{2a}, R^{2b}, R^{2c}, R^{2d}, R^{2e}, R^{2f}, R^{2g}, and R^{2h} are H. In certain embodiments, at least three selected from the group consisting of R^{2a}, R^{2b}, R^{2c}, R^{2d}, R^{2e}, R^{2f}, R^{2g}, and R^{2h} are H. In certain embodiments, at least four selected from the group consisting of R^{2a}, R^{2b}, R^{2c}, R^{2d}, R^{2e}, R^{2f}, R^{2g}, and R^{2h} are H. In certain embodiments, at least five selected from the group consisting of R^{2a}, R^{2b}, R^{2c}, R^{2d}, R^{2e}, R^{2f}, R^{2g}, and R^{2h} are H. In certain embodiments, at least six selected from the group consisting of R^{2a}, R^{2b}, R^{2c}, R^{2d}, R^{2e}, R^{2f}, R^{2g}, and R^{2h} are H. In certain embodiments, at least seven selected from the group consisting of R^{2a}, R^{2b}, R^{2c}, R^{2d}, R^{2e}, R^{2f}, R^{2g}, and R^{2h} are H. In certain embodiments, each of R^{2a}, R^{2b}, R^{2c}, R^{2d}, R^{2e}, R^{2f}, R^{2g}, and R^{2h} are H.

In certain embodiments, L is —CH₂—. In certain embodiments, L is —(CH₂)₂—. In certain embodiments, L is —(CH₂)₃—. In certain embodiments, L is —(CH₂)₁₀—. In certain embodiments, L is —(CH₂)₂O—. In certain embodiments, L is —(CH₂)₃O—. In certain embodiments, L is —CH₂CH(OR⁵)CH₂—. In certain embodiments, L is —(CH₂)₂NR^{3c}—. In certain embodiments, L is

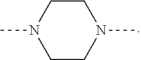

In certain embodiments, L is

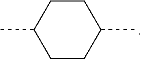

In certain embodiments, L is

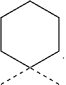

For instances of L which are asymmetric (e.g., —(CH₂)₃O—) it is understood that the present disclosure encompasses both possible orientations (e.g., —(CH₂)₃O— and —O(CH₂)₃—).

In certain embodiments, the ionizable lipid of Formula (I) is:

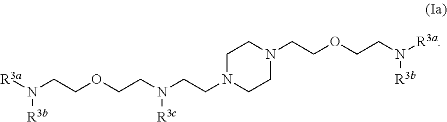

In certain embodiments, the ionizable lipid of Formula (I) is:

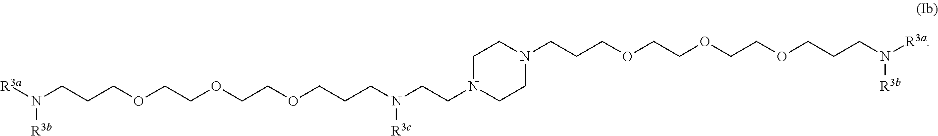

In certain embodiments, the ionizable lipid of Formula (I) is

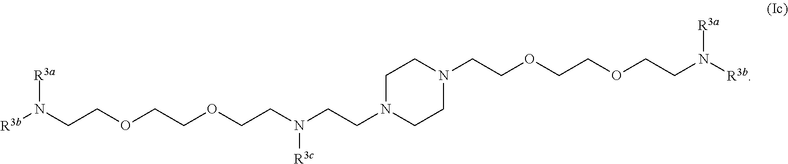

In certain embodiments, the ionizable lipid of Formula (I) is:

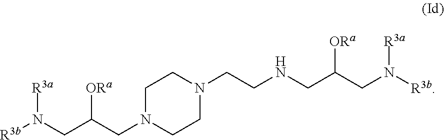

In certain embodiments, the ionizable lipid of Formula (I) is:

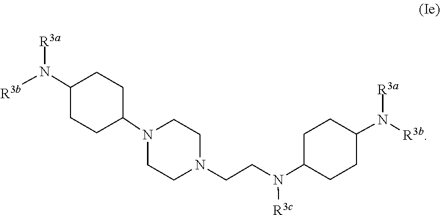

In certain embodiments, the ionizable lipid of Formula (I) is:

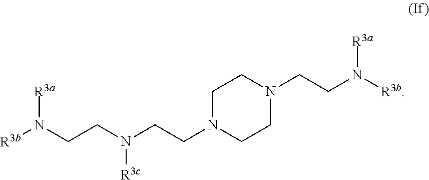

In certain embodiments, the ionizable lipid of Formula (I) is:

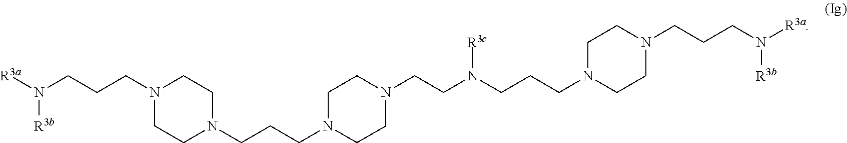

In certain embodiments, the ionizable lipid of Formula (I) is:

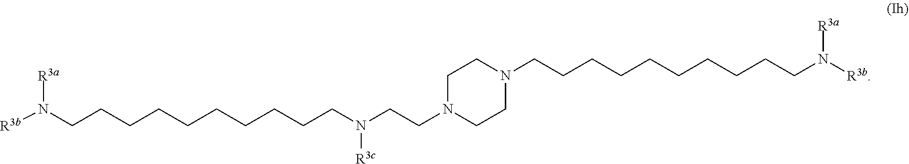

In certain embodiments, the ionizable lipid of Formula (I) is:

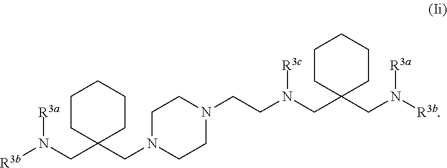

In certain embodiments, R^{3a} is H. In certain embodiments, R^{3a} is —CH₂CH(OH) (optionally substituted C₁-C₂₈ alkyl). In certain embodiments, R^{3a} is —CH₂CH(OH) (optionally substituted C₂-C₂₈ alkenyl). In certain embodiments, R^{3a} is —CH₂CH₂C(═O)O (optionally substituted C₁-C₂₈ alkyl). In certain embodiments, R^{3a} is —CH₂CH₂C(═O)NH (optionally substituted C₁-C₂₈ alkyl). In certain embodiments, R^{3b} is H. In certain embodiments, R^{3b} is —CH₂CH(OH) (optionally substituted C₁-C₂₈ alkyl). In certain embodiments, R^{3b} is —CH₂CH(OH) (optionally substituted C₂-C₂₈ alkenyl). In certain embodiments, R^{3b} is —CH₂CH₂C(═O)O (optionally substituted C₁-C₂₈ alkyl). In certain embodiments, R^{3b} is —CH₂CH₂C(═O)NH (optionally substituted C₁-C₂₈ alkyl). In certain embodiments, R^{3c} is H. In certain embodiments, R^{3c} is —CH₂CH(OH) (optionally substituted C₁-C₂₈ alkyl). In certain embodiments, R^{3c} is —CH₂CH(OH) (optionally substituted C₂-C₂₈ alkenyl). In certain embodiments, R^{3c} is —CH₂CH₂C(═O)O (optionally substituted C₁-C₂₈ alkyl). In certain embodiments, R^{3c} is —CH₂CH₂C(═O)NH (optionally substituted C₁-C₂₈ alkyl).

In certain embodiments, R^{3a} is —CH₂CH(OH)(CH₂)₉CH₃. In certain embodiments, R^{3a} is —CH₂CH(OH)(CH₂)₁₁CH₃. In certain embodiments, R^{3a} is —CH₂CH(OH)(CH₂)₁₃CH₃. In certain embodiments, R^{3b} is —CH₂CH(OH)(CH₂)₉CH₃. In certain embodiments, R^{3b} is —CH₂CH(OH)(CH₂)₁₁CH₃. In certain embodiments, R^{3b} is —CH₂CH(OH)(CH₂)₁₃CH₃. In certain embodiments, R^{3c} is —CH₂CH(OH)(CH₂)₉CH₃. In certain embodiments, R^{3c} is —CH₂CH(OH)(CH₂)₁₁CH₃. In certain embodiments, R^{3c} is —CH₂CH(OH)(CH₂)₁₃CH₃.

In certain embodiments, each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, optionally substituted heteroalkylenyl, optionally substituted cycloalkylenyl, and optionally substituted heterocycloalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C1-C₆ alkyl, C₃-C₈ cycloalkyl, C₁-C₆ haloalkyl, benzyl, and phenyl.

In certain embodiments, the ionizable lipid of Formula (I) is:

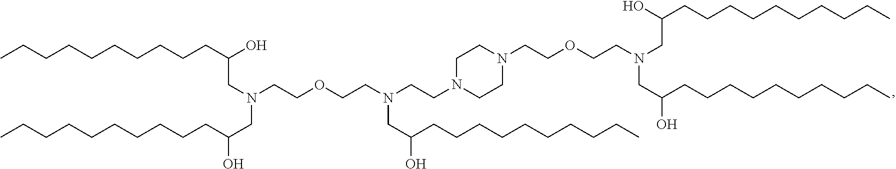 [SMILES: CCCCCCCCCCC(O)CN(CCOCCN(CC(O)CCCCCCCCCC)CC(O)CCCCCCCCCC)CCN1CCN(CCOCCN(CC(O)CCCCCCCCCC)CC(O)CCCCCCCCCC)CC1]

1,1′-((2-(2-(4-(2-((2-(2-(bis(2-hydroxydodecyl)amino)ethoxy)ethyl)(2-hydroxydodecyl)amino)ethyl)piperazin-1-yl)ethoxy)ethyl)azanediyl)bis(dodecan-2-ol) (A4).

In certain embodiments, the ionizable lipid of Formula (I) is:

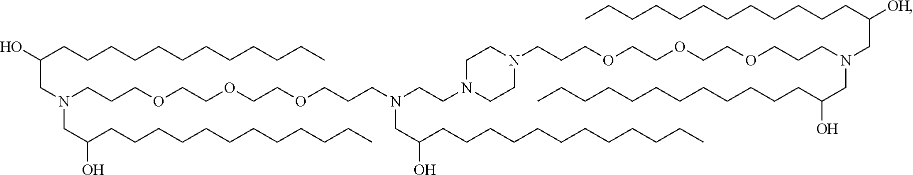 [SMILES: CCCCCCCCCCCCC(O)CN(CCCOCCOCCOCCCN(CC(O)CCCCCCCCCCCC)CC(O)CCCCCCCCCCCC)CCN1CCN(CCCOCCOCCOCCCN(CC(O)CCCCCCCCCCCC)CC(O)CCCCCCCCCCCC)CC1]

15-(2-(4-(16-hydroxy-14-(2-hydroxytetradecyl)-4,7,10-trioxa-14-azaoctacosyl)piperazin-1-yl)ethyl)-29-(2-hydroxytetradecyl)-19,22,25-trioxa-15,29-diazatritetracontane-13,31-diol (B5).

In certain embodiments, the ionizable lipid of Formula (I) is:

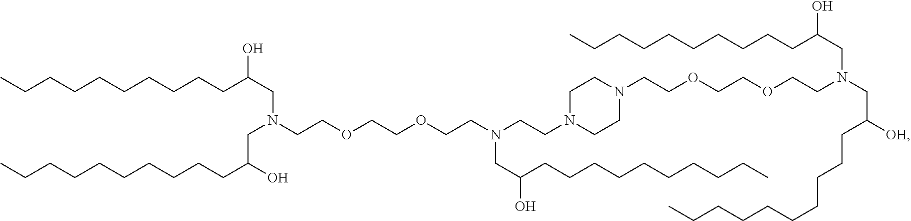 [SMILES: CCCCCCCCCCC(O)CN(CCOCCOCCN(CC(O)CCCCCCCCCC)CC(O)CCCCCCCCCC)CCN1CCN(CCOCCOCCN(CC(O)CCCCCCCCCC)CC(O)CCCCCCCCCC)CC1]

13-(2-(4-(2-(2-(2-(bis(2-hydroxydodecyl)amino)ethoxy)ethoxy)ethyl)piperazin-1-yl)ethyl)-22-(2-hydroxydodecyl)-16,19-dioxa-13,22-diazatetratriacontane-11,24-diol (A2).

In certain embodiments, the ionizable lipid of Formula (I) is:

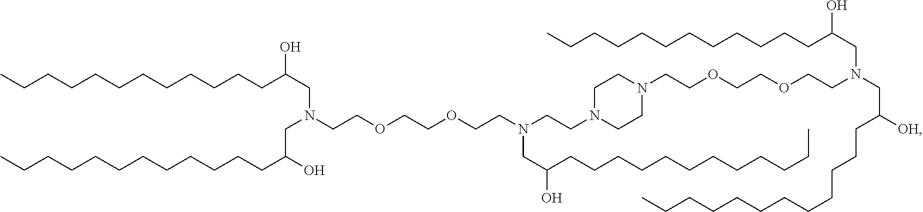 [SMILES: CCCCCCCCCCCCC(O)CN(CCOCCOCCN(CC(O)CCCCCCCCCCCC)CC(O)CCCCCCCCCCCC)CCN1CCN(CCOCCOCCN(CC(O)CCCCCCCCCCCC)CC(O)CCCCCCCCCCCC)CC1]

15-(2-(4-(2-(2-(2-(bis(2-hydroxytetradecyl)amino)ethoxy)ethoxy)ethyl)piperazin-1-yl)ethyl)-24-(2-hydroxytetradecyl)-18,21-dioxa-15,24-diazaoctatriacontane-13,26-diol (B2).

In certain embodiments, the ionizable lipid of Formula (I) is:

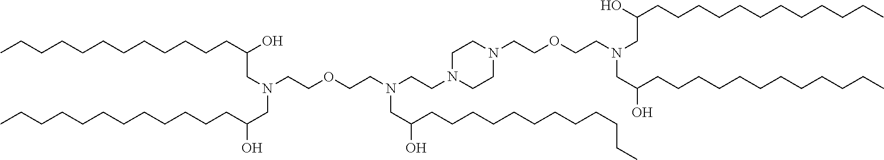 [SMILES: CCCCCCCCCCCCC(O)CN(CCOCCN(CC(O)CCCCCCCCCCCC)CC(O)CCCCCCCCCCCC)CCN1CCN(CCOCCN(CC(O)CCCCCCCCCCCC)CC(O)CCCCCCCCCCCC)CC1]

1,1′-((2-(2-(4-(2-((2-(2-(bis(2-hydroxytetradecyl)amino)ethoxy)ethyl)2-hydroxytetradecyl)amino)ethyl)piperazin-1-yl)ethoxy)ethyl)azanediyl)bis(tetradecan-2-ol), (C14-494).
   Ionizable Lipids and/or Cationic Lipids

The scope of ionizable lipids contemplated for use in the present disclosure is not limited to ionizable lipids of Formula (I). In the lipid nanoparticles of the disclosure, the cationic lipid or ionizable lipid may comprise, e.g., one or more of the following: (6Z,9Z,28Z,31Z)-heptatriaconta-6,9,28,31-tetraen-19-yl 4-(dimethylamino)butanoate (DLinMC3DMA), [(4-hydroxybutyl)azanediyl]di(hexane-6,1-diyl) bis(2-hexyldecanoate) (ALC-0315), heptadecan-9-yl 8-{(2-hydroxyethyl)[6-oxo-6-(undecyloxy)hexyl]amino}octanoate (SM-102), 1,1′-[[2-[4-[2-[[2-[bis(2-hydroxydodecyl)amino]ethyl](2-hydroxydodecyl)amino]ethyl]-1-piperazinyl]ethyl]imino]bis-2-dodecanol (C12-200), 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-K-C2-DMA; “XTC2”), 2,2-dilinoleyl-4-(3-45 dimethylaminopropyl)-[1,3]-dioxolane (D Lin-K-C3-DMA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-Nmethylpepiazino-[1,3]-dioxolane (DLin-K-MPZ), 2,2-dili-noleyl-4-dimethylaminomethyl-[1,3]-dioxolane (DLin-K-DMA), 1,2-dilinoleylcarbamoyloxy-3-dimethylaminopropane (D Lin-C-DAP), 1,2-dilinoleyoxy-3-(dimethylaminoacetoxypropane (DLin-DAC), 1-2dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-2-DMAP), 1,2-dilinoleyloxy-3-trimethylaminopropane chloride salt (DLin-TMA·Cl), 1,2-dilinoleoyl-3-trimethylaminopropane chloride salt (DLin-TAP·Cl), 1,2-dilinoleyloxy-3-(N-methylpiperazino)propane (D Lin-MPZ), 3-(N,N-dilinoleylamino)-1,2-propanediol (D LinAP), 3-(N,N-dioleylamino)-1,2-propanedio (DOAP), 1,2-dilinoleyloxo-3-(2-N,N-dimethylamino)ethoxypropane (D Lin-EG-DMA), N,N-dioleyl-N,N-dimethylanrmonium chloride (DODAC), 1,2-dioleyloxy-N,N-dimethylaminopropane (DODMA), 1,2-distearyloxy-N,N-dimethylaminopropane (DSD MA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N, N-trimethylammonium chloride (DOTAP), 3-(N—(N′,N′dimethylaminoethane)-carbamoyl)cholesterol (DC-Chol), N-(1,2-dimyristyloxyprop-3-yl)-N,N-dimethyl-N-hydroxyethyl anrmonium bromide (DMRIE), 2,3-dioleyloxy-N-[2 (spermine-carboxamidoethyl]-N,N-dimethy 1-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy) propane (CpLinDMA), N,N-dimethyl-3,4-dioleyloxybenzylamine (DMOBA), 1,2-N,N′dioleylcarbamyl-3-dimethylaminopropane (DOcarbDAP), 1,2-N,N′-dilinoleylcarbamyl-3-dimethylaminopropane (DLincarbDAP), or mixtures thereof. In certain embodiments, the cationic lipid is DLinDMA, DLin-K-C2-DMA (“XTC2”), or mixtures thereof. The ionizable lipids are not limited to those recited herein, and can further include ionizable lipids known to those skilled in the art, or described in PCT Application No. PCT/US2020/056255 and/or PCT Application No. PCT/US2020/056252, the disclosures of which are herein incorporated by reference in its entirety.

The synthesis of cationic lipids such as DLin-K-C2-DMA (“XTC2”), DLin-K-C3-DMA, DLin-K-C4-DMA, DLin-K6-DMA, and DLin-K-MPZ, as well as additional cationic lipids, is described in U.S. Application Publication No. US 2011/0256175, the disclosure of which is herein incorporated by reference in its entirety for all purposes. The synthesis of cationic lipids such as DLin-K-DMA, DLin-CDAP, DLin-DAC, DLin-MA, DLinDAP, DLin-S-DMA, DLin-2-DMAP, DLin-TMA·Cl, DLin-TAP·Cl, DLin-MPZ, DLinAP, DOAP, and DLin-EG-DMA, as well as additional cationic lipids, is described in PCT Application No. PCT/US08/88676, filed Dec. 31, 2008, the disclosure of which is herein incorporated by reference in its entirety for all purposes. The synthesis of cationic lipids such as CLinDMA, as well as additional cationic lipids, is described in U.S. Patent Publication No. 20060240554, the disclosure of which is herein incorporated by reference in its entirety for all purposes.

### Non-Cationic Lipid

In the nucleic acid-lipid particles of the present disclosure, the non-cationic lipid may comprise, e.g., one or more anionic lipids and/or neutral lipids. In some embodiments, the non-cationic lipid comprises one of the following neutral lipid components: (1) cholesterol or a derivative thereof (2) a phospholipid; or (3) a mixture of a phospholipid and cholesterol or a derivative thereof.

Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof. The synthesis of cholesteryl-2′-hydroxyethyl ether is known to one skilled in the art and described in U.S. Pat. Nos. 8,058,069, 8,492,359, 8,822,668, 9,364,435, 9,504,651, and 11,141,378, all of which are hereby incorporated herein in their entireties for all purposes.

Non-limiting examples of non-cationic lipids include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), ioleoylphosphatidylethanolamine (DOPE), palmitoyloleoylphosphatidylcholine (POPC), palmitoyloleoylphosphatidylethanolamine (POPE), palmitoyloleyolphosphatidylglycerol (POPG), dioleoylphosphatidylethanolamine 4-(N-maleimidomethyl)-cyclohexane-1-carboxylate DOPE-mal), dipalmitoylphosphatidylethanolamine (DPPE), dimyristoylphosphatidylethanolamine (DMPE), distearoylphosphatidylethanolamine (DSPE), monomethylphosphatidylethanolamine, dimethylphosphatidylethanolamine, dielaidoylphosphatidylethanolamine (DEPE), stearoyloleoylphosphatidylethanolamine (SOPE), lysophosphatidylcholine, dilinoleoylphosphatidylcholine, and mixtures thereof.

Other diacylphosphatidylcholine and diacylphosphatidylethanolamine phospholipids can also be used. The acyl groups in these lipids can be, for example, acyl groups derived from fatty acids having C₁₀-C₂₄ carbon chains, e.g., lauroyl, myristoyl, palmitoyl, stearoyl, or oleoyl. Additional examples of non-cationic lipids include sterols such as cholesterol and derivatives thereof such as cholestanol, cholestanone, cholestenone, coprostanol, cholesteryl-2′-hydroxyethyl ether, cholesteryl-4′-hydroxybutyl ether, and mixtures thereof. In certain embodiments, the phospholipid is DPPC, DSPC, or mixtures thereof.

### Conjugated Lipid

In the nucleic acid-lipid particles of the present disclosure, the conjugated lipid that inhibits aggregation of particles may comprise, e.g., one or more of the following: a polyethyleneglycol (PEG) lipid conjugate, a polyamide (ATTA)-lipid conjugate, a cationic-polymer-lipid conjugates (CPLs), or mixtures thereof. In some embodiments, the nucleic acid-lipid particles comprise either a PEG-lipid conjugate or an ATTA-lipid conjugate.

PEG is a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following: monomethoxypolyethylene glycol (MePEGOH), monomethoxypolyethylene glycolsuccinate (MePEGS), monomethoxypolyethylene glycolsuccinimidyl succinate (MePEG-S—NHS), monomethoxypolyethylene glycolamine (MePEG-NH₂), monomethoxypolyethylene glycoltresylate (MePEG-TRES), and monomethoxypolyethylene glycolimidazolylcarbonyl (MePEG-IM). Other PEGs such as those described in U.S. Pat. Nos. 6,774,180 and 7,053,150 (e.g., mPEG (20 KDa) amine) are also useful for preparing the PEG-lipid conjugates of the present disclosure. The disclosures of these patents are herein incorporated by reference in their entirety for all purposes. In addition, monomethoxypolyethyleneglycolacetic acid (MePEG-CH₂COOH) is particularly useful for preparing PEG-lipid conjugates including, e.g., PEG-DAA conjugates.

In certain embodiments, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or mixtures thereof. The PEGDAA conjugate may be PEG-dilauryloxypropyl (C₁₂), a PEG-dimyristyloxypropyl (C₁₄), a PEG-dipalmityloxypropyl (C₁₆), a PEG-distearyloxypropyl (Cis), or mixtures thereof.

Additional PEG-lipid conjugates suitable for use in the disclosure include, but are not limited to, mPEG2000-1,2-diO-alkyl-sn3-carbomoylglyceride (PEG-C-DOMG). The synthesis of PEG-C-DOMG is described in PCT Application No. PCT/US08/88676, filed Dec. 31, 2008, the disclosure of which is herein incorporated by reference in its entirety for all purposes. Yet additional PEG-lipid conjugates suitable for use in the disclosure include, without limitation, 1-[8′-(1,2-dimyristoyl-3-propanoxy)-carboxamido-3′,6′-dioxaoctanyl]carbamoyl-methyl-poly(ethylene glycol) (2 KPEG-DMG). The synthesis of 2 KPEG-DMG is described in U.S. Pat. No. 7,404,969, the disclosure of which is herein incorporated by reference in its entirety for all purposes.

The PEG moiety of the PEG-lipid conjugates described herein may comprise an average molecular weight ranging from about 550 daltons to about 10,000 daltons. In certain instances, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons, etc.). In some embodiments, the PEG moiety has an average molecular weight of about 2,000 daltons or about 750 daltons.

In addition to the foregoing, it will be readily apparent to those of skill in the art that other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses such as hydroxymethylcellulose or hydroxyethylcellulose.

In addition to the foregoing components, the particles (e.g., LNP) of the present disclosure can further comprise cationic poly(ethylene glycol) (PEG) lipids or CPLs (e.g., Chen et al., Bioconj. Chem., 11:433-437 (2000)). Suitable SPLPs and SPLP-CPLs for use in the present disclosure, and methods of making and using SPLPs and SPLP-CPLs, are disclosed, e.g., in U.S. Pat. No. 6,852,334 and PCT Publication No. WO 00/62813, the disclosures of which are herein incorporated by reference in their entirety for all purposes.

In certain instances, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle.

In the lipid nanoparticles of the present disclosure, the active agent or therapeutic agent may be fully encapsulated within the lipid portion of the particle, thereby protecting the active agent or therapeutic agent from enzymatic degradation. In some embodiments, a nucleic acid-lipid particle comprising a nucleic acid such as a messenger RNA (i.e., mRNA) is fully encapsulated within the lipid portion of the particle, thereby protecting the nucleic acid from nuclease degradation. In certain instances, the nucleic acid in the nucleic acid-lipid particle is not substantially degraded after exposure of the particle to a nuclease at 37° C. for at least about 20, 30, 45, or 60 minutes. In certain other instances, the nucleic acid in the nucleic acid-lipid particle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours. In other embodiments, the active agent or therapeutic agent (e.g., nucleic acid such as siRNA) is complexed with the lipid portion of the particle. One of the benefits of the formulations of the present disclosure is that the lipid particle compositions are substantially non-toxic to mammals such as humans.

### Lipid Nanoparticle (LNP) Compositions

In one aspect, the present disclosure provides a lipid nanoparticle (LNP). In certain embodiments, the LNP comprises at least one ionizable lipid. In certain embodiments, the LNP comprises at least one helper or neutral lipid. In certain embodiments, the LNP comprises cholesterol and/or a derivative thereof. In certain embodiments, the LNP comprises at least one polymer conjugated lipid. In certain embodiments, the LNP comprises at least one cargo molecule. In certain embodiments, the at least one cargo molecule is at least partially encapsulated in the LNP.

In certain embodiments, the ionizable lipid of Formula (I), or a salt, solvate, stereoisomer, or isotopologue thereof:

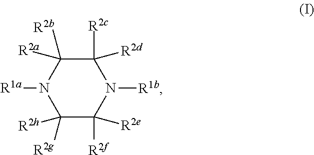

wherein:
R^{1a} and R^{1b} are each independently

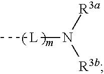

R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₇-C₁₃ aralkyl, optionally substituted C₆-C₁₀ aryl, and optionally substituted C₂-C₁₀ heteroaryl;
each occurrence of R^{3a}, R^{3b}, and R^{3c} is independently selected from the group consisting of H, -(optionally substituted C₁-C₆ alkylenyl)-C(═O)OR⁴, -(optionally substituted C₁-C₆ alkylenyl)-C(═O)N(R⁴)(R⁵), -(optionally substituted C₁-C₆ alkylenyl)-C(═O)R⁴, -(optionally substituted C₁-C₆ alkylenyl)-(R⁴), —C(═O)OR⁴, —C(═O)N(R⁴)(R⁵), —C(═O)R⁴, and R⁴,
   wherein no more than one of each occurrence of R^{3a}, R^{3b}, and R^{3c} is H;
R⁴ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁵ is selected from the group consisting of H and optionally substituted C₁-C₆ alkyl;
each occurrence of L is independently selected from the group consisting of -(optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, —X-(optionally substituted C₁-C₁₂ alkylenyl)-, —X-(optionally substituted C₂-C₁₂ alkenylenyl)-, —X-(optionally substituted C₁-C₁₂ alkynylenyl)-, —X-(optionally substituted C₁-C₁₂ heteroalkylenyl)-, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—; and
each occurrence of m is independently an integer selected from the group consisting of 1, 2, 3, and 4.

In certain embodiments, at least one selected from the group consisting of R^{2a}, R^{2b}, R^{2c}, R^{2d}, R^{2e}, R^{2f}, R^{2g}, and R^{2h} is H. In certain embodiments, at least two selected from the group consisting of R^{2a}, R^{2b}, R^{2c}, R^{2d}, R^{2e}, R^{2f}, R^{2g}, and R^{2h} are H. In certain embodiments, at least three selected from the group consisting of R^{2a}, R^{2b}, R^{2c}, R^{2d}, R^{2e}, R^{2f}, R^{2g}, and R^{2h} are H. In certain embodiments, at least four selected from the group consisting of R^{2a}, R^{2b}, R^{2c}, R^{2d}, R^{2e}, R^{2f}, R^{2g}, and R^{2h} are H. In certain embodiments, at least five selected from the group consisting of R^{2a}, R^{2b}, R^{2c}, R^{2d}, R^{2e}, R^{2f}, R^{2g}, and R^{2h} are H. In certain embodiments, at least six selected from the group consisting of R^{2a}, R^{2b}, R^{2c}, R^{2d}, R^{2e}, R^{2f}, R^{2g}, and R^{2h} are H. In certain embodiments, at least seven selected from the group consisting of R^{2a}, R^{2b}, R^{2c}, R^{2d}, R^{2e}, R^{2f}, R^{2g}, and R^{2h} are H. In certain embodiments, each of R^{2a}, R^{2b}, R^{2c}, R^{2d}, R^{2e}, R^{2f}, R^{2g}, and R^{2h} are H.

In certain embodiments, L is —CH₂—. In certain embodiments, L is —(CH₂)₂—. In certain embodiments, L is —(CH₂)₃—. In certain embodiments, L is —(CH₂)₁₀—. In certain embodiments, L is —(CH₂)₂O—. In certain embodiments, L is —(CH₂)₃O—. In certain embodiments, L is —CH₂CH(OR⁵)CH₂—. In certain embodiments, L is —(CH₂)₂NR^{3c}—. In certain embodiments, L is

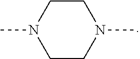

In certain embodiments, L is

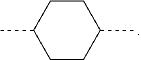

In certain embodiments, L is

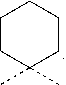

For instances of L which are asymmetric (e.g., —(CH₂)₃O—) it is understood that the present disclosure encompasses both possible orientations (e.g., —(CH₂)₃O— and —O(CH₂)₃—).

In certain embodiments, the ionizable lipid of Formula (I) is:

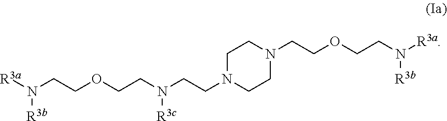

In certain embodiments, the ionizable lipid of Formula (I) is

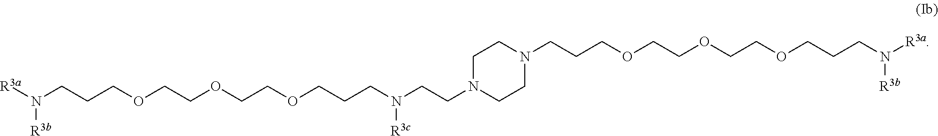

In certain embodiments, the ionizable lipid of Formula (I) is:

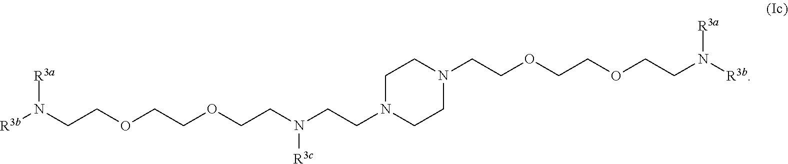

In certain embodiments, the ionizable lipid of Formula (I) is:

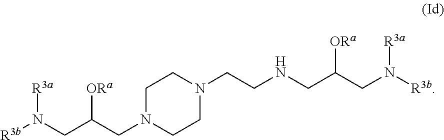

In certain embodiments, the ionizable lipid of Formula (I) is:

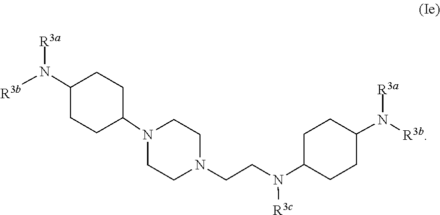

In certain embodiments, the ionizable lipid of Formula (I) is:

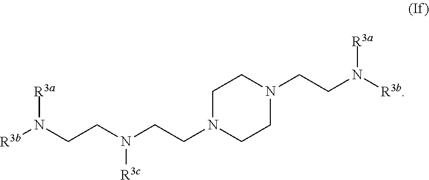

In certain embodiments, the ionizable lipid of Formula (I) is:

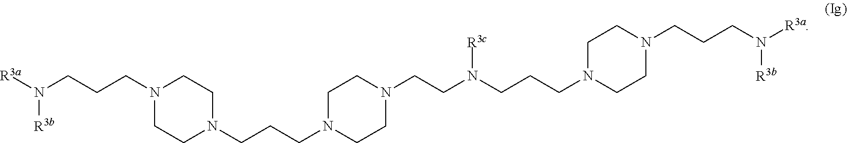

In certain embodiments, the ionizable lipid of Formula (I) is:

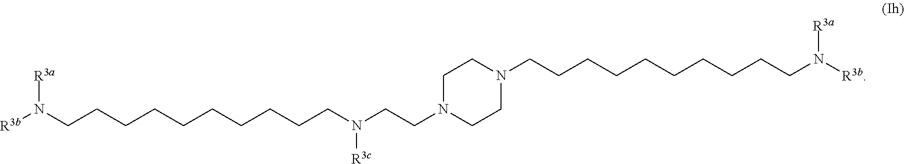

In certain embodiments, the ionizable lipid of Formula (I) is:

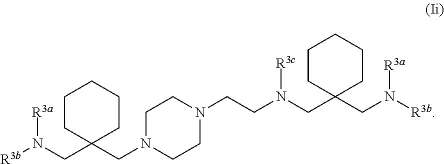

In certain embodiments, R^{3a} is H. In certain embodiments, R^{3a} is —CH₂CH(OH) (optionally substituted C₁-C₂₈ alkyl). In certain embodiments, R^{3a} is —CH₂CH(OH) (optionally substituted C₂-C₂₈ alkenyl). In certain embodiments, R^{3a} is —CH₂CH₂C(═O)O (optionally substituted C₁-C₂₈ alkyl). In certain embodiments, R^{3a} is —CH₂CH₂C(═O)NH (optionally substituted C₁-C₂₈ alkyl). In certain embodiments, R³¹ is H. In certain embodiments, R^{3b} is —CH₂CH(OH) (optionally substituted C₁-C₂₈ alkyl). In certain embodiments, R^{3b} is —CH₂CH(OH) (optionally substituted C₂-C₂₈ alkenyl). In certain embodiments, R^{3b} is —CH₂CH₂C(═O)O (optionally substituted C₁-C₂₈ alkyl). In certain embodiments, R^{3b} is —CH₂CH₂C(═O)NH (optionally substituted C₁-C₂₈ alkyl). In certain embodiments, R^{3c} is H. In certain embodiments, R^{3c} is —CH₂CH(OH) (optionally substituted C₁-C₂₈ alkyl). In certain embodiments, R^{3c} is —CH₂CH(OH) (optionally substituted C₂-C₂₈ alkenyl).

In certain embodiments, R^{3c} is —CH₂CH₂C(═O)O (optionally substituted C₁-C₂₈ alkyl). In certain embodiments, R^{3c} is —CH₂CH₂C(═O)NH (optionally substituted C₁-C₂₈ alkyl).

In certain embodiments, R^{3a} is —CH₂CH(OH)(CH₂)₉CH₃. In certain embodiments, R^{3a} is —CH₂CH(OH)(CH₂)₁₁CH₃. In certain embodiments, R^{3a} is —CH₂CH(OH)(CH₂)₁₃CH₃. In certain embodiments, R^{3b} is —CH₂CH(OH)(CH₂)₉CH₃. In certain embodiments, R^{3b} is —CH₂CH(OH)(CH₂)₁₁CH₃. In certain embodiments, R^{3b} is —CH₂CH(OH)(CH₂)₁₃CH₃. In certain embodiments, R^{3c} is —CH₂CH(OH)(CH₂)₉CH₃. In certain embodiments, R^{3c} is —CH₂CH(OH)(CH₂)₁₁CH₃. In certain embodiments, R^{3c} is —CH₂CH(OH)(CH₂)₁₃CH₃.

In certain embodiments, each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, optionally substituted heteroalkylenyl, optionally substituted cycloalkylenyl, and optionally substituted heterocycloalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R″)(R″″), C(═O)R″, C(═O)OR″, OC(═O)OR″, C(═O)N(R″)(R″″), S(═O)₂N(R″)(R″″), N(R″)C(═O)R″″, N(R″)S(═O)₂R″″, C₂-C₈ heteroaryl, and phenyl optionally substituted with at least one halogen, wherein each occurrence of R″ and R″″ is independently selected from the group consisting of H, C₁-C₆ alkyl, C₃-C₈ cycloalkyl, C₁-C₆ haloalkyl, benzyl, and phenyl.

In certain embodiments, the ionizable lipid of Formula (I) is:

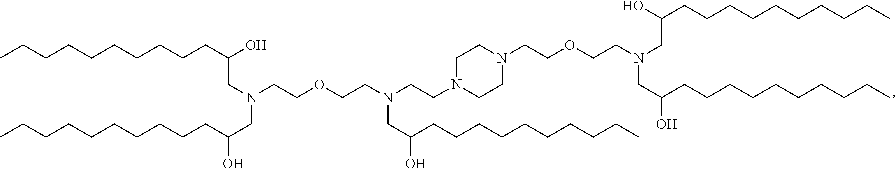 [SMILES: CCCCCCCCCCC(O)CN(CCOCCN(CC(O)CCCCCCCCCC)CC(O)CCCCCCCCCC)CCN1CCN(CCOCCN(CC(O)CCCCCCCCCC)CC(O)CCCCCCCCCC)CC1]

1,1″-((2-(2-(4-(2-((2-(2-(bis(2-hydroxydodecyl)amino)ethoxy)ethyl)(2-hydroxydodecyl)amino)ethyl)piperazin-1-yl)ethoxy)ethyl)azanediyl)bis(dodecan-2-ol) (A4).

In certain embodiments, the ionizable lipid of Formula (I) is:

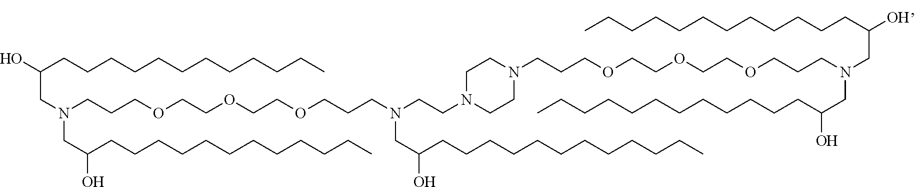 [SMILES: CCCCCCCCCCCCC(O)CN(CCCOCCOCCOCCCN(CC(O)CCCCCCCCCCCC)CC(O)CCCCCCCCCCCC)CCN1CCN(CCCOCCOCCOCCCN(CC(O)CCCCCCCCCCCC)CC(O)CCCCCCCCCCCC)CC1]

15-(2-(4-(16-hydroxy-14-(2-hydroxytetradecyl)-4,7,10-trioxa-14-azaoctacosyl)piperazin-1-yl)ethyl)-29-(2-hydroxytetradecyl)-19,22,25-trioxa-15,29-diazatritetracontane-13,31-diol (B5).

In certain embodiments, the ionizable lipid of Formula (I) is:

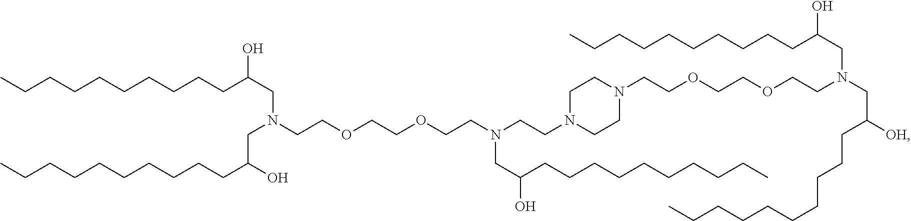 [SMILES: CCCCCCCCCCC(O)CN(CCOCCOCCN(CC(O)CCCCCCCCCC)CC(O)CCCCCCCCCC)CCN1CCN(CCOCCOCCN(CC(O)CCCCCCCCCC)CC(O)CCCCCCCCCC)CC1]

13-(2-(4-(2-(2-(2-(bis(2-hydroxydodecyl)amino)ethoxy)ethoxy)ethyl)piperazin-1-yl)ethyl)-22-(2-hydroxydodecyl)-16,19-dioxa-13,22-diazatetratriacontane-11,24-diol (A2).

In certain embodiments, the ionizable lipid of Formula (I) is:

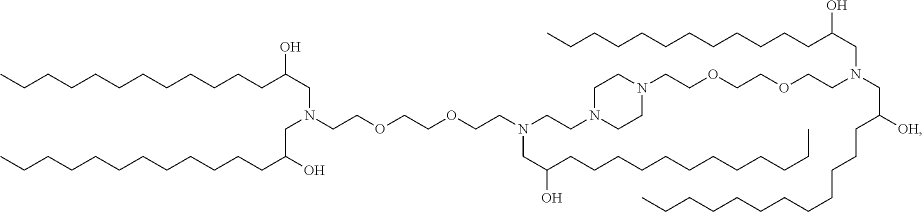 [SMILES: CCCCCCCCCCCCC(O)CN(CCOCCOCCN(CC(O)CCCCCCCCCCCC)CC(O)CCCCCCCCCCCC)CCN1CCN(CCOCCOCCN(CC(O)CCCCCCCCCCCC)CC(O)CCCCCCCCCCCC)CC1]

15-(2-(4-(2-(2-(2-(bis(2-hydroxytetradecyl)amino)ethoxy)ethoxy)ethyl)piperazin-1-yl)ethyl)-24-(2-hydroxytetradecyl)-18,21-dioxa-15,24-diazaoctatriacontane-13,26-diol (B2).

In certain embodiments, the ionizable lipid of Formula (I) is:

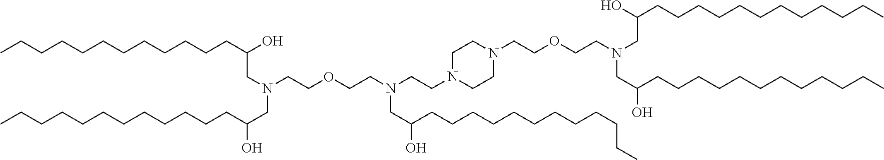 [SMILES: CCCCCCCCCCCCC(O)CN(CCOCCN(CC(O)CCCCCCCCCCCC)CC(O)CCCCCCCCCCCC)CCN1CCN(CCOCCN(CC(O)CCCCCCCCCCCC)CC(O)CCCCCCCCCCCC)CC1]

1,1′-((2-(2-(4-(2-((2-(2-(bis(2-hydroxytetradecyl)amino)ethoxy)ethyl)(2-hydroxytetradecyl)amino)ethyl)piperazin-1-yl)ethoxy)ethyl)azanediyl)bis(tetradecan-2-ol), (C14-494).

In certain embodiments, the at least one ionizable lipid comprises less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP.

In certain embodiments, the at least one ionizable lipid comprises more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP.

In certain embodiments, the at least one ionizable lipid comprises about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, the at least one ionizable lipid comprises less than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, the at least one ionizable lipid comprises more than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, the at least one ionizable lipid comprises about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, the at least one ionizable lipid comprises less than about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, the at least one ionizable lipid comprises more than about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, the at least one ionizable lipid comprises about 32.4 mol % of the LNP. In certain embodiments, the at least one ionizable lipid comprises about 35 mol % of the LNP. In certain embodiments, the at least one ionizable lipid comprises about 49 mol % of the LNP. In certain embodiments, the at least one ionizable lipid comprises about 51 mol % of the LNP. In certain embodiments, the at least one ionizable lipid comprises about 55 mol % of the LNP.

In certain embodiments, the helper lipid comprises dioleoylphosphatidylethanolamine (DOPE) and distearoylphosphatidylcholine (DSPC). In certain embodiments, the helper lipid is dioleoylphosphatidylethanolamine (DOPE). In certain embodiments, the helper lipid is distearoylphosphatidylcholine (DSPC).

In certain embodiments, the at least one helper lipid comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mol % of the LNP. In certain embodiments, the at least one helper lipid comprises less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mol % of the LNP. In certain embodiments, the at least one helper lipid comprises more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mol % of the LNP.

In certain embodiments, the at least one helper lipid comprises about 1, 2, 3, 4, 5, 6, 7, 8, 9, 10, 11, 12, 13, 14, 15, 16, 17, 18, 19, 20, 21, 22, 23, 24, or about 25 mol % of the LNP. In certain embodiments, the at least one helper lipid comprises less than about 1, 2, 3, 4, 5, 6, 7, 8, 9, 10, 11, 12, 13, 14, 15, 16, 17, 18, 19, 20, 21, 22, 23, 24, or about 25 mol % of the LNP. In certain embodiments, the at least one helper lipid comprises more than about 1, 2, 3, 4, 5, 6, 7, 8, 9, 10, 11, 12, 13, 14, 15, 16, 17, 18, 19, 20, 21, 22, 23, 24, or about 25 mol % of the LNP.

In certain embodiments, the at least one helper lipid comprises about 33 mol % of the LNP. In certain embodiments, the at least one helper lipid comprises about 29 mol % of the LNP. In certain embodiments, the at least one helper lipid comprises about 22.2 mol % of the LNP. In certain embodiments, the at least one helper lipid comprises about 16 mol % of the LNP. In certain embodiments, the at least one helper lipid comprises about 14 mol % of the LNP. In certain embodiments, the at least one helper lipid comprises about 14.5 mol % of the LNP. In certain embodiments, the at least one helper lipid comprises about 13 mol % of the LNP. In certain embodiments, the at least one helper lipid comprises about 11.5 mol % of the LNP. In certain embodiments, the at least one helper lipid comprises about 10 mol % of the LNP.

In certain embodiments, the LNP comprises about 14 mol % DOPE. In certain embodiments, the LNP comprises about 16 mol % DOPE. In certain embodiments, the LNP comprises about 22.2 mol % DOPE. In certain embodiments, the LNP comprises about 29 mol % DOPE. In certain embodiments, the LNP comprises about 33 mol % DOPE.

In certain embodiments, cholesterol comprises about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or about 70 mol % of the LNP.

In certain embodiments, cholesterol comprises less than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or about 70 mol % of the LNP.

In certain embodiments, cholesterol and/or a derivative thereof comprises more than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or about 70 mol % of the LNP.

In certain embodiments, cholesterol and/or a derivative thereof comprises about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, cholesterol and/or a derivative thereof comprises less than about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, cholesterol and/or a derivative thereof comprises more than about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, cholesterol and/or a derivative thereof comprises about 15 mol % of the LNP. In certain embodiments, cholesterol and/or a derivative thereof comprises about 16 mol % of the LNP. In certain embodiments, cholesterol and/or a derivative thereof comprises about 31.5 mol % of the LNP. In certain embodiments, cholesterol and/or a derivative thereof comprises about 33 mol % of the LNP. In certain embodiments, cholesterol and/or a derivative thereof comprises about 43.1 mol % of the LNP. In certain embodiments, cholesterol and/or a derivative thereof comprises about 46.5 mol % of the LNP. In certain embodiments, cholesterol and/or a derivative thereof comprises about 61.5 mol % of the LNP.

In certain embodiments, the cholesterol and/or a derivative thereof comprises cholesterol. In certain embodiments, the cholesterol and/or a derivative thereof comprises 24-α-methyl-cholesterol (campesterol). In certain embodiments, the cholesterol and/or a derivative thereof comprises 24-α-ethyl-cholestanol (stigmastanol). In certain embodiments, the cholesterol and/or a derivative thereof comprises 24-α-ethyl-cholesterol (β-sitosterol).

In certain embodiments, the at least one polymer conjugated lipid comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or about 20.0 mol % of the LNP.

In certain embodiments, the at least one polymer conjugated lipid comprises less than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or about 20.0 mol % of the LNP.

In certain embodiments, the at least one polymer conjugated lipid comprises more than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or about 20.0 mol % of the LNP.

In certain embodiments, the at least one polymer conjugated lipid comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5.0 mol % of the LNP.

In certain embodiments, the at least one polymer conjugated lipid comprises less than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5.0 mol % of the LNP.

In certain embodiments, the at least one conjugated lipid comprises more than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5.0 mol % of the LNP.

In certain embodiments, the at least one polymer conjugated lipid comprises about 1.6 mol % of the LNP. In certain embodiments, the at least one polymer conjugated lipid comprises about 1.8 mol % of the LNP. In certain embodiments, the at least one polymer conjugated lipid comprises about 1.9 mol % of the LNP. In certain embodiments, the at least one polymer conjugated lipid comprises about 2.3 mol % of the LNP. In certain embodiments, the at least one polymer conjugated lipid comprises about 2.5 mol % of the LNP.

In certain embodiments, the at least one polymer conjugated lipid comprises a polyethylene glycol (PEG)-conjugated lipid. In certain embodiments, the at least one polymer conjugated lipid comprises C14-PEG. In certain embodiments, C14-PEG comprises:

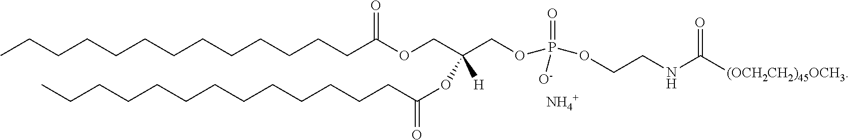

In certain embodiments, the LNP has a molar ratio of (a):(b):(c):(d) of about 30:20:10:1. In certain embodiments, the LNP has a molar ratio of (a):(b):(c):(d) of about 30:16:8:1. In certain embodiments, the LNP has a molar ratio of (a):(b):(c):(d) of about 55:15:35:2. In certain embodiments, the LNP has a molar ratio of (a):(b):(c):(d) of about 35:16:46.5:2.5. In certain embodiments, the LNP has a molar ratio of (a):(b):(c):(d) of about 35:24:46.5:2.5. In certain embodiments, the LNP has a molar ratio of (a):(b):(c):(d) of about 36:16:61.5:2.5.

In certain embodiments, the LNP comprises (a):(b):(c):(d) having a molar percentage of about 49.18:32.79:16.39:1.64. In certain embodiments, the LNP comprises (a):(b):(c):(d) having a molar percentage of about 54.55:29.09:14.55:1.82. In certain embodiments, the LNP comprises (a):(b):(c):(d) having a molar percentage of about 51.40:14.02:32.71:1.87. In certain embodiments, the LNP comprises (a):(b):(c):(d) having a molar percentage of about 35:16:46.5:2.5. In certain embodiments, the LNP comprises (a):(b):(c):(d) having a molar percentage of 32.4:22.2:43.1:2.3.

In certain embodiments, the LNP comprises C12-494, DOPE, cholesterol, and C14-PEG₂ₖ having a molar ratio of 35:16:46.5:2.5. In certain embodiments, the LNP consists essentially of C12-494, DOPE, cholesterol, and C14-PEG₂ₖ having a molar ratio of 35:16:46.5:2.5, and the at least one cargo molecule. In certain embodiments, the LNP consists of C12-494, DOPE, cholesterol, and C14-PEG₂ₖ having a molar ratio of 35:16:46.5:2.5, and the at least one cargo molecule.

In certain embodiments, the LNP comprises C12-494, DOPE, campesterol, and C14-PEG₂ₖ having a molar ratio of 35:16:46.5:2.5. In certain embodiments, the LNP consists essentially of C12-494, DOPE, campesterol, and C14-PEG₂ₖ having a molar ratio of 35:16:46.5:2.5, and the at least one cargo molecule. In certain embodiments, the LNP consists of C12-494, DOPE, campesterol, and C14-PEG₂ₖ having a molar ratio of 35:16:46.5:2.5, and the at least one cargo molecule.

In certain embodiments, the LNP comprises C12-494, DOPE, β-sitosterol, and C14-PEG₂ₖ having a molar ratio of 35:16:46.5:2.5. In certain embodiments, the LNP consists essentially of C12-494, DOPE, β-sitosterol, and C14-PEG₂ₖ having a molar ratio of 35:16:46.5:2.5, and the at least one cargo molecule. In certain embodiments, the LNP consists of C12-494, DOPE, β-sitosterol, and C14-PEG₂ₖ having a molar ratio of 35:16:46.5:2.5, and the at least one cargo molecule.

In certain embodiments, the LNP comprises C12-494, DOPE, stigmastanol, and C14-PEG₂ₖ having a molar ratio of 35:16:46.5:2.5. In certain embodiments, the LNP consists essentially of C12-494, DOPE, stigmastanol, and C14-PEG₂ₖ having a molar ratio of 35:16:46.5:2.5, and the at least one cargo molecule. In certain embodiments, the LNP consists of C12-494, DOPE, stigmastanol, and C14-PEG₂ₖ having a molar ratio of 35:16:46.5:2.5, and the at least one cargo molecule.

In certain embodiments, The LNP further comprises at least one cargo molecule.

In certain embodiments, the cargo is at least one selected from the group consisting of a nucleic acid, small molecule, protein, therapeutic agent, antibody, and any combinations thereof.

In certain embodiments, the cargo is a nucleic acid. In certain embodiments, the nucleic acid is DNA or RNA. In certain embodiments, the nucleic acid is selected from the group consisting of mRNA, cDNA, pDNA, microRNA, siRNA, modified RNA, antagomir, antisense molecule, and any combinations thereof. In certain embodiments, the cargo is at least partially encapsulated in the LNP. In certain embodiments, the cargo is mRNA.

In certain embodiments, LNP has a weight ratio of ionizable lipid to mRNA of about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, and about 20:1. In certain embodiments, the LNP has a weight ratio of ionizable lipid to mRNA of about 10:1.

In certain embodiments, the mRNA encodes VEGF.

In certain embodiments, the LNP is selectively delivered to the placenta of a subject.

In another aspect, the present disclosure provides a lipid nanoparticle (LNP) composition. In certain embodiments, the LNP composition comprises (a) at least one ionizable lipid. In certain embodiments, the LNP composition comprises (b) at least one helper lipid. In certain embodiments, the LNP composition comprises (c) at least one cholesterol lipid. In certain embodiments, the LNP composition comprises (d) at least one polymer conjugated lipid and/or a modified derivative thereof. In certain embodiments, the LNP composition comprises (e) an epidermal growth factor (EGFR) targeting domain. In certain embodiments, the EGFR targeting domain is covalently conjugated to at least one component of the LNP.

In certain embodiments, the ionizable lipid of Formula (I) is:

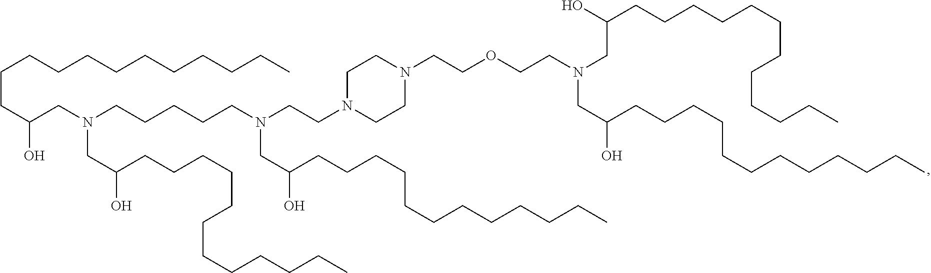 [SMILES: CCCCCCCCCCCCC(O)CN(CCCCCN(CC(O)CCCCCCCCCCCC)CC(O)CCCCCCCCCCCC)CCN1CCN(CCOCCN(CC(O)CCCCCCCCCCCC)CC(O)CCCCCCCCCCCC)CC1]

1,1′-((2-(2-(4-(2-((2-(2-(bis(2-hydroxytetradecyl)amino)ethoxy)ethyl)(2-hydroxytetradecyl)amino)ethyl)piperazin-1-yl)ethoxy)ethyl)azanediyl)bis(tetradecan-2-ol) (C14-494).

In certain embodiments, the at least one ionizable lipid comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP.

In certain embodiments, the at least one ionizable lipid comprises less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP.

In certain embodiments, the at least one ionizable lipid comprises more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP.

In certain embodiments, the at least one ionizable lipid comprises about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, the at least one ionizable lipid comprises less than about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, the at least one ionizable lipid comprises more than about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, the at least one ionizable lipid comprises about 35 mol % of the LNP. In certain embodiments, the at least one ionizable lipid comprises about 38.8 mol % of the LNP. In certain embodiments, the at least one ionizable lipid comprises about 42.5 mol % of the LNP. In certain embodiments, the at least one ionizable lipid comprises about 46.3 mol % of the LNP. In certain embodiments, the at least one ionizable lipid comprises about 50 mol % of the LNP.

In certain embodiments, the helper lipid comprises dioleoylphosphatidylethanolamine (DOPE) and distearoylphosphatidylcholine (DSPC). In certain embodiments, the helper lipid is dioleoylphosphatidylethanolamine (DOPE). In certain embodiments, the helper lipid is distearoylphosphatidylcholine (DSPC).

In certain embodiments, the at least one helper lipid comprises about 1, 2, 3, 4, 5, 6, 7, 8, 9, 10, 11, 12, 13, 14, 15, 16, 17, 18, 19, 20, 21, 22, 23, 24, or about 25 mol % of the LNP. In certain embodiments, the at least one helper lipid comprises less than about 1, 2, 3, 4, 5, 6, 7, 8, 9, 10, 11, 12, 13, 14, 15, 16, 17, 18, 19, 20, 21, 22, 23, 24, or about 25 mol % of the LNP. In certain embodiments, the at least one helper lipid comprises more than about 1, 2, 3, 4, 5, 6, 7, 8, 9, 10, 11, 12, 13, 14, 15, 16, 17, 18, 19, 20, 21, 22, 23, 24, or about 25 mol % of the LNP.

In certain embodiments, the at least one helper lipid comprises about 16 mol % of the LNP. In certain embodiments, the at least one helper lipid comprises about 14.5 mol % of the LNP. In certain embodiments, the at least one helper lipid comprises about 13 mol % of the LNP. In certain embodiments, the at least one helper lipid comprises about 11.5 mol % of the LNP. In certain embodiments, the at least one helper lipid comprises about 10 mol % of the LNP.

In certain embodiments, the LNP comprises about 16 mol % DOPE. In certain embodiments the LNP comprises about 3.6 mol % DSPC and about 10.9 mol % DOPE. In certain embodiments, the LNP comprises about 6.5 mol % DSPC and about 6.5 mol % DOPE. In certain embodiments, the LNP comprises about 8.6 mol % DSPC and about 2.9 mol % DOPE. In certain embodiments, the LNP comprises about 10 mol % DSPC.

In certain embodiments, cholesterol comprises about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, cholesterol and/or a derivative thereof comprises less than about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, cholesterol and/or a derivative thereof comprises more than about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or about 60 mol % of the LNP.

In certain embodiments, cholesterol and/or a derivative thereof comprises about 46.5 mol % of the LNP. In certain embodiments, cholesterol and/or a derivative thereof comprises about 44.5 mol % of the LNP. In certain embodiments, cholesterol and/or a derivative thereof comprises about 42.5 mol % of the LNP. In certain embodiments, cholesterol and/or a derivative thereof comprises about 40.5 mol % of the LNP. In certain embodiments, cholesterol and/or a derivative thereof comprises about 38.5 mol % of the LNP.

In certain embodiments, the an epidermal growth factor (EGFR) targeting domain is covalently conjugated to the at least one polymer conjugated lipid.

In certain embodiments, the targeting domain comprises at least one selected from the group consisting of a polypeptide, a polynucleotide, and a small molecule.

In certain embodiments, the targeting domain comprises a polypeptide. In certain embodiments, the polypeptide is an antibody. In certain embodiments, the antibody is EGFR IgG1. In certain embodiments, the antibody shares at least 85% sequence homology with SEQ ID NO:1. In certain embodiments, the antibody shares at least 90% sequence homology with SEQ ID NO:1. In certain embodiments, the antibody shares at least 95% sequence homology with SEQ ID NO:1. In certain embodiments, the antibody shares at least 96% sequence homology with SEQ ID NO:1. In certain embodiments, the antibody shares at least 97% sequence homology with SEQ ID NO:1. In certain embodiments, the antibody shares at least 98% sequence homology with SEQ ID NO:1. In certain embodiments, the antibody shares at least 99% sequence homology with SEQ ID NO:1. In certain embodiments, the antibody shares 100% sequence homology with SEQ ID NO:1. In certain embodiments, the EGFR IgG1 comprises human-reactive EGFR IgG1 clone AY13. In certain embodiments, the EGFR IgG1 comprises mouse-reactive EGFR IgG1 clone 30H45L48.

In certain embodiments, the at least one polymer conjugated lipid comprises a polyethylene glycol (PEG) conjugated lipid and an EGFR-PEG-conjugated lipid (EGFR-PEG).

In certain embodiments, the EGFR targeting domain is covalently conjugated to the PEG conjugated lipid via a linker comprising a moiety formed by a click reaction.

In certain embodiments, the click reaction is selected from the group consisting of a [3+2] cycloaddition and a [4+2] cycloaddition. In certain embodiments, the [3+2] cycloaddition is selected from the group consisting of a strain-promoted azide-alkyne cycloaddition (SPAAC), a Cu(I)-catalyzed azide-alkyne cycloaddition (CuAAC), and a strain-promoted alkyne-nitrone cycloaddition (SPANC). In certain embodiments, the [4+2] cycloaddition is selected from the group consisting of a Diels-Alder reaction and an alkene/tetrazine inverse-demand Diels-Alder reaction. In certain embodiments, the moiety comprises a 1,2,3-triazole.

In certain embodiments, the linker has a first terminus which is covalently conjugated to a functional group of a side chain residue or a terminal residue of the polypeptide comprising the epidermal growth factor (EGFR) targeting domain. In certain embodiments, the linker has a second terminus which is covalently conjugated to a terminal hydroxyl of the PEG conjugated lipid. In certain embodiments, the linker has a first terminus which is covalently conjugated to a functional group of a side chain residue or a terminal residue of the polypeptide comprising the epidermal growth factor (EGFR) targeting domain and the linker has a second terminus which is covalently conjugated to a terminal hydroxyl of the PEG conjugated lipid.

In certain embodiments, the linker is selected from the group consisting of

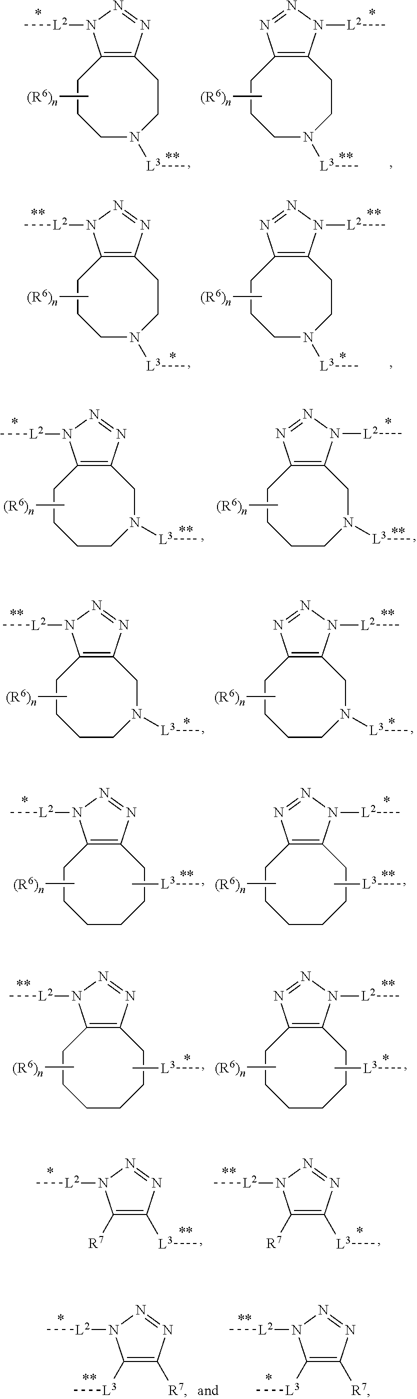

wherein:
L² and L³ are each independently a bond or at least one divalent substituent selected from the group consisting of —C(═O)—, —N(R^{a})—, —O—, —S—, optionally substituted C₁-C₁₂ alkylenyl, optionally substituted C₃-C₁₂ heterocycloalkylenyl, optionally substituted C₂-C₁₂ heteroalkylenyl, and optionally substituted C₂-C₁₂ heterocycloalkylenyl;
R⁶ is selected from the group consisting of optionally substituted C₁-C₆ alkyl, C₂-C₆ heteroalkyl, optionally substituted C₃-C₈ cycloalkyl, optionally substituted phenyl, optionally substituted benzyl, optionally substituted C₂-C₉ heterocyclyl, halogen, OR^{a}, N(R^{a})(R^{b}), SR^{a}, CN, and NO₂,
wherein two adjacent R⁶ substituents may combine with the atoms to which they are bound to form an optionally substituted phenyl, optionally substituted C₃-C₈ cycloalkyl, or optionally substituted C₂-C₉ heterocyclyl;
each occurrence of R^{a} and R^{b} is independently selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
R⁷ is selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
n is an integer from 0 to 11;
* indicates a bond between the linker and the EGFR targeting domain; and
** indicates a bond between the linker and the polymer conjugated lipid.

In certain embodiments, each occurrence of optionally substituted alkyl, optionally substituted heteroalkyl, optionally substituted cycloalkyl, optionally substituted heterocycloalkyl, optionally substituted heterocyclyl, optionally substituted alkenyl, optionally substituted alkynyl, optionally substituted aryl, optionally substituted heteroaryl, optionally substituted aralkyl, optionally substituted alkylenyl, optionally substituted heteroalkylenyl, optionally substituted cycloalkylenyl, and optionally substituted heterocycloalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

In certain embodiments, the linker is

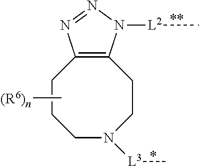

In certain embodiments, L² comprises a bond. In certain embodiments, L² comprises —C(═O)—. In certain embodiments, L² comprises —CH₂—. In certain embodiments, L² comprises —NH—. In certain embodiments, L² comprises —CH₂. In certain embodiments, L² comprises —O—. In certain embodiments, L² comprises —C(═O)—(CH₂)₃—C(═O)NH—(CH₂)₂—(OCH₂CH₂)₁₋₁₀₀—C(═O)—.

In certain embodiments, L² is a bond. In certain embodiments, L² is —C(═O)—. In certain embodiments, L² is —CH₂—. In certain embodiments, L² is —NH—. In certain embodiments, L² is —CH₂. In certain embodiments, L² is —O—. In certain embodiments, L² is —C(═O)—(CH₂)₃—C(═O)NH—(CH₂)₂—(OCH₂CH₂)₁₋₁₀₀—C(═O)—.

In certain embodiments, L³ comprises a bond. In certain embodiments, L³ comprises —C(═O)—. In certain embodiments, L³ comprises —CH₂—. In certain embodiments, L³ comprises —NH—. In certain embodiments, L³ comprises —CH₂. In certain embodiments, L³ comprises —O—. In certain embodiments, L³ comprises —C(═O)—(CH₂)₃—C(═O)NH—(CH₂)₂—(OCH₂CH₂)₁₋₁₀₀—C(═O)—.

In certain embodiments, L³ is a bond. In certain embodiments, L³ is —C(═O)—. In certain embodiments, L³ is —CH₂—. In certain embodiments, L³ is —NH—. In certain embodiments, L³ is —CH₂. In certain embodiments, L³ is —O—. In certain embodiments, L³ is —C(═O)—(CH₂)₃—C(═O)NH—(CH₂)₂—(OCH₂CH₂)₁₋₁₀₀—C(═O)—.

In certain embodiments, the linker comprises:

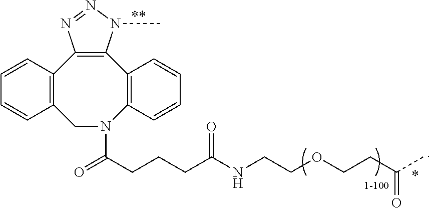

In certain embodiments, the at least one conjugated lipid comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5.0 mol % of the LNP.

In certain embodiments, the at least one conjugated lipid comprises less than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5.0 mol % of the LNP.

In certain embodiments, the at least one conjugated lipid comprises more than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5.0 mol % of the LNP.

In certain embodiments, the at least one conjugated lipid comprises about 2.5 mol % of the LNP. In certain embodiments, the at least one conjugated lipid comprises about 2.25 mol % of the LNP. In certain embodiments, the at least one conjugated lipid comprises about 2.0 mol % of the LNP. In certain embodiments, the at least one conjugated lipid comprises about 1.7 mol % of the LNP. In certain embodiments, the at least one conjugated lipid comprises about 1.5 mol % of the LNP.

In certain embodiments, the EGFR-PEG-conjugated lipid (EGFR-PEG) and the polyethylene glycol (PEG) conjugated lipid have a ratio of about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or about 1:20 (EGFR-PEG:PEG).

In certain embodiments, the EGFR-PEG-conjugated lipid (EGFR-PEG) and the polyethylene glycol (PEG) conjugated lipid have a ratio of less than about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or about 1:20 (EGFR-PEG:PEG).

In certain embodiments, the EGFR-PEG-conjugated lipid (EGFR-PEG) and the polyethylene glycol (PEG) conjugated lipid have a ratio of more than about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or about 1:20 (EGFR-PEG:PEG).

In certain embodiments, the EGFR-PEG-conjugated lipid (EGFR-PEG) and the polyethylene glycol (PEG) conjugated lipid have a ratio of about 1:2, 1:3, 1:5, or about 1:7 (EGFR-PEG:PEG).

In certain embodiments, the at least one polymer conjugated lipid comprises C14-PEG. In certain embodiments, C14-PEG comprises:

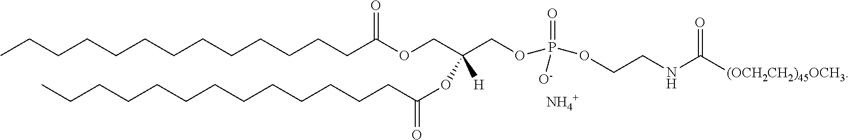

In certain embodiments, the EGFR-PEG-conjugated lipid (i.e., EGFR targeting domain-PEG-conjugated lipid) comprises:

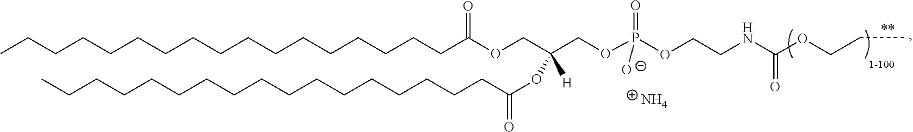

wherein ** indicates a bond between the EGFR-PEG-conjugated lipid and the linker.

In certain embodiments, the LNP has a molar ratio of (a):(b):(c):(d) of about 35:16:46.5:2.5. In certain embodiments, the LNP has a molar ratio of (a):(b):(c):(d) of about 38.8:14.5:44.5:2.25. In certain embodiments, the LNP has a molar ratio of (a):(b):(c):(d) of about 42.5:13:42.5:2.0. In certain embodiments, the LNP has a molar ratio of (a):(b):(c):(d) of about 46.3:11.5:40.5:1.75. In certain embodiments, the LNP has a molar ratio of (a):(b):(c):(d) of about 50:10:38.5:1.5.

In certain embodiments, the LNP has a molar ratio of (a):(b):(c):PEG:EGFR-PEG of about 35:16:46.5:2.1875:0.3125. In certain embodiments, the LNP has a molar ratio of (a):(b):(c):PEG:EGFR-PEG of about 35:16:46.5:2.083:0.4167. In certain embodiments, the LNP has a molar ratio of (a):(b):(c):PEG:EGFR-PEG of about 35:16:46.5:1.875:0.625. In certain embodiments, the LNP has a molar ratio of (a):(b):(c):PEG:EGFR-PEG of about 35:16:46.5:1.667:0.833.

In certain embodiments, the LNP further comprises at least one cargo molecule.

In certain embodiments, the cargo is at least one selected from the group consisting of a nucleic acid, small molecule, protein, therapeutic agent, antibody, and any combinations thereof.

In certain embodiments, the cargo is a nucleic acid. In certain embodiments, the nucleic acid is DNA or RNA. In certain embodiments, the nucleic acid is selected from the group consisting of mRNA, cDNA, pDNA, microRNA, siRNA, modified RNA, antagomir, antisense molecule, and any combinations thereof. In certain embodiments, the cargo is at least partially encapsulated in the LNP. In certain embodiments, the cargo is mRNA.

In certain embodiments, the LNP has a weight ratio of ionizable lipid to mRNA of about 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, and about 20:1. In certain embodiments, the LNP has a weight ratio of ionizable lipid to mRNA of about 10:1.

In certain embodiments, the LNP is selectively delivered to the placenta of a subject.

### Cargo

In one aspect, the present disclosure relates to LNPs comprising at least one cargo molecule at least partially encapsulated therein. In certain embodiments, the at least one cargo is fully encapsulated therein.

### Small Molecule Therapeutic Agents

In various embodiments, the agent is a therapeutic agent. In various embodiments, the therapeutic agent is a small molecule. When the therapeutic agent is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In certain embodiments, a small molecule therapeutic agents comprises an organic molecule, inorganic molecule, biomolecule, synthetic molecule, and the like.

Combinatorial libraries of molecularly diverse chemical compounds potentially useful in treating a variety of diseases and conditions are well known in the art, as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development. In some embodiments of the disclosure, the therapeutic agent is synthesized and/or identified using combinatorial techniques.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores. In some embodiments of the disclosure, the therapeutic agent is synthesized via small library synthesis.

The small molecule and small molecule compounds described herein may be present as salts even if salts are not depicted, and it is understood that the disclosure embraces all salts and solvates of the therapeutic agents depicted here, as well as the non-salt and non-solvate form of the therapeutic agents, as is well understood by the skilled artisan. In some embodiments, the salts of the therapeutic agents of the disclosure are pharmaceutically acceptable salts.

Where tautomeric forms may be present for any of the therapeutic agents described herein, each and every tautomeric form is intended to be included in the present disclosure, even though only one or some of the tautomeric forms may be explicitly depicted. For example, when a 2-hydroxypyridyl moiety is depicted, the corresponding 2-pyridone tautomer is also intended.

The disclosure also includes any or all of the stereochemical forms, including any enantiomeric or diastereomeric forms of the therapeutic agents described. The recitation of the structure or name herein is intended to embrace all possible stereoisomers of therapeutic agents depicted. All forms of the therapeutic agents are also embraced by the disclosure, such as crystalline or non-crystalline forms of the therapeutic agent. Compositions comprising a therapeutic agents of the disclosure are also intended, such as a composition of substantially pure therapeutic agent, including a specific stereochemical form thereof, or a composition comprising mixtures of therapeutic agents of the disclosure in any ratio, including two or more stereochemical forms, such as in a racemic or non-racemic mixture.

The disclosure also includes any or all active analog or derivative, such as a prodrug, of any therapeutic agent described herein. In certain embodiments, the therapeutic agent is a prodrug. In certain embodiments, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule therapeutic agents described herein are derivatives or analogs of known therapeutic agents, as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be carbocyclic or heterocyclic.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative of any of a small molecule inhibitor in accordance with the present disclosure can be used to treat a disease or disorder.

In certain embodiments, the small molecule therapeutic agents described herein can independently be derivatized, or analogs prepared therefrom, by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Nucleic Acid Therapeutic Agents

In certain embodiments, the composition of the disclosure comprises an in vitro transcribed (IVT) RNA molecule. For example, in certain embodiments, the composition of the disclosure comprises an IVT RNA molecule which encodes an agent. In certain embodiments, the IVT RNA molecule of the present composition is a nucleoside-modified mRNA molecule. In certain embodiments, the agent is for targeting an immune cell to a pathogen or a tumor cell of interest. In certain embodiments, the IVT RNA molecule encodes a chimeric antigen receptor (CAR).

In some embodiments, the CAR is specific for binding to one or more antigens. In some embodiments, the antigen comprises at least one viral antigen, a bacterial antigen, a fungal antigen, a parasitic antigen, an influenza antigen, a tumor-associated antigen, a tumor-specific antigen, or any combination thereof.

However, the present disclosure is not limited to any particular agent or combination of agents. In certain embodiments, the composition comprises an adjuvant. In certain embodiments, the composition comprises a nucleic acid molecule encoding an adjuvant. In certain embodiments, the composition comprises a nucleoside-modified RNA encoding an adjuvant.

In certain embodiments, the composition comprises at least one RNA molecule encoding a combination of at least two agents. In certain embodiments, the composition comprises a combination of two or more RNA molecules encoding a combination of two or more agents.

In certain embodiments, the present disclosure provides a method for inducing an immune response in a subject. For example, the method can be used to provide immunity in the subject against a virus, bacteria, fungus, parasite, cancer, or the like. In some embodiments, the method comprises administering to the subject a composition comprising one or more LNP molecule formulated for in vivo targeting of an immune cell comprising one or more RNA encoding at least one antigen, an adjuvant, or a combination thereof.

In certain embodiments, the present disclosure provides a method for gene editing of an immune cell of a subject. For example, the method can be used to provide one or more component of a gene editing system (e.g., a component of a CRISPR system) to an immune cell of a subject. In some embodiments, the method comprises administering to the subject a composition comprising one or more ionizable LNP molecule formulated for targeted T cell delivery comprising one or more nucleoside-modified RNA molecule for gene editing.

In certain embodiments, the method comprises administration of the composition to a subject. In certain embodiments, the method comprises administering a plurality of doses to the subject. In some embodiments, the method comprises administering a single dose of the composition, where the single dose is effective in delivery of the target therapeutic agent.

In other related aspects, the therapeutic agent is an isolated nucleic acid. In certain embodiments, the isolated nucleic acid molecule is one of a DNA molecule or an RNA molecule. In certain embodiments, the isolated nucleic acid molecule is a cDNA, mRNA, siRNA, shRNA or miRNA molecule. In certain embodiments, the isolated nucleic acid molecule encodes a therapeutic peptide such a thrombomodulin, endothelial protein C receptor (EPCR), anti-thrombotic proteins including plasminogen activators and their mutants, antioxidant proteins including catalase, superoxide dismutase (SOD) and iron-sequestering proteins. In some embodiments, the therapeutic agent is an siRNA, miRNA, shRNA, or an antisense molecule, which inhibits a targeted nucleic acid including those encoding proteins that are involved in aggravation of the pathological processes.

In certain embodiments, the nucleic acid comprises a promoter/regulatory sequence such that the nucleic acid is capable of directing expression of the nucleic acid. Thus, the disclosure encompasses expression vectors and methods for the introduction of exogenous nucleic acid into cells with concomitant expression of the exogenous nucleic acid in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In certain embodiments, siRNA is used to decrease the level of a targeted protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, PA (2003); and Gregory J. Hannon, Ed., RNAi A Guide to Gene Silencing, Cold Spring Harbor Laboratory Press, Cold Spring Harbor, NY (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present disclosure also includes methods of decreasing levels of PTPN22 using RNAi technology.

In one aspect, the disclosure includes a vector comprising an siRNA or an antisense polynucleotide. Preferably, the siRNA or antisense polynucleotide is capable of inhibiting the expression of a target polypeptide. The incorporation of a desired polynucleotide into a vector and the choice of vectors are well-known in the art as described in, for example, Sambrook et al. (2012), and in Ausubel et al. (1997), and elsewhere herein.

In certain embodiments, the expression vectors described herein encode a short hairpin RNA (shRNA) therapeutic agents. shRNA molecules are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleave the shRNA to form siRNA.

In order to assess the expression of the siRNA, shRNA, or antisense polynucleotide, the expression vector to be introduced into a cell can also contain either a selectable marker gene or a reporter gene or both to facilitate identification of expressing cells from the population of cells sought to be transfected or infected using a the delivery vehicle of the disclosure. In other embodiments, the selectable marker may be carried on a separate piece of DNA and also be contained within the delivery vehicle. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in one aspect, the delivery vehicle may contain a vector, comprising the nucleotide sequence or the construct to be delivered. The choice of the vector will depend on the host cell in which it is to be subsequently introduced. In a particular embodiment, the vector of the disclosure is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present disclosure to produce polynucleotides, or their cognate polypeptides. Many such systems are commercially and widely available.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the disclosure or the gene construct of the disclosure can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In certain embodiments, the recombinant expression vectors may also contain nucleic acid molecules, which encode a peptide or peptidomimetic.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of host cells. Suitable selectable marker genes are genes encoding proteins such as G418 and hygromycin, which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrawal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queuosine, and wybutosine and the like, as well as acetyl-methyl-, thio- and other modified forms of adenine, cytidine, guanine, thymine, and uridine.

In certain embodiments of the disclosure, an antisense nucleic acid sequence, which is expressed by a plasmid vector is used as a therapeutic agent to inhibit the expression of a target protein. The antisense expressing vector is used to transfect a mammalian cell or the mammal itself, thereby causing reduced endogenous expression of the target protein.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the disclosure may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the disclosure include oligonucleotide derivatives known in the art which have improved biological activity compared to unmodified oligonucleotides (see U.S. Pat. No. 5,023,243).

In certain embodiments of the disclosure, a ribozyme is used as a therapeutic agent to inhibit expression of a target protein. Ribozymes useful for inhibiting the expression of a target molecule may be designed by incorporating target sequences into the basic ribozyme structure, which are complementary, for example, to the mRNA sequence encoding the target molecule. Ribozymes targeting the target molecule, may be synthesized using commercially available reagents (Applied Biosystems, Inc., Foster City, CA) or they may be genetically expressed from DNA encoding them.

In certain embodiments, the therapeutic agent may comprise one or more components of a CRISPR-Cas system, where a guide RNA (gRNA) targeted to a gene encoding a target molecule, and a CRISPR-associated (Cas) peptide form a complex to induce mutations within the targeted gene. In certain embodiments, the therapeutic agent comprises a gRNA or a nucleic acid molecule encoding a gRNA. In certain embodiments, the therapeutic agent comprises a Cas peptide or a nucleic acid molecule encoding a Cas peptide.

In certain embodiments, the agent comprises a miRNA or a mimic of a miRNA. In certain embodiments, the agent comprises a nucleic acid molecule that encodes a miRNA or mimic of a miRNA.

MiRNAs are small non-coding RNA molecules that are capable of causing post-transcriptional silencing of specific genes in cells by the inhibition of translation or through degradation of the targeted mRNA. A miRNA can be completely complementary or can have a region of noncomplementary with a target nucleic acid, consequently resulting in a “bulge” at the region of non-complementarity. A miRNA can inhibit gene expression by repressing translation, such as when the miRNA is not completely complementary to the target nucleic acid, or by causing target RNA degradation, which is believed to occur only when the miRNA binds its target with perfect complementarity. The disclosure also can include double-stranded precursors of miRNA. A miRNA or pri-miRNA can be 18-100 nucleotides in length, or from 18-80 nucleotides in length. Mature miRNAs can have a length of 19-30 nucleotides, or 21-25 nucleotides, particularly 21, 22, 23, 24, or 25 nucleotides. MiRNA precursors typically have a length of about 70-100 nucleotides and have a hairpin conformation. miRNAs are generated in vivo from pre-miRNAs by the enzymes Dicer and Drosha, which specifically process long pre-miRNA into functional miRNA. The hairpin or mature microRNAs, or pri-microRNA agents featured in the disclosure can be synthesized in vivo by a cell-based system or in vitro by chemical synthesis.

In various embodiments, the agent comprises an oligonucleotide that comprises the nucleotide sequence of a disease-associated miRNA. In certain embodiments, the oligonucleotide comprises the nucleotide sequence of a disease-associated miRNA in a pre-microRNA, mature or hairpin form. In other embodiments, a combination of oligonucleotides comprising a sequence of one or more disease-associated miRNAs, any pre-miRNA, any fragment, or any combination thereof is envisioned.

MiRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism.

Modifications can also increase sequence specificity, and consequently decrease off-site targeting. Methods of synthesis and chemical modifications are described in greater detail below.

If desired, miRNA molecules may be modified to stabilize the miRNAs against degradation, to enhance half-life, or to otherwise improve efficacy. Desirable modifications are described, for example, in U.S. Patent Publication Nos. 20070213292, 20060287260, 20060035254. 20060008822. and 2005028824, each of which is hereby incorporated by reference in its entirety.

For increased nuclease resistance and/or binding affinity to the target, the single-stranded oligonucleotide agents featured in the disclosure can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleotide modifications can also increase binding affinity to the target. The inclusion of pyranose sugars in the oligonucleotide backbone can also decrease endonucleolytic cleavage. An oligonucleotide can be further modified by including a 3′ cationic group, or by inverting the nucleoside at the 3′-terminus with a 3-3′ linkage. In another alternative, the 3′-terminus can be blocked with an aminoalkyl group. Other 3′ conjugates can inhibit 3′-5′ exonucleolytic cleavage. While not being bound by theory, a 3′ may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose, and so forth) can block 3′-5′-exonucleases.

In certain embodiments, the miRNA includes a 2′-modified oligonucleotide containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅Q. This modification also increases the nuclease resistance of the modified oligonucleotide. It is understood that the methods and reagents of the present disclosure may be used in conjunction with any technologies that may be developed to enhance the stability or efficacy of an inhibitory nucleic acid molecule.

miRNA molecules include nucleotide oligomers containing modified backbones or non-natural internucleoside linkages. Oligomers having modified backbones include those that retain a phosphorus atom in the backbone and those that do not have a phosphorus atom in the backbone. For the purposes of this disclosure, modified oligonucleotides that do not have a phosphorus atom in their internucleoside backbone are also considered to be nucleotide oligomers. Nucleotide oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriest-ers, and boranophosphates. Various salts, mixed salts and free acid forms are also included.

A miRNA described herein, which may be in the mature or hairpin form, may be provided as a naked oligonucleotide. In some cases, it may be desirable to utilize a formulation that aids in the delivery of a miRNA or other nucleotide oligomer to cells (see, e.g., U.S. Pat. Nos. 5,656,611, 5,753,613, 5,785,992, 6,120,798, 6,221,959, 6,346,613, and 6,353,055, each of which is hereby incorporated by reference).

In some examples, the miRNA composition is at least partially crystalline, uniformly crystalline, and/or anhydrous (e.g., less than 80, 50, 30, 20, or 10% water). In another example, the miRNA composition is in an aqueous phase, e.g., in a solution that includes water. The aqueous phase or the crystalline compositions can be incorporated into a delivery vehicle, e.g., a liposome (particularly for the aqueous phase), or a particle (e.g., a microparticle as can be appropriate for a crystalline composition). Generally, the miRNA composition is formulated in a manner that is compatible with the intended method of administration. A miRNA composition can be formulated in combination with another agent, e.g., another therapeutic agent or an agent that stabilizes an oligonucleotide agent, e.g., a protein that complexes with the oligonucleotide agent. Still other agents include chelators, e.g., EDTA (e.g., to remove divalent cations such as Mg), salts, and RNAse inhibitors (e.g., a broad specificity RNAse inhibitor). In certain embodiments, the miRNA composition includes another miRNA, e.g., a second miRNA composition (e.g., a microRNA that is distinct from the first). Still other preparations can include at least three, five, ten, twenty, fifty, or a hundred or more different oligonucleotide species.

In certain embodiments, the composition comprises an oligonucleotide composition that mimics the activity of a miRNA. In certain embodiments, the composition comprises oligonucleotides having nucleobase identity to the nucleobase sequence of a miRNA, and are thus designed to mimic the activity of the miRNA. In certain embodiments, the oligonucleotide composition that mimics miRNA activity comprises a double-stranded RNA molecule which mimics the mature miRNA hairpins or processed miRNA duplexes.

In certain embodiments, the oligonucleotide shares identity with endogenous miRNA or miRNA precursor nucleobase sequences. An oligonucleotide selected for inclusion in a composition of the present disclosure may be one of a number of lengths. Such an oligonucleotide can be from 7 to 100 linked nucleosides in length. For example, an oligonucleotide sharing nucleobase identity with a miRNA may be from 7 to 30 linked nucleosides in length. An oligonucleotide sharing identity with a miRNA precursor may be up to 100 linked nucleosides in length. In certain embodiments, an oligonucleotide comprises 7 to 30 linked nucleosides. In certain embodiments, an oligonucleotide comprises 7, 8, 9, 10, 11, 12, 13, 14, 15, 16, 17, 18, 19, 20, 21, 22, 23, 24, 25, 26, 28, 29, or 30 linked nucleotides. In certain embodiments, an oligonucleotide comprises 19 to 23 linked nucleosides. In certain embodiments, an oligonucleotide is from 40 up to 50, 60, 70, 80, 90, or 100 linked nucleosides in length.

In certain embodiments, an oligonucleotide has a sequence that has a certain identity to a miRNA or a precursor thereof. Nucleobase sequences of mature miRNAs and their corresponding stem-loop sequences described herein are the sequences found in miRBase, an online searchable database of miRNA sequences and annotation. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence. The compositions of the present disclosure encompass oligomeric compound comprising oligonucleotides having a certain identity to any nucleobase sequence version of a miRNAs described herein.

In certain embodiments, an oligonucleotide has a nucleobase sequence at least 60%, 65%, 70%, 75%, 80%, 85%, 90%, 95%, 97%, 98% or 99% identical to the miRNA over a region of 7, 8, 9, 10, 11, 12, 13, 14, 15, 16, 17, 18, 19, 20, 21, 22, 23, 24, 25, 26, 27, 28, 29, or 30 nucleobases. Accordingly, in certain embodiments the nucleobase sequence of an oligonucleotide may have one or more non-identical nucleobases with respect to the miRNA.

In certain embodiments, the composition comprises a nucleic acid molecule encoding a miRNA, precursor, mimic, or fragment thereof. For example, the composition may comprise a viral vector, plasmid, cosmid, or other expression vector suitable for expressing the miRNA, precursor, mimic, or fragment thereof in a desired mammalian cell or tissue.

### Polypeptide Therapeutic Agents

In other related aspects, the therapeutic agent includes an isolated peptide that modulates a target. For example, In certain embodiments, the peptide of the disclosure inhibits or activates a target directly by binding to the target thereby modulating the normal functional activity of the target. In certain embodiments, the peptide of the disclosure modulates the target by competing with endogenous proteins. In certain embodiments, the peptide of the disclosure modulates the activity of the target by acting as a transdominant negative mutant.

The variants of the polypeptide therapeutic agents may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present disclosure, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

### CAR Agents

In certain embodiments, the mRNA molecule of the disclosure encodes a chimeric antigen receptor (CAR). In certain embodiments, the CAR comprises an antigen binding domain. In certain embodiments, the antigen binding domain is a targeting domain, wherein the targeting domain directs the T cell expressing the CAR to a specific cell or tissue of interest. For example, In certain embodiments, the targeting domain comprises an antibody, antibody fragment, or peptide that specifically binds to an expressed on a pathogenic organism or a tumor cell thereby directing the T cell expressing the CAR to a cell or tissue expressing the antigen.

In certain embodiments, the disclosure relates to an immune cell targeted LNP comprising an agent, wherein the agent comprises a nucleic acid sequence encoding a chimeric antigen receptor (CAR). In certain embodiments, agent comprises an mRNA molecule encoding a CAR. In certain embodiments, the agent comprises a modified nucleoside mRNA molecule encoding a CAR.

In various embodiments, the CAR can be a “first generation,” “second generation,” “third generation,” “fourth generation” or “fifth generation” CAR (see, for example, Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. (2009); Hollyman et al., J. Immunother. 32:169-180 (2009)).

“First generation” CARs for use in the disclosure comprise an antigen binding domain, for example, a single-chain variable fragment (scFv), fused to a transmembrane domain, which is fused to a cytoplasmic/intracellular domain of the T cell receptor chain. “First generation” CARs typically have the intracellular domain from the CD3ζ-chain, which is the primary transmitter of signals from endogenous T cell receptors (TCRs). “First generation” CARs can provide de novo antigen recognition and cause activation of both CD4+ and CD8+ T cells through their CD3ζ chain signaling domain in a single fusion molecule, independent of HLA-mediated antigen presentation.

“Second-generation” CARs for use in the disclosure comprise an antigen binding domain, for example, a single-chain variable fragment (scFv), fused to an intracellular signaling domain capable of activating T cells and a co-stimulatory domain designed to augment T cell potency and persistence (Sadelain et al., Cancer Discov. 3:388-398 (2013)). CAR design can therefore combine antigen recognition with signal transduction, two functions that are physiologically borne by two separate complexes, the TCR heterodimer and the CD3 complex. “Second generation” CARs include an intracellular domain from various co-stimulatory molecules, for example, CD28, 4-1BB, ICOS, OX40, and the like, in the cytoplasmic tail of the CAR to provide additional signals to the cell.

“Second generation” CARs provide both co-stimulation, for example, by CD28 or 4-1BB domains, and activation, for example, by a CD3ζ signaling domain. Preclinical studies have indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL) (Davila et al., Oncoimmunol. 1(9):1577-1583 (2012)).

“Third generation” CARs provide multiple co-stimulation, for example, by comprising both CD28 and 4-1BB domains, and activation, for example, by comprising a CD3ζ activation domain.

“Fourth generation” CARs provide co-stimulation, for example, by CD28 or 4-1BB domains, and activation, for example, by a CD3ζ signaling domain in addition to a constitutive or inducible chemokine component.

“Fifth generation” CARs provide co-stimulation, for example, by CD28 or 4-1BB domains, and activation, for example, by a CD3ζ signaling domain, a constitutive or inducible chemokine component, and an intracellular domain of a cytokine receptor, for example, IL-2RP.

In various embodiments, the CAR can be included in a multivalent CAR system, for example, a DualCAR or “TandemCAR” system. Multivalent CAR systems include systems or cells comprising multiple CARs and systems or cells comprising bivalent/bispecific CARs targeting more than one antigen.

In the embodiments disclosed herein, the CARs generally comprise an antigen binding domain, a transmembrane domain and an intracellular domain, as described above. In a particular non-limiting embodiment, the antigen-binding domain is an scFv specific for binding to a surface antigen of a target cell of interest (e.g., a pathogen or tumor cell.)

### Combinations

In certain embodiments, the composition of the present disclosure comprises a combination of agents described herein. In certain embodiments, a composition comprising a combination of agents described herein has an additive effect, wherein the overall effect of the combination is approximately equal to the sum of the effects of each individual agent. In other embodiments, a composition comprising a combination of agents described herein has a synergistic effect, wherein the overall effect of the combination is greater than the sum of the effects of each individual agent.

A composition comprising a combination of agents comprises individual agents in any suitable ratio. For example, In certain embodiments, the composition comprises a 1:1 ratio of two individual agents. However, the combination is not limited to any particular ratio. Rather any ratio that is shown to be effective is encompassed.

### Cell Targeting Domain

In various embodiments of the disclosure, the LNP of the disclosure is conjugated to a targeting domain specific for binding to a receptor of a target cell.

In certain embodiments, the target cell is a stem cell. Exemplary stem cells that can be targeted by the compositions of the disclosure include, but are not limited to, hematopoietic stem cells and stem cells related to hematopoietic stem cells (e.g., myeloid stem cells and lymphoid stem cells.)

In certain embodiments, the target cell is a peripheral blood mononuclear cell (PBMC).

In one cell the target cell is an immune cell. Exemplary immune cells that can be targeted according by the compositions of the disclosure include, but are not limited to, T cells, B cells, NK cells, antigen-presenting cells, dendritic cells, macrophages, monocytes, neutrophils, eosinophils, and basophils. In certain embodiments, the immune cell is a T cell. In some embodiments, T cells that can be targeted using the compositions of the disclosure can be CD4+ or CD8+ and can include, but are not limited to, T helper cells (CD4+), cytotoxic T cells (also referred to as cytotoxic T lymphocytes, CTL; CD8− T cells), and memory T cells, including central memory T cells (TCM), stem memory T cells (TSCM), stem-cell-like memory T cells (or stem-like memory T cells), and effector memory T cells, for example, T_{EM} cells and T_{EMRA} (CD45RA+) cells, effector T cells, Th1 cells, Th2 cells, Th9 cells, Th7 cells, Th22 cells, Tfh (follicular helper) cells, T regulatory cells, natural killer T cells, mucosal associated invariant T cells (MAIT), and γδ T cells. Major T cell subtypes include T_{N} (naive), T_{SCM} (stem cell memory), T_{CM} (central memory), T_{TM} (Transitional Memory), T_{EM} (Effector memory), and T_{TE}(Terminal Effector), TCR-transgenic T cells, T-cells redirected for universal cytokine-mediated killing (TRUCK), Tumor infiltrating T cells (TIL), CAR-T cells or any T cell that can be used for treating a disease or disorder.

In certain embodiments, the T cells of the disclosure are immunostimulatory cells, i.e., cells that mediate an immune response. Exemplary T cells that are immunostimulatory include, but are not limited to, T helper cells (CD4+), cytotoxic T cells (also referred to as cytotoxic T lymphocytes, CTL; CD8+ T cells), and memory T cells, including central memory T cells (TCM), stem memory T cells (TSCM), stem-cell-like memory T cells (or stem-like memory T cells), and effector memory T cells, for example, TEM cells and TEMRA (CD45RA+) cells, effector T cells, Th1 cells, Th2 cells, Th9 cells, Th7 cells, Th22 cells, Tfh (follicular helper) cells, natural killer T cells, mucosal associated invariant T cells (MAIT), and γδ T cells.

In certain embodiments, the T cell targeting domain binds to CD1, CD2, CD3, CD4, CD5, CD7, CD8, CD16, CD25, CD26, CD27, CD28, CD30, CD38, CD39, CD40L, CD44, CD45, CD62L, CD69, CD73, CD80, CD83, CD86, CD95, CD103, CD119, CD126, CD150, CD153, CD154, CD161, CD183, CD223, CD254, CD275, CD45RA, CXCR3, CXCR5, FasL, IL18R1, CTLA-4, OX40, GITR, LAG3, ICOS, PD-1, leu-12, TCR, TLR1, TLR2, TLR3, TLR4, TLR6, NKG2D, CCR, CCR1, CCR2, CCR4, CCR6, or CCR7.

In certain embodiments, the present disclosure relates to compositions comprising a combination of delivery vehicles conjugated to immune cell targeting domains for targeting multiple immune cells. In certain embodiments, the combination comprises two or more immune cell targeted delivery vehicles, targeting two or more immune cell antigens. In certain embodiments, the two or more immune cell antigens are selected from CD1, CD2, CD3, CD4, CD5, CD7, CD8, CD16, CD25, CD26, CD27, CD28, CD30, CD38, CD39, CD40L, CD44, CD45, CD62L, CD69, CD73, CD80, CD83, CD86, CD95, CD103, CD119, CD126, CD150, CD153, CD154, CD161, CD183, CD223, CD254, CD275, CD45RA, CXCR3, CXCR5, FasL, IL18R1, CTLA-4, OX40, GITR, LAG3, ICOS, PD-1, leu-12, TCR, TLR1, TLR2, TLR3, TLR4, TLR6, NKG2D, CCR, CCR1, CCR2, CCR4, CCR6, and CCR7. In certain embodiments, the combination comprises two or more T cell targeted delivery vehicles, targeting a surface antigen of a CD4+ T cell and a surface antigen of a CD8+ T cell. In certain embodiments, the combination comprises two or more T cell targeted delivery vehicles, targeting CD4 and CD8.

In certain embodiments, the targeting domain is conjugated to the LNP of the disclosure. Exemplary methods of conjugation can include, but are not limited to, covalent bonds, electrostatic interactions, and hydrophobic (“van der Waals”) interactions. In certain embodiments, the conjugation is a reversible conjugation, such that the delivery vehicle can be disassociated from the targeting domain upon exposure to certain conditions or chemical agents. In some embodiments, the conjugation is an irreversible conjugation, such that under normal conditions the delivery vehicle does not dissociate from the targeting domain.

In some embodiments, the conjugation comprises a covalent bond between an activated polymer conjugated lipid and the targeting domain. The term “activated polymer conjugated lipid” refers to a molecule comprising a lipid portion and a polymer portion that has been activated via functionalization of a polymer conjugated lipid with a first coupling group. In certain embodiments, the activated polymer conjugated lipid comprises a first coupling group capable of reacting with a second coupling group. In certain embodiments, the activated polymer conjugated lipid is an activated pegylated lipid. In certain embodiments, the first coupling group is bound to the lipid portion of the pegylated lipid. In some embodiments, the first coupling group is bound to the polyethylene glycol portion of the pegylated lipid. In certain embodiments, the second functional group is covalently attached to the targeting domain.

The first coupling group and second coupling group can be any functional groups known to those of skill in the art to together form a covalent bond, for example under mild reaction conditions or physiological conditions. In some embodiments, the first coupling group or second coupling group are selected from the group consisting of maleimides, N-hydroxysuccinimide (NHS) esters, carbodiimides, hydrazide, pentafluorophenyl (PFP) esters, phosphines, hydroxymethyl phosphines, psoralen, imidoesters, pyridyl disulfide, isocyanates, vinyl sulfones, alpha-haloacetyls, aryl azides, acyl azides, alkyl azides, diazirines, benzophenone, epoxides, carbonates, anhydrides, sulfonyl chlorides, cyclooctyne, aldehydes, and sulfhydryl groups. In some embodiments, the first coupling group or second coupling group is selected from the group consisting of free amines (—NH₂), free sulfhydryl groups (—SH), free hydroxide groups (—OH), carboxylates, hydrazides, and alkoxyamines. In some embodiments, the first coupling group is a functional group that is reactive toward sulfhydryl groups, such as maleimide, pyridyl disulfide, or a haloacetyl. In certain embodiments, the first coupling group is a maleimide.

In certain embodiments, the second coupling group is a sulfhydryl group. The sulfhydryl group can be installed on the targeting domain using any method known to those of skill in the art. In certain embodiments, the sulfhydryl group is present on a free cysteine residue. In certain embodiments, the sulfhydryl group is revealed via reduction of a disulfide on the targeting domain, such as through reaction with 2-mercaptoethylamine. In certain embodiments, the sulfhydryl group is installed via a chemical reaction, such as the reaction between a free amine and 2-iminothilane or N-succinimidyl S-acetylthioacetate (SATA).

In some embodiments, the polymer conjugated lipid and targeting domain are functionalized with groups used in “click” chemistry. Bioorthogonal “click” chemistry comprises the reaction between a functional group with a 1,3-dipole, such as an azide, a nitrile oxide, a nitrone, an isocyanide, and the link, with an alkene or an alkyne dipolarophiles. Exemplary dipolarophiles include any strained cycloalkenes and cycloalkynes known to those of skill in the art, including, but not limited to, cyclooctynes, dibenzocyclooctynes, monofluorinated cyclcooctynes, difluorinated cyclooctynes, and biarylazacyclooctynone.

In certain embodiments, the targeting domain is conjugated to the LNP using maleimide conjugation.

### Targeting Domain

In certain embodiments, the composition comprises a targeting domain that directs the delivery vehicle to a target immune cell. The targeting domain may comprise a nucleic acid, peptide, antibody, small molecule, organic molecule, inorganic molecule, glycan, sugar, hormone, and the like that targets the particle to a site in particular need of the therapeutic agent. In certain embodiments, the particle comprises multivalent targeting, wherein the particle comprises multiple targeting mechanisms described herein. In certain embodiments, the targeting domain of the delivery vehicle specifically binds to a target associated with a site in need of an agent comprised within the delivery vehicle. For example, the targeting domain may be chosen to recognize a ligand that acts as a cell surface marker on target cells associated with a particular disease state. Such a target can be a protein, protein fragment, antigen, or other biomolecule that is associated with the targeted site. In some embodiments, the targeting domain is an affinity ligand which specifically binds to a target. In certain embodiments, the target (e.g. antigen) associated with a site in need of a treatment with an agent. In some embodiments, the targeting domain may be co-polymerized with the composition comprising the delivery vehicle. In some embodiments, the targeting domain may be covalently attached to the composition comprising the delivery vehicle, such as through a chemical reaction between the targeting domain and the composition comprising the delivery vehicle. In some embodiments, the targeting domain is an additive in the delivery vehicle. Targeting domains of the instant disclosure include, but are not limited to, antibodies, antibody fragments, proteins, peptides, and nucleic acids.

In various embodiments, the targeting domain binds to a cell surface molecule of a cell of interest. For example, in various embodiments, the targeting domain binds to a cell surface molecule of an endothelial cell, a stem cell, or an immune cell.

### Peptides

In certain embodiments, the targeting domain of the disclosure comprises a peptide. In certain embodiments, the peptide targeting domain specifically binds to a target of interest.

The peptides of the present disclosure may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The variants of the peptides according to the present disclosure may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present disclosure, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one peptide to a sequence of a second peptide. Variants are defined to include peptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence. The present disclosure includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The peptides of the disclosure can be post-translationally modified. For example, post-translational modifications that fall within the scope of the present disclosure include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the disclosure may include unnatural amino acids formed by post-translational modification or by introducing unnatural amino acids during translation.

### Antibodies

In certain embodiments, the targeting domain of the disclosure comprises an antibody, or antibody fragment. In certain embodiments, the antibody targeting domain specifically binds to a target of interest. Such antibodies include polyclonal antibodies, monoclonal antibodies, Fab and single chain Fv (scFv) fragments thereof, bispecific antibodies, heteroconjugates, human and humanized antibodies.

The antibodies may be intact monoclonal or polyclonal antibodies, and immunologically active fragments (e.g., a Fab or (Fab)2 fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Such antibodies may be produced in a variety of ways, including hybridoma cultures, recombinant expression in bacteria or mammalian cell cultures, and recombinant expression in transgenic animals. The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmacokinetic activity may be generated in a bacterial expression system. Single chain Fv fragments show low immunogenicity.

### Methods

In another aspect, the present disclosure provides a method of delivering a cargo to the placenta of a pregnant subject, the method comprising administering to the subject a therapeutically effective amount of at least one lipid nanoparticle (LNP) of the present disclosure. In certain embodiments, the LNP comprises at least one ionizable lipid. In certain embodiments, the LNP comprises at least one helper or neutral lipid. In certain embodiments, the LNP comprises cholesterol and/or a derivative thereof. In certain embodiments, the LNP comprises at least one polymer conjugated lipid and/or a modified derivative thereof. In certain embodiments, the LNP comprises at least one cargo molecule. In certain embodiments, the at least one cargo molecule is at least partially encapsulated in the LNP. In certain embodiments, the LNP comprises an epidermal growth factor (EGFR) targeting domain. In certain embodiments, the EGFR targeting domain is covalently conjugated to at least one component of the LNP.

In certain embodiments, the LNP comprises at least one LNP of the present disclosure.

In certain embodiments, the cargo is at least one selected from the group consisting of a nucleic acid, small molecule, protein, therapeutic agent, antibody, and any combinations thereof.

In certain embodiments, the cargo is a nucleic acid. In certain embodiments, the nucleic acid is DNA or RNA. In certain embodiments, the nucleic acid is selected from the group consisting of mRNA, cDNA, pDNA, microRNA, siRNA, modified RNA, antagomir, antisense molecule, and any combinations thereof.

In certain embodiments, the cargo is mRNA. In certain embodiments, the mRNA encodes VEGF.

In certain embodiments, the LNP is administered as a pharmaceutical composition. In certain embodiments, the pharmaceutical composition comprises at least one pharmaceutically acceptable carrier.

In another aspect, the present disclosure provides a method of treating, preventing, and/or ameliorating a placental disease and/or disorder in a subject in need thereof, the method comprising administering to a subject a therapeutically effective amount of at least one lipid nanoparticle (LNP). In certain embodiments, the LNP comprises at least one ionizable lipid. In certain embodiments, the LNP comprises at least one helper lipid. In certain embodiments, the LNP comprises cholesterol and/or a derivative thereof. In certain embodiments, the LNP comprises at least one polymer conjugated lipid. In certain embodiments, the LNP comprises at least one cargo molecule. In certain embodiments, the at least one cargo molecule is at least partially encapsulated in the LNP.

In certain embodiments, the placental disease and/or disorder is pre-eclampsia. In certain embodiments, the placental disease and/or disorder is fetal growth restriction (FGR). In certain embodiments, the placental disease and/or disorder is intrauterine growth restriction (IUGR). In certain embodiments, the placental disease and/or disorder is placenta previa. In certain embodiments, the placental disease and/or disorder is placenta accreta. In certain embodiments, the placental disease and/or disorder is placenta increta. In certain embodiments, the placental disease and/or disorder is placenta percreta.

In certain embodiments, the LNP comprises at least one LNP of the present disclosure.

In certain embodiments, the cargo is at least one selected from the group consisting of a nucleic acid, small molecule, protein, therapeutic agent, antibody, and any combinations thereof.

In certain embodiments, the cargo is a nucleic acid. In certain embodiments, the nucleic acid is DNA or RNA. In certain embodiments, the nucleic acid is selected from the group consisting of mRNA, cDNA, pDNA, microRNA, siRNA, modified RNA, antagomir, antisense molecule, and any combinations thereof.

In certain embodiments, the cargo is mRNA. In certain embodiments, the mRNA encodes VEGF.

In certain embodiments, the LNP is administered as a pharmaceutical composition. In certain embodiments, the pharmaceutical composition comprises at least one pharmaceutically acceptable carrier.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as non-human primates, cattle, pigs, horses, sheep, cats, and dogs.

Pharmaceutical compositions that are useful in the methods of the disclosure may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the disclosure may further comprise one or more additional pharmaceutically active agents.

Controlled- or sustained-release formulations of a pharmaceutical composition of the disclosure may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the disclosure may be prepared, packaged, or sold in a formulation suitable for pulmonary administration via the buccal cavity. Such a formulation may comprise dry particles which comprise the active ingredient and which have a diameter in the range from about 0.5 to about 7 micrometers, and preferably from about 1 to about 6 micrometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 micrometers and at least 95% of the particles by number have a diameter less than 7 micrometers. More preferably, at least 95% of the particles by weight have a diameter greater than 1 micrometer and at least 90% of the particles by number have a diameter less than 6 micrometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the disclosure are known in the art and described, for example in Remington's Pharmaceutical Sciences (1985, Genaro, ed., Mack Publishing Co., Easton, PA), which is incorporated herein by reference.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder contemplated herein. An effective amount of therapeutic (i.e., composition) necessary to achieve a therapeutic effect may vary according to factors such as the activity of the particular therapeutic employed; the time of administration; the rate of excretion of the composition; the duration of the treatment; other drugs, compounds or materials used in combination with the composition; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic composition of the disclosure is from about 0.01 mg/kg to 100 mg/kg of body weight/per day of active agent (i.e., nucleic acid). One of ordinary skill in the art would be able to study the relevant factors and make the determination regarding the effective amount of the therapeutic composition without undue experimentation.

The composition may be administered to an animal as frequently as several times daily, or it may be administered less frequently, such as once a day, once a week, once every two weeks, once a month, or even less frequently, such as once every several months or even once a year or less. It is understood that the amount of composition dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic composition to produce the desired therapeutic effect in association with the required pharmaceutical vehicle. The dosage unit forms of the disclosure are dictated by and directly dependent on (a) the unique characteristics of the therapeutic composition and the particular therapeutic effect to be achieved, and (b) the limitations inherent in the art of compounding/formulating such a therapeutic composition for the treatment of a disease or disorder in a patient.

In certain embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It will be readily apparent to one skilled in the art that the frequency of administration of the various combination compositions of the disclosure will vary from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physician taking all other factors about the patient into account.

The amount of active agent of the composition(s) of the disclosure for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of active agent (i.e., nucleic acid) present in the composition of the disclosure is from about 0.5 μg and about 5,000 mg. In some embodiments, a dose of active agent present in the composition of the disclosure used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of the composition of the disclosure, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Administration

Routes of administration of any of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intracerebroventricular, subcutaneous, intravenous, intraperitoneal, intramuscular, intrasternal injection, and kidney dialytic infusion techniques. In certain embodiments, the composition of the present disclosure is administered intracerebroventricularly.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### EXAMPLES

Various embodiments of the present application can be better understood by reference to the following Examples which are offered by way of illustration. The scope of the present application is not limited to the Examples given herein.

### Materials and Methods

### Ionizable Lipid Synthesis

All ionizable lipids excluding DLin-MC3-DMA (Cayman Chemical, Ann Arbor, MI) were synthesized utilizing methods known to those of ordinary skill in the art. Briefly, the polyamine core (Enamine, Kyiv, Ukraine), denoted here as 1 (482), 2 (488), 3 (490), 4 (494), 5 (497), 480, 493, 489, or 200, was combined with excess epoxide, including epoxydodecane (C12, denoted as A), epoxytetradecane (C14, denoted as B), or epoxyhexadecane (C16, denoted as C) (MilliporeSigma, Burlington, MA), in 4 mL glass scintillation vials under gentle stirring with a magnetic stir bar for 2 days at 80° C. The ethanol was evaporated using a Rotovap R-300 rotary evaporator (Buchi, New Castle, DE) and used for LNP formulation.

The polyamine cores used in this work were 3-[4-(3-{[2-(4-{3-[4-(3-aminopropyl)piperazin-1-yl]propyl}piperazin-1 yl)ethyl]amino}propyl)piperazin-1-yl]propan-1-amine (denoted as 480), 1-N-{2-[4-(4-aminocyclohexyl)piperazin-1-yl]ethyl}cyclohexane-1,4-diamine (denoted as 482), {2-[2-(2-aminoethoxy)ethoxy]ethyl}[2-(4-{2-[2-(2-aminoethoxy)ethoxy]ethyl}piperazin-1-yl)ethyl]amine (denoted as 488), 10-(4-{2-[(10-aminodecyl)amino]ethyl}piperazin-1-yl)decan-1-amine (denoted as 489), 3-(4-{2-[(3-amino-2-ethoxypropyl)amino]ethyl}piperazin-1-yl)-2-ethoxypropan-1-amine (denoted as 490), [1-({[2-(4-{[1-(aminomethyl)cyclohexyl]methyl}piperazin-1-yl)ethyl]amino}methyl)cyclohexyl]methanamine (denoted as 493), 2-{2-[4-(2-{[2-(2-aminoethoxy)ethyl]amino}ethyl)piperazin-1-yl]ethoxy}ethan-1-amine (denoted as 494), and 1-[4-(3-{2-[2-(3-aminopropoxy)ethoxy]ethoxy}propyl)piperazin-1-yl]-7,10,13-trioxa-3-azahexadecan-16-amine (denoted as 497).

The epoxide tails used herein were 1,2-epoxydodecane (denoted as C12), 1,2-epoxytetradecane (denoted as C14), and 1,2-epoxyhexadecane (denoted as C16). After 2 d, the ethanol was evaporated using a Rotovapor R-300 rotary evaporator (Buchi, New Castle, DE) to isolate crude product. Liquid chromatography-mass spectrometry (LC-MS) spectra were acquired to confirm molecular identity using an SQD equipped with an Acquity UPLC (Milford, MA) using a C8 column with a 2 min wash followed by a gradient mobile phase from 50% water (1% trifluoroacetic acid) and 50% acetonitrile (1% trifluoroacetic acid) to 100% acetonitrile.

The nomenclature used to describe the ionizable lipids herein comprises the identity of the polyamine core (e.g., “494” or “4”) and the epoxide with which the polyamine core is functionalized (e.g., “C12” or “A”) by nucleophilic addition of each primary (i.e., addition to two epoxides) or secondary amine (i.e., addition to one epoxide) present in the polyamine core.

For example, in the context of ionizable lipids, the terms “C12-494” and “A4” are used interchangeably herein to refer to:

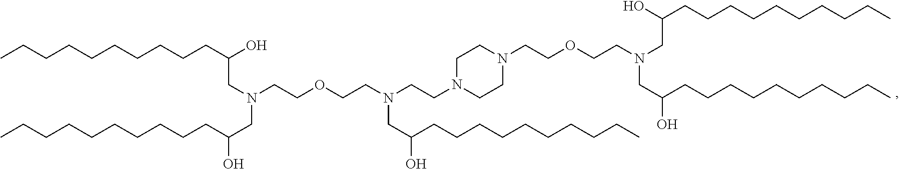 [SMILES: CCCCCCCCCCC(O)CN(CCOCCN(CC(O)CCCCCCCCCC)CC(O)CCCCCCCCCC)CCN1CCN(CCOCCN(CC(O)CCCCCCCCCC)CC(O)CCCCCCCCCC)CC1]

1,1′-((2-(2-(4-(2-((2-(2-(bis(2-hydroxydodecyl)amino)ethoxy)ethyl)(2-hydroxydodecyl)amino)ethyl)piperazin-1-yl)ethoxy)ethyl)azanediyl)bis(dodecan-2-ol).

The following ionizable lipids described herein, utilizing the nomenclature defined herein, have been characterized by mass spectroscopy (Table 1).

TABLE 1
Selected characterization data for exemplary
ionizable lipids of the present disclosure

| Ionizable Lipid | Calculated Mass (m/z) | Observed Mass (m/z) |
|---|---|---|
| C12-480 | 1417.41 | 1418.90 |
| C12-482 | 1244.90 | 1245.69 |
| C12-488 | 1313.16 | 1313.72 |
| C12-489 | 1361.38 | 1361.92 |
| C12-490 | 1253.11 | 1253.67 |
| C12-493 | 1301.24 | 1301.79 |
| C12-494 | 1225.05 | 1225.58 |
| C12-497 | 1457.37 | 1457.86 |
| C14-480 | 1557.66 | 1559.05 |
| C14-482 | 1385.15 | 1385.87 |
| C14-488 | 1453.41 | 1453.95 |
| C14-489 | 1501.63 | 1502.11 |
| C14-490 | 1393.36 | 1393.89 |
| C14-493 | 1441.49 | 1441.97 |
| C14-494 | 1365.30 | 1365.84 |
| C14-497 | 1597.62 | 1599.00 |
| C16-480 | 1697.91 | 1698.40 |
| C16-482 | 1525.40 | 1526.06 |
| C16-488 | 1593.66 | 1594.06 |
| C16-489 | 1641.88 | 1642.26 |
| C16-490 | 1533.61 | 1535.03 |
| C16-493 | 1581.74 | 1583.00 |
| C16-494 | 1505.55 | 1506.03 |
| C16-497 | 1737.87 | 1739.15 |
| C12-200 | 1136.90 | 1137.55 |

mRNA and b-DNA Synthesis

Luciferase mRNA was synthesized using in vitro transcription with linearized plasmids (pLuc19) encoding codon-optimized firefly luciferase and T7 RNA polymerase (Megascript, Ambion) as previously described in the literature. mRNA was transcribed with the pseudouridine modification and 130 nucleotide-long poly(A) tails. RNA was capped using the m7G capping kit with 2′-O-methyltransferase (ScriptCap, CellScript) to obtain the cap previously described as cap1. Finally, mRNA was purified by fast protein liquid chromatography (FPLC) using an Akta Purifier (GE Healthcare) as previously described in the literature. Correct mRNA synthesis was validated by denaturing or native agarose gel electrophoresis and mRNA was stored frozen at −80° C. for later use. mCherry and VEGF mRNA were synthesized using similar methods, by replacing the pLuc19 template with templates containing coding sequences for the appropriate gene.

61 nucleotide b-DNAs were designed and synthesized and purified by Integrated DNA Technologies (Coralville, IA). Briefly, the 61 b-DNA nucleotide sequence contained five phosphorothioate bonds at each end with a 10 nucleotide barcode region in the center. 10 additional random nucleotides were included at the 3′ end of the barcode region and the 5′ and 3′ ends of each b-DNA contained priming sites for Illumina adapters (Illumina, San Diego, CA).

For VEGF mRNA production, dsDNA IVT template was purchased from Integrated DNA Technologies as a gBlock consisting of a T7 RNA polymerase promoter, a 5′ UTR derived from tobacco etch virus, a mouse codon-optimized VEGF₁₆₄-A coding sequence, and a 3′ UTR derived from xenopus globin. The template was amplified via PCR with Q5 High-Fidelity DNA Polymerase (New England BioLabs, Ipswich, MA) and purified using Monarch PCR & DNA Cleanup spin columns (New England BioLabs). IVT was performed using the HiScribe T7 High Yield RNA Synthesis kit (New England BioLabs) and full pseudouridine substitution was achieved by replacing UTP with N1-Methylpseudouridine-5′-Triphosphate (TriLink Biotechnologies, San Diego, CA). Co-transcriptional capping was achieved using the CleanCap Reagent AG Cap1 analog (TriLink Biotechnologies). RNA was purified using Monarch RNA Cleanup spin columns (New England BioLabs). RNA transcripts were polyadenylated using E. coli Poly(A) Polymerase (New England BioLabs) and purified once again using RNA Cleanup spin columns. mRNA product integrity was validated using native agarose gel electrophoresis and stored at −80° C. for later use.

### LNP Formulation and Characterization (General)

Unless otherwise indicated, LNPs were formulated at a 10:1 ratio of ionizable lipid: mRNA weight ratio. In one non-limiting example, the ionizable lipid was combined in an ethanol phase with cholesterol (MilliporeSigma), 1,2-dioleoyl-sn-glycero-3-phosphoethanolamine (DOPE, Avanti Polar Lipids, Alabaster, AL), and 1,2-dimyristoyl-sn-glycero-3-phosphoethanolamine-N-[methoxy(polyethylene glycol)-200](ammonium salt) (C14-PEG2000, Avanti Polar Lipids) at a molar ratio of 35:16:46.5:2.5 to a total volume of 112.5 μL. A separate aqueous phase was prepared with 25 μg of luciferase mRNA in 10 mM citrate buffer (pH=3) to a total volume of 337.5 μL. With a syringe pump, the ethanol and aqueous phases were combined to form LNPs via chaotic mixing using a microfluidic device designed with herringbone features as previously described in the literature. LNPs were dialyzed against 1×PBS with a molecular weight cutoff of 20 kDa for 2 h, sterilized using a 0.22 μm filter, and stored at 4° C. for later use. All materials were prepared and handled ribonuclease-free throughout synthesis, formulation, and characterization steps.

For dynamic light scattering (DLS) measurements, 10 μL of LNP solution was diluted 100-fold in 1×PBS in 4.5 mL disposable cuvettes (Thermo Fisher Scientific, Waltham, MA). Three measurements each with at least 10 runs were recorded for each sample using a Zetasizer Nano (Malvern Instruments, Malvern, UK). Z-average diameter and polydispersity index (PDI) are reported as mean±standard deviation (n=3 measurements). Surface ionization measurements to calculate the pKa of each LNP formulation were performed as previously described in the literature. Buffered solution containing 150 mM sodium chloride, 20 mM sodium phosphate, 20 mM ammonium acetate, and 25 mM ammonium citrate was adjusted to pH 2 to 12 in increments of 0.5. 125 μL of each pH-adjusted solution and 5 μL of each LNP formulation were plated in triplicate in black 96-well plates. 6-(p-toluidinyl)naphthalene-2-sulfonic acid (TNS) was then added to each well to a final TNS concentration of 6 M. The fluorescence intensity was read on an Infinite 200 Pro plate reader (Tecan, Morrisville, NC) at an excitation wavelength of 322 nm and an emission wavelength of 431 nm. Using univariate least squares linear regression, the pKa was taken as the pH corresponding to half-maximum fluorescence intensity (i.e., 50% protonation). Encapsulation efficiencies of each LNP formulation were measured using a Quant-iTRiboGreen (Thermo Fisher Scientific) assay as previously described in the literature. Each LNP sample was diluted to approximately 2 ng/μL in two microcentrifuge tubes containing either 1×tris-EDTA (TE) buffer or TE buffer supplemented with 0.1% (v/v) Triton X-100 (MilliporeSigma) and allowed to incubate for 20 min to achieve lysis of LNPs by Triton X-100. LNPs in TE buffer, LNPs in Triton X-100, and mRNA standards were then plated in triplicate in black 96-well plates and the RiboGreen fluorescent detection reagent was added per the manufacturer's instructions. Five min later, fluorescence intensity was read on an Infinite 200 Pro plate reader (Tecan) at an excitation wavelength of 490 nm and an emission wavelength of 520 nm. RNA content was estimated by comparison to a standard curve estimated using univariate least squares linear regression (LSLR). Encapsulation efficiency was calculated as (B−A)/B*100 where A is the measured RNA content in TE buffer (i.e., free/unencapsulated RNA) and B is the measured RNA content in Triton X-100 (i.e., total RNA). Encapsulation efficiencies are reported as mean±standard deviation (n=3 technical replicates).

b-DNA Library LNP Formulation and Characterization

A library of 98 LNPs were each formulated at a 10:1 molar ratio of ionizable lipid to nucleic acid with a unique ionizable lipid and excipient formulation (Table 7). For all LNPs excluding LNP 98, the ionizable lipid was combined in an ethanol phase with cholesterol (MilliporeSigma), 1,2-dioleoyl-sn-glycero-3-phosphoethanolamine (DOPE, Avanti Polar Lipids, Alabaster, AL), and 1,2-dimyristoyl-sn-glycero-3-phosphoethanolamine-N-[methoxy(polyethylene glycol)-2000](ammonium salt) (C14-PEG₂₀₀₀, Avanti Polar Lipids) to a total volume of 112.5 μL. For LNP 98, the ionizable lipid Dlin-MC3-DMA was combined in an ethanol phase with cholesterol (MilliporeSigma), 1,2-distearoyl-sn-glycero-3-phosphocholine (DSPC, Avanti Polar Lipids), and 1,2-dimyristoyl-rac-glycero-3-methoxypolyethylene glycol-2000 (DMG-PEG, Avanti Polar Lipids) to 112.5 μL. A separate aqueous phase was prepared with 25 μg of either barcoded DNA (b-DNA), luciferase mRNA, or VEGF mRNA in 10 mM citrate buffer to a total volume of 337.5 μL. For high-throughput screening with b-DNA, ethanol and aqueous phases were combined via pipette mixing to form LNPs. For luciferase mRNA and VEGF mRNA LNPs, ethanol and aqueous phases were combined via chaotic mixing using a microfluidic device designed with herringbone features to form LNPs. LNPs were dialyzed against 1×PBS in either Pierce 96-well microdialysis plates (ThermoFisher Scientific) or Slide-A-Lyzer G2 dialysis cassettes with a molecular weight cutoff filter of 20 kDa (Thermo Fisher Scientific) for 2 h, sterilized using 0.22 μm filters, and stored at 4° C. for later use.

For dynamic light scattering (DLS) measurements, 10 μL of LNP was diluted 100× in 1×PBS in 4.5 mL disposable cuvettes (Thermo Fisher Scientific). For zeta potential measurements, 20 μL of LNP was diluted 50× in ultrapure water in disposable folded capillary cells (Malvern Instruments, Malvern, UK). For high-throughput screening, z-average diameter, polydispersity index, and zeta potential are reported as the mean from 3 measurements for each sample (Table 8) recorded using a Zetasizer Nano (Malvern Instruments, Malvern, UK). For LNPs #55 and #98, mean zeta potential of 4 distinct formulations (with at least 3 measurements per formulation) is reported and a nested t test was used to compare means.

b-DNA and mRNA encapsulation efficiencies of each LNP formulation was measured using a Quant-iT-OliGreen (Thermo Fisher Scientific) assay and a Quant-iT-RiboGreen (Thermo Fisher Scientific) assay, respectively. Each LNP sample was diluted 100× in either 1×tris-EDTA (TE) buffer or TE buffer containing 0.1% (v/v) Triton X-100 (MilliporeSigma). LNPs in TE buffer, LNPs in Triton X-100, and standards were plated in quadruplicate in black 96-well plates and either the OliGreen or RiboGreen fluorescent detection reagent was added to each well per the manufacturer's instructions. Fluorescent intensity was measured using an Infinite 200 Pro plate reader (Tecan, Morrisville, NC). Encapsulated b-DNA and mRNA was estimated by comparison to a standard curve estimated using univariate least squares linear regression. Encapsulation efficiency was calculated as (B−A)/B×100 where A is the measured nucleic acid content in TE buffer and B is the measured nucleic acid content in Triton X-100. For high-throughput screening, encapsulation efficiency is reported as the mean from 4 measurements for each sample (Table 7). All materials were prepared and handled nuclease-free throughout synthesis, formulation, and characterization steps.

b-DNA Library Preparation and Next Generation Sequencing

A constant volume of each of the 98 LNP formulations encapsulating a unique b-DNA oligomer was used to generate the injection pool, which was then combined with a naked, unencapsulated b-DNA oligo. The pooled LNPs were administered to non-pregnant mice and gestational day E16 pregnant mice via tail vein injection. 6 h following administration, mice were euthanized with CO₂ and the heart, lung, liver, kidneys, spleen, uterus, fetuses, and placentas were collected into SPEX 5 mL polyethylene vials with 9.5 mm steel grinding balls (SPEX Sample Prep, Metuchen, NJ) on dry ice. Organs were snap frozen using liquid nitrogen and a 2010 Geno/Grinder (SPEX Sample Prep) was used to generate powdered tissue homogenates by two consecutive homogenization cycles of 30 s at a speed of 750 strokes/min.

Approximately 50 mg of powdered tissue was weighed out into 1.5 mL tubes and incubated in 750 μL of lysis buffer containing 100 mM Tris-HCl, 5 mM EDTA, 0.2% SDS, 200 mM NaCl, and 0.2 mg/mL proteinase K (ThermoFisher Scientific) overnight at 37° C. 1 μL of 4 mg/mL RNAse A (Promega, Madison, WI) was added to each sample and incubated for 1 h at 37° C. and spun for 5 min at 15,000 g. Oligos were extracted from 300 μL of sample supernatant using an Oligo Clean and Concentrator kit (Zymo Research, Irvine, CA) according to manufacturer's instructions. PCR on eluted DNA was performed using Phusion High-Fidelity DNA Polymerase for 35 cycles (New England BioLabs).

PCR products were purified using gel electrophoresis with a 3% agarose gel (ThermoFisher Scientific) in Tris-acetate-EDTA running buffer (ThermoFisher Scientific). Amplified b-DNA was excised from the gel and purified using a Zymo Gel DNA Recovery kit (Zymo Research) according to the manufacturer's instructions. The pooled, uninjected library of LNP formulations was processed using the same protocol above and amplified with a unique reverse primer (i.e., organ barcode). The next generation sequencing library was balanced and pooled using a Quant-iT-PicoGreen assay (ThermoFisher Scientific). Quality control using an Agilent bioAnalyzer system was performed to check library purity and measure concentration for loading the flow cell at a concentration of 4 nM. Next generation sequencing was performed using multiplexed runs on the Illumina MiSeq (Illumina).

Normalized delivery of a particular b-DNA LNP to a particular organ was calculated as the ratio of two frequencies. Briefly, within one organ sample, the sequencing reads from each b-DNA were divided by the sum of reads from all b-DNAs in the organ sample. Similarly, within the uninjected LNP pool, the sequencing reads from each b-DNA were divided by the sum of reads from all b-DNAs in the uninjected pool. Normalized delivery for each b-DNA to a particular organ was calculated as the ratio of these two frequencies. To generate volcano plots, one-way ANOVAs were performed for each organ to compare normalized delivery across LNP formulations. Post hoc Student's t tests to the C12-200 LNP using the Holm-Bonferroni method were used to correct for multiple comparisons. To generate correlation coefficient heatmaps, the squared Pearson's correlation coefficient of mean normalized delivery was calculated for each pair of organs. All analysis and generation of plots for b-DNA LNP delivery was performed using Version 2023.03.0+386 of RStudio.

In Vitro LNP-Mediated Luciferase mRNA Delivery in BeWo Placental Cells

BeWo b30 syncytiotrophoblast cells were cultured i 501 n Dulbecco's Modified Eagle Medium with L-glutamine (DMEM, Gibco, Dublin, Ireland) supplemented with 10% (v/v) FBS (Gibco) and 1% (v/v) penicillin-streptomycin (Gibco). Cells were plated at 50,000 cells per well in 100 μL of medium in tissue culture treated 96-well plates and were left to adhere overnight. To evaluate the in vitro luciferase expression mediated by the 17-LNP library, the BeWo cells were treated at a dose of 50 ng of mRNA per 50,000 cells. As a positive control, the transfection reagent Lipofectamine MessengerMAX (Thermo Fisher Scientific) was combined with luciferase mRNA for 10 min as per the manufacturer's protocol and was used to treat cells at the same dose of mRNA. 24 h after treatment with LNPs or Lipofectamine, excess medium was removed and 50 μL of 1× reporter lysis buffer (Promega, Madison, WI) was added to each well, followed by 100 μL of luciferase assay substrate (Promega) was added to each well. After 10 min of incubation, luminescence intensity was quantified using an Infinite 200 Pro plate reader (Tecan). The luminescence signal for each condition was normalized by dividing by the luminescence signal from untreated cells. To evaluate cytotoxicity of LNP formulations, additional plates were seeded with cells and dosed with LNPs as described above. After 24 h, 100 μL of CellTiter-Glo (Promega) was added to each well and the luminescence corresponding to ATP concentration was quantified using a plate reader following 10 min of incubation. Luminescence for each group was normalized by dividing by the luminescence signal from untreated cells. Luciferase expression and percent viability are reported as mean±standard deviation (n≥8 independent observations from 3 biological replicates).

### Animal Experiments

Non-pregnant female mice (6-8 weeks old, approximately 25 g average weight) and time-dated pregnant female mice (varied age, approximately 30 g average weight) were purchased from the Jackson Laboratory (Bar Harbor, ME).

Luciferase mRNA LNP Delivery in Non-Pregnant and Pregnant Mice

Non-pregnant female and gestational age E16 pregnant mice were injected with luciferase mRNA LNPs or PBS via tail vein injection at a dose of 0.6 mg mRNA/kg body mass. 6 h later, luciferase imaging was performed using an in vivo imaging system (IVIS, PerkinElmer, Waltham, MA). 10 min before imaging, mice were injected intraperitoneally with D-luciferin and potassium salt (Biotium, Fremont, CA) at 150 mg reagent/kg body weight. Then, mice were euthanized with CO₂ and the heart, lung, liver, kidney, spleen, and uterus were removed and imaged. For pregnant mice, the uterus was subsequently dissected to remove the fetuses and placentas for imaging. To quantify luminescence flux, the Living Image Software (PerkinElmer) was used to place a rectangular region of interest (ROI) around the organ or fetus of interest. An equal size ROI was placed in an area without any luminescent signal in the same image. Normalized flux was calculated by dividing the flux from the organ or fetus ROI by the flux from the background ROI. Reported bioluminescence for the heart, lung, liver, kidney, spleen, and uterus for non-pregnant and pregnant mice represents the mean±standard error of the mean (SEM) (n=3 biological replicates). The spleen to liver ratio was calculated by dividing the normalized liver bioluminescence values by the normalized spleen bioluminescence values for each treatment group; the reported values represent the mean±SEM (n=3 biological replicates). The reported bioluminescence for fetuses and placentas represents the mean±SEM (n≥18 fetuses or placentas from 3 biological replicates).

Characterizing Cell-Level Delivery of 552 mCherry mRNA LNPs to the Placenta

Gestational age E16 pregnant mice were injected with mCherry mRNA LNPs or PBS via tail vein injection at a dose of 1 mg mRNA/kg of body mass. 12 h later, mice were euthanized with CO₂ and the uterus was removed. The placentas were dissected from the uterus and immediately placed in 2 mL of deionized H₂O on ice. Placentas and the 2 mL of water were passed through 100 μm cell strainers (MilliporeSigma) to generate cell suspensions. Cell suspensions were subsequently treated with 20 μL of 2000 U/mL DNase I (New England Biolabs, Ipswich, MA) and 200 μL of 10×DNase I buffer (New England Biolabs) for 30 minutes at room temperature to prevent cell aggregation mediated by released genomic DNA from lysed cells. Next, 2 mL of ACK lysis buffer (Thermo Fisher Scientific) was added to cell suspensions for 5 min to lyse red blood cells. Cells were centrifuged at 300 g for 5 min, supernatant was removed, and cells were resuspended in 2 mL of 1×PBS with 2 mM EDTA. 0.5 mL of the resuspended cells was taken for subsequent blocking and immunofluorescent staining. 0.5 μL of TruStain FcX™ PLUS (ant-mouse CD16/32) antibody (Biolegend, San Diego, CA) was added to cells for 10 minutes on ice. Next, samples were stained for cell surface markers for 30 min in the dark at 4° C. with 2 μL of Brilliant Violet 421 (BV421) anti-mouse CD45 antibody (Biolegend, cat. #03134) and 1 μL of FITC anti-mouse CD31 antibody (Biolegend, cat. #02506). Following two washes and resuspension in 1×PBS with 2 mM EDTA, cells were fixed and permeabilized for intracellular staining using the Cyto-Fast™ Fix/Perm Buffer set (Biolegend) per manufacturer's instructions. Cells were stained intracellularly with Alexa Fluor (AF700) anti-mouse cytokeratin 7 (Novus Biologicals, Littleton, CO, cat. #NBP2-47940AF700). Data was acquired using a BD LSR II flow cytometer equipped with violet, blue, green, and red lasers. At least 10,000 events corresponding to singlets were acquired for all experimental samples. Thresholds for positivity were determined using fluorescence-minus-one (FMO) controls, with a representative gating scheme is shown in FIG. 9. The reported measurements for percent mCherry positive endothelial cells and trophoblasts represent the mean±SEM (n≥4 independent observations from 1 distinct biological replicate).

In vivo LNP-mediated VEGF mRNA delivery and toxicity Gestational age E16 pregnant mice were injected with VEGF mRNA LNPs or PBS via tail vein injection at a dose of 1 mg mRNA/kg body mass. Either 6 h or 48 h later, blood was collected via retro orbital bleeding into Microtainer blood collection tubes (BD, Franklin Lakes, NJ). Blood was allowed to clot for 2 h at room temperature before centrifuging for 20 min at 2000 g. Serum was removed, aliquoted, and stored at −20° C. for later use. After blood collection, mice were euthanized via CO₂, the uterus was removed, and placentas were dissected. Where permitted by litter size, at least three placentas were individually placed in 5 mL polyethylene vials containing a 9.5 mm steel grinding ball (SPEX Sample Prep, Metuchen, NJ) and prefilled with 1 mL of 1×PBS. Similarly, livers were removed, cut into small pieces, and placed into two 5 mL polyethylene vials prefilled with 1 mL of 1×PBS. A 2010 Geno/Grinder (SPEX Sample Prep) was used to generate tissue homogenates by two consecutive homogenization cycles of 30 s at a speed of 750 strokes/minute. Tissue homogenates were transferred to 1.5 mL Eppendorf tubes and frozen at −20° C. After two freeze thaw cycles to lyse cell membranes, samples were centrifuged for 5 min at 5000 g and the supernatant was collected 597 and stored at −20° C. for later use. A mouse VEGF Quantikine ELISA kit (R & D Systems, Minneapolis, MN) was used to evaluate VEGF levels 6 h and 48 h following LNP or PBS treatment in the serum, placentas, and livers per the manufacturer's instructions. The measured VEGF concentration for placentas and livers was normalized to the mass of total protein in the tissue homogenate as measured via absorbance measurements at a wavelength of 260 nm on an Infinite Pro 200 plate reader using a NanoQuant plate (Tecan). The reported measurements for VEGF concentration represent the mean±SEM (for serum: n=6 independent observations from 3 distinct biological replicates, for livers: n=18 independent observations from 3 distinct biological replicates, for placentas: n≥18 independent observations from 3 distinct biological replicates). To assess VEGF mRNA LNP-mediated liver toxicity, serum alanine transaminase (ALT) and aspartate aminotransferase (AST) enzyme levels were evaluated 48 h following LNP or PBS administration using colorimetric assay kits (Cayman Chemical) per the manufacturer's instructions. The reported measurements for ALT and AST levels represent the mean±SEM (n=6 independent observations from 3 distinct biological replicates). Placental inflammation was evaluated 48 h after treatment with VEGF mRNA LNPs or PBS using a colorimetric mouse cytokine ELISA kit (Signosis, Santa Clara, CA) per the manufacturer's instructions. Placenta tissue homogenate for each mouse was pooled from at least three placentas. For each cytokine, the reported measurements for relative cytokine concentration are normalized to the average optical density measurements from the PBS-treated group. Data represent the mean±SEM (n=3 independent observations per cytokine).

In Vitro Luciferase mRNA LNP Delivery and Cellular Uptake

The Hep G2 hepatocellular carcinoma and Jurkat T cell leukemia cell lines were purchased from ATCC; the BeWo b30 choriocarcinoma cell line was provided by Dr. Dan Huh at the University of Pennsylvania. All cell lines tested negative for mycoplasma at the University of Pennsylvania's Cell Center. Hep G2 and BeWo b30 cells were cultured in Dulbecco's Modified Eagle Medium with L-glutamine (DMEM, Gibco, Dublin, Ireland) supplemented with 10% v/v fetal bovine serum (FBS, Gibco) and 1% v/v penicillin-streptomycin (Gibco). Jurkat cells were cultured in Roswell Park Memorial Institute (RPMI) 1640 media with L-glutamine (Gibco) with the same supplements.

For luciferase mRNA expression experiments, cells were plated in Opti-MEM Reduced Serum Medium (Gibco) in 96-well plates (ThermoFisher Scientific) at densities of 10,000 cells/100 μL (Hep G2), 60,000 cells/100 μL (Jurkat), or 20,000 cells/100 μL (BeWo b30). Hep G2 and BeWo b30 cells were plated and allowed to adhere overnight before treating. Luciferase mRNA LNPs were incubated with either human recombinant apolipoprotein E4 (ApoE4, ThermoFisher Scientific) or mouse recombinant β2-GPI (ThermoFisher Scientific) at doses of 0, 0.1, 0.25, 0.5, 0.75, and 1 μg of protein per μg of lipid for 15 min at 37° C. with gentle shaking at 300 rpm. Hep G2 cells were treated with 5 ng of encapsulated luciferase mRNA Jurkat and BeWo b30 cells were treated with 20 ng of encapsulated luciferase mRNA. 24 h after LNP treatment, medium was removed and 50 μL of 1× reporter lysis buffer (Promega, Madison, WI) was added to each well followed by 100 μL of luciferase assay substrate (Promega). After 10 min of incubation, luminescence intensity was quantified using an Infinite 200 Pro plate reader (Tecan). Normalized luciferase expression was calculated first by subtracting luminescence signal from wells containing untreated cells and then by dividing by the mean luminescence signal from cells treated with uncoated (i.e., no protein) LNPs. Normalized luciferase expression is reported as mean±SEM of n=5 biological replicates with n=5 technical replicates each. Nested one-way ANOVAs with post hoc Student's t tests using the Holm-Šidák correction for multiple comparisons were used to compare normalized luciferase expression to uncoated LNPs across protein concentrations.

For cellular uptake confocal microscopy experiments, Hep G2, Jurkat, and BeWo b30 cells were plated in Opti-MEM Reduced Serum Medium in Nunc Lab-Tek II 4-Chamber slides (ThermoFisher Scientific) at densities of 200,000 cells/1 mL (Hep G2 and BeWo b30) or 400,000 cells/1 mL (Jurkat). Hep G2 and BeWo b30 cells were plated and allowed to adhere overnight before treating. For Jurkat cells, chambers were coated with 50 μg/mL poly-L-lysine (MilliporeSigma) in 10 mM Tris-HCl (ThermoFisher Scientific) for 30 min and then allowed to dry overnight. Luciferase mRNA LNPs were labeled with 1% (v/v) Vybrant DiD Cell-Labeling Solution (ThermoFisher Scientific) for 15 min at 25° C. with gentle shaking at 300 rpm. DiD-labeled luciferase mRNA LNPs were incubated with either human recombinant ApoE4 or mouse recombinant β2-GPI at doses of 0 or 0.75 μg of protein per μg of lipid for 15 min at 37° C. with gentle shaking at 300 rpm. Hep G2, Jurkat, and BeWo b30 cells were treated with 200 ng of encapsulated luciferase mRNA. 30 min after treatment, medium was removed, cells were washed with 1×PBS, and fixed with 4% paraformaldehyde (MilliporeSigma) for 15 min. Cells were then washed with 1×PBS and counterstained with 1 μg/mL DAPI (ThermoFisher Scientific) for 5 min. Finally, slides were mounted to glass coverslips (ThermoFisher Scientific) with ProLong Diamond Antifade Mountant (ThermoFisher Scientific) overnight before imaging. Slides were imaged using a Leica Stellaris 5 confocal laser scanning microscope equipped with a fixed 405 nm DAPI laser and an extended tunable white light laser with a 20× objective.

For cellular uptake flow cytometry experiments, Hep G2, Jurkat, and BeWo b30 cells were plated in Opti-MEM Reduced Serum Medium in 96-well plates at a density of 50,000 cells/100 μL. Hep G2 and BeWo b30 cells were plated and allowed to adhere overnight before treating. Luciferase mRNA LNPs were labeled with DiD and coated with protein as described above and used to treat Hep G2, Jurkat, and BeWo b30 cells with 50 ng of encapsulated luciferase mRNA. 30 min after LNP treatment, medium was removed, and cells were washed with 1×PBS with 2 mM EDTA. Single cell suspensions were analyzed via flow cytometry for DiD fluorescence intensity using the Guava easyCyte flow cytometer equipped with blue, green, and red lasers (Luminex, Austin, TX). At least 10,000 events corresponding to singlets were acquired for all experimental samples. The percent of DiD⁺ cells is reported as mean±SEM of n=4 biological replicates with n=6 technical replicates each. Representative histograms are shown from samples with values for the percent of DiD⁺ cells closest to the mean for each treatment group. Nested one-way ANOVAs with post hoc Student's t tests using the Holm-Šidák correction for multiple comparisons were used to compare the percent of DiD⁺ cells across treatment groups.

Biodistribution and Luciferase mRNA Expression in Healthy and Pre-Eclamptic Pregnant Mice

LNPs encapsulating luciferase mRNA were labeled with DiD as described above and administered to non-pregnant and gestational day E16 pregnant mice via tail vein injection at a dose of 1 mg mRNA/kg body mass. 12 h following administration, organs were dissected, and luminescence imaging was performed as described above; fluorescence intensity imaging was also performed using an in vivo imaging system with a filter pair for DiD. Luminescent and fluorescent flux were calculated using ROIs as described above. Reported luminescent and fluorescent flux for the lung, liver, and spleen represents the mean±SEM (n=4 biological replicates). Reported luminescent and fluorescent flux for the fetuses and placentas represents the mean±SEM for each mouse (n=4 biological replicates each with n=5 to 10 fetuses and placentas depending on litter size). Statistical analyses for luminescent and fluorescent flux are the same as those described above.

Following imaging, spleens and placentas were collected into 2 mL of dH₂O and placed on ice. Organs were passed through 100 μm cell strainers (MilliporeSigma) to generate cell suspensions. Placenta cell suspensions were treated with 1% of 2000 U/mL DNase I (New England BioLabs) and 10% of 10×DNase I buffer (New England BioLabs) for 30 min at room temperature. ACK lysis buffer (ThermoFisher Scientific) was then added to spleen and placenta cell suspensions for 5 min, cells were spun at 500 g for 5 min, and the supernatant was removed. ACK lysis was repeated until red blood cells were removed, and the resulting pellet was resuspended in 1×PBS with 2 mM EDTA. 0.5 μL of TruStain FcX PLUS (anti-mouse CD16/32) antibody (BioLegend, San Diego, CA) was added to each sample for 10 min at 4° C.

Spleen samples were stained for cell surface markers for 30 min at 4° C. with 5 μL of Brilliant Violent 421 anti-mouse CD45 antibody, 5 μL of Brilliant Violet 711 anti-mouse CD19 antibody, 2 μL of FITC anti-mouse CD3 antibody, 2 μL of PE anti-mouse CD11c antibody, and 5 μL of PE-Cy7 anti-mouse CD11b antibody (BioLegend). Placenta samples were stained for cell surface markers for 30 min at 4° C. with 3 μL of Brilliant Violet 421 anti-mouse CD45 antibody and 1.5 μL of FITC anti-mouse CD31 antibody (BioLegend). Placenta samples were then washed, fixed, and permeabilized using the Cyto-Fast Fix/Perm buffer kit (BioLegend) per the manufacturer's instructions. Placental cells were then stained intracellularly with 3 μL of PE anti-mouse cytokeratin 7 antibody (Novus Biologicals, Littleton, CO).

Data was acquired using a BD LSR II flow cytometer (BD, Franklin Lakes, NJ) equipped with violet, blue, green, and red lasers. At least 50,000 events corresponding to singlets were acquired for all experimental samples. Thresholds for positivity were determined using fluorescence-minus-one (FMO) controls. For cell types in the spleen, the percent of DiD⁺ cells are reported as the mean±SEM (n=4 biological replicates). Representative histograms are shown from spleen CD3⁺ T cells with values for the percent of DiD+ cells closest to the mean for each treatment group. One-way ANOVAs with post hoc Student's t tests using the Holm-Šidák correction for multiple comparisons were used to compare the percent of DiD⁺ cells across treatment groups. For cell types in the placenta, the percent of DiD⁺ cells are reported as the mean±SEM (n=4 biological replicates with n=4 to 6 placentas each depending on litter size). Representative histograms are shown from placenta CD45⁺ immune cells with values for the percent of DiD⁺ cells closest to the mean for each treatment group. Nested one-way ANOVAs with post hoc Student's t tests using the Holm-Šidák correction for multiple comparisons were used to compare the percent of DiD⁺ cells across treatment groups.

Placentas were also collected for immunofluorescent analysis; after dissection, placentas were immediately placed in 10% neutral buffered formalin (MilliporeSigma) for 24 h. Samples were then dehydrated in ethanol, embedded in paraffin wax, and sectioned into 4 μm sections longitudinally from the center of the placenta. Sections were then stained for cytokeratin 7 (primary: rabbit anti-mouse cytokeratin 7 antibody (Abcam, Cambridge, United Kingdom); secondary: PE donkey anti-rabbit antibody (BioLegend)) or endomucin (primary: goat anti-mouse endomucin antibody (R & D Systems, Minneapolis, MN; secondary: FITC donkey anti-goat antibody (Abcam)) and counterstained with DAPI. Slides were then mounted to coverslips and imaged using a Leica Stellaris 5 confocal laser scanning microscope with a 20× objective.

### Pre-Eclampsia Model and Immunophenotyping

Time-dated pregnant mice arrived on gestational day E5 and were randomly divided into 3 cohorts; on E7, 200 μL of either 1×PBS or lipopolysaccharide (LPS, MilliporeSigma) was administered via intraperitoneal injection at a dose of 1 μg reagent/kg body mass to induce an early onset model of pre-eclampsia. Mice were weighed daily from gestational day E6 to E16 and daily weight change was calculated for gestational days E7 to E16 by subtracting the initial body weight measurement from E6. Daily weight change is reported as mean±SEM (n=8 biological replicates). Pregnant mice were trained using the CODA Monitor Noninvasive Blood Pressure System (Kent Scientific Corporation, Torrington, CT) on gestational days E5 and E6 before recording a preliminary blood pressure reading on E7. Maternal mean arterial pressure (MAP) was recorded daily at the same time of day from E7 to E16. Reported blood pressure measurements represent the mean±SEM (n=8 biological replicates averaged from n=2 to 4 readings each). Two-way ANOVAs with post hoc Student's t tests using the Holm-Šidák correction for multiple comparisons were used to compare daily weight change or mean blood pressure across treatment groups and gestational days.

LNPs encapsulating VEGF mRNA were administered to mice via tail vein injection at a dose of 1 mg mRNA/kg body mass. 6 h later, blood was collected retro-orbitally with non-heparinized capillary tubes (ThermoFisher Scientific) into Microtainer blood collection tubes (BD). Blood was allowed to clot for 2 h at room temperature before spinning for 20 min at 2000 g to collect serum, which was subsequently stored at −20° C. for further analysis. Similarly, at the study endpoint, blood was collected retro-orbitally for serum and flow cytometry analysis before euthanizing the mice with CO₂ and dissecting fetuses, placentas, and spleens. Fetuses and placentas were weighed individually with a precision balance (Mettler Toledo, Columbus, OH). As litter size typically varies from 5 to 10 pups in C57BL6 mice, normalized fetal and placental weight were calculated by multiplying by the litter size. Normalized fetal and placental weight are plotted using violin plots where long dashed lines represent the median and short dashed lines represent the 25^{th} and 75^{th} percentile (n=8 biological replicates with n=5 to 10 fetuses and placentas each depending on litter size). Nested one-way ANOVAs with post hoc Student's t tests using the Holm-Šidák correction for multiple comparisons were used to compare fetal or placental weight across treatment groups. Representative images of fetuses are shown from fetuses with weight measurements closest to the median for each treatment group.

Spleens and two to three placentas per mouse, depending on litter size, were prepared for immunophenotyping analysis via flow cytometry as described above. Similarly, blood was prepared for flow cytometry with multiple rounds of red blood cell lysis using ACK lysis buffer until cell pellets were clear. Blood and spleen cell pellets were resuspended in 1×PBS with 1% BSA and placenta cell pellets were resuspended in 1×PBS with 2 mM EDTA. 0.5 μL of TruStain FcX PLUS (anti-mouse CD16/32) antibody (BioLegend) was added to each sample for 10 min at 4° C. Blood, spleen, and placenta cell samples were stained for surface markers for 30 min at 4° C. with 1 μL of Spark 387 anti-mouse CD8 antibody, 2 μL of Brilliant Violet 421 anti-mouse CD4 antibody, 2 μL of Brilliant Violet 711 anti-mouse CD19 antibody, 2 μL PerCP anti-mouse CD45 antibody, 2 μL PE anti-mouse CD25 antibody, 2 μL PE-Cy7 anti-mouse CD11b antibody, 1 μL AlexaFluor 700 anti-mouse CD11c antibody, and 2 μL APC anti-mouse CD3 antibody (BioLegend).

Data was acquired using a BD LSRFortessa flow cytometer equipped with ultraviolet, violet, blue, yellow/green, and red lasers. At least 50,000 events corresponding to singlets were acquired for all spleen and placenta experimental samples and at least 10,000 events corresponding to singlets were acquired for blood experimental samples. Thresholds for positivity were determined using fluorescence-minus-one (FMO) controls. For cell types in the blood and spleen, the cell population frequencies are reported as the mean±SEM (n=8 biological replicates). One-way ANOVAs with post hoc Student's t tests using the Holm-Šidák correction for multiple comparisons were used to compare cell population frequencies across treatment groups. For cell types in the placenta, cell population frequencies are reported as the mean±SEM (n=8 biological replicates with n=2 to 3 placentas each depending on litter size). Nested one-way ANOVAs with post hoc Student's t tests using the Holm-Šidák correction for multiple comparisons were used to compare the cell population frequencies across treatment groups.

After weight measurements, three placentas per mouse were fixed for preparing histological sections as described above and stained using hematoxylin and eosin (H&E) stains to visualize placental vasculature. An EVOS FL Auto 2 widefield microscope (ThermoFisher Scientific) was used to image H&E-stained placental sections in the labyrinth region of the placenta. Mean blood vessel area was calculated as previously described. Briefly, two H&E-stained sections were prepared for each placenta and three images were taken in the labyrinth region for each section. The analyze particles tool in ImageJ was used to calculate the mean vessel area for each image. Reported mean blood vessel area represents the mean±SEM (n=8 biological replicates with 3 placentas per mouse, 2 sections per placenta, and 3 images per section for a total of 144 images per treatment group. A nested one-way ANOVA with post hoc Student's t tests using the Holm-Šidák correction for multiple comparisons was used to compare mean blood vessel area across treatment groups.

Mouse Quantikine ELISA kits (R & D systems) were used to evaluate VEGF, sFlt-1, TNF-α, IL-6, and IFN-γ levels in serum 6 h after LNP administration (E1.5) and at the study endpoint (E17) per the manufacturer's instructions. Colorimetric assay kits (Cayman Chemical) were used to evaluate alanine transaminase (ALT) and aspartate aminotransferase (AST) levels in serum 6 h after LNP administration (E11.5) and at the study endpoint (E17) per the manufacturer's instructions. The reported levels in serum represent the mean±SEM (n=8 biological replicates). Two-way ANOVAs with post hoc Student's t tests using the Holm-Šidák correction for multiple comparisons was used to compare serum levels across treatment groups and gestational days.

### Histology

Where permitted by litter size, at least three placentas per mouse were collected for histological analysis to assess morphological changes in placental structure and vascularization 48 h after treatment with VEGF mRNA LNPs or PBS. After dissection, placentas were immediately placed in cold 10% neutral buffered formalin (MilliporeSigma) and fixed for 24 h. Samples were then dehydrated in ethanol and embedded in paraffin wax. Two 4 m sections were taken longitudinally from the center of the placenta and stained using either hematoxylin and eosin (H&E) or rabbit anti-human CD31 antibody (Cell Signaling Technology, Danvers, MA) followed by horseradish peroxidase (HRP) chromogenic detection. Brightfield images were taken using an EVOS FL Auto 2 microscope (Thermo Fisher) with 4×, 20×, or 40× objectives. For 40×H&E images, total blood vessel area was quantified using ImageJ using the analyze particles tool. For 20×CD31 images, fetal blood vessel area was quantified using ImageJ by adjusting the color threshold to identify only brown CD31+ stained regions and using the analyze particles tool. The reported measurements for both total and fetal blood vessel area represent the median and 25th and 75th quartiles denoted with dashed lines (n≥54 independent observations from 3 distinct biological replicates).

### Protein Modification and Purification

Anti-human or anti-mouse EGFR antibodies (Biolegend, San Diego, CA, USA; ThermoFisher Scientific, Waltham, MA) were first concentrated using 10 kDa molecular weight filter columns in azide-free phosphate buffered saline (PBS). Antibodies were then functionalized with DBCO via reaction with a 30-fold molar excess of TFP-PEG(4)-DBCO (ThermoFisher Scientific, Waltham, MA) in anhydrous DMSO for 2 hours at room temperature. Unreacted TFP-PEG(4)-DBCO was removed using 40K Zeba Dye and Biotin Removal spin columns (ThermoFisher Scientific, Waltham, MA). Final protein concentration was measured using a Qubit Protein Quantification Assay (ThermoFisher Scientific, Waltham, MA). The purified DBCO-labeled antibodies were stored at 4° C. for later use.

### Statistical Analysis

All tests of significance were performed at the a=0.05 significance level unless otherwise stated. For both in vitro luciferase expression and percent viability, one-way analyses of variance (ANOVAs) with post hoc Student's t tests using the Holm-Šidák correction for multiple comparisons were used to compare responses across formulation to Lipofectamine-treated cells. GraphPad Prism's ROUT method with Q=5% was used to identify outliers, which were subsequently removed from the analysis. For dose-dependent in vitro luciferase expression and percent cell viability, two-way ANOVAs with post hoc Student's t tests were used to compare responses across formulation and dose. For in vivo luciferase expression in non-reproductive organs and the spleen to liver ratio, two-way ANOVAs with post hoc Student's t tests were used to compare response across treatment groups and organ. For in vivo luciferase expression in the uterus, fetuses, and placentas one-way ANOVAs with post hoc Student's t tests was used to compare responses across treatment groups. For serum, liver, and placenta VEGF levels, ALT and AST measurements, and total and fetal blood vessel areas, one-way ANOVAs with post hoc Student's t were used to compare responses across treatment groups. For cytokine levels, a two-way ANOVA with post hoc Student's t tests was used to compare responses across treatment groups and cytokine type.

### Generation of Antibody Conjugated LNPs

To functionalize LNPs with antibody, DBCO-labeled antibody was incubated with azide-containing LNPs at a 5-fold molar excess for 4 hours at 25° C. with gentle shaking and then left to incubate overnight at 4° C. to complete the reaction. Antibody-conjugated LNPs were purified using size exclusion chromatography. Briefly, a column was packed with Sepharose CL-6B (Sigma, St. Louis, MO) and rinsed with 1×PBS to clear ethanol from the system. Ab-LNPs were passed through the column and collected in ˜200 μL fractions. Collected fractions were measured via A260/A280 reading on an Infinite 200 Pro plate reader (Tecan, Morrisville, NC); all fractions containing mRNA were pooled and concentrated using 100 kDa filters. The final Ab-LNP solution was stored at 4° C. for later use.

In Vitro LNP-Mediated Luciferase mRNA Delivery to JEG-3 Placental Cells

JEG-3 choriocarcinoma cells (ATCC #HTB-36) were cultured in Dulbecco's Modified Eagle Medium supplemented with 10% FBS (Gibco) and 1% penicillin-streptomycin (Gibco) and maintained at 37° C. and 5% CO₂. For all experiments, JEG-3 cells were plated at a density of 50,000 cells/well in 100 μL Opti-MEM Reduced Serum Medium (Gibco) in tissue-culture treated 96-well plates and then left to adhere overnight. To assess LNP-mediated luciferase expression in vitro, cells were treated with 50 ng mRNA per 50,000 cells. After 24 hours, media was removed, and cells were incubated with 0.1% Triton-X for 3 minutes. 100 μL of luciferase assay substrate (Promega) was then added to each well, and cells were left to incubate at room temperature for 10 minutes. Luminescence was detected using an Infinite 200 Pro plate reader (Tecan). Normalized luciferase expression for each treatment group was calculated by first subtracting the background readings from untreated cells and then by dividing by the average luminescence signal from the control azide formulation (A1) treated wells. Normalized luciferase expression is reported as mean±standard deviation of the mean (SEM) of n=5 biological replicates (averaged from n=5 technical replicates each).

To evaluate LNP-mediated cytotoxicity, JEG-3 cells were plated and dosed as described above. After 24 hours, 100 μL of CellTiter-Glo (Promega) was added to each well. Cells were incubated for 10 minutes at room temperature, and luminescence was quantified using a plate reader. The luminescence signal for each treatment group was normalized to untreated wells. Percent cell viability is reported as mean±standard deviation of the mean (SEM) of n=5 biological replicates (averaged from n=5 technical replicates each).

For dose response experiments, JEG-3 cells were plated and dosed at 10, 25, 50, 100, and 250 ng mRNA per 50,000 cells. Luciferase expression and cytotoxicity were measured as described above. Normalized luciferase expression is reported as mean±SEM of n=5 biological replicates (averaged from n=3 technical replicates each) and percent cell viability is reported as mean±SEM of n=5 biological replicates (averaged from n=3 technical replicates each).

### Sterol Lipid Nanoparticle (LNP) Formulation and Characterization

LNPs were formulated at a weight ratio of 10:1 of ionizable lipid to mRNA. Ionizable lipid (C12-494, C12-200, SM-102 or MC3 (Cayman Chemical, Ann Arbor, MI)), phospholipid (1,2-dioleoyl-sn-glycero-3-phosphoethanolamine (DOPE) or 1,2-distearoyl-sn-glycero-3-phosphocholine (DSPC) (Avanti Polar Lipids, Alabaster, AL)), sterol (cholesterol (Sigma Aldrich, St. Louis, MO), campesterol, β-sitosterol or stigmastanol (Cayman Chemical)), and lipid-PEG (1,2-dimyristoyl-sn-glycero-3-phosphoethanolamine-N-[methoxy(polyethylene glycol)-2000](ammonium salt) (C14-PEG₂ₖ), 1,2-dimyristoyl-rac-glycero-3-methoxypolyethylene glycol-2000 (DMG-PEG₂ₖ) or 1,2-distearoyl-sn-glycero-3-phosphoethanolamine-N-[biotinyl(polyethylene glycol)-2000](ammonium salt) (DSPE-PEG₂ₖ Biotin) (Avanti Polar Lipids), were combined in an ethanol phase at various molar ratios specified in Table 10 and Table 12. mRNA with the N¹-methylpseudouridine modifications (Trilink Biotechnologies, San Diego, CA) was dissolved in 10 mM citric acid buffer (pH 3) to produce the aqueous phase. Syringe pumps (Harvard Apparatus, Holliston, MA) were used to combine the ethanol and aqueous phases via chaotic mixing in a microfluidic device at a 1:3 volumetric ratio. Chaotic mixing was induced through herringbone features on the microfluidic device as previously described in the literature. After synthesis, LNPs were dialyzed against 1×PBS for 2 h in cassettes with a 20 kDa molecular weight cut off (Thermo Fisher Scientific, Waltham, MA) and sterilized with 0.22 μm filters. LNPs were stored at 4° C. until use.

Encapsulation efficiencies of each LNP formulation was measured using a Quant-iT-RiboGreen RNA assay (ThermoFisher Scientific). Briefly, each LNP formulation was diluted 80-fold in either 1×tris-EDTA (TE) buffer or TE buffer with 0.1% Triton X-100 (Millipore Sigma) and incubated for 20 minutes at room temperature to ensure particle lysis. LNPs in TE or Triton X-100 and mRNA standards were plated in quadruplicate in black 96-well plates and the RiboGreen reagent was added to each well. After 5 minutes of incubation at room temperature, the fluorescence intensity was read on a plate reader at an excitation wavelength of 480 nm and an emission wavelength of 520 nm. Encapsulation efficiencies were calculated as [(RNA content in TE buffer-RNA content in Triton X-100)/RNA content in Triton X-100]*100. Encapsulation efficiencies are reported as mean±standard deviation of n=4 replicates.

Particle size as determined by Dynamic light scattering (DLS) were conducted using a DynaPro® Plate Reader III (Wyatt Technology, Santa Barbara, CA). LNPs were diluted in 1×PBS for DLS measurements and for each sample, three measurements with at least five runs were recorded. Z-average size and polydispersity index (PDI) are reported as mean±standard deviation of n=3 replicates. Zeta potential measurements were conducted using a Zetasizer Nano (Malvern Instruments). LNPs were diluted in water at for zeta potential measurements, three measurements with at least 10 runs were recorded.

### Atomic Force Microscopy of Lipid Nanoparticles

To formulate LNPs for AFM, SM-102, C12-200 and C12-494 LNPs were formulated with a 1:10 molar substitution of DSPE-PEG₂ₖ Biotin with C14-PEG₂ₖ and MC3 LNPs were formulated with a 1:8 molar substitution of DSPE-PEG₂ₖ Biotin with DMG-PEG₂ₖ. DLS and ribogreen were performed as described elsewhere herein to confirm the integrity of the LNPs. LNPs were then deposited onto a glass slide to enable stiffness characterization by mechanical contact. LNP immobilization and synthesis of biotinylated PLL-PEG was adapted from previously described protocols. Briefly, a hydrophobic pen was used to encircle a small ring with a 1 cm diameter onto a glass coverslip to create a sampling area. A solution of PLL-PEG-biotin was added to the sampling area and allowed to incubate for 20 min prior to rinsing with 1×PBS. A solution of neutravidin at 1 mg/mL in 1×PBS was then added to each ring for 10 min prior to rinsing with 1×PBS. Finally, biotin-modified LNPs were added to each ring for 10 min and then rinsed with 1×PBS. Additional 1×PBS was added to the sampling area to enable force-depth collection in liquid.

Force indentation data was collected on an Asylum MFP-3D SA atomic force microscope (Asylum Research, Santa Barbara, CA, USA) using a cantilever with a diamond-like carbon spherical indenter probe with a 40 nm diameter and a spring constant, k=0.2 N/m (Nano Tools, Munich, Germany) calibrated by the thermal noise method. The AFM was performed in contact mode with a trigger force of 5 nN. At least 15 independent force-depth cycles were collected at different sample locations on for each of the LNPs. For all C12-494 cholesterol analog LNPs, AFM measurements were performed on two different batches of LNPs. The Young's modulus (E) was determined by fitting the force-distance curve by the Hertz model through instrument-integrated analysis. The Young's modulus is reported as mean±standard deviation for each LNP. A one-way ANOVA with post hoc Student's t tests using the Holm-Šidák correction for multiple comparisons was used to compare Young's modulus across LNP formulations.

In Vitro mCherry mRNA LNP Delivery and Cellular Uptake to Trophoblast Cells

BeWo b30 cells were cultured in Dulbecco's Modified Eagle Medium (Gibco, Dublin, Ireland) supplemented with 10% FBS (Gibco) and 1% penicillin-streptomycin (Gibco) and maintained at 37° C. and 5% CO₂. For flow cytometry experiments, BeWo b30 cells were plated at 100,000 cells per well in 300 μL of media in 48-well plates and left to adhere overnight. mCherry mRNA LNPs were labeled with 1% (v/v) DiR Cell-Labeling Solution (Thermo Fisher Scientific) for 15 min at 25° C. with gentle shaking at 300 rpm. BeWo b30 cells were treated with LNPs at a dose of 100 ng of mRNA. 1, 4, or 18 h after treatment with LNPs, medium was removed and the cells were washed with 1×PBS with 2 mM EDTA. Single cell suspensions were analyzed via flow cytometry for DiR and mCherry fluorescence using a BD LSR II flow cytometer equipped with violet, blue, green and red lasers. For each sample, at least 10,000 events within the singlet gate were collected. Normalized DiR or mCherry median fluorescent intensity (MFI) for each treatment group was quantified by normalizing to the cells treated with Chol LNPs at each time point. Normalized MFI is reported as mean±standard deviation of n=4 biological replicates (averaged from n=3 technical replicates). A nested one-way ANOVA with post hoc Student's t tests using the Holm-Šidák correction for multiple comparisons was used to compare normalized MFI across treatment groups to the Chol LNP group.

To quantify cytotoxicity of the cells following 24 h of treatment with LNPs, BeWo b30 cells were plated at 50,000 cells per well in 100 μL of media in 96-well plates and left to adhere overnight. Cells were dosed with 50 ng of luciferase mRNA LNPs and 24 h of treatment with LNPs, 100 μL of CellTiter-Glo (Promega, Madison, WI) was added to each well. After 10 minutes of incubation at room temperature, the luminescence signal was measured using a plate reader (Tecan, Morisville, NC) and the luminescence signal for each treatment group was normalized to untreated wells. Percent cell viability is reported as mean±standard deviation of n=4 biological replicates (averaged from n=3 technical replicates). A one-way ANOVA with post hoc Student's t tests using the Holm-Šidák correction for multiple comparisons were used to compare cell viability across treatment groups.

In Vitro LNP-Mediated Luciferase mRNA Delivery to Trophoblast Cells

BeWo b30 cells were plated at 50,000 cells per well in 100 μL of media in 96-well plates and left to adhere overnight. BeWo b30 cells were treated with luciferase mRNA LNPs at a dose of 50 ng of mRNA. 24 h after treatment with LNPs, medium was removed from each well and the cells were resuspended in 50 μL of 0.1% Triton-X and 100 μL of luciferase assay substrate (Promega). After 5 minutes of incubation at room temperature, the luminescence signal was measured using a plate reader (Tecan). Relative luminescence signal was calculated by first subtracting the background readings from untreated cells and then dividing by the average luminescence signal of cells treated with Chol LNPs. Relative luciferase expression is reported as mean±standard deviation of n=4 biological replicates (averaged from n=4 technical replicates). A nested one-way ANOVA with post hoc Student's t tests using the Holm-Šidák correction for multiple comparisons were used to compare cell viability across treatment groups.

### In Vitro Small Molecule Endocytosis Inhibitor Assays

BeWo b30 cells were plated at 50,000 cells per well in 100 μL of media in 96-well plates and left to adhere overnight. The inhibitors amiloride (AMI, inhibitor of macropinocytosis, 200 mM, Sigma Aldrich), chlorpromazine (CPZ, inhibitor of clathrin-mediated endocytosis, 2000 μM, Sigma Aldrich), genistein (GEN, inhibitor of caveolae-mediated endocytosis, 20 mM, Sigma Aldrich), methyl-β-cyclodextrin (MβCD, inhibitor of lipid-raft mediated endocytosis, 500 mM, Sigma Aldrich) and bafilomycin A1 (BAF A1, inhibitor of endosomal acidification, 200 nM, Sigma Aldrich) were dissolved in DMSO at their respective concentrations. 30 min prior to treatment with luciferase mRNA LNPs, 1 μL of each inhibitor was added to cells. Additionally, replicates of cells were treated with no inhibitor or 1 μL of DMSO to account for differences in LNP-mediated mRNA delivery because of enhanced cell permeability due to the presence of DMSO. After incubation with each inhibitor, cells were resuspended in fresh DMEM medium and dosed with 50 ng of luciferase mRNA LNPs. 24 h after treatment with LNPs, the luminescence signal and cytotoxicity was measured as previously described. Relative luminescence signal was calculated by first subtracting the background readings from untreated cells and then dividing by the average luminescence signal of cells treated with LNPs without inhibitor for each LNP treatment group. Relative luciferase expression is reported as mean standard deviation of n=4 biological replicates (averaged from n=3 technical replicates). Percent cell viability is reported as mean±standard deviation of n=3 biological replicates (averaged from n=3 technical replicates). A two-way ANOVA with post hoc Student's t tests using the Holm-Šidák correction for multiple comparisons was used to compare the results across treatment groups and inhibitors.

### LNP-Mediated Inflammation and Toxicity in Pregnant Mice

Serum stored from LNP biodistribution studies were used for the following LNP inflammation and toxicity studies. A colorimetric Mouse Inflammation ELISA Strip (Signosis, Santa Clara, CA) was used to evaluate IL-1α, IL-1β, G-CSF, GM-CSF, MCP-1, MIP-1α, SCF and RANTES in serum 6 h after LNP administration per the manufacturer's instructions. For each cytokine, the reported measurements for relative cytokine concentration are normalized to the average optical density measurements from the PBS-treated group. Relative cytokine concentrations are reported as mean±standard deviation from n=4 biological replicates (from n=1 technical replicate per cytokine). A two-way ANOVA with post hoc Student's t tests using the Holm-Šidák correction for multiple comparisons was used to compare relative cytokine concentrations across treatment groups and cytokines.

### Example 1: Design and Characterization of LNP Library

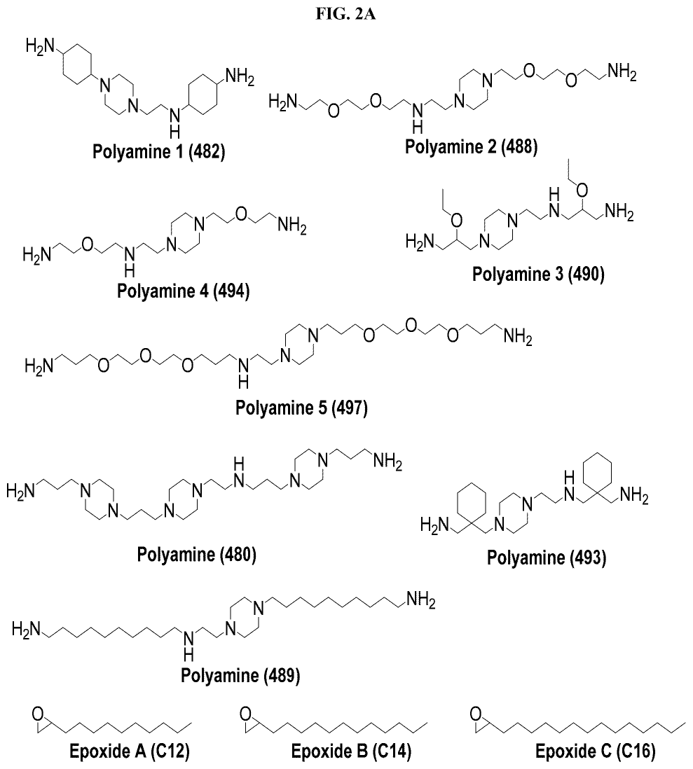

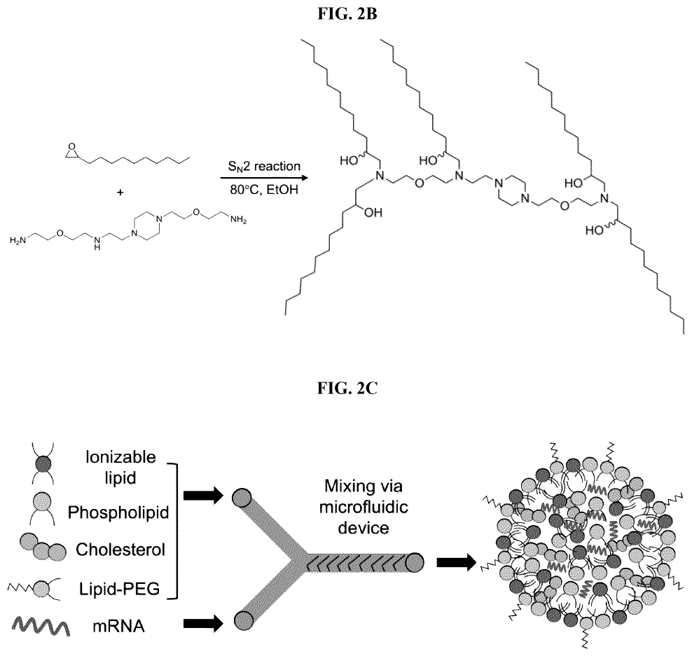

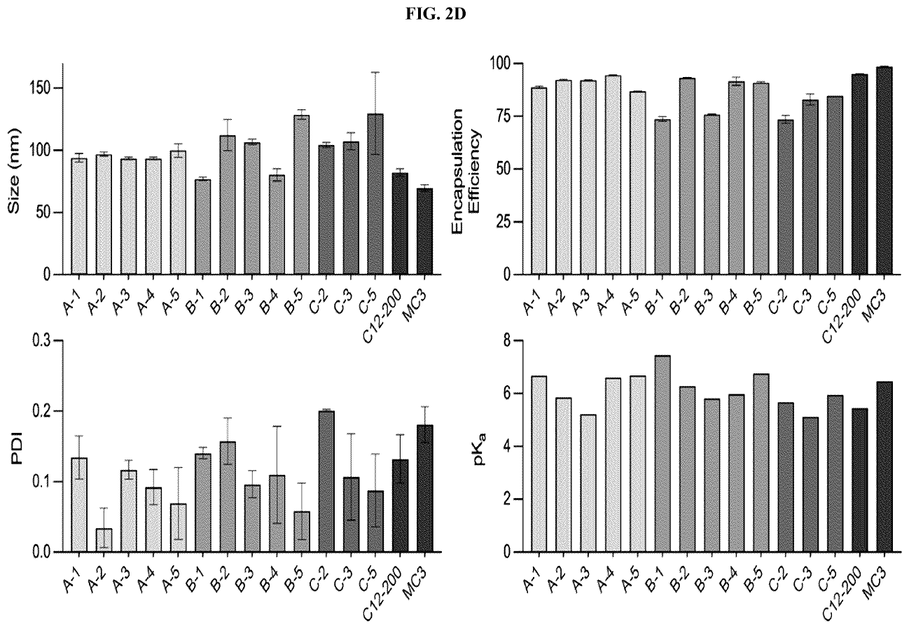

To engineer LNPs for mRNA delivery to the placenta during pregnancy, library of 15 LNPs were formulated each with a distinct ionizable lipid (i.e., ionizable lipids A1-A5, B1-B5, and C1-C5). Specifically, ionizable lipids were synthesized, as previously described utilizing methods known to those of ordinary skill in the art, by nucleophilic addition of an amine core (e.g., 1, 2, 3, 4, or 5) to one or more alkyl-substituted epoxides (e.g., A-C12, B-C14, and C-C16) (FIGS. 2A-2B). Each of these ionizable lipids was combined with 1,2-dioleoyl-sn-glycero-3-phosphoethanolamine (DOPE), cholesterol, and lipid-anchored poly(ethylene glycol) (PEG), at a molar ratio of 35:16:46.5:2.5, to formulate LNPs via chaotic mixing with an aqueous phase of luciferase mRNA in a microfluidic device (FIG. 2C). LNPs are referred to throughout by the ionizable lipid (e.g., A1). LNPs A1-A5, B1-B5, and C1-C5 differ only with regard to the identity of the ionizable lipid (i.e. the same ratio of components are used in each formulation).

Each of these lipid excipients play a key role in LNP formulation, intracellular uptake, and delivery. The ionizable lipid enables mRNA encapsulation and endosomal escape for potent intracellular delivery, the phospholipid or helper lipid DOPE promotes LNP membrane formation, cholesterol enhances membrane stability, and lipid-PEG limits rapid clearance and immune cell opsonization.

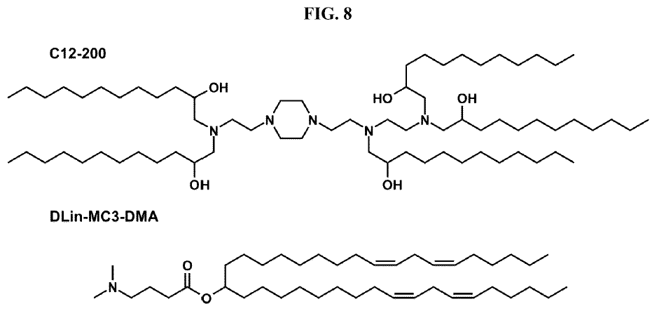

In addition to the library of 15 LNPs each with their own ionizable lipid, two control LNPs were formulated with ionizable lipids C12-200 and DLin-MC3-DMA which serve as industry standard lipids for comparison (FIG. 8). Following formulation, the LNPs were characterized the hydrodynamic size, polydispersity index (PDI), encapsulation efficiency, and pKa of the LNP library (FIG. 2D). Fifteen of the seventeen LNPs were less than 120 nm and the same number had PDIs less than 0.3. Ten of the seventeen LNPs had mRNA encapsulation efficiencies greater than 85%. Interestingly these formulations primarily had ionizable lipids with C12 or C14 epoxide tails. Finally, the pKa, or the pH at which the LNP is 50% protonated, was characterized for each LNP. LNP pKa depends largely on the ionizable lipid component and a value <7.0 indicates the ability of the LNP to escape the acidic environment of the endosome following endocytosis. In the endosome, LNPs become protonated causing their membrane lipids to fuse with the anionic lipids of the endosome and release their mRNA cargo into the cytosol. The observed pKa values for the LNP library ranged from 5.11 to 7.45 indicating the ionizable nature of the LNPs of the present disclosure for potent intracellular mRNA delivery.

### Example 2: MRNA LNP Delivery to Placental Cells In Vitro

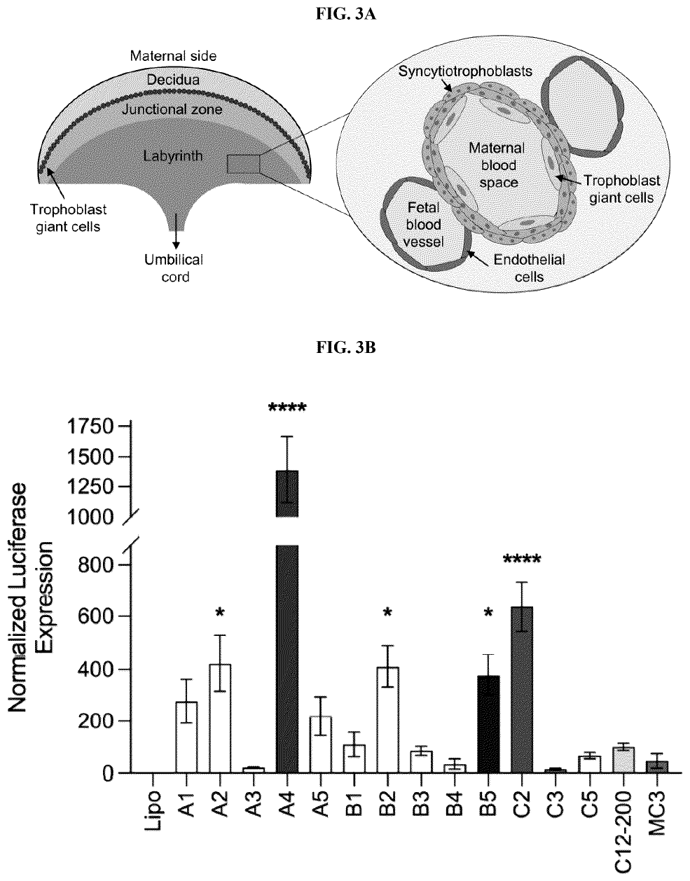

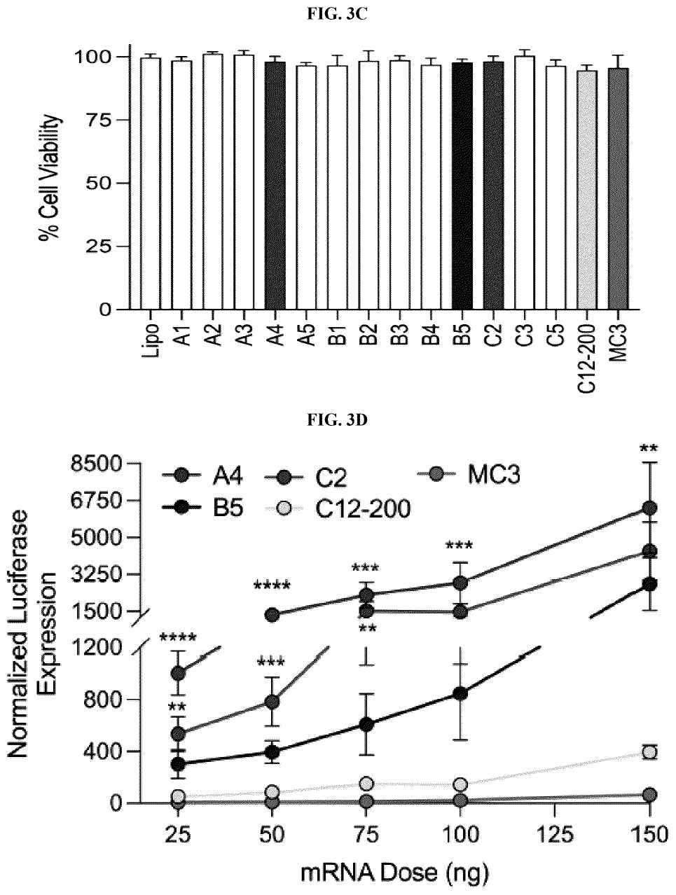

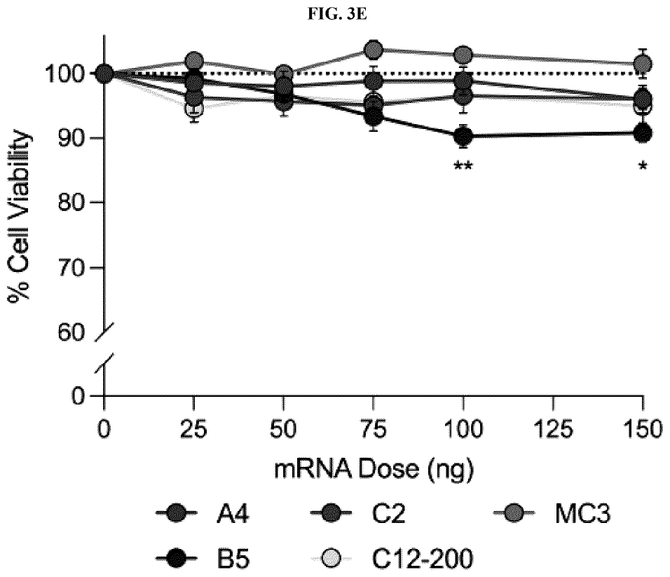

Next, the in vitro mRNA transfection efficiency of the LNP library was evaluated in placental cells. BeWo b30 cells, an immortalized human syncytiotrophoblast cell line, which are often used for in vitro models of the placenta, were selected for evaluation. While there are many differences between the mouse and human placenta, syncytiotrophoblasts are one of the major cell types in the placenta of both species. In the mouse placenta, there are three distinct cell regions from the maternal to fetal side (FIG. 3A).

First, is the decidua which is a thick mucosal membrane that houses placental immune cells and regulates trophoblast invasion into the uterus. Second, is the junctional zone which is separated from the decidua by trophoblast giant cells (TGCs) and is responsible for the main endocrine functions of the placenta. Finally, the labyrinth is where the majority of nutrient and gas exchange occurs between maternal and fetal blood.

Within the labyrinth, the maternal blood spaces are separated from fetal blood vessels by a layer of trophoblasts, including syncytiotrophoblasts and TGCs, as well as a layer of fetal endothelial cells. Both syncytiotrophoblasts and endothelial cells secrete proteins such as vascular endothelial growth factor (VEGF) that mediate vascularization in the placenta and impact the efficiency of oxygen transport to the fetus. Therefore, in vitro mRNA delivery was assessed in syncytiotrophoblast cells for applications in mediating placental vasodilation. LNPs or Lipofectamine were used to treat BeWo cells with 50 ng of luciferase mRNA per 50,000 cells.

Lipofectamine is often considered a gold standard transfection reagent for in vitro nucleic acid delivery. Luciferase expression as a measure of functional mRNA delivery was evaluated in BeWo b30 cells 24 h following treatment with LNPs or Lipofectamine. Eight LNPs from the seventeen LNP library had significantly higher luciferase expression than Lipofectamine (FIG. 3B). Of these top performers, four had the C12 epoxide tail, three had the C14 epoxide tail, and only one had the C16 epoxide tail. Additionally, all polyamine cores except core 3 are represented by the top eight performing LNPs.

Cell viability at 24 h was also evaluated following treatment and only two LNPs had significant decreases in cell viability compared to Lipofectamine (FIG. 3C). Five of the eight top-performing LNPs were selected for dose-dependent evaluation of luciferase expression and cell viability (FIGS. 3D-3E). Surprisingly, LNP A1 which was perhaps the lead candidate in the library screen, demonstrated toxicity starting at 100 ng of luciferase mRNA per 50,000 cells. LNPs A2, A4, and B2 all performed well in a dose-dependent manner, but LNP A4 was selected as a lead candidate for further evaluation as it had the lowest variability across multiple biological replicates. LNP B5, which performed well at low doses but poorly at higher doses, and C12-200, an industry standard ionizable lipid, were also selected for further evaluation.

### Example 3: LNPs Mediate Higher Non-Hepatic mRNA Delivery than Benchmark C12-200

### LNPs in Non-Pregnant and Pregnant Mice

LNPs A4, B5, and C12-200 were evaluated in vivo for luciferase expression to non-pregnant and pregnant mice. Previously, ionizable LNPs such as those utilizing the C12-200 lipid have been shown to deliver mRNA predominantly to the liver upon intravenous administration due to the first pass hepatic clearance effect and high blood flow in the liver. However, it was hypothesized that LNPs capable of delivering mRNA to non-hepatic organs upon intravenous administration may mediate delivery to the placenta based on the increased blood flow to the placenta during pregnancy. Therefore, luciferase mRNA delivery to the maternal organs (e.g., heart, lung, liver, kidney, spleen, and uterus) was first assessed in non-pregnant and pregnant mice.

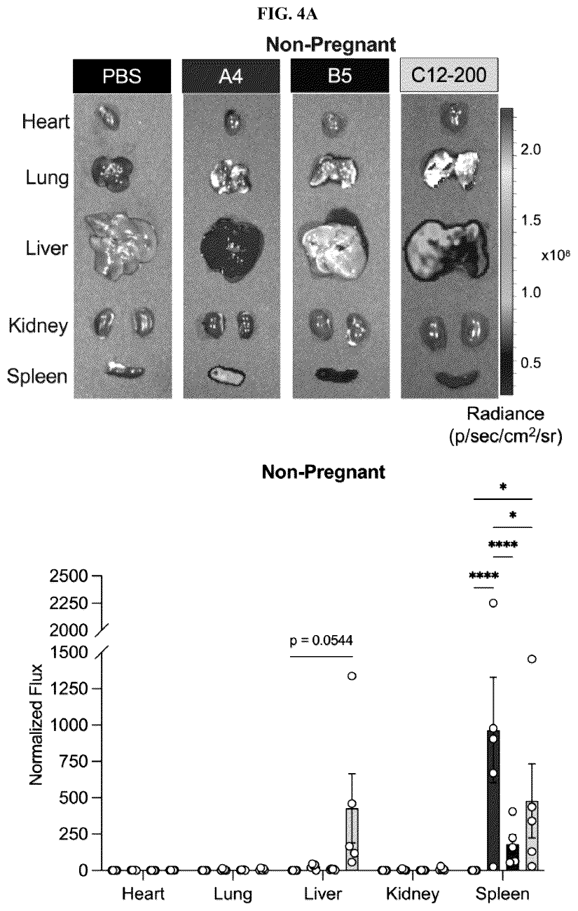

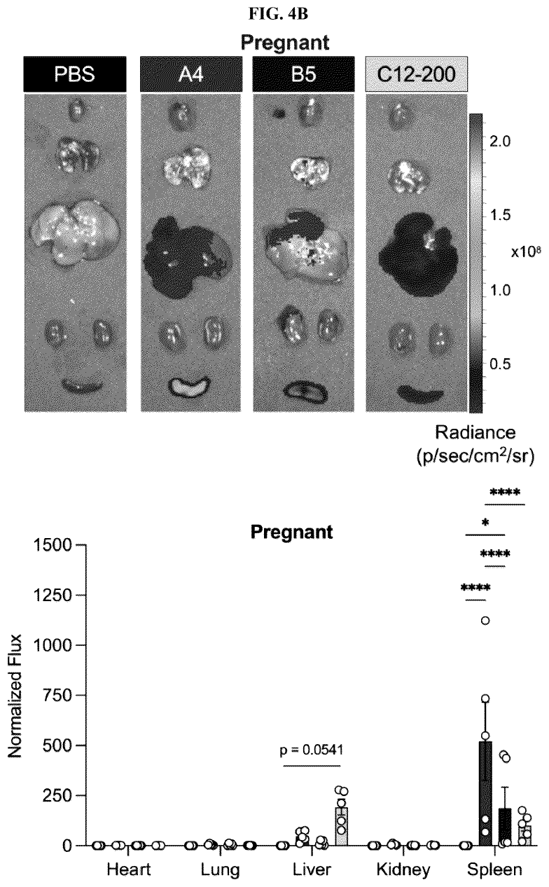

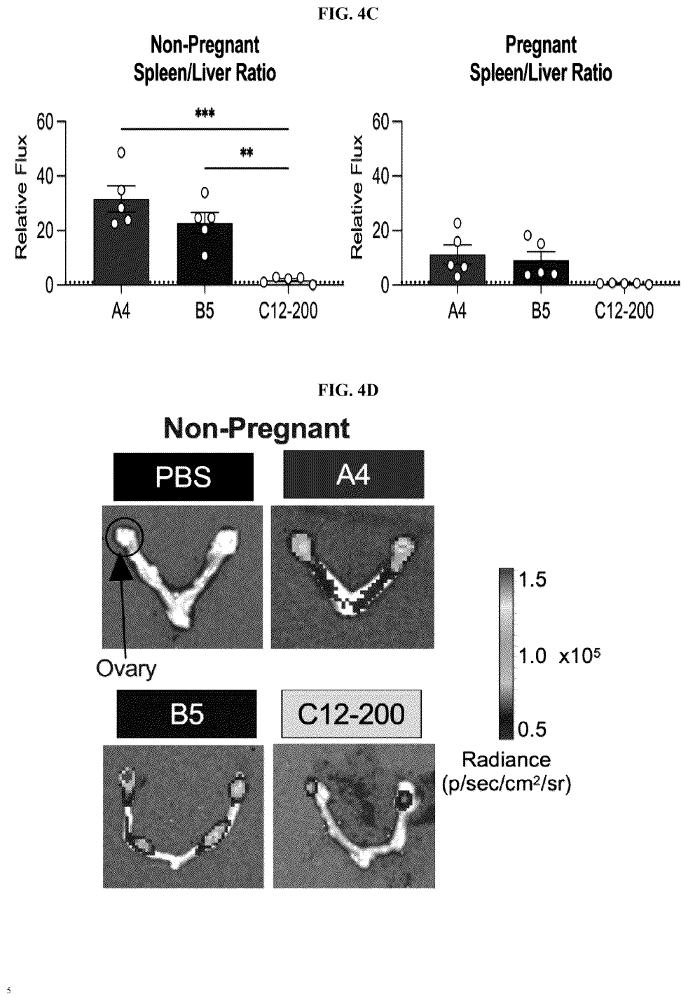

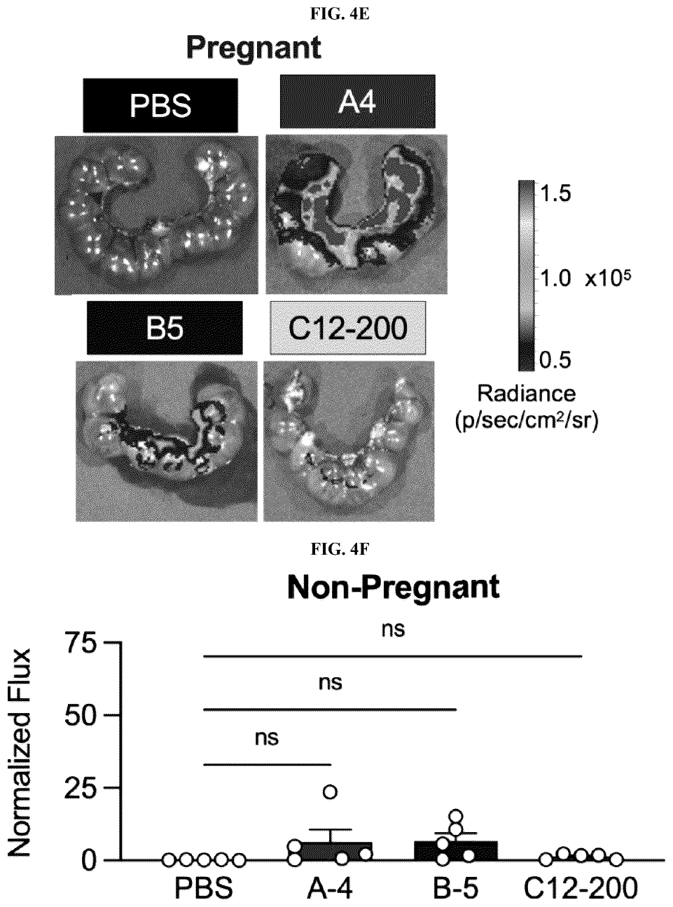

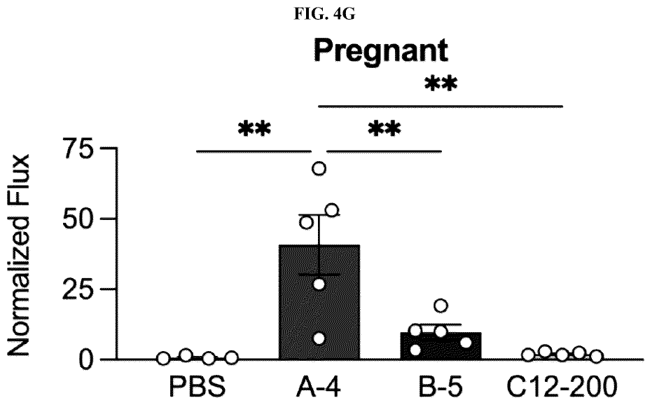

Non-pregnant and gestational day E16 pregnant mice were treated with PBS or LNPs at a dose of 0.6 mg/kg of luciferase mRNA via tail vein injection. 6 h later, mice were injected with luciferin, euthanized, and their non-reproductive organs were removed. An in vivo imaging system (IVIS) was used to measure and quantify luciferase expression in each of the organs using regions of interest (ROIs). In both non-pregnant and pregnant mice, C12-200 LNPs mediated significantly higher delivery to the liver than LNPs A4 and B5 (FIGS. 4A-4B). Instead, LNP A4 mediated significantly higher delivery to the spleen than LNPs B5 and C12-200 in both non-pregnant and pregnant mice. These results suggest the ability of LNP A4 to deliver mRNA to non-hepatic organs such as the spleen. There were no significant differences in the normalized luciferase expression values between non-pregnant and pregnant mice for all treatment groups and any of the organs evaluated.

The differences in non-hepatic delivery between non-pregnant and pregnant mice were further evaluated by dividing the bioluminescent flux measurements from the spleen by the liver measurements to create a spleen to liver ratio for all the LNP treatment conditions. LNPs A4 and B5 had significantly higher spleen/liver ratios in non-pregnant mice than C12-200, demonstrating these formulations mediated greater spleen than liver delivery (FIG. 4C). Interestingly, the spleen/liver ratios for LNPs A4 and B5 were significantly higher in non-pregnant mice than pregnant mice, suggesting less relative spleen delivery during pregnancy when compared to liver delivery.

Next, the mRNA LNP-mediated luciferase expression in the uterus and ovaries of both non-pregnant and pregnant mice was explored. Upon imaging, there appeared to be luciferase expression in both the uterus and ovaries of LNP-treated non-pregnant mice (FIG. 4D), however the quantified bioluminescent flux was very low compared to the other organs of interest and none of the LNP groups were significant compared to PBS (FIG. 4F). In the case of pregnant mice, the uterus was dissected from the mouse and imaged intact, with the fetuses and placentas still inside. Significant luciferase expression was observed in the uterus of the LNP A4 treated group compared to PBS (FIG. 4E and FIG. 4G).

### Example 4: Potent and Selective LNP-Mediated mRNA Delivery to the Placenta in Pregnant Mice

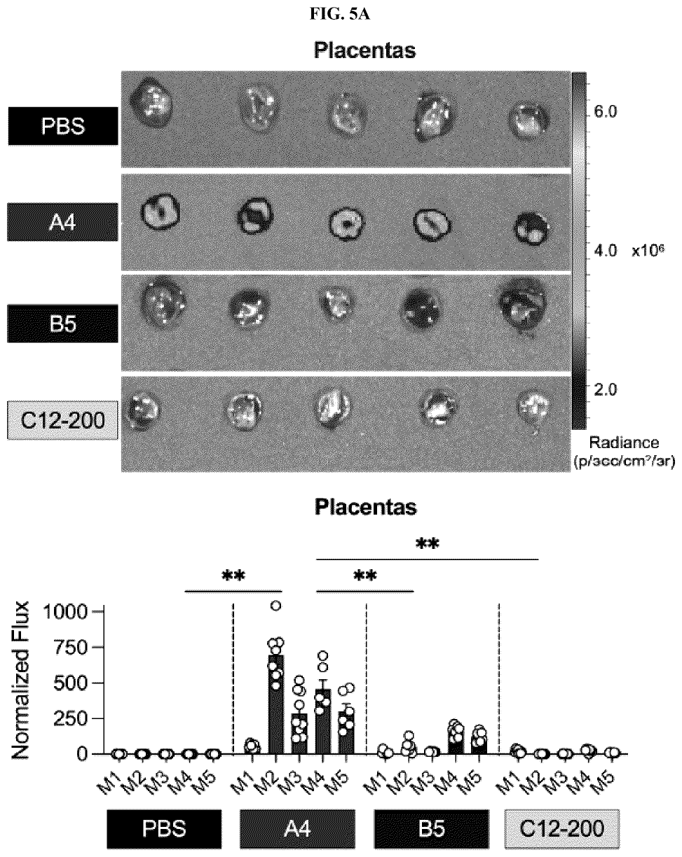

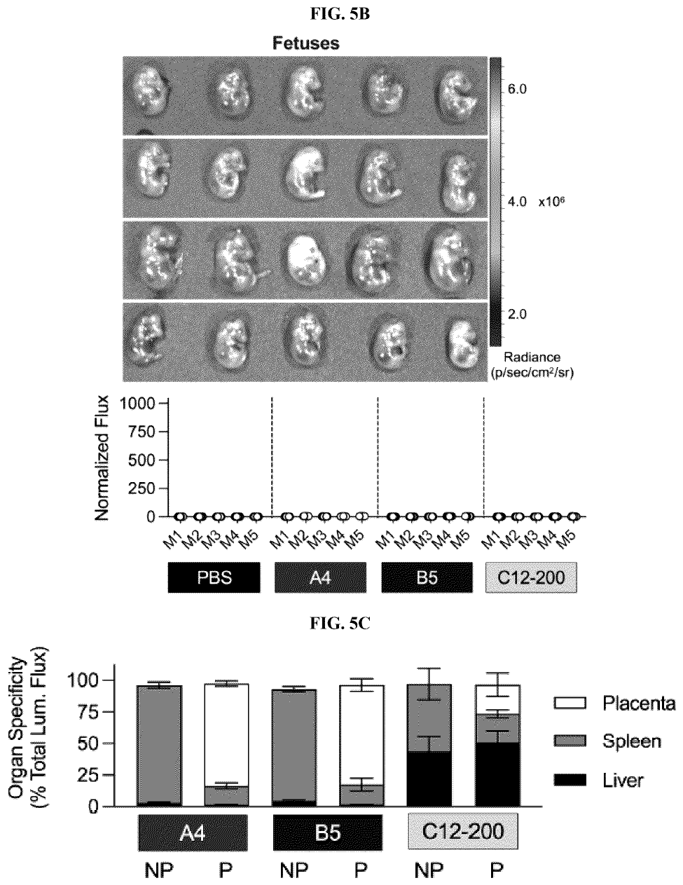

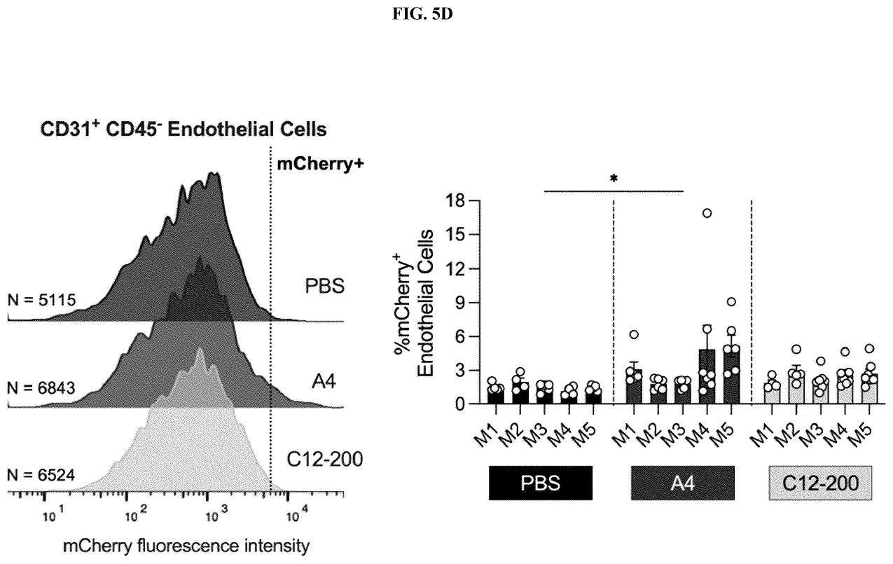

The uterus of pregnant mice was dissected to remove and image the placentas and fetuses. For all LNP treated groups, there was significant bioluminescent flux in the placentas compared to the PBS treated placentas (FIG. 5A). In agreement with the in vitro results described herein, LNP A4 mediated significantly higher in vivo luciferase mRNA delivery to the placenta compared to LNPs B5 and C12-200. There was no luciferase expression in the fetuses for any of the LNP treatment groups suggesting the safety of this LNP platform for mRNA delivery to the placenta (FIG. 5B).

The organ specificity of each mRNA LNP in both non-pregnant and pregnant mice was evaluated by totaling luminescent flux measurements from the liver, spleen, and placenta (if applicable) and calculated the percent of total luminescent flux for each organ (FIG. 5C). Interestingly, the percent liver delivery remained about the same between non-pregnant and pregnant mice for all three LNP formulations. For LNPs A4 and B5, less than 10% and 17%, respectively, of total luminescent flux was from the liver, indicating the non-hepatic specificity of these LNPs.

In contrast, using the C12-200 formulation, around 56% of the total luminescent flux was from the liver. When non-pregnant and pregnant mice were compared, the only non-hepatic delivery for non-pregnant mice was observed in the spleen, which then becomes partitioned between the spleen and placenta in pregnant mice. For LNP A4, about 62% of the total luminescent flux was from placental delivery in comparison to 45% for LNP B5 and 34% for C12-200. With both the highest magnitude of delivery and the highest specificity to the placenta, LNP A4 was shown to be the lead candidate for placental mRNA delivery.

In addition to demonstrating luciferase mRNA delivery to the placenta, mCherry mRNA was encapsulated into LNPs A4, B5, and C12-200 for a proof-of-concept evaluation of in vivo cellular level mRNA delivery to the mouse placenta. Twelve hours (12 h) after PBS or LNP administration in pregnant mice, placentas were dissected and cells were isolated and stained for endothelial cells (CD31+/CD45−), immune cells (CD31+/CD45+), as well as trophoblasts using the intracellular pan-trophoblast marker cytokeratin-7 (CK7+/CD31−/CD45−). First, mCherry expression was evaluated in endothelial cells for each treatment group (FIG. 5D) and mean percent positivity rates of 3.1, 2.2 and 1.7 were observed for LNPs A4, B5, and C12-200 respectively. For trophoblasts, the percent mCherry positivity was 3.2, 2.9, and 1.74 for LNPs A4, B5, and C12-200 (FIG. 5E).

These results suggest the ability of the LNPs of the present disclosure to deliver to the two major cell types of the placenta (i.e., trophoblasts and endothelial cells). Additionally, it appears these results with mCherry mRNA follow the same general trend which was observed with luciferase mRNA wherein LNP A4 had the highest placental specificity and C12-200 had the lowest placental specificity.

### Example 5: MRNA LNPs Mediate VEGF Expression with Minimal Toxicity In Vivo

Next, the functional delivery of a clinically relevant mRNA for placental disorders was evaluated using LNP A4 and the industry standard C12-200 LNP. Specifically, VEGF-A mRNA was chosen, as both recombinant VEGF protein and adenovirus-mediated gene therapies have been explored for placental disorders such as pre-eclampsia and fetal growth restriction. To this end, healthy, gestational age E16 pregnant mice were treated with either PBS or VEGF mRNA LNPs. 6 and 48 h following LNP administration, VEGF expression was evaluated in serum, livers, and placentas.

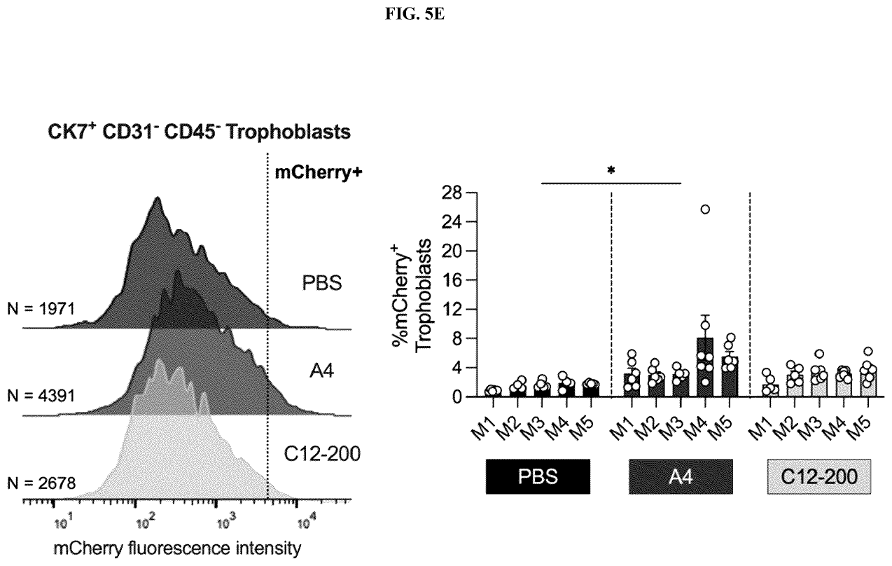

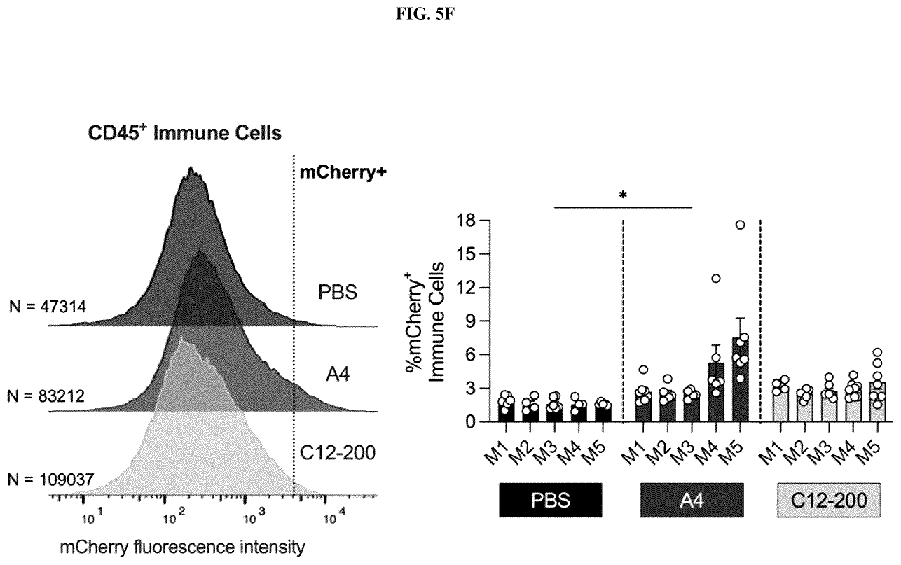

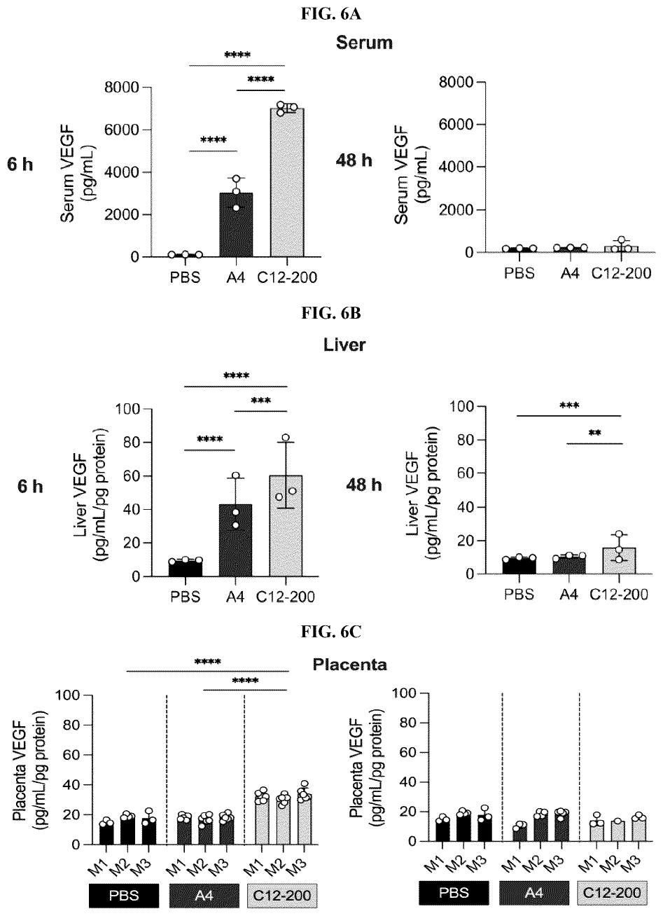

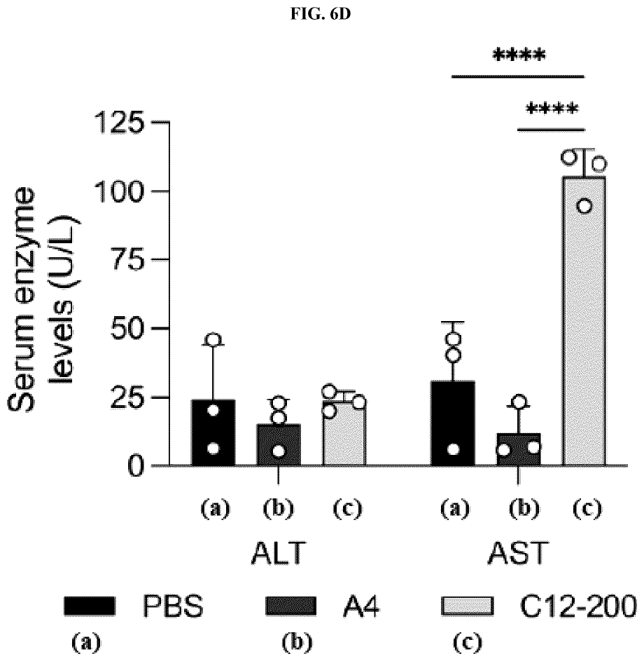

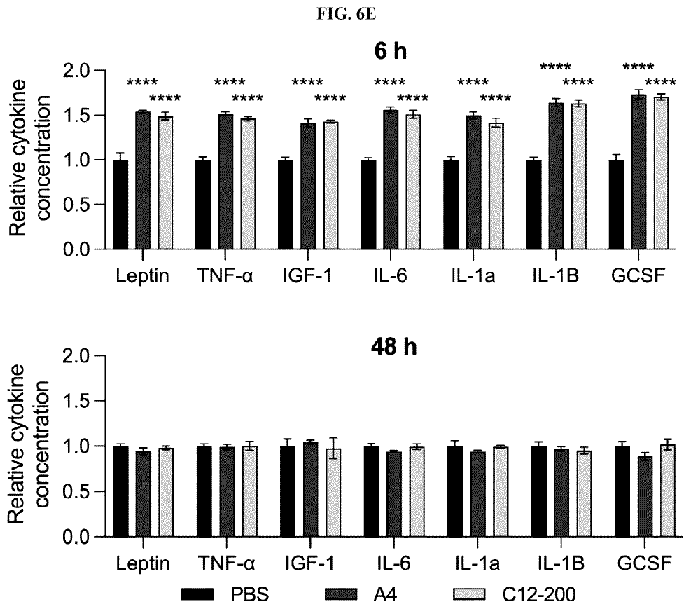

Six hours (6 h) following LNP treatment, VEGF serum levels for both LNP A4 and C12-200 were significantly elevated compared to PBS, with significantly higher VEGF levels for the C12-200 LNP group than LNP A4 (FIG. 6A). At 48 h, serum levels for both LNP-treated groups returned to baseline, demonstrating the transient nature of VEGF mRNA therapy. Next, in the liver at 6 h, LNP A4 and C12-200 mediated significant VEGF expression in the liver, with C12-200 mediating higher expression than LNP A4 (FIG. 6B). At 48 h, VEGF expression in the liver decreased substantially; however, there was still significant VEGF expression for the C12-200 LNP compared to PBS and LNP A4. In the placenta 6 h after LNP administration, there was significant VEGF expression for only the C12-200 LNP-treated mice compared to the LNP A4 and PBS groups. At 48 h, the VEGF levels in the placenta were at baseline for all groups.

Next, LNP-mediated liver toxicity was assessed by measuring serum levels of the secreted liver enzymes alanine aminotransferase (ALT) and aspartate aminotransferase (AST). These enzymes are often used to assess nanoparticle-mediated toxicity, as elevated levels of either enzyme can indicate hepatic injury due to high nanoparticle accumulation in the liver. 48 h after LNP administration, there were no significant changes in ALT levels compared to PBS for either of the LNP treatment groups (FIG. 6D). However, C12-200 LNPs resulted in significantly higher AST levels compared to PBS (3.5 fold) and LNP A4 (8.7 fold), perhaps suggesting some liver toxicity of the C12-200 LNPs. These results correlate with the high luciferase and VEGF expression in the liver for mice treated with C12-200 LNPs and demonstrate the benefit of delivery platforms such as LNP A4 which have increased specificity to the placenta.

LNP-mediated inflammation was also assessed in the placenta 48 h following LNP administration. Ensuring that the LNP platform disclosed herein induces minimal inflammation and immune system activation in the placenta is critical for translating the therapy to treat disorders such as pre-eclampsia, as pre-existing inflammation in the placenta is key marker of the disorder. To this end, seven cytokines that have been shown to mediate inflammation in the placenta were selected for assessment, and the relative concentration of each cytokine in LNP-treated mice compared to the PBS-treated mice. There were no significant increases in the relative concentrations of any of the seven cytokines between the LNP and PBS treated groups (FIG. 6E). These results suggest the safety of LNPs for mRNA delivery to the placenta.

### Example 6: VEGF mRNA LNPs Mediate Vasodilation in the Placenta

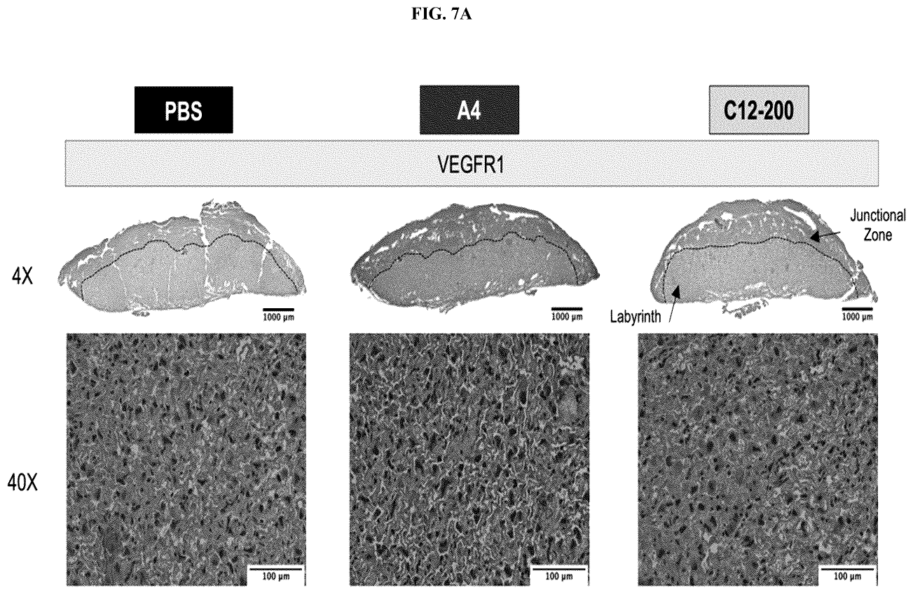

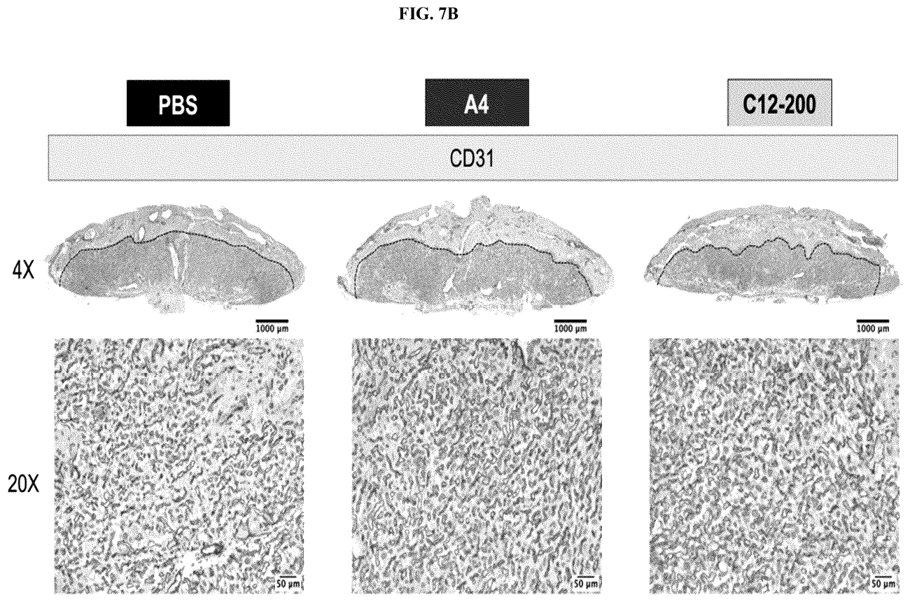

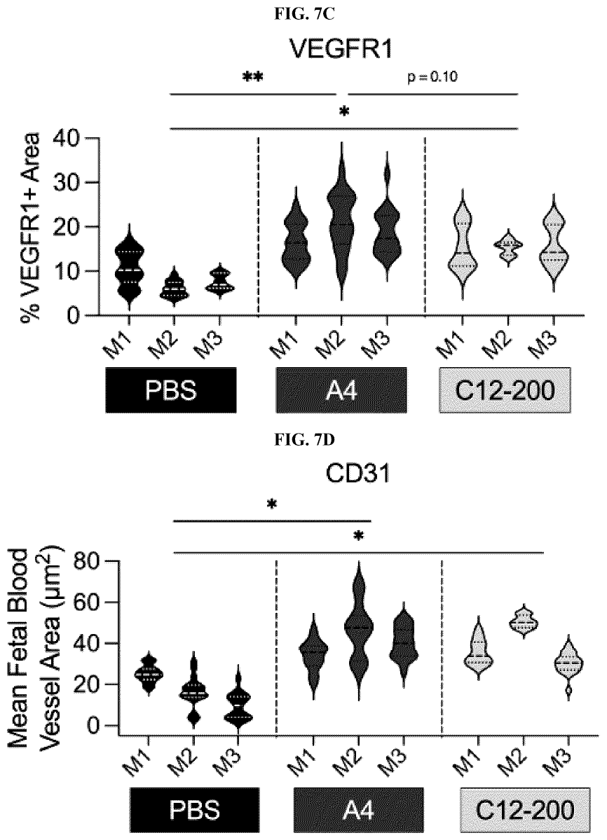

In addition to assessing VEGF expression, functional VEGF-mediated vasodilation in the placenta 48 h after LNP treatment was also assessed by quantifying blood vessel area from hematoxylin and eosin (H&E) or CD31 stained placentas. First, H&E stained placentas were evaluated using 4× and 40× objectives. At 4×, there were no visible changes in placental morphology in either the labyrinth or junctional zone (FIG. 7A). Images taken in the labyrinth region at 40× depict blood vessel spaces (both maternal and fetal) as white regions filled with red blood cells. Large purple stained nuclei represent trophoblasts, while small purple stained nuclei are fetal endothelial cells. Compared to PBS-treated mice, there is a visible increase in the blood vessel area for both LNP A4 and C12-200 treated placentas.

Interestingly, for the LNP A4 group, the increase in blood vessel area appears more homogenous, while for mice treated with C12-200 LNPs, some blood vessels are excessively dilated, and others are unaffected. It was hypothesized that these differences might be due to the local versus systemic expression of VEGF for the A4 and C12-200 LNPs, respectively. To test this hypothesis, immunohistochemistry was used to stain placental sections for CD31 using chromogenic detection with horseradish peroxidase so that CD31 positive cells appear brown. The endothelial cells surrounding fetal blood spaces are the only cells in the labyrinth region that will stain positive for CD31; maternal blood spaces surrounded by syncytiotrophoblasts will not (FIG. 3A).

Images taken with the 4× objective clearly show the divide between the junctional zone which has very few endothelial cells and the labyrinth which is rich in fetal endothelial cells to mediate oxygen and nutrient transport between maternal and fetal circulation (FIG. 7B). Images of CD31 stained placentas taken in the labyrinth region at 20× demonstrate not only increased intensity of CD31 staining, but an increase in fetal blood vessel area for LNP-treated mice. The particle analysis tool in ImageJ was used to quantify these differences in blood vessel area for both H&E and CD31 stained placental sections. H&E stained placentas can be used to quantify total (i.e., both fetal and maternal) blood vessel area, which increased significantly for mice treated with A4 and C12-200 LNPs compared to PBS mice (FIG. 7C). In fact, total blood vessel area was higher for the C12-200 LNP group than the A4 LNP group. However, when quantifying fetal blood vessel area from CD31 stained placentas, LNP A4 outperformed the C12-200 LNP and mediated significantly higher fetal blood vessel vasodilation (FIG. 7D).

Without wishing to be bound by theory, these results suggest that delivery platforms such as LNP A4 which are capable of local delivery to the placenta also mediate vasodilation in both maternal and fetal blood spaces of the placenta, while C12-200 LNPs mediate more vasodilation in maternal blood spaces due to primarily systemic VEGF expression by the liver.

### Example 7: Formulation and Characterization of Further Optimized LNP Libraries

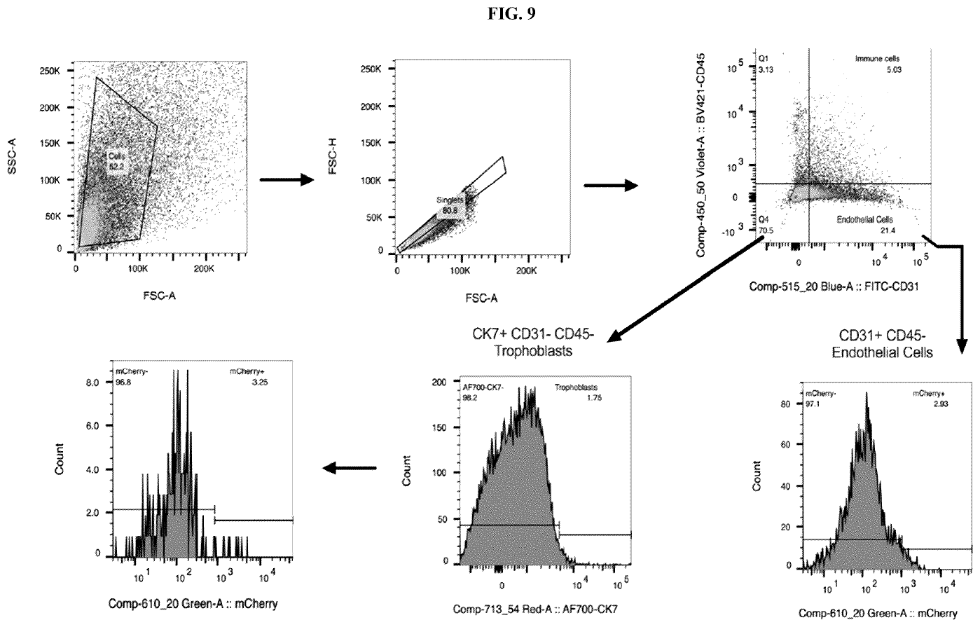

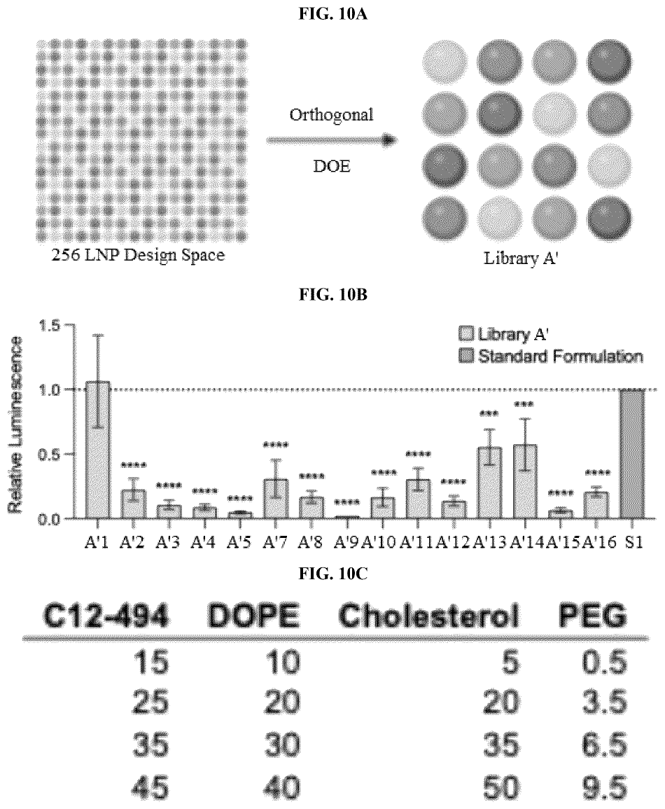

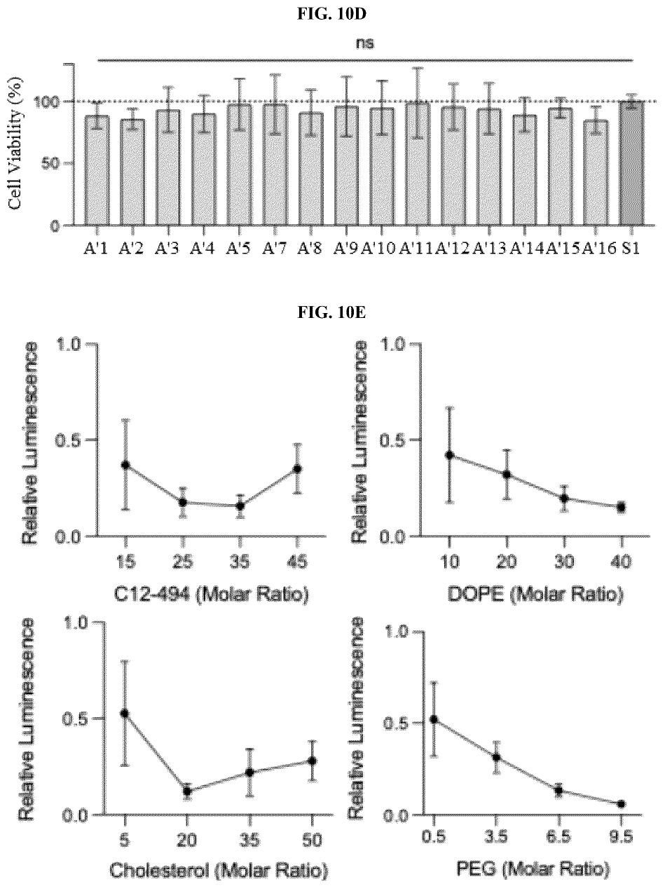

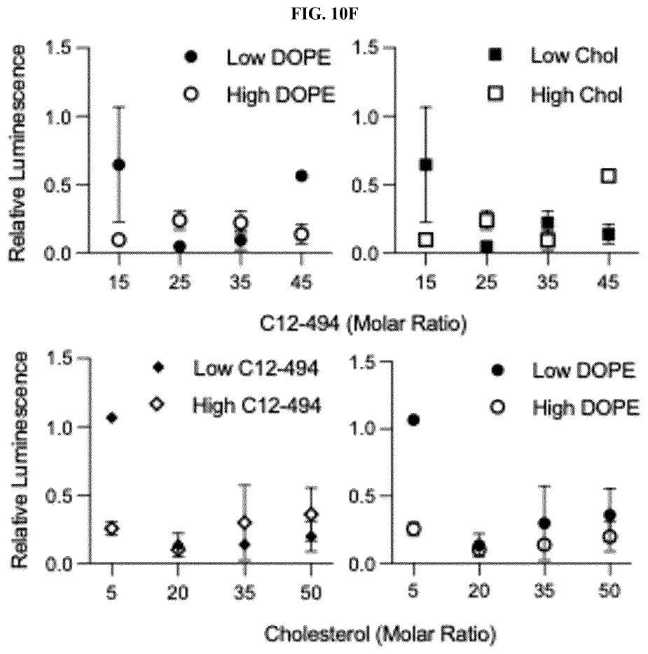

To optimize the LNP parameters for placental mRNA delivery, an initial library of LNPs was designed using orthogonal design of experiments (DOE), each of the four excipients was evaluated at four levels of excipient molar ratios to form a library of 16 representative formulations (FIG. 10A and Tables 1-2). Specifically, ionizable lipid C12-494 (i.e., ionizable lipid indicated herein as A4) was varied between 15-45%, DOPE between 10-40%, cholesterol between 5-50%, and PEG (i.e., PEG-conjugate) between 0.5-9.5% (FIG. 10B). Throughout experimentation, the optimized LNPs for placental mRNA delivery were compared to the previously optimized formulation S1 (i.e., LNP A4), which is formulated at molar ratios of 35:16:46.5:2.5 for ionizable lipid (i.e., ionizable lipid A4): DOPE:cholesterol:PEG-conjugated lipid (PEG).

TABLE 2
LNP formulations in Libraries A′, B′, and C′ as a molar ratio

| Name | C12-494 | DOPE | Chol. | PEG |
|---|---|---|---|---|
| A′1 | 15 | 10 | 5 | 0.5 |
| A′2 | 15 | 20 | 20 | 3.5 |
| A′3 | 15 | 30 | 35 | 6.5 |
| A′4 | 15 | 40 | 50 | 9.5 |
| A′5 | 25 | 10 | 20 | 6.5 |
| A′6 | 25 | 20 | 5 | 9.5 |
| A′7 | 25 | 30 | 50 | 0.5 |
| A′8 | 25 | 40 | 35 | 3.5 |
| A′9 | 35 | 10 | 35 | 9.5 |
| A′10 | 35 | 20 | 50 | 6.5 |
| A′11 | 35 | 30 | 5 | 3.5 |
| A′12 | 35 | 40 | 20 | 0.5 |
| A′13 | 45 | 10 | 50 | 3.5 |
| A′14 | 45 | 20 | 35 | 0.5 |
| A′15 | 45 | 30 | 20 | 9.5 |
| A′16 | 45 | 40 | 5 | 6.5 |
| S1 (A4) | 35 | 16 | 46.5 | 2.5 |
| B′1 | 10 | 8 | 4 | 0.5 |
| B′2 | 10 | 12 | 4 | 0.5 |
| B′3 | 10 | 8 | 8 | 0.5 |
| B′4 | 10 | 12 | 8 | 0.5 |
| B′5 | 15 | 8 | 4 | 0.5 |
| B′6 | 15 | 12 | 4 | 0.5 |
| B′7 | 15 | 8 | 8 | 0.5 |
| B′8 | 15 | 12 | 8 | 0.5 |
| C′1 | 45 | 15 | 35 | 2 |
| C′2 | 45 | 20 | 35 | 2 |
| C′3 | 45 | 15 | 45 | 2 |
| C′4 | 45 | 20 | 45 | 2 |
| C′5 | 55 | 15 | 35 | 2 |
| C′6 | 55 | 20 | 35 | 2 |
| C′7 | 55 | 15 | 45 | 2 |
| C′8 | 55 | 20 | 45 | 2 |

TABLE 3
LNP formulations in Libraries A′, B′, and C′ as a molar percentage

| Name | C12-494 | DOPE | Chol. | PEG |
|---|---|---|---|---|
| A′1 | 49.18 | 32.79 | 16.39 | 1.64 |
| A′2 | 25.64 | 34.19 | 34.19 | 5.98 |
| A′3 | 17.34 | 34.68 | 40.46 | 7.51 |
| A′4 | 13.10 | 34.93 | 43.67 | 8.30 |
| A′5 | 40.65 | 16.26 | 32.52 | 10.57 |
| A′6 | 42.02 | 33.61 | 8.40 | 15.97 |
| A′7 | 23.70 | 28.44 | 47.39 | 0.47 |
| A′8 | 24.15 | 38.65 | 33.82 | 3.38 |
| A′9 | 39.11 | 11.17 | 39.11 | 10.61 |
| A′10 | 31.39 | 17.94 | 44.84 | 5.83 |
| A′11 | 47.62 | 40.82 | 6.80 | 4.76 |
| A′12 | 36.65 | 41.88 | 20.94 | 0.52 |
| A′13 | 41.47 | 9.22 | 46.08 | 3.23 |
| A′14 | 44.78 | 19.90 | 34.83 | 0.50 |
| A′15 | 43.06 | 28.71 | 19.14 | 9.09 |
| A′16 | 46.63 | 41.45 | 5.18 | 6.74 |
| S1 | 35.00 | 16.00 | 46.50 | 2.50 |
| B′1 | 44.44 | 35.56 | 17.78 | 2.22 |
| B′2 | 37.74 | 45.28 | 15.09 | 1.89 |
| B′3 | 37.74 | 30.19 | 30.19 | 1.89 |
| B′4 | 32.79 | 39.34 | 26.23 | 1.64 |
| B′5 | 54.55 | 29.09 | 14.55 | 1.82 |
| B′6 | 47.62 | 38.10 | 12.70 | 1.59 |
| B′7 | 47.62 | 25.40 | 25.40 | 1.59 |
| B′8 | 42.25 | 33.80 | 22.54 | 1.41 |
| C′1 | 46.39 | 15.46 | 36.08 | 2.06 |
| C′2 | 44.12 | 19.61 | 34.31 | 1.96 |
| C′3 | 42.06 | 14.02 | 42.06 | 1.87 |
| C′4 | 40.18 | 17.86 | 40.18 | 1.79 |
| C′5 | 51.40 | 14.02 | 32.71 | 1.87 |
| C′6 | 49.11 | 17.86 | 31.25 | 1.79 |
| C′7 | 47.01 | 12.82 | 38.46 | 1.71 |
| C′8 | 45.08 | 16.39 | 36.89 | 1.64 |

To formulate the LNPs described herein, the ionizable lipid C12-494 was synthesized and combined with DOPE, cholesterol and lipid-anchored PEG in ethanol. This ethanol phase was then chaotically mixed in a microfluidic device with an aqueous phase containing mRNA to formulate each LNP. To vary the excipient composition in each LNP, the amount of each excipient added to the ethanol phase was varied to achieve the desired molar ratios and formulation. After formulation, library A′ was characterized for size, zeta potential, mRNA concentration, and mRNA encapsulation efficiency (Table 4). The z-average size of the LNPs in library A′ ranged from 63.7 to 133.2 nm where 15 of the 16 LNP formulations had a polydispersity index (PDI) under 0.3, indicating uniform LNP formulation. The surface charge for most of the LNPs in library A′ hovered around neutral (between −5 and 5 mV) with the exception of LNP A′14 which had a zeta potential of -18.1 mV. mRNA concentrations ranged from 23.5 to 45.7 ng/μL while mRNA encapsulation efficiencies varied from 0 to 85%. For many of the LNPs in library A′, the size, PDI, surface charge, mRNA concentration and encapsulation efficiencies were comparable to that of the initial lead LNP formulation, S1.

Table 4. LNP characterization for Library A′

TABLE 4
LNP characterization for Library A′

| | Z-avg size | Polydispersity | Zeta Potential | mRNA conc. | Encapsulation |
|---|---|---|---|---|---|
| Name | (nm) | Index (PDI) | (mV) | (ng/μL) | Efficiency (%) |
| A′1 | 89.5 ± 1.7 | 0.18 ± 0.02 | 4.91 ± 0.57 | 30.2 ± 0.4 | 81.8 ± 0.4 |
| A′2 | 100.2 ± 6.6 | 0.12 ± 0.10 | 1.68 ± 1.66 | 35.1 ± 0.4 | 62.8 ± 2.7 |
| A′3 | 99.3 ± 3.3 | 0.25 ± 0.06 | 1.54 ± 2.31 | 45.7 ± 0.7 | 70.1 ± 2.1 |
| A′4 | 89.0 ± 6.8 | 0.35 ± 0.07 | 1.80 ± 0.64 | 41.6 ± 0.7 | 79.0 ± 1.1 |
| A′5 | 81.0 ± 3.4 | 0.12 ± 0.02 | −2.76 ± 0.68 | 37.9 ± 0.3 | 35.1 ± 0.6 |
| A′6 | 81.2 ± 0.9 | 0.28 ± 0.05 | −2.39 ± 0.15 | 35.4 ± 0.7 | 0.0 ± 0.0 |
| A′7 | 129.7 ± 0.8 | 0.05 ± 0.05 | 4.82 ± 2.37 | 33.8 ± 3.0 | 76.6 ± 3.5 |
| A′8 | 133.2 ± 1.3 | 0.25 ± 0.03 | 4.41 ± 0.73 | 34.8 ± 0.3 | 81.1 ± 0.4 |
| A′9 | 63.7 ± 5.4 | 0.28 ± 0.04 | −3.34 ± 0.41 | 37.8 ± 0.4 | 35.5 ± 0.4 |
| A′10 | 92.1 ± 4.7 | 0.22 ± 0.02 | −3.30 ± 0.25 | 38.3 ± 0.6 | 76.4 ± 1.4 |
| A′11 | 89.3 ± 5.1 | 0.22 ± 0.01 | −1.32 ± 0.34 | 36.9 ± 0.6 | 64.5 ± 0.8 |
| A′12 | 117.2 ± 4.5 | 0.09 ± 0.12 | −1.85 ± 2.87 | 28.4 ± 1.4 | 71.0 ± 1.4 |
| A′13 | 88.1 ± 6.5 | 0.20 ± 0.05 | −0.28 ± 0.09 | 38.4 ± 0.8 | 85.3 ± 1.2 |
| A′14 | 105.5 ± 2.1 | 0.17 ± 0.02 | −18.07 ± 1.55 | 23.5 ± 1.2 | 66.9 ± 3.5 |
| A′15 | 89.9 ± 1.3 | 0.21 ± 0.05 | −4.92 ± 0.34 | 37.7 ± 0.4 | 33.1 ± 2.1 |
| A′16 | 78.2 ± 5.9 | 0.25 ± 0.04 | −4.52 ± 1.14 | 38.1 ± 0.5 | 30.2 ± 1.6 |
| S1 | 107.9 ± 5.0 | 0.25 ± 0.01 | 0.20 ± 1.24 | 28.6 ± 0.4 | 88.3 ± 0.5 |

### Example 8: Evaluation of Library A′ for In Vitro mRNA LNP Delivery to Trophoblasts

For both in vitro and in vivo screening, luciferase mRNA was encapsulated into LNPs as a model mRNA cargo, where luminescence signal correlates to functional mRNA delivery. For in vitro library screening, BeWo b30 trophoblast cells, a human choriocarcinoma cells line, were used as they have been previously used to model the placental barrier and evaluate nanoparticle uptake in the placenta. To assess mRNA delivery to trophoblasts, BeWo b30 cells were treated with the LNPs from library A′ and S1. However, since LNP A′6 exhibited 0% encapsulation efficiency, it was removed from the library screen. 24 hours after treatment, luciferase expression and cell viability were measured for all treatment groups and compared to the S1 formulation.

All of the LNP formulations in library A′, except for A′1, displayed significantly reduced mRNA delivery compared to S1. LNP A′1 was the only LNP formulation that exhibited comparable mRNA delivery to trophoblasts compared to S1 (FIG. 10C). Additionally, none of the LNPs in library A′ and S1 exhibited cytotoxicity in the BeWo b30 cells 24 hours after LNP treatment (FIG. 10D).

While the formulations in library A′ significantly enhanced mRNA delivery over the S1 formulation, the relationship between the different excipient molar ratios and mRNA delivery were investigated to identify trends that lead to improved mRNA delivery. To study these relationships, the luminescence signal for all LNPs with the same molar ratio of a specific excipient were plotted (FIG. 10E). This revealed that LNPs with decreased molar ratios of DOPE and PEG led to improved mRNA delivery in BeWo b30 cells. Additionally, LNPs at both the lowest and highest molar ratios of C12-494 (A4 ionizable lipid) and LNPs at the lowest molar ratio of cholesterol led to the highest luminescence signal. Given these trends, two libraries were subsequently designed, one with low molar ratios of C12-494 and one with high molar ratios.

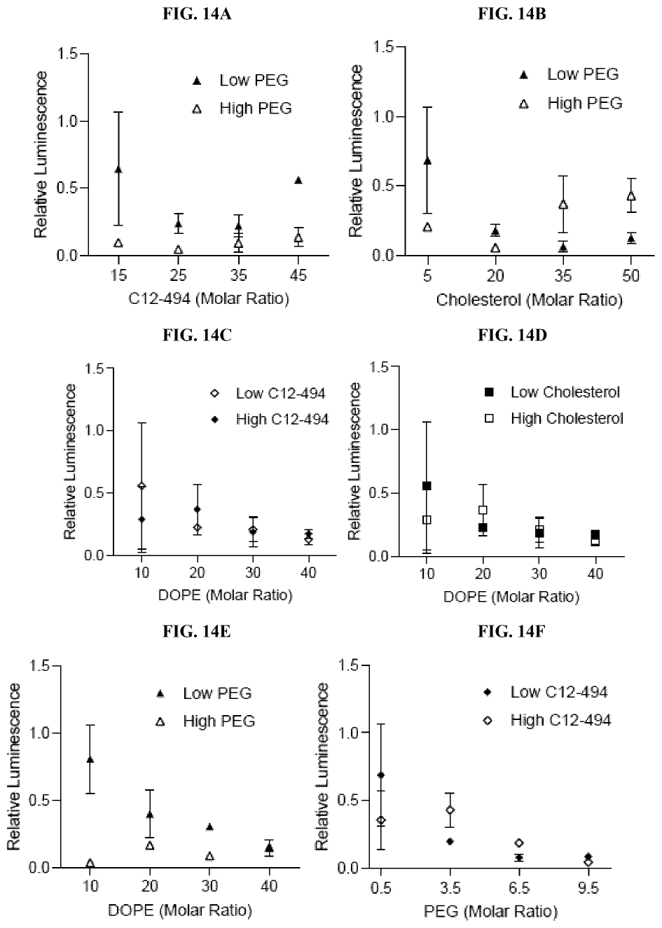

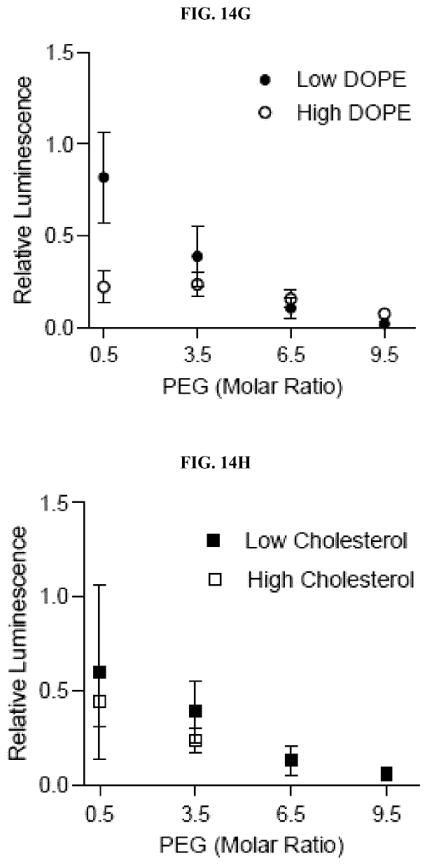

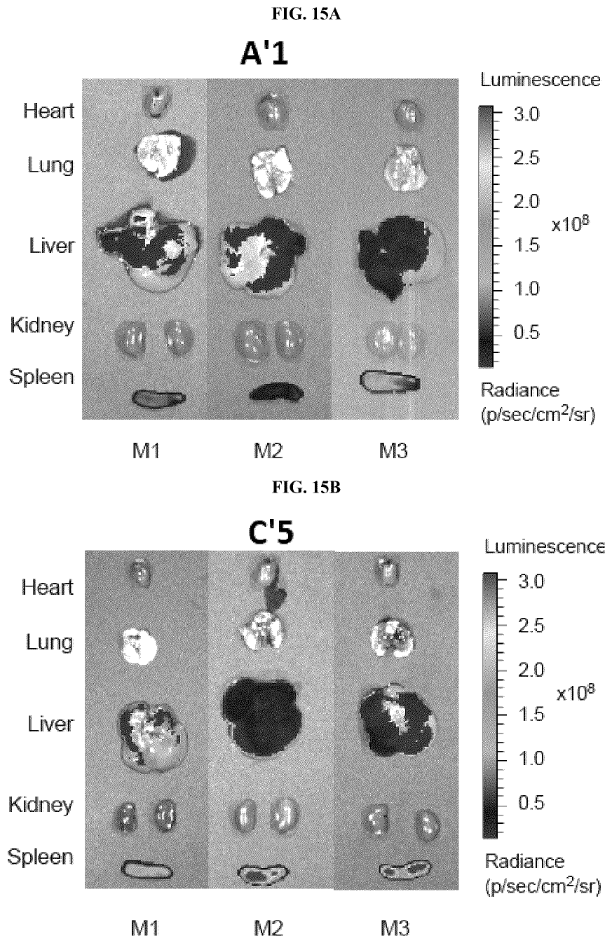

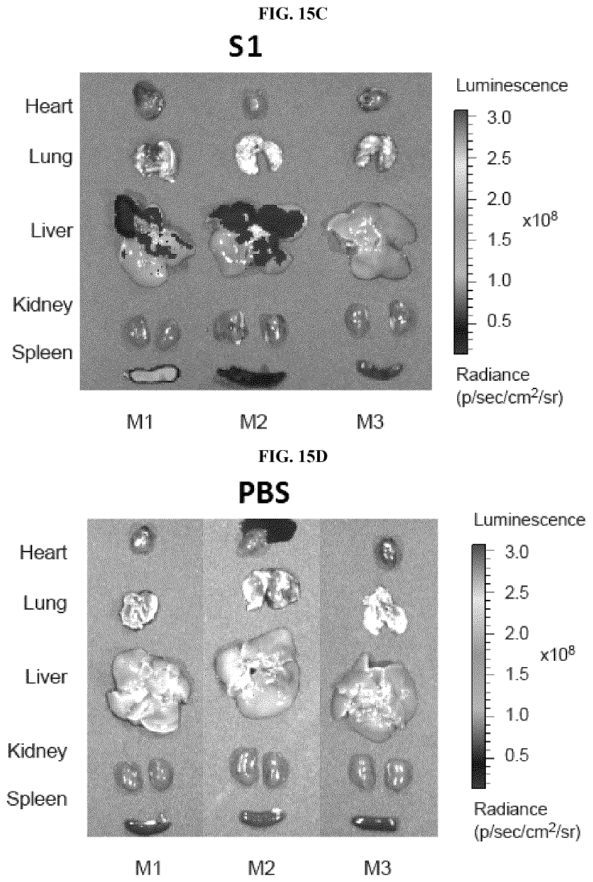

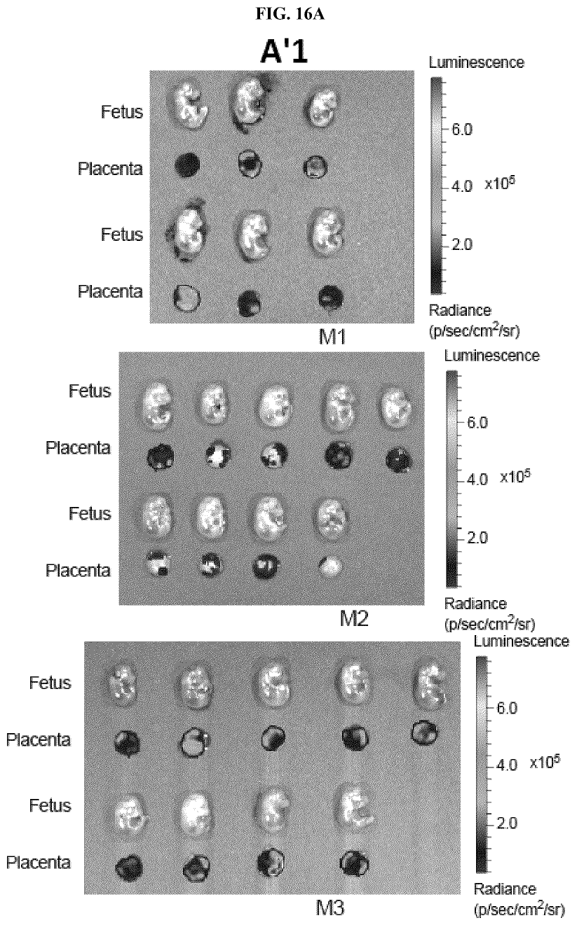

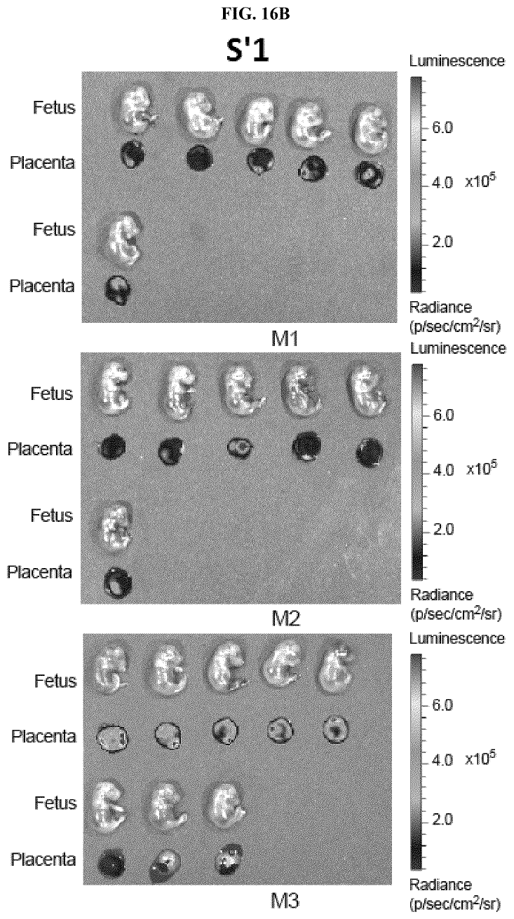

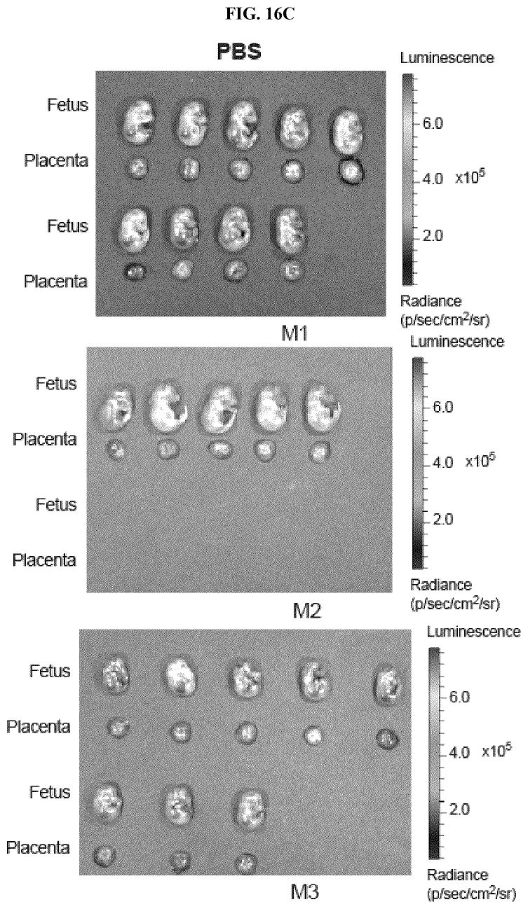

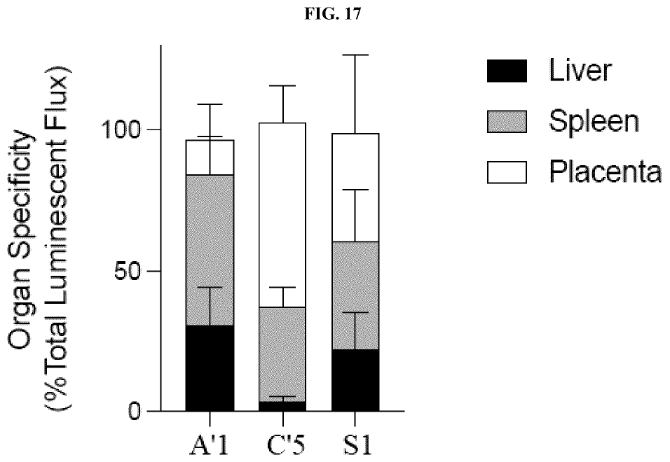

Additionally, to further inform this library design, the impact of two excipient molar ratios on trends in mRNA delivery was investigated. To do this, the average luminescence signal of LNPs with the same molar ratio of one excipient were plotted with either the two lower or higher molar ratios of a second excipient (FIG. 10F and FIGS. 14A-14H). These relationships revealed that at both low and high molar ratios of C12-494, mRNA delivery was improved with lower amounts of DOPE and PEG.

However, at low molar ratios of C12-494, mRNA delivery was improved with lower ratios of cholesterol while high molar ratios of C12-494 had improved delivery with higher ratios of cholesterol. Similarly, low molar ratios of cholesterol benefitted from lower ratios of C12-494 while high molar ratios of cholesterol had improved delivery with high molar ratios of C12-494. Additionally, mRNA delivery was improved with lower amounts of DOPE across all molar ratios of cholesterol. Interestingly, higher molar ratios of cholesterol saw improved delivery with higher amounts of PEG (FIGS. 14A-14H).

### Example 9: Further Optimization of LNP Excipients Enhances In Vitro mRNA LNP Delivery to Trophoblasts

Using the observed trends and DOE, two sequential 8-LNP libraries were designed within a narrowed range of excipient molar ratios (FIG. 11A). Library B′ was designed with lower molar ratios of C12-494, DOPE, cholesterol and a low ratio of PEG, which was held constant, while library C was designed with higher molar ratios of C12-494 and cholesterol, lower molar ratios of DOPE and again, the PEG molar ratio was held constant (FIG. 11B-11C and Tables 1-2). Both library B′ and C′ were characterized for size, zeta potential, mRNA concentration, and mRNA encapsulation efficiencies following formulation (Table 5).

TABLE 5
LNP characterization for Libraries B′ and C′

| | Z-avg size | Polydispersity | Zeta Potential | mRNA conc. | Encapsulation |
|---|---|---|---|---|---|
| Name | (nm) | Index (PDI) | (mV) | (ng/μL) | Efficiency (%) |
| B′1 | 127.4 ± 7.0 | 0.30 ± 0.01 | 1.94 ± 0.63 | 25.1 ± 0.8 | 91.0 ± 0.6 |
| B′2 | 125.3 ± 5.5 | 0.32 ± 0.02 | −0.28 ± 0.65 | 27.1 ± 0.9 | 91.2 ± 0.9 |
| B′3 | 110.7 ± 6.8 | 0.33 ± 0.04 | 3.03 ± 0.44 | 34.1 ± 1.3 | 93.0 ± 0.9 |
| B′4 | 69.7 ± 1.5 | 0.26 ± 0.01 | −5.89 ± 0.83 | 39.0 ± 0.9 | 48.7 ± 3.3 |
| B′5 | 134.0 ± 0.6 | 0.22 ± 0.00 | 0.75 ± 0.96 | 11.6 ± 0.7 | 81.3 ± 0.3 |
| B′6 | 85.5 ± 3.3 | 0.26 ± 0.01 | −0.98 ± 1.22 | 37.0 ± 2.8 | 87.2 ± 0.9 |
| B′7 | 145.7 ± 13.3 | 0.29 ± 0.03 | 1.30 ± 0.33 | 33.2 ± 1.1 | 92.4 ± 1.2 |
| B′8 | 65.3 ± 12.5 | 0.17 ± 0.01 | −2.08 ± 0.15 | 39.2 ± 1.2 | 36.0 ± 2.0 |
| C′1 | 124.4 ± 0.3 | 0.33 ± 0.02 | 6.42 ± 0.60 | 30.35 ± 0.4 | 90.0 ± 0.4 |
| C′2 | 157.1 ± 1.9 | 0.31 ± 0.02 | 3.22 ± 0.20 | 28.80 ± 0.9 | 90.2 ± 0.2 |
| C′3 | 120.5 ± 1.9 | 0.31 ± 0.02 | 4.43 ± 0.68 | 31.65 ± 0.5 | 90.1 ± 0.3 |
| C′4 | 117.3 ± 3.9 | 0.33 ± 0.01 | 4.81 ± 0.83 | 32.48 ± 0.3 | 92.0 ± 0.3 |
| C′5 | 135.7 ± 3.9 | 0.29 ± 0.01 | 3.08 ± 0.79 | 27.23 ± 1.8 | 88.1 ± 0.4 |
| C′6 | 138.4 ± 2.6 | 0.21 ± 0.02 | 1.28 ± 0.46 | 28.67 ± 0.9 | 86.8 ± 0.3 |
| C′7 | 153.8 ± 4.6 | 0.27 ± 0.02 | 6.88 ± 1.09 | 29.07 ± 1.1 | 88.0 ± 0.1 |
| C′8 | 140.4 ± 2.0 | 0.32 ± 0.02 | 5.32 ± 0.57 | 34.67 ± 0.9 | 90.4 ± 0.3 |

The LNPs in library B′ had a z-average size between 65.3 nm and 145.7 nm with PDIs less than 0.33. The zeta potential across the library varied from −5.89 to 3.03 mV. mRNA concentrations for LNPs in library B′ were measured between 11.6 and 39.2 ng/μL and encapsulation efficiencies were generally higher across library B′ compared to library A′ with 6 out of the 8 LNPs having encapsulation efficiencies greater than 80%. The LNPs in library C′ were larger in size than both library A′ and B′, with z-average sizes ranging from 117.3 to 157.1 nm. PDIs across the library were less than 0.33 and the zeta potential for all LNP formulations was positive, varying from 1.28 to 6.88 mV. mRNA encapsulations varied between 28.67 and 34.67 ng/μL and average encapsulation efficiencies were higher than both library A′ and B′, with all formulations having encapsulation efficiencies greater than 85%.

TABLE 6
pKₐ values for LNPs evaluated in a dose response

| Name | pKₐ |
|---|---|
| A′1 | 5.829 |
| B′5 | 6.103 |
| C′5 | 5.437 |
| S1 | 5.921 |

Both library B′ and C′ were screened in BeWo b30 cells to evaluate mRNA delivery and cytotoxicity in trophoblasts. Compared to S1, library B′ contained one particle with significantly improved mRNA delivery, LNP B′5, while all LNPs in library C had increased luciferase expression compared to S1, with 4 LNPs demonstrating significantly higher mRNA delivery compared to S1 (FIG. 11D). The top performing LNP in library C′, C′5 exhibited a four-fold increase in mRNA delivery compared to S1 while the top performing LNPs in library B′ and A′, B′5 and A′1, only exhibited a three-fold and one-fold improvement in mRNA delivery compared to S1, respectively. Additionally, none of the LNPs in library B′ or C′ demonstrated any cytotoxicity in BeWo b30 cells (FIG. 11E).

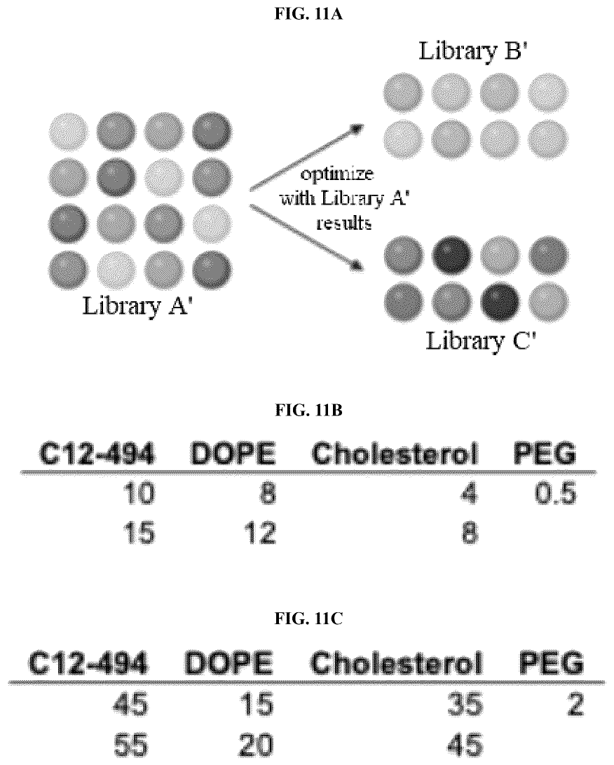

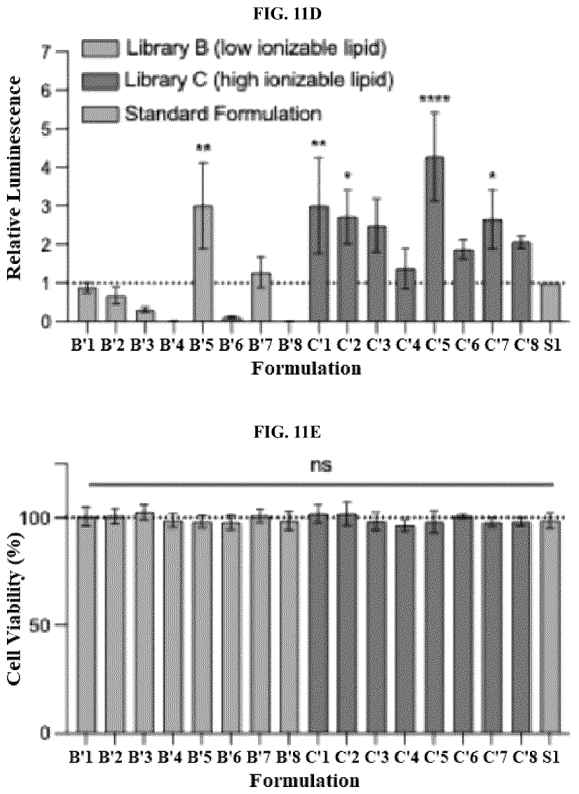

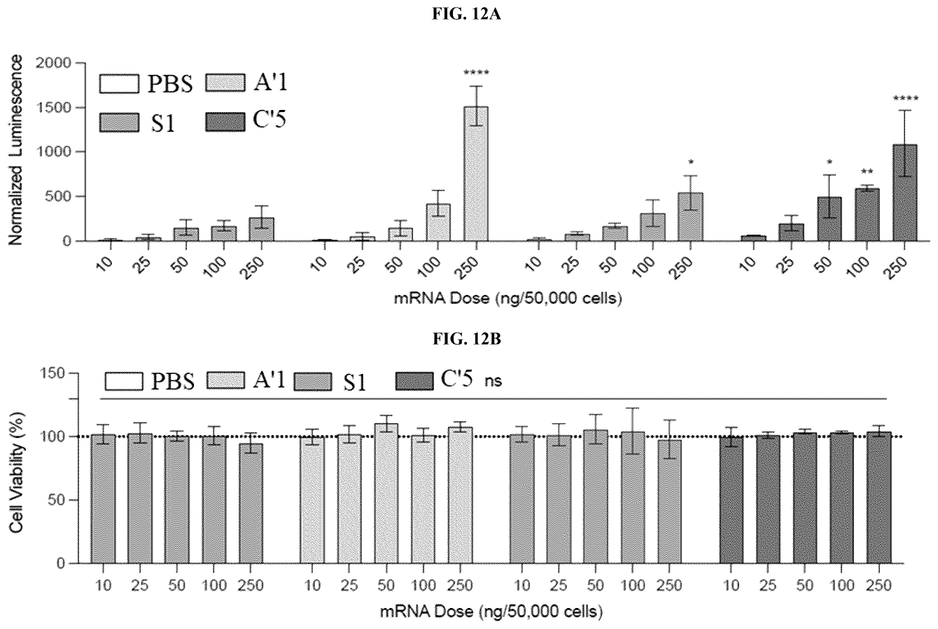

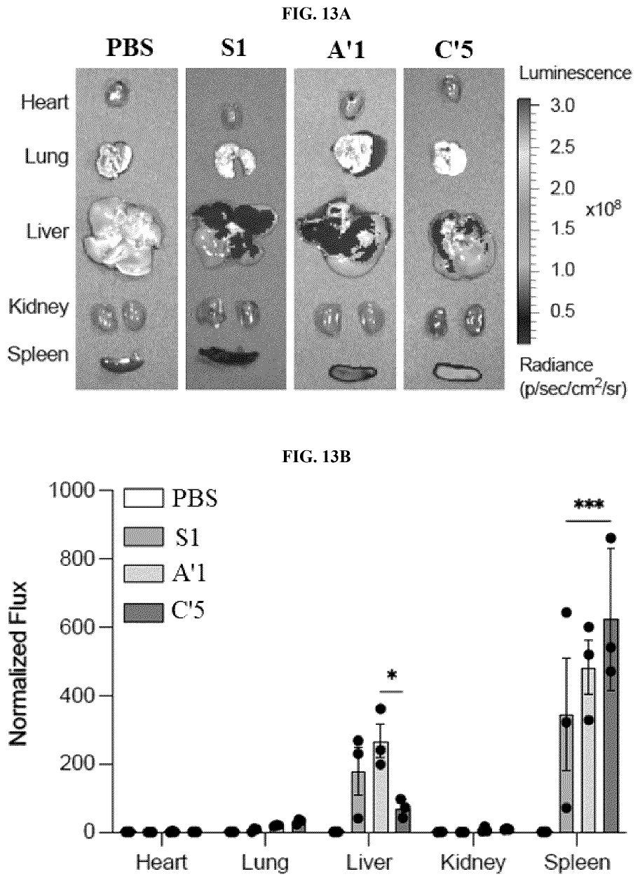

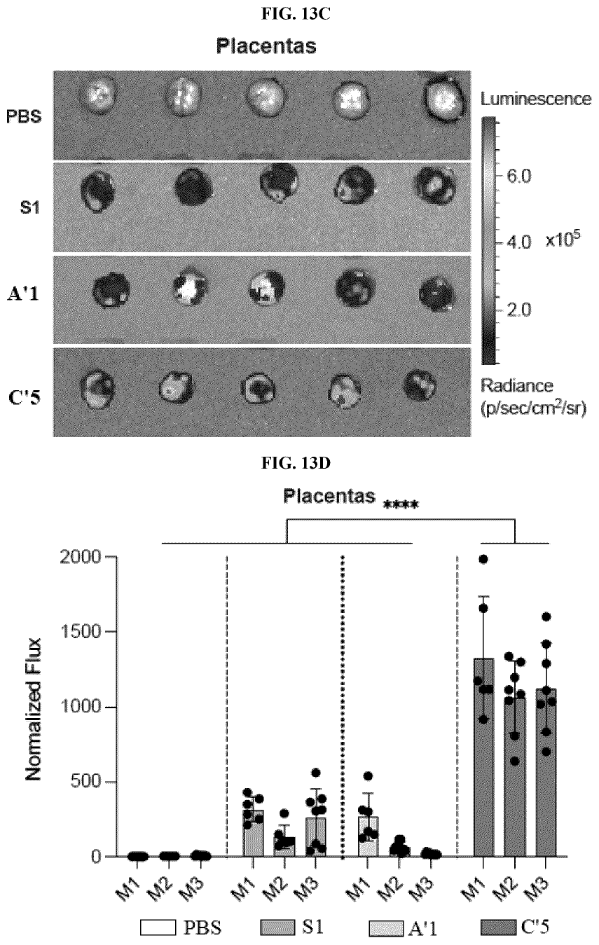

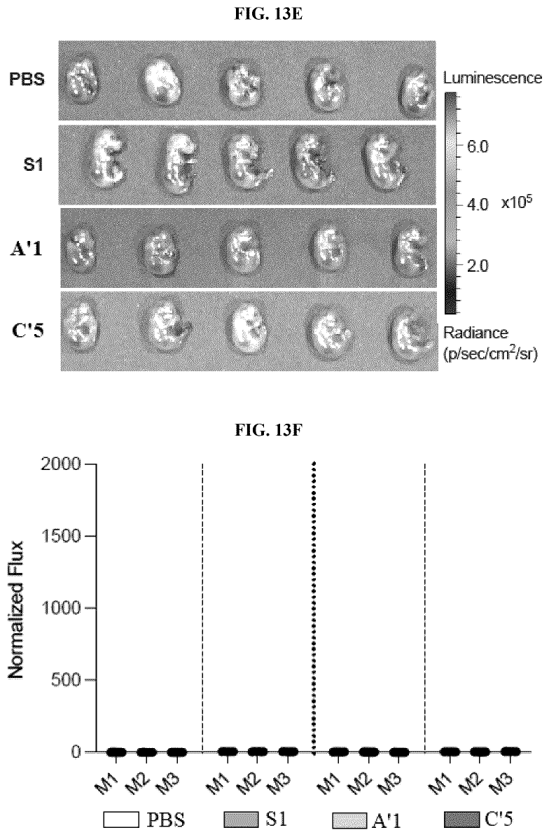

To confirm the results of the initial library screens, the top performing LNPs from each library (i.e., A′1, B′5 and C′5), along with S1, were further evaluated in a dose-dependent manner for in vitro luciferase expression and cell viability in BeWo b30 cells 24 hours after treatment. Across all doses tested, LNP C′5 demonstrated improved luciferase expression compared to LNP S1 (FIG. 12A). At lower doses, LNPs A′1 and B′5 showed similar or slightly improved luciferase expression compared to S1 while at the highest dose evaluated, both LNPs had significantly higher luciferase expression compared to S1. Interestingly, while LNP A′1 had comparable luciferase expression to S1 across the lower doses, it exhibited the greatest improvement in luciferase expression at the highest dose. Additionally, none of the LNPs exhibited any cytotoxicity at the different doses that were tested (FIG. 13B).

Since LNP C′5 exhibited the most consistent improvement in mRNA delivery across all doses, this LNP was selected for further evaluation in vivo. Additionally, given the strong improvement in mRNA delivery for LNP A′1 at the highest dose, it was also selected for further evaluation in vivo for mRNA delivery to the placenta.

### Example 10: Optimized LNP C′5 Achieves Greater Placental mRNA LNP Delivery Compared to S1 in Pregnant Mice

Pregnant mice on gestational day E16 were treated with PBS or LNPs S1, A′1, and C′5 at a dose of 0.6 mg/kg of luciferase mRNA via a tail vein injection. 6 hours after treatment, mice were injected via an intraperitoneal injection of luciferin, euthanized and the maternal organs, placentas and fetuses were removed for bioluminescence imaging by an in vivo imaging system (IVIS) (FIG. 13A, FIG. 13B, and FIG. 13E). Bioluminescence signal in each organ was measured and quantified through regions of interest (ROI). In the maternal organs, bioluminescence signal was highest in the liver and the spleen for all LNP-treated mice (FIGS. 13A-13B). In the liver, LNP A′1 exhibited greater mRNA delivery compared to S1 while LNP C′5 demonstrated reduced mRNA delivery compared to both A′1 and S1. In the spleen, both LNP A′1 and C′5 exhibited increased mRNA delivery compared to S1, demonstrating the ability of these optimized LNPs to achieve improved extrahepatic delivery compared to the initial lead formulation.

Differences in the observed mRNA delivery of each LNP formulation may be attributed to their excipient compositions. As previously mentioned, LNP S1 comprises a molar ratio of 35 (C12-494):16 (DOPE):46.5 (cholesterol):2.5 (PEG). On the other hand, LNP A′1 has increased amounts of C12-494 and DOPE and reduced levels of cholesterol and PEG and LNP C′5 has increased amounts of C12-494 and decreased amounts of cholesterol compared to S1 (Table 3). Previously, DOPE has been shown to influence mRNA delivery to the liver compared to other phospholipids such as DSPC. Thus, the increased DOPE content in LNP A′1 may be driving increased mRNA delivery to the liver compared to the other formulations.

Earlier studies using the C12-494 ionizable lipid to formulate LNPs found higher spleen than liver mRNA delivery in pregnant mice. It was hypothesized that extrahepatic splenic mRNA delivery could be attributed to the lipid structure of C12-494, where the ester linkages impact the overall electronegativity of the lipid and potentially contribute to the observed splenic mRNA delivery. Additionally, earlier studies have described splenic mRNA delivery following intravenous injection with an ionizable lipid also containing ester linkages, which were attributed to the potential for these linkages to be degraded in the liver, but remain intact in the splenic environment. Since splenic mRNA delivery is increased from LNP S1 to A′1 to C′5, which correlates with increasing C12-494 content in the formulation, mRNA delivery to the spleen may be enhanced with increased ionizable lipid content. However, more work is needed to further elucidate the exact mechanisms behind shifts in biodistribution due to changes in excipient molar ratios.

Next, placentas and the fetuses were imaged and bioluminescence signal quantified in each treatment group. Only LNP C′5 significantly improved mRNA delivery to the placenta compared to S1, consistent with the in vitro results where LNP C′5 was a lead candidate for mRNA delivery to trophoblasts. Despite having promising results in vitro, LNP A′1 did not show a significant improvement with regard to mRNA delivery to the placenta in vivo. Additionally, there was no bioluminescence signal in the fetuses for the LNP treated groups, suggesting that the LNPs remain in the placenta and do not enter fetal circulation, likely due to their >100 nm size, which is expected prevent placental transport.

Organ specificity for each LNP treatment was also evaluated by summing the luminescent flux from the maternal organs, placentas and fetuses and calculating the percent of total flux for each organ (FIGS. 13A-13F). Across the treatment groups, LNP A′1 had the greatest percentage of luminescent flux in the spleen and liver with only about 20% of the signal found in the placentas. LNP S1 also had strong signal in the liver and spleen, but had greater specificity to the placenta, with 38% of the total luminescent flux found in the placentas. However, top performing LNP C′5 had the greatest specificity to the placenta with 65% of the total luminescent flux found in the placentas, 34% in the spleen, and less than 1% in the liver. The low specificity to the liver indicates the ability of LNP C′5 to enhance extrahepatic delivery of mRNA, particularly to the placenta in pregnant mice.

### Example 11: High-Throughput In Vivo Screen for Extrahepatic LNP Delivery

Over the last several years, preclinical work in the LNP field has shifted focus towards developing platforms for nucleic acid delivery to extrahepatic tissues including the lung, spleen, bone, and pancreas. These works have employed different approaches to achieve extrahepatic LNP delivery including the design of novel ionizable lipid structures, altering the standard four-component excipient composition, or through the inclusion of additional charge-based excipients into the LNP formulation. As the traditional four-component LNP systems pose certain advantages including simplified manufacturing, a 98 LNP library was designed with 24 unique ionizable lipids synthesized via simple S_{N}2 reaction chemistry from 8 polyamine cores and 3 epoxide tails. For the remaining LNPs in the library, 12 of the ionizable lipids were further explored by varying the excipient composition (Table 7). The C12-200 (LNP 97) and DLin-MC3-DMA (LNP 98) industry standard LNP formulations were also included as liver-tropic LNP controls.

TABLE 7
b-DNA Library LNP formulations as a molar ratio

| LNP | Ionizable | Ionizable | | Choles- | Lipid- |
|---|---|---|---|---|---|
| No. | Lipid | lipid | DOPE | terol | PEG |
| 1 | C12-480 | 35 | 16 | 46.5 | 2.5 |
| 2 | C12-482 | 35 | 16 | 46.5 | 2.5 |
| 3 | C12-488 | 35 | 16 | 46.5 | 2.5 |
| 4 | C12-489 | 35 | 16 | 46.5 | 2.5 |
| 5 | C12-490 | 35 | 16 | 46.5 | 2.5 |
| 6 | C12-493 | 35 | 16 | 46.5 | 2.5 |
| 7 | C12-494 | 35 | 16 | 46.5 | 2.5 |
| 8 | C12-497 | 35 | 16 | 46.5 | 2.5 |
| 9 | C14-480 | 35 | 16 | 46.5 | 2.5 |
| 10 | C14-482 | 35 | 16 | 46.5 | 2.5 |
| 11 | C14-488 | 35 | 16 | 46.5 | 2.5 |
| 12 | C14-489 | 35 | 16 | 46.5 | 2.5 |
| 13 | C14-490 | 35 | 16 | 46.5 | 2.5 |
| 14 | C14-493 | 35 | 16 | 46.5 | 2.5 |
| 15 | C14-494 | 35 | 16 | 46.5 | 2.5 |
| 16 | C14-497 | 35 | 16 | 46.5 | 2.5 |
| 17 | C16-480 | 35 | 16 | 46.5 | 2.5 |
| 18 | C16-482 | 35 | 16 | 46.5 | 2.5 |
| 19 | C16-488 | 35 | 16 | 46.5 | 2.5 |
| 20 | C16-489 | 35 | 16 | 46.5 | 2.5 |
| 21 | C16-490 | 35 | 16 | 46.5 | 2.5 |
| 22 | C16-493 | 35 | 16 | 46.5 | 2.5 |
| 23 | C16-494 | 35 | 16 | 46.5 | 2.5 |
| 24 | C16-497 | 35 | 16 | 46.5 | 2.5 |
| 25 | C12-480 | 52.5 | 16 | 46.5 | 2.5 |
| 26 | C12-482 | 52.5 | 16 | 46.5 | 2.5 |
| 27 | C12-494 | 52.5 | 16 | 46.5 | 2.5 |
| 28 | C12-497 | 52.5 | 16 | 46.5 | 2.5 |
| 29 | C14-480 | 52.5 | 16 | 46.5 | 2.5 |
| 30 | C14-482 | 52.5 | 16 | 46.5 | 2.5 |
| 31 | C14-494 | 52.5 | 16 | 46.5 | 2.5 |
| 32 | C14-497 | 52.5 | 16 | 46.5 | 2.5 |
| 33 | C16-480 | 52.5 | 16 | 46.5 | 2.5 |
| 34 | C16-482 | 52.5 | 16 | 46.5 | 2.5 |
| 35 | C16-494 | 52.5 | 16 | 46.5 | 2.5 |
| 36 | C16-497 | 52.5 | 16 | 46.5 | 2.5 |
| 37 | C12-480 | 17.5 | 16 | 46.5 | 2.5 |
| 38 | C12-482 | 17.5 | 16 | 46.5 | 2.5 |
| 39 | C12-494 | 17.5 | 16 | 46.5 | 2.5 |
| 40 | C12-497 | 17.5 | 16 | 46.5 | 2.5 |
| 41 | C14-480 | 17.5 | 16 | 46.5 | 2.5 |
| 42 | C14-482 | 17.5 | 16 | 46.5 | 2.5 |
| 43 | C14-494 | 17.5 | 16 | 46.5 | 2.5 |
| 44 | C14-497 | 17.5 | 16 | 46.5 | 2.5 |
| 45 | C16-480 | 17.5 | 16 | 46.5 | 2.5 |
| 46 | C16-482 | 17.5 | 16 | 46.5 | 2.5 |
| 47 | C16-494 | 17.5 | 16 | 46.5 | 2.5 |
| 48 | C16-497 | 17.5 | 16 | 46.5 | 2.5 |
| 49 | C12-480 | 35 | 24 | 46.5 | 2.5 |
| 50 | C12-482 | 35 | 24 | 46.5 | 2.5 |
| 51 | C12-494 | 35 | 24 | 46.5 | 2.5 |
| 52 | C12-497 | 35 | 24 | 46.5 | 2.5 |
| 53 | C14-480 | 35 | 24 | 46.5 | 2.5 |
| 54 | C14-482 | 35 | 24 | 46.5 | 2.5 |
| 55 | C14-494 | 35 | 24 | 46.5 | 2.5 |
| 56 | C14-497 | 35 | 24 | 46.5 | 2.5 |
| 57 | C16-480 | 35 | 24 | 46.5 | 2.5 |
| 58 | C16-482 | 35 | 24 | 46.5 | 2.5 |
| 59 | C16-494 | 35 | 24 | 46.5 | 2.5 |
| 60 | C16-497 | 35 | 24 | 46.5 | 2.5 |
| 61 | C12-480 | 35 | 8 | 46.5 | 2.5 |
| 62 | C12-482 | 35 | 8 | 46.5 | 2.5 |
| 63 | C12-494 | 35 | 8 | 46.5 | 2.5 |
| 64 | C12-497 | 35 | 8 | 46.5 | 2.5 |
| 65 | C14-480 | 35 | 8 | 46.5 | 2.5 |
| 66 | C14-482 | 35 | 8 | 46.5 | 2.5 |
| 67 | C14-494 | 35 | 8 | 46.5 | 2.5 |
| 68 | C14-497 | 35 | 8 | 46.5 | 2.5 |
| 69 | C16-480 | 35 | 8 | 46.5 | 2.5 |
| 70 | C16-482 | 35 | 8 | 46.5 | 2.5 |
| 71 | C16-494 | 35 | 8 | 46.5 | 2.5 |
| 72 | C16-497 | 35 | 8 | 46.5 | 2.5 |
| 73 | C12-480 | 35 | 16 | 69.75 | 2.5 |
| 74 | C12-482 | 35 | 16 | 23.25 | 2.5 |
| 75 | C12-494 | 35 | 16 | 23.25 | 2.5 |
| 76 | C12-497 | 35 | 16 | 23.25 | 2.5 |
| 77 | C14-480 | 35 | 16 | 23.25 | 2.5 |
| 78 | C14-482 | 35 | 16 | 23.25 | 2.5 |
| 79 | C14-494 | 35 | 16 | 23.25 | 2.5 |
| 80 | C14-497 | 35 | 16 | 23.25 | 2.5 |
| 81 | C16-480 | 35 | 16 | 23.25 | 2.5 |
| 82 | C16-482 | 35 | 16 | 23.25 | 2.5 |
| 83 | C16-494 | 35 | 16 | 23.25 | 2.5 |
| 84 | C16-497 | 35 | 16 | 23.25 | 2.5 |
| 85 | C12-480 | 35 | 16 | 46.5 | 5 |
| 86 | C12-482 | 35 | 16 | 46.5 | 5 |
| 87 | C12-494 | 35 | 16 | 46.5 | 5 |
| 88 | C12-497 | 35 | 16 | 46.5 | 5 |
| 89 | C14-480 | 35 | 16 | 46.5 | 5 |
| 90 | C14-482 | 35 | 16 | 46.5 | 5 |
| 91 | C14-494 | 35 | 16 | 46.5 | 5 |
| 92 | C14-497 | 35 | 16 | 46.5 | 5 |
| 93 | C16-480 | 35 | 16 | 46.5 | 5 |
| 94 | C16-482 | 35 | 16 | 46.5 | 5 |
| 95 | C16-494 | 35 | 16 | 46.5 | 5 |
| 96 | C16-497 | 35 | 16 | 46.5 | 5 |
| 97 | C12-200 | 35 | 16 | 46.5 | 2.5 |
| 98 | DLin-MC3- | 50 | 10 (DSPC) | 38.5 | 1.5 (DMG- |
| | DMA | | | | PEG) |

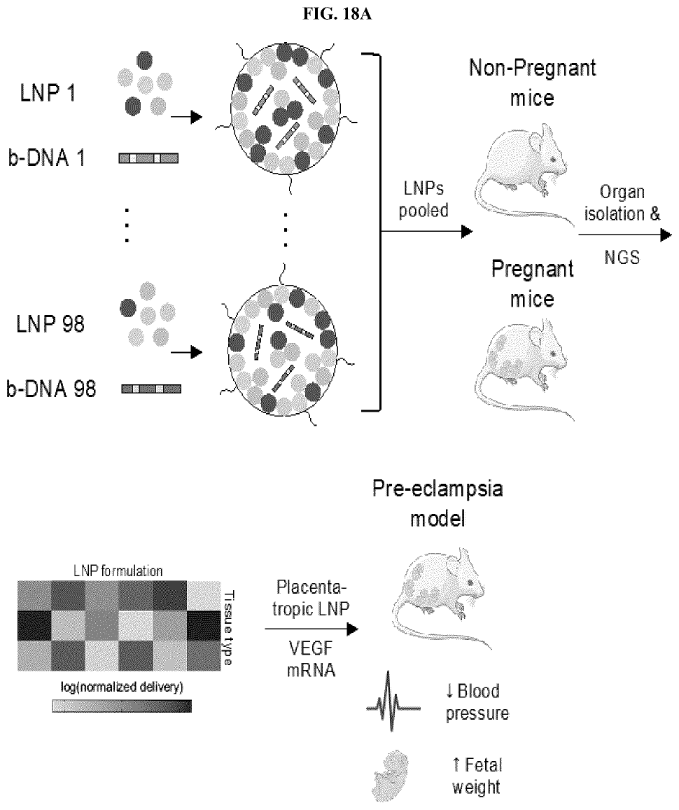

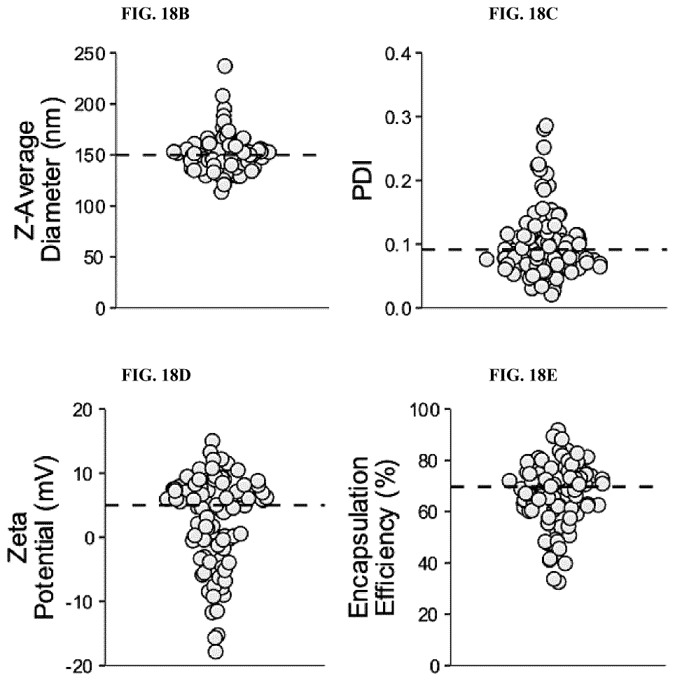

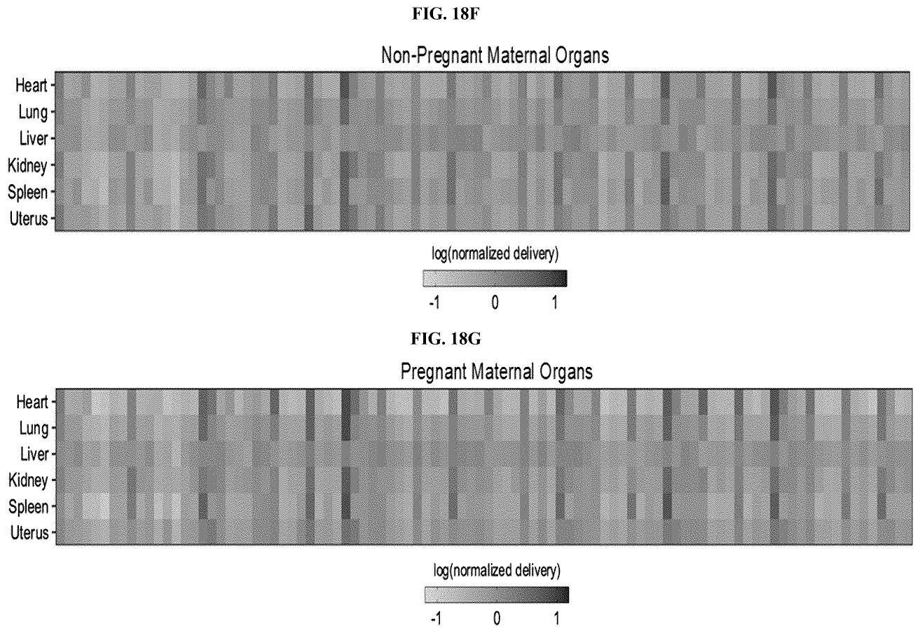

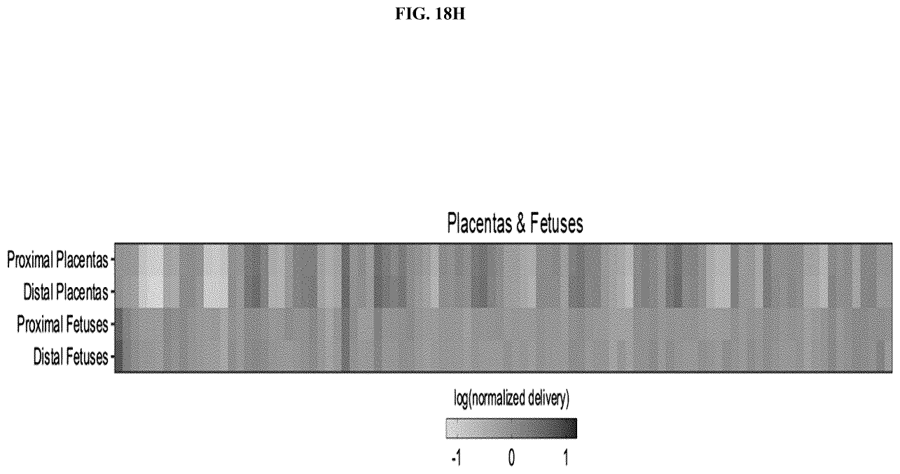

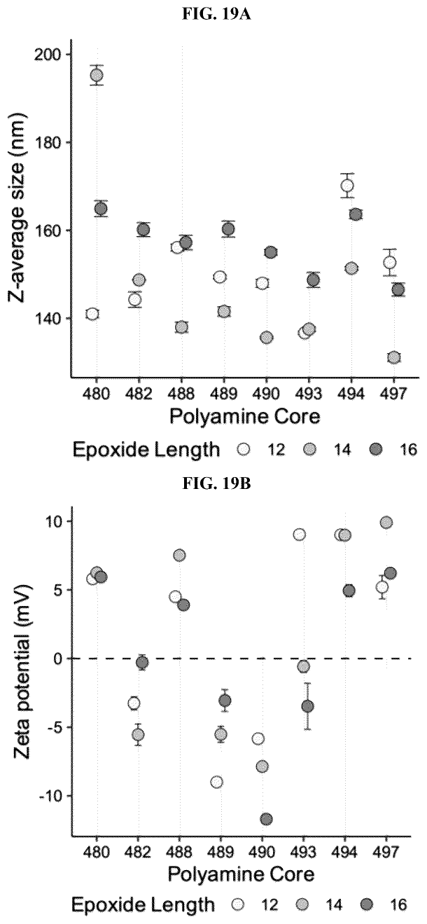

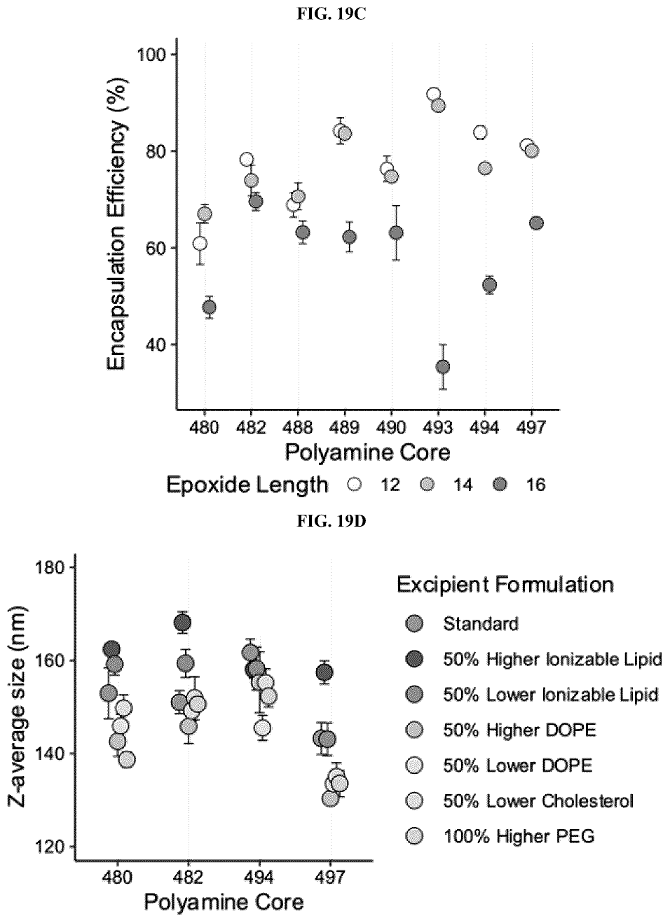

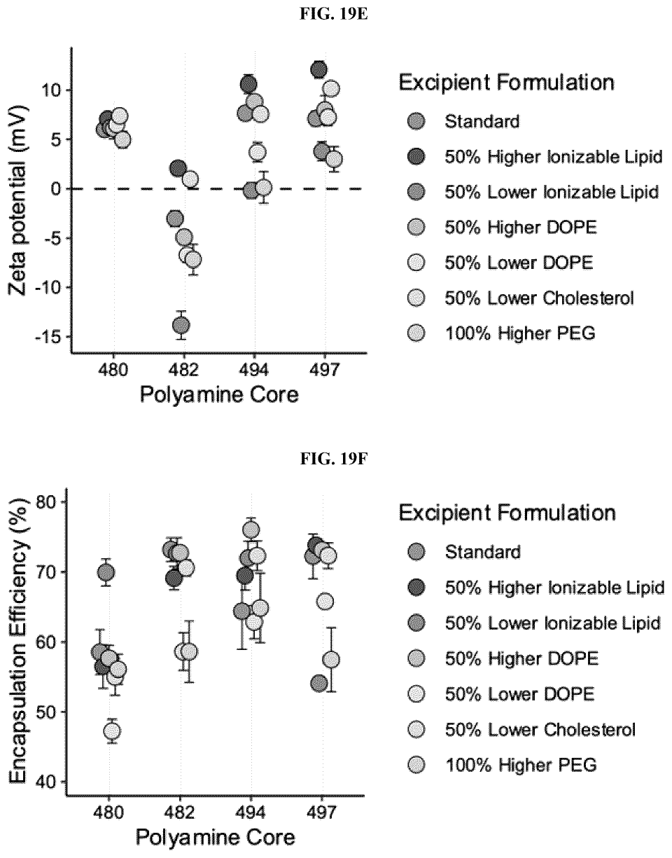

While the use of b-DNAs has been explored extensively for high-throughput evaluations of LNP biodistribution, the present disclosure represents the first to use this screening platform to identify extrahepatic formulations for LNP delivery in pregnant mice. The lipid phase containing ionizable lipid, phospholipid (1,2-dioleoyl-sn-glycero-3-phosphoethanolamine, DOPE), cholesterol, and lipid-PEG (C14-PEG₂₀₀₀) for each LNP in the library (Table 3) was used to formulate LNPs via pipette mixing where each formulation encapsulated a unique 61 nucleotide b-DNA sequences (FIG. 18A). LNPs were characterized by their hydrodynamic diameter (FIG. 18B, Table 8), polydispersity index (FIG. 18C, Table 8), surface zeta potential (FIG. 18D, Table 8), and encapsulation efficiency (FIG. 18D, Table 8). In terms of size, LNPs with C16 epoxide tails (FIG. 19A) or higher amounts of ionizable lipid (FIG. 19D) were generally larger, while LNPs with higher amounts of lipid-PEG were generally smaller (FIG. 19D). Surface charge seemed to depend on both polyamine core and excipient composition where the 480 and 497 cores produced LNPs with positive zeta potentials (FIG. 19B and FIG. 19E), while LNPs with high ionizable lipid content were more positively charged and LNPs with low ionizable lipid content were more negatively charged than the standard excipient formulations (FIG. 19E). Finally, LNPs with C16 epoxide tail lengths (FIG. 19C) and lower amounts of DOPE phospholipid (FIG. 19F) exhibited lower encapsulation efficiencies.

TABLE 8
Exemplary characterization data for certain
LNPs of the present disclosure

| | Mean | Mean | | Mean |
|---|---|---|---|---|
| LNP | z-average | polydispersity | Mean zeta | encapsulation |
| No. | diameter (nm) | index (PDI) | potential (mV) | efficiency (%) |
| 1 | 141.0 | 0.071 | 5.80 | 60.9 |
| 2 | 144.3 | 0.092 | −3.26 | 78.3 |
| 3 | 156.1 | 0.107 | 4.50 | 68.9 |
| 4 | 149.4 | 0.119 | −9.00 | 84.2 |
| 5 | 148.0 | 0.136 | −5.84 | 76.4 |
| 6 | 136.7 | 0.154 | 9.04 | 91.8 |
| 7 | 170.2 | 0.092 | 9.02 | 83.9 |
| 8 | 152.7 | 0.192 | 5.20 | 81.2 |
| 9 | 195.3 | 0.252 | 6.25 | 67.0 |
| 10 | 148.7 | 0.110 | −5.55 | 73.9 |
| 11 | 138.0 | 0.127 | 7.52 | 70.6 |
| 12 | 141.6 | 0.106 | −5.52 | 83.7 |
| 13 | 135.6 | 0.093 | −7.86 | 74.7 |
| 14 | 137.5 | 0.114 | −0.58 | 89.4 |
| 15 | 151.4 | 0.064 | 8.98 | 76.5 |
| 16 | 130.6 | 0.109 | 9.91 | 80.1 |
| 17 | 164.9 | 0.036 | 5.94 | 47.7 |
| 18 | 160.2 | 0.044 | −0.29 | 69.6 |
| 19 | 157.2 | 0.036 | 3.90 | 63.2 |
| 20 | 160.3 | 0.078 | −3.06 | 62.3 |
| 21 | 155.0 | 0.076 | −11.70 | 63.1 |
| 22 | 148.7 | 0.090 | −3.48 | 32.6 |
| 23 | 163.6 | 0.062 | 4.95 | 52.3 |
| 24 | 146.5 | 0.031 | 6.21 | 65.1 |
| 25 | 163.3 | 0.070 | 5.96 | 65.4 |
| 26 | 176.9 | 0.121 | 4.73 | 72.0 |
| 27 | 161.9 | 0.115 | 11.53 | 75.0 |
| 28 | 162.2 | 0.026 | 15.03 | 75.5 |
| 29 | 188.3 | 0.085 | 7.33 | 59.1 |
| 30 | 161.1 | 0.075 | 0.87 | 72.7 |
| 31 | 208.0 | 0.144 | 13.27 | 71.9 |
| 32 | 162.6 | 0.211 | 12.20 | 70.7 |
| 33 | 161.5 | 0.058 | 7.92 | 45.0 |
| 34 | 166.5 | 0.070 | 0.59 | 62.6 |
| 35 | 154.3 | 0.103 | 6.96 | 61.6 |
| 36 | 147.6 | 0.064 | 8.99 | 75.3 |
| 37 | 164.4 | 0.191 | 6.59 | 74.5 |
| 38 | 170.8 | 0.223 | −8.48 | 79.4 |
| 39 | 182.9 | 0.281 | 1.73 | 81.3 |
| 40 | 154.5 | 0.286 | 6.64 | 81.9 |
| 41 | 237.1 | 0.217 | 5.04 | 73.1 |
| 42 | 155.6 | 0.118 | −17.83 | 74.3 |
| 43 | 152.7 | 0.124 | 0.85 | 77.3 |
| 44 | 137.8 | 0.149 | 1.88 | 81.0 |
| 45 | 154.0 | 0.092 | 5.89 | 62.3 |
| 46 | 151.8 | 0.046 | −7.48 | 64.3 |
| 47 | 149.9 | 0.021 | −5.46 | 66.6 |
| 48 | 137.0 | 0.077 | −0.97 | 54.1 |
| 49 | 130.2 | 0.116 | 5.10 | 60.2 |
| 50 | 131.5 | 0.080 | 0.89 | 70.9 |
| 51 | 129.3 | 0.156 | 4.88 | 80.2 |
| 52 | 129.3 | 0.107 | 11.18 | 76.9 |
| 53 | 148.1 | 0.053 | 9.25 | 62.0 |
| 54 | 153.1 | 0.071 | −2.51 | 75.8 |
| 55 | 169.6 | 0.082 | 3.81 | 79.4 |
| 56 | 129.7 | 0.092 | 9.74 | 71.7 |
| 57 | 149.6 | 0.078 | 4.57 | 50.7 |
| 58 | 153.0 | 0.056 | −1.59 | 71.5 |
| 59 | 167.0 | 0.034 | 3.70 | 70.3 |
| 60 | 132.1 | 0.065 | 3.95 | 70.7 |
| 61 | 137.4 | 0.085 | 5.31 | 49.2 |
| 62 | 150.2 | 0.068 | −0.54 | 64.6 |
| 63 | 136.7 | 0.068 | 3.19 | 71.7 |
| 64 | 113.7 | 0.147 | 9.47 | 68.5 |
| 65 | 145.9 | 0.092 | 7.29 | 42.7 |
| 66 | 141.9 | 0.078 | −2.24 | 62.9 |
| 67 | 145.4 | 0.059 | −0.29 | 61.4 |
| 68 | 136.2 | 0.102 | 3.47 | 65.4 |
| 69 | 154.5 | 0.096 | 5.84 | 33.7 |
| 70 | 155.5 | 0.067 | −3.16 | 48.3 |
| 71 | 154.5 | 0.056 | −2.66 | 55.5 |
| 72 | 130.9 | 0.076 | 5.01 | 63.4 |
| 73 | 143.4 | 0.047 | 7.96 | 54.3 |
| 74 | 136.2 | 0.066 | 3.13 | 74.8 |
| 75 | 149.9 | 0.058 | 3.26 | 72.6 |
| 76 | 134.2 | 0.133 | 9.20 | 66.9 |
| 77 | 144.7 | 0.080 | 8.36 | 60.5 |
| 78 | 151.4 | 0.094 | 2.70 | 69.8 |
| 79 | 149.3 | 0.103 | 5.36 | 79.5 |
| 80 | 125.5 | 0.110 | 9.87 | 79.8 |
| 81 | 161.2 | 0.094 | 7.53 | 39.8 |
| 82 | 168.2 | 0.068 | 4.48 | 67.1 |
| 83 | 166.6 | 0.100 | 6.51 | 64.8 |
| 84 | 145.6 | 0.068 | 8.89 | 72.9 |
| 85 | 143.6 | 0.118 | 7.75 | 57.8 |
| 86 | 148.2 | 0.145 | 1.55 | 66.2 |
| 87 | 144.4 | 0.225 | 5.87 | 78.3 |
| 88 | 121.0 | 0.185 | 8.16 | 73.3 |
| 89 | 139.5 | 0.127 | 1.66 | 62.2 |
| 90 | 152.3 | 0.078 | −6.80 | 68.5 |
| 91 | 159.3 | 0.130 | −0.38 | 70.7 |
| 92 | 140.1 | 0.129 | 0.56 | 57.5 |
| 93 | 133.1 | 0.084 | 6.14 | 48.3 |
| 94 | 151.4 | 0.061 | −11.50 | 41.2 |
| 95 | 153.1 | 0.079 | −5.12 | 45.6 |
| 96 | 134.9 | 0.113 | 0.29 | 41.6 |
| 97 | 158.5 | 0.051 | −3.92 | 82.8 |
| 98 | 173.4 | 0.056 | −15.67 | 88.2 |

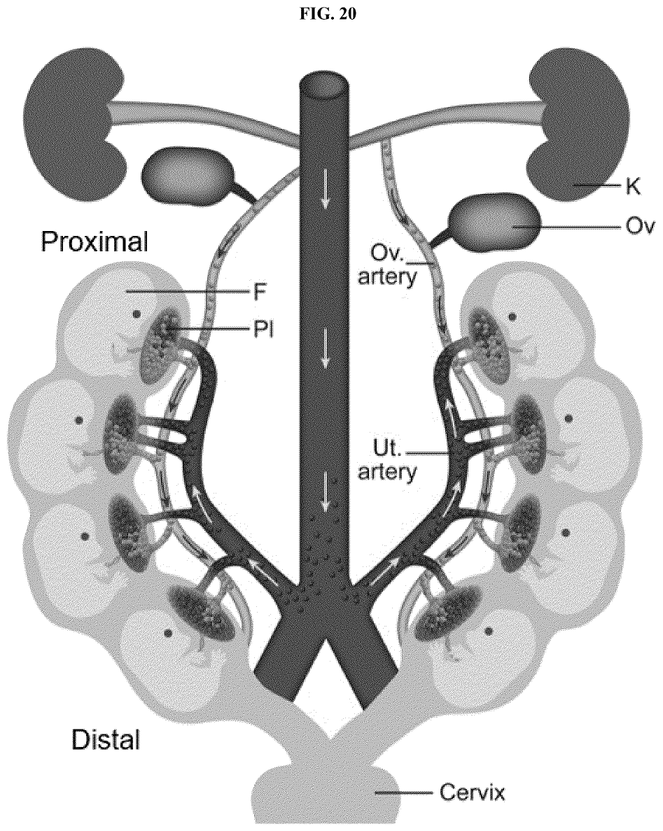

These 98 LNPs were pooled along with an unencapsulated b-DNA and administered to non-pregnant and gestational day E16 pregnant mice via tail vein injection (FIG. 18A). 6 h post administration, mice were euthanized, and the heart, lung, liver, kidneys, spleen, uterus, and fetuses and placentas were collected. It has been previously hypothesized that nanoparticle biodistribution to the placenta and fetus in pregnant mice might vary based on their location in the uterine horn due to two distinct blood supplies in the uterus. Here, proximal placentas/fetuses which are located closest to the ovaries are distinguished from distal placentas/fetuses which are located closest to the cervix (FIG. 20). Organs were processed to extract b-DNA which was subsequently amplified and used to detect LNP delivery by next generation sequencing (FIG. 18A). LNP delivery to the non-pregnant maternal organs (FIG. 18F), pregnant maternal organs (FIG. 18G), and the placentas and fetuses (FIG. 18H) is displayed using heatmaps. In the liver and fetuses, LNP delivery does not vary substantially across the library; however, in the extrahepatic organs as well as the placentas, some LNPs are clearly enriched while others are depleted.

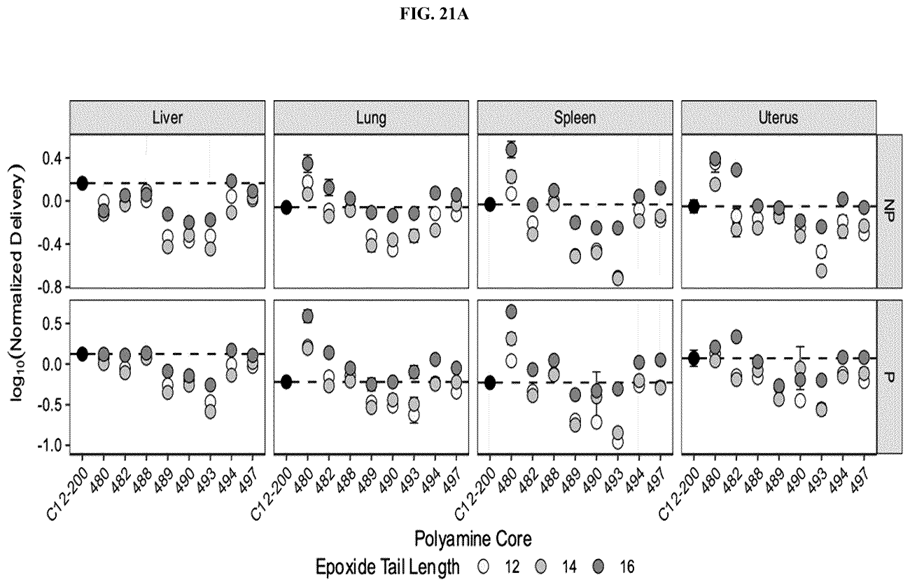

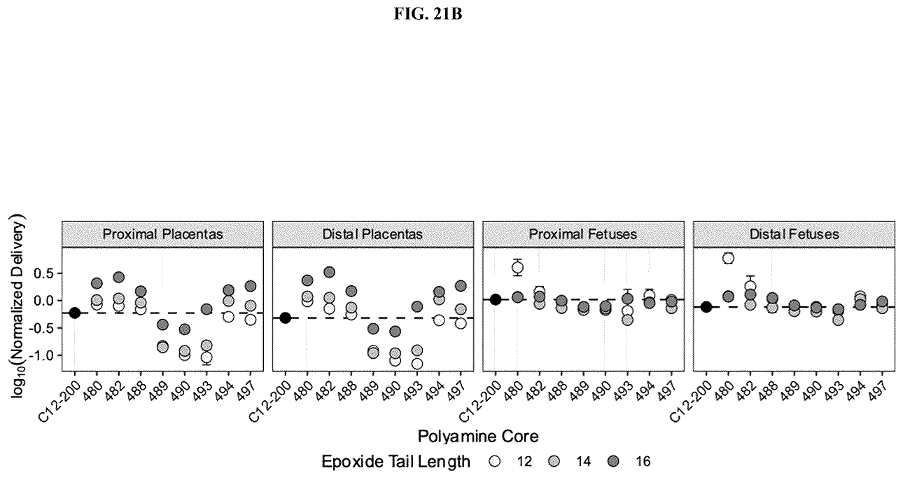

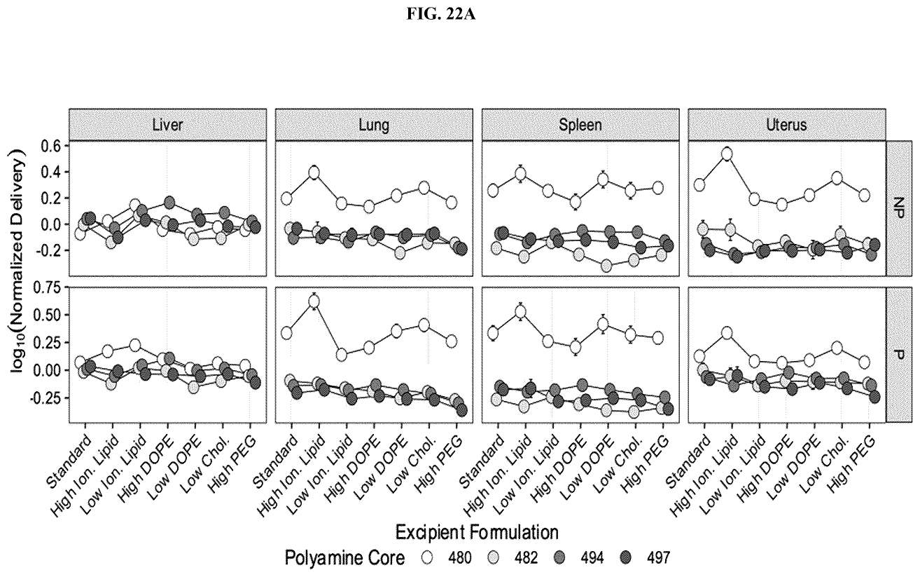

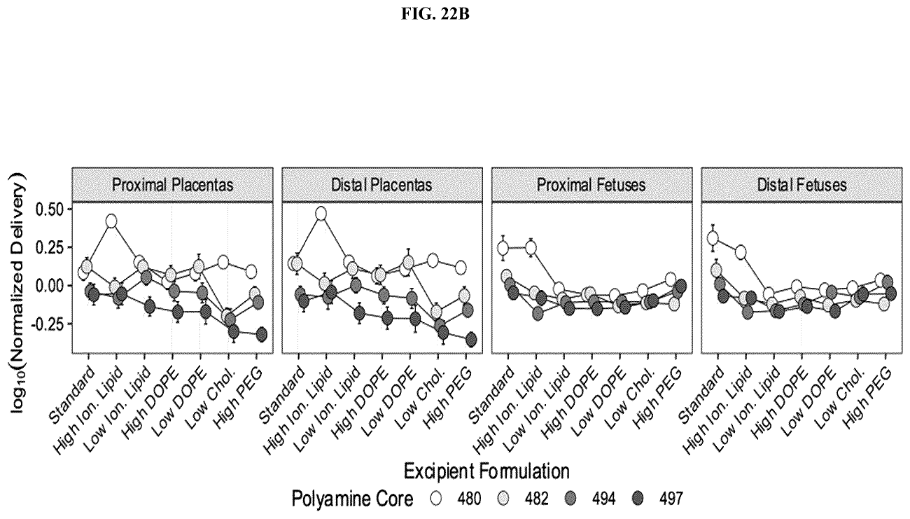

Elucidation of the effect of changing the ionizable lipid polyamine core and epoxide tail length was sought, as well as the excipient composition on LNP delivery. Several of the ionizable lipid polyamine cores enabled extrahepatic LNP delivery, including the 480, 482, 488, 494, and 497 cores for delivery to the placentas (FIGS. 21A-21B). Interestingly, LNPs containing ionizable lipids with C16 epoxide tails enabled higher delivery than those with C12 epoxide tails despite having lower encapsulation efficiencies (FIG. 19C and FIGS. 21A-21B). In the lung, spleen, uterus, and placentas, increasing the amount of ionizable lipid appears to improve LNP delivery, particularly for LNPs with ionizable lipids containing the 480 polyamine core (FIGS. 22A-22B). For LNPs containing ionizable lipids with the 482, 494, and 497 polyamine cores, decreasing the amount of cholesterol depletes LNP delivery to the placentas (FIG. 22B).

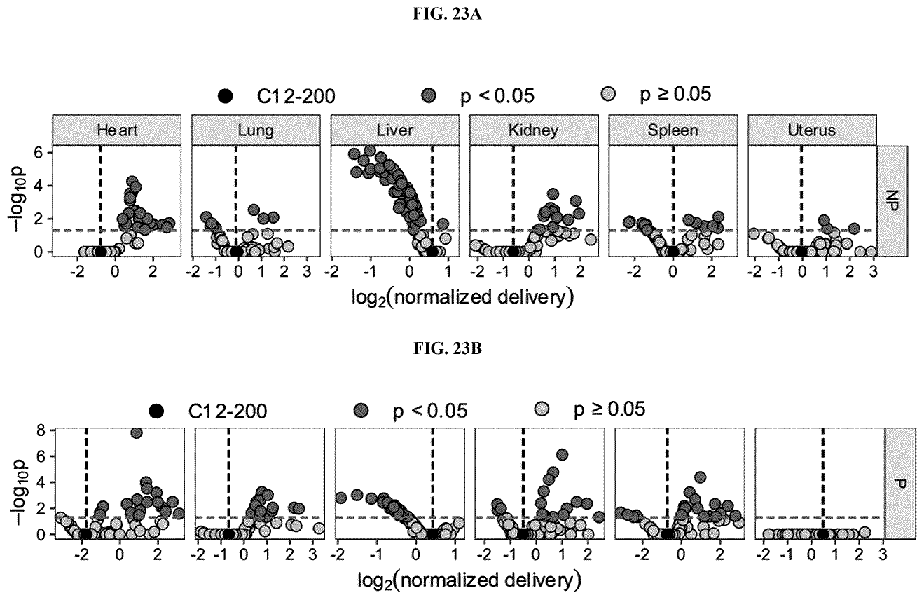

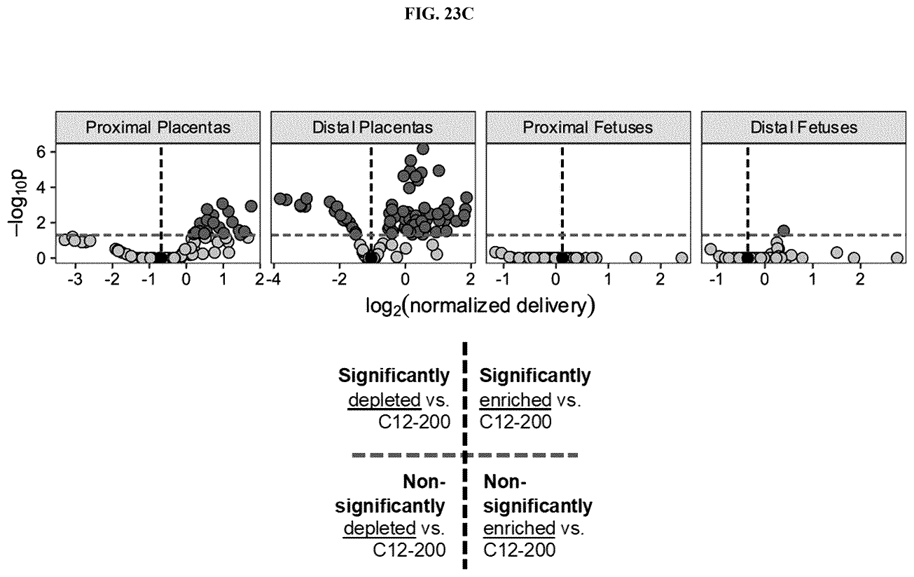

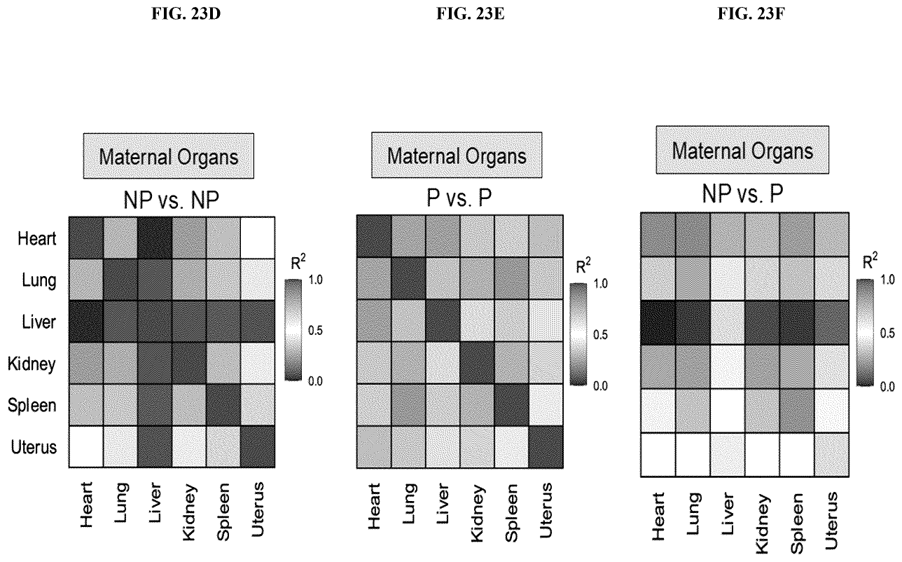

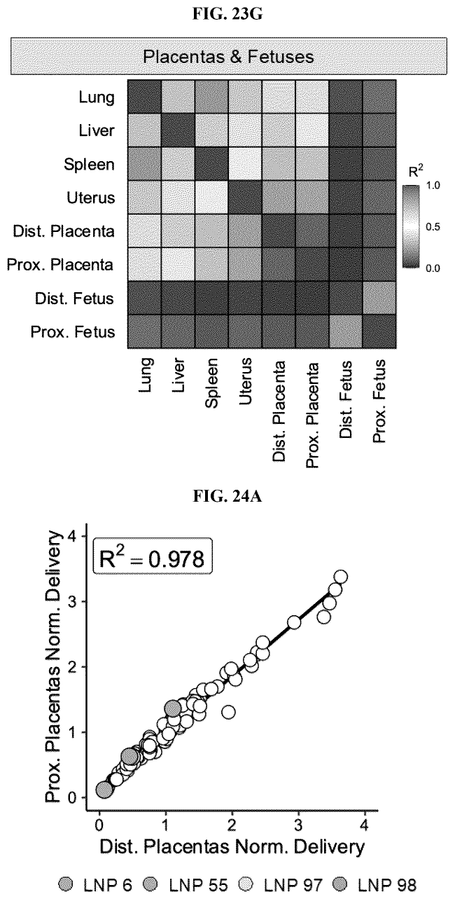

To further examine the potential for extrahepatic LNP delivery, volcano plots were constructed where LNPs in the upper half of the plots are either significantly depleted (top left quadrant) or significantly enriched (top right quadrant) in a particular organ compared to the C12-200 industry standard LNP formulation (FIGS. 23A-23C). As the C12-200 LNP demonstrates strong liver tropism, unsurprisingly 41 and 12 of the LNP formulations described herein are depleted in the non-pregnant and pregnant liver, respectively (FIGS. 23A-23B). Evidence of extrahepatic LNP delivery in the heart, lung, kidneys, and spleen was observed; more LNP formulations are significantly enriched in the pregnant lung and spleen (16 and 16, respectively) than the non-pregnant lung and spleen (4 and 6, respectively) (FIGS. 23A-23B). Perhaps most encouraging, 52 LNPs mediated significantly higher delivery to the distal placentas compared to the C12-200 formulation (FIG. 23C).

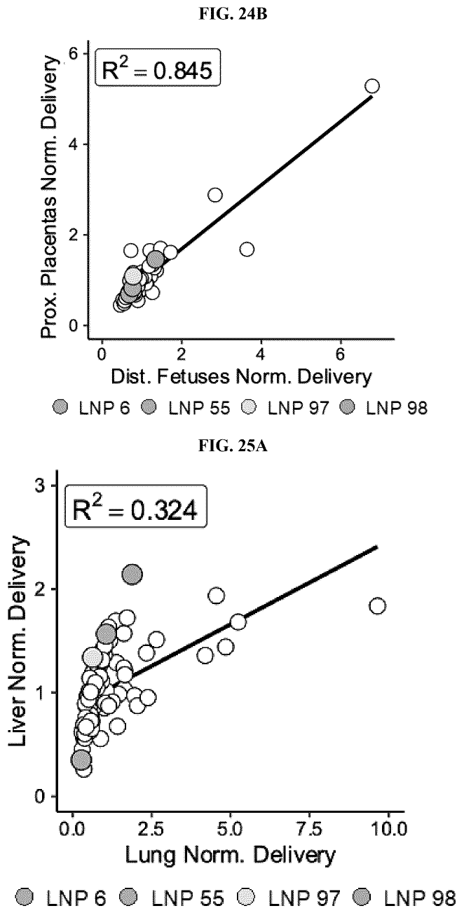

By calculating the correlation coefficient of normalized delivery for every pair of organs, the relationship between hepatic and extrahepatic delivery can be understood (FIGS. 23D-23G). In both non-pregnant (FIG. 23D) and pregnant mice (FIG. 23E), LNP delivery to the liver demonstrates weak correlation with delivery to the extrahepatic organs; however, delivery to one extrahepatic organ generally correlates strongly with delivery to another extrahepatic organ. Interestingly, this effect does not hold in the pregnant uterus, where LNP delivery does not correlate strongly with any other organ (FIG. 23E), perhaps due to the strong blood supply to the placenta and fetus during pregnancy. Comparing organ pairs between non-pregnant and pregnant mice, LNP delivery to the liver and uterus demonstrates poor correlation with the rest of the maternal organs (FIG. 23F). Delivery to placentas and fetuses is generally weakly correlated with other maternal organs (FIG. 23G). Correlation in normalized delivery between distal and proximal placentas and distal and proximal fetuses was very strong, with squared correlation coefficients of 0.98 and 0.85, respectively (FIG. 23G and FIGS. 24A-24B). These results suggest that the location of a placenta/fetus in the uterine horn does not appear to affect LNP delivery.

### Example 12: Barcoding Identifies Placenta-Tropic mRNA LNP Formulation

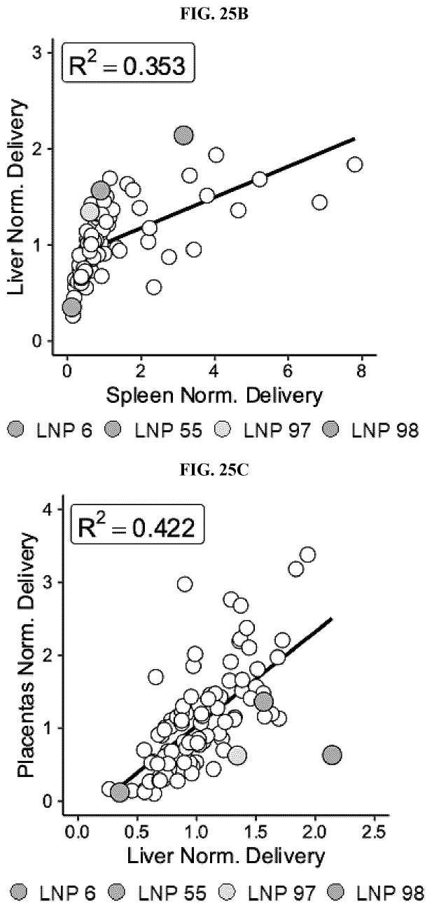

As is customary in studies using high-throughput screening, validation of the results from the pooled library was next sought by individually evaluating LNP formulations and their ability to deliver luciferase mRNA in vivo. As it is important to validate the use of high-throughput screening to identify both hits and poor-performing LNPs, LNP 6 was selected as a negative control which ranked in the bottom 5^{th} percentile for delivery to the lung, liver, spleen, and placentas (FIGS. 25A-25D). LNP 55 was selected as a placenta-tropic LNP formulation, aiming to maximize delivery to the placentas while minimizing delivery to the fetuses (FIGS. 25C-25D). Finally, LNPs 97 (C12-200) and 98 (DLin-MC3-DMA) were selected, which are industry standard LNP formulations, that demonstrated potent delivery to the liver with minimal placental delivery using pooled, high-throughput screening (FIGS. 25A-25D).

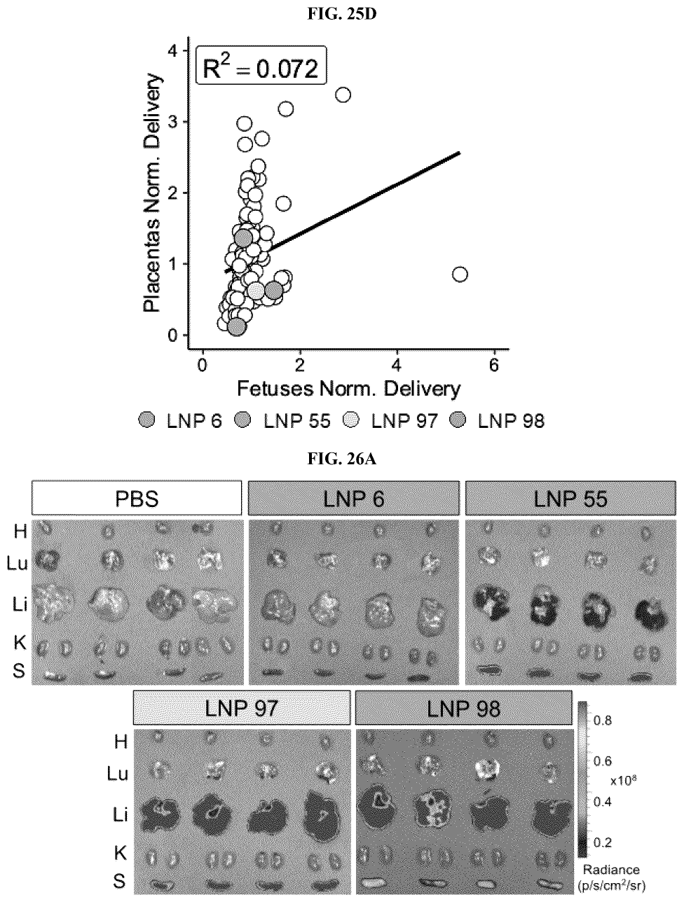

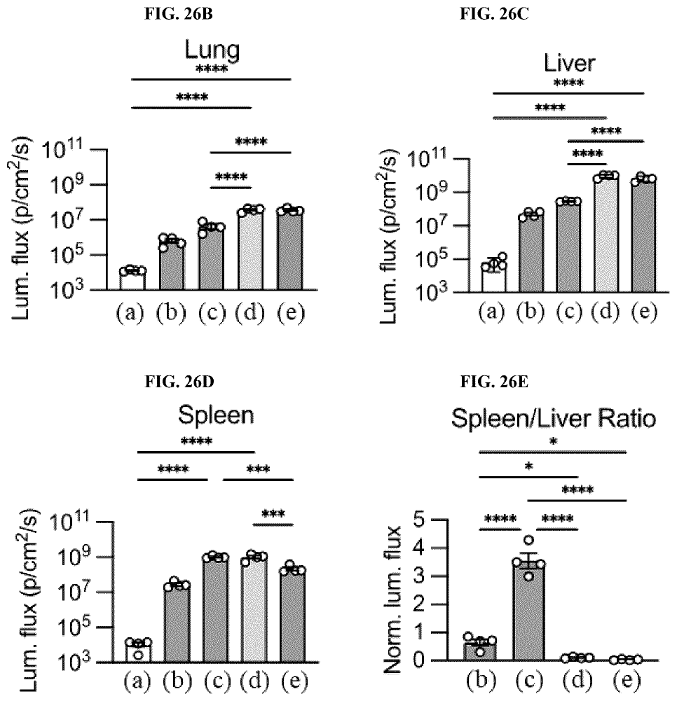

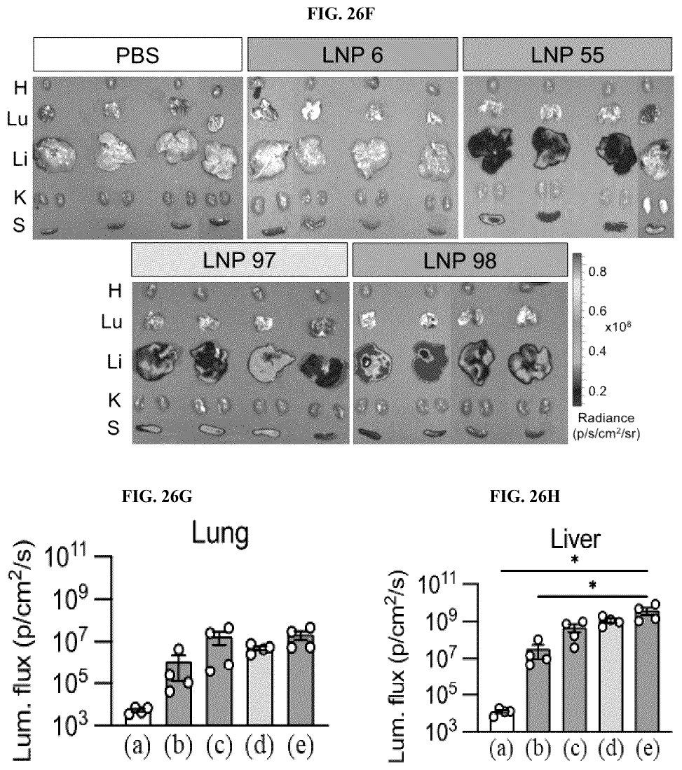

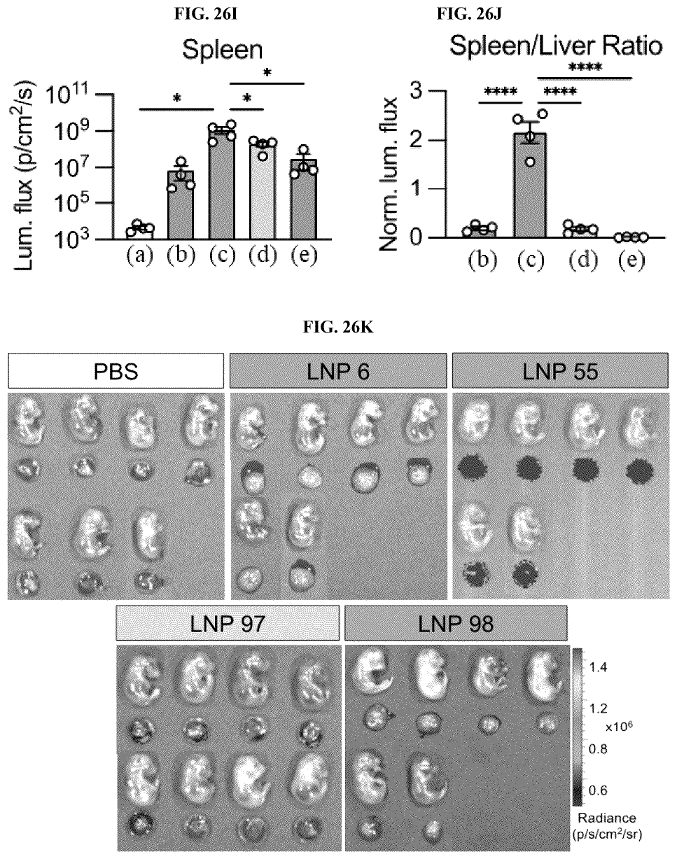

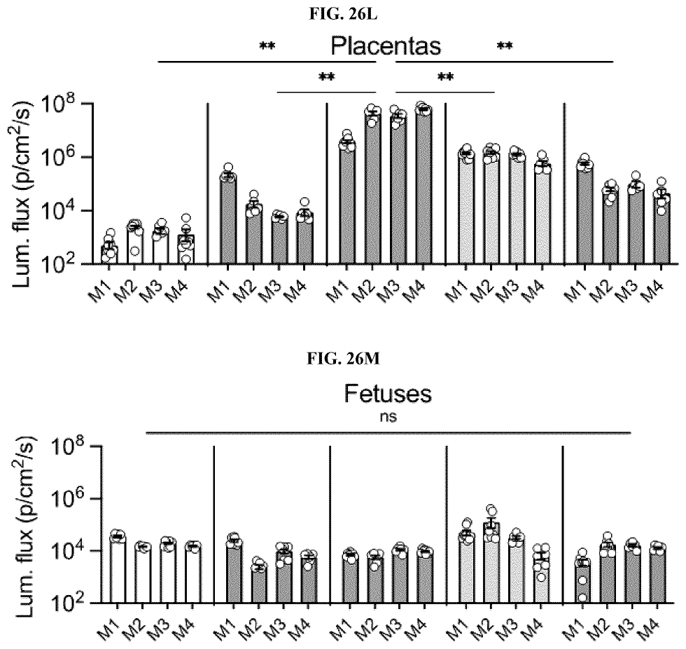

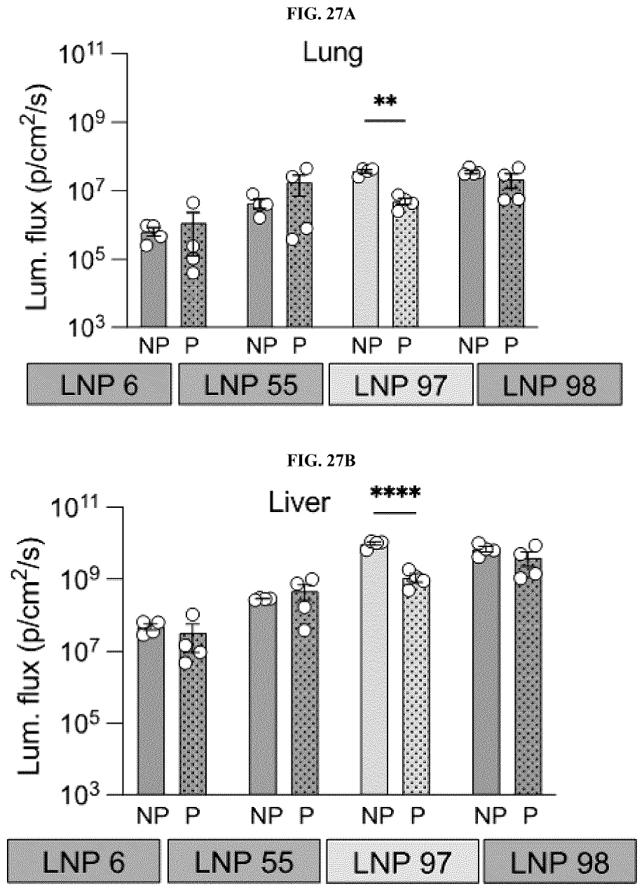

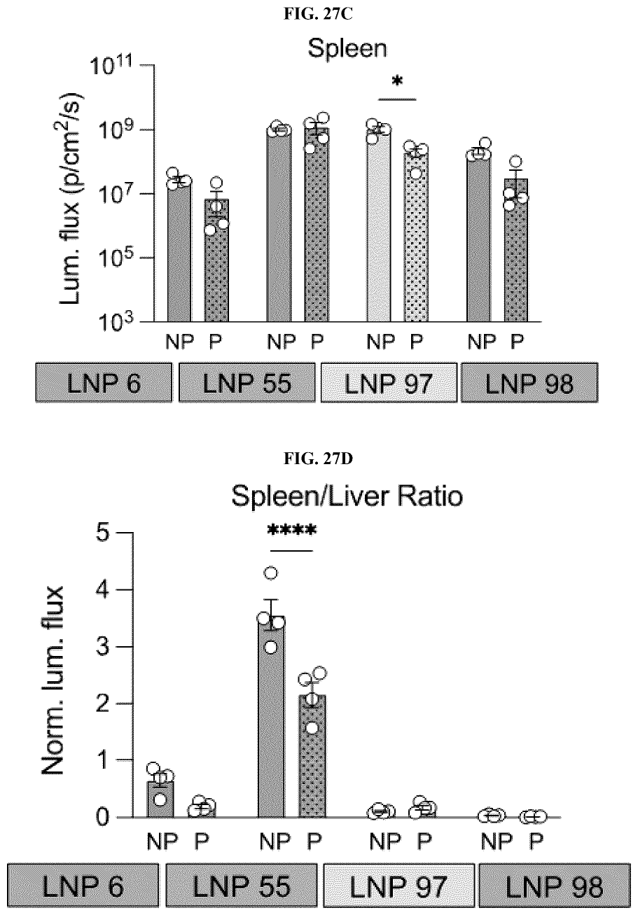

LNPs 6, 55, 97, and 98 were formulated with luciferase mRNA and administered to non-pregnant and pregnant mice at a dose of 0.6 mg of mRNA/kg via tail vein injection. 6 h following administration, mice were injected with D-Luciferin and euthanized in order to dissect the maternal organs as well as the placentas and fetuses for bioluminescent imaging using an in vivo imaging system (IVIS) (FIG. 26A, FIG. 26F, and FIG. 26K). Quantification of bioluminescent flux in the non-pregnant and pregnant maternal organs indicated luciferase mRNA LNP delivery to the lung (FIG. 26B and FIG. 26G), liver (FIG. 26C and FIG. 26H), and spleen (FIG. 26D and FIG. 26I). Validating the results from high-throughput screening, LNP 6 served as an effective negative control, enabling the lowest luciferase expression in the maternal organs and placentas of all the LNPs tested (FIGS. 26A-26M). LNPs 97 and 98 mediated luciferase mRNA delivery to the lung in non-pregnant mice, but not in pregnant mice (FIG. 26B and FIG. 26G). These industry standard LNPs performed as expected, both mediating significantly higher (****p<0.0001) luciferase expression in the liver than LNP 55 in non-pregnant mice (FIG. 26C). This phenomenon is consistent with the high-throughput b-DNA screening results that identified LNP 98 as the most enriched LNP in both the non-pregnant and pregnant liver (FIGS. 25A-25C). Interesting, luminescent flux values in the lung (**p<0.01), liver (****p<0.0001), and spleen (*p<0.05) are significantly lower for LNP 97 in pregnant mice than non-pregnant mice (FIGS. 27A-27C).

LNP 55 enabled potent extrahepatic luciferase mRNA delivery to the spleen, with significantly higher (*p<0.05) luminescent flux values than LNPs 97 and 98 in pregnant mice (FIG. 26I). To quantify extrahepatic delivery, the spleen to liver ratio was calculated by dividing the luminescent flux values from the spleen by those in the liver for each treatment group. In both non-pregnant and pregnant mice, the spleen to liver ratio for LNP 55 is significantly higher (****p<0.0001) than LNPs 6, 97, and 98 (FIG. 26E and FIG. 26J). Although, it was observed that this spleen to liver ratio was significantly lower (****p<0.0001) in pregnant mice than non-pregnant mice for LNP 55 (FIG. 27D), perhaps suggesting that some of this extrahepatic LNP delivery was being shunted elsewhere, namely the placenta.

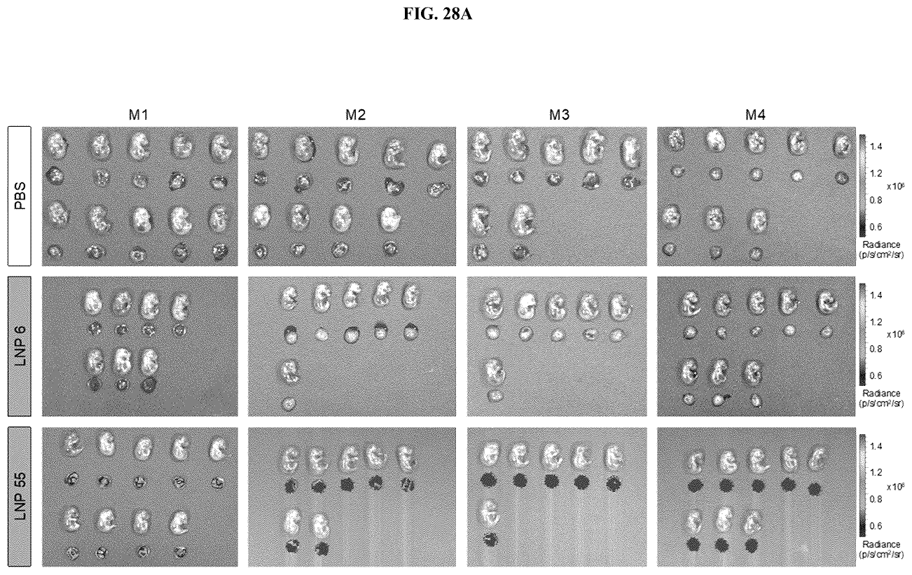

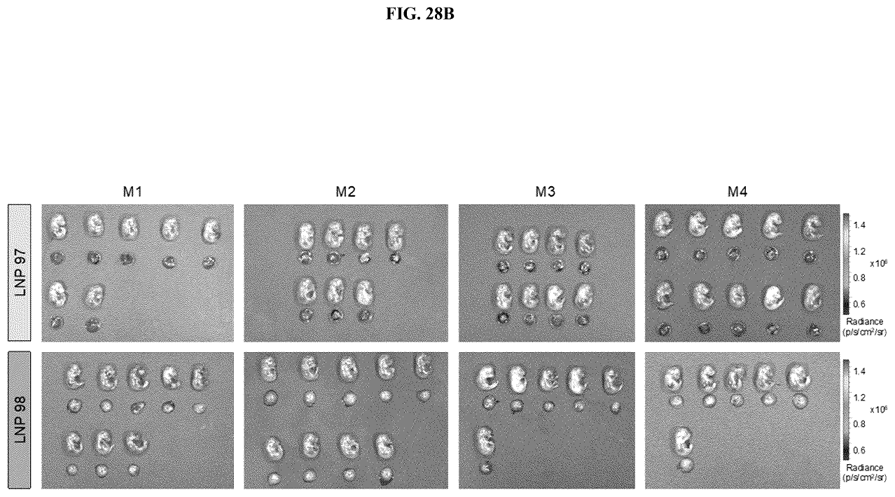

LNP 55 promoted significantly higher (**p<0.01) luciferase mRNA delivery to the placenta (FIG. 26K, FIG. 26L, and FIGS. 28A-28B) than the other three LNP treatment groups tested here, consistent with results from the b-DNA high-throughput screen. Unsurprisingly, luciferase expression in the fetuses for any of the LNP treatment groups was not observed (FIG. 26K FIG. 26M, and FIGS. 28A-28B), in agreement with previous observations regarding mRNA LNP delivery in pregnant mice. Without wishing to be bound by any theory, it has been reasoned that the lack of luciferase expression in the fetuses is due to the tight cellular barriers in the placenta, preventing mRNA LNPs on the order of 100 nm in diameter from navigating the 20 nm transtrophoblastic channels that separate maternal and fetal circulation.

Using high-throughput in vivo screening, a placenta-tropic LNP was identified that mediated more than an order of magnitude improvement in luciferase mRNA delivery to the placenta compared to two industry standard LNP formulations, namely C12-200 (28-fold improvement) and DLin-MC3-DMA (150-fold improvement).

### Example 13: Endogenous Targeting Mechanism for Placental LNP Delivery

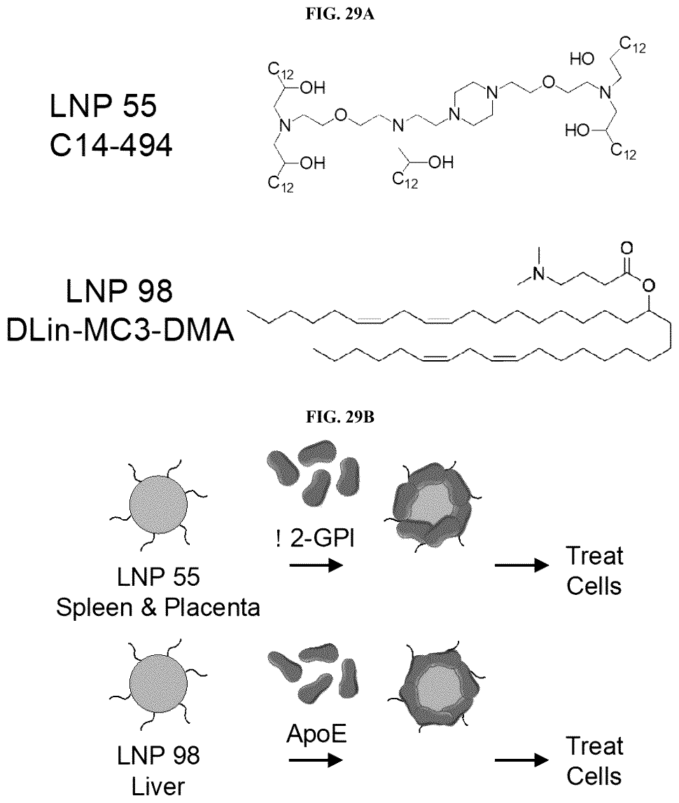

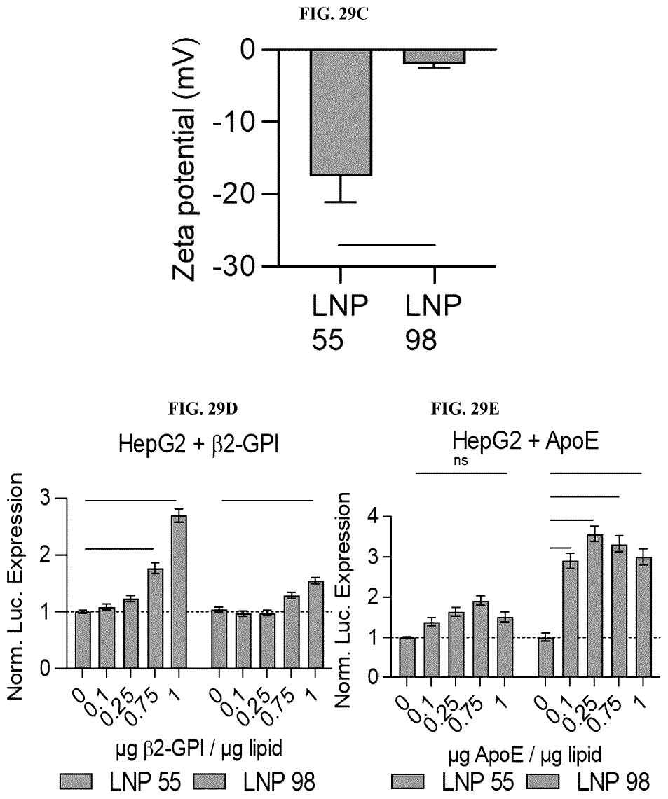

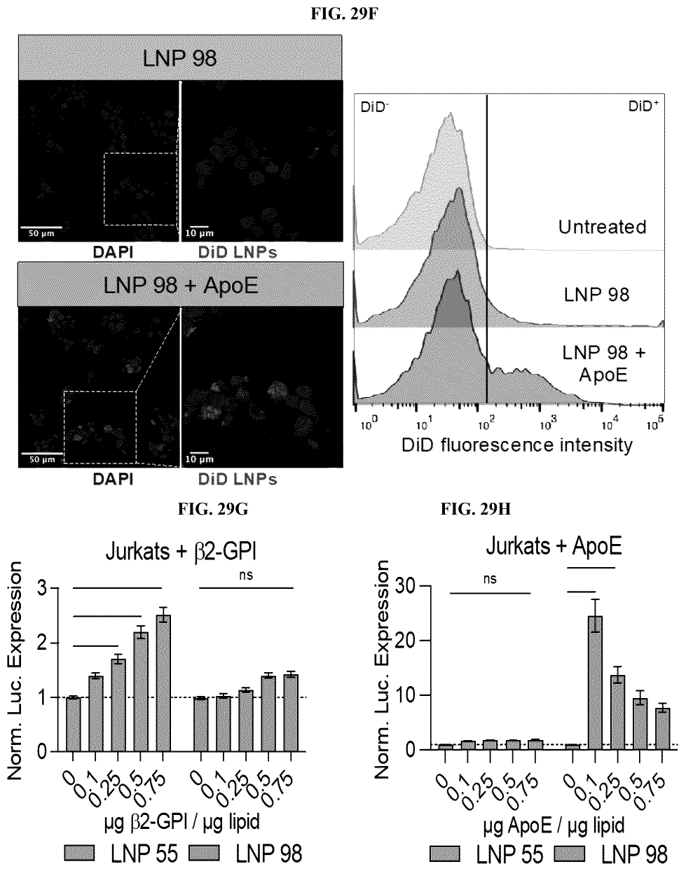

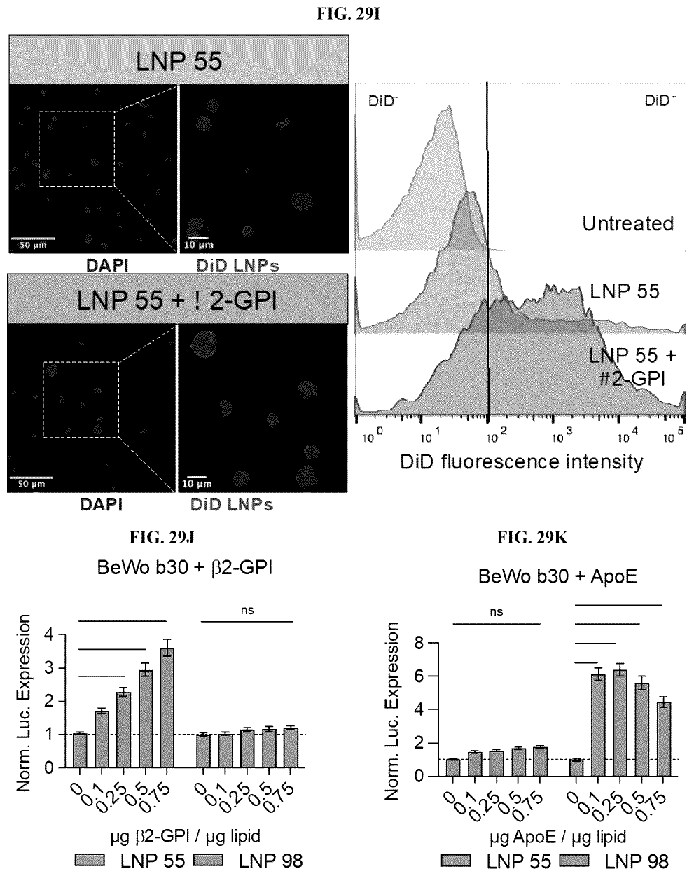

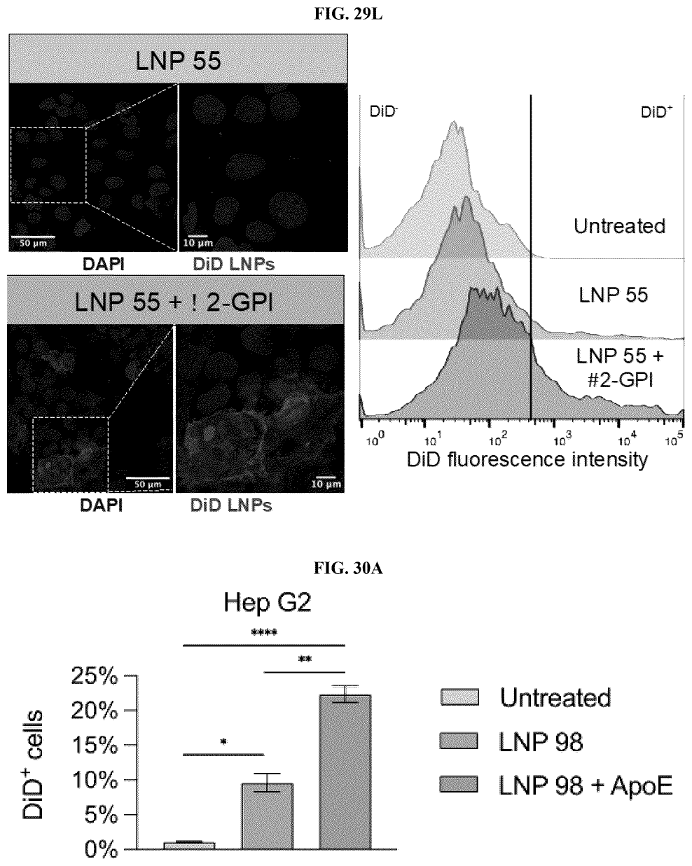

Next, a potential mechanism by which this traditional four component LNP enabled potent luciferase mRNA delivery to the placenta was examined. While mechanistic studies establishing robust structure-function relationships are limited, it is well understood that many LNP platforms achieve potent liver tropism when administered systemically due to the formation of an apolipoprotein E (ApoE)-rich protein corona. This is true for the Onpattro LNP therapeutic for hereditary transthyretin amyloidosis containing the DLin-MC3-DMA ionizable lipid that promotes ApoE binding and preferential hepatocyte targeting (FIG. 29A). Recent work has proposed that incorporating an anionic phospholipid (i.e., 1,2-dioleoyl-sn-glycero-3-phosphate, 18PA) as a fifth component in the LNP formulation can promote spleen tropism upon intravenous administration through the formation of a β2-glycoprotein I (β2-GPI)-rich protein corona. As luciferase expression in the spleen alongside the strong placental tropism with LNP 55 was observed, it was hypothesized that the ionizable lipid structure in this formulation could be promoting β2-GPI binding (FIG. 29A); particularly, the ether linkages in the structure could impart some electronegativity to the lipid overall, serving a similar role as an anionic lipid.

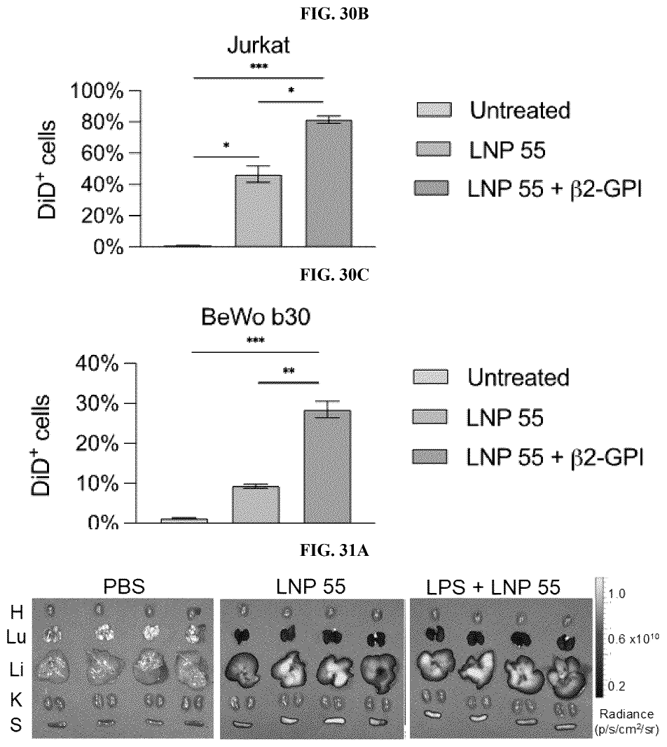

LNP 55 and the industry standard DLin-MC3-DMA formulation (LNP 98) were formulated and pre-incubated these LNPs in either β2-GPI or ApoE to evaluate the effect of protein adsorption on luciferase expression and intracellular uptake (FIG. 29B). Hep G2 cells were used as a model liver cell line and measured luciferase expression 24 h after treating cells with LNPs pre-incubated in increasing amounts of either protein. With β2-GPI, an approximately 2-2.5 fold improvement in luciferase expression was observed for LNP 55, but only at higher amounts of protein; conversely, β2-GPI only promoted luciferase expression about 1.5-fold for LNP 98 at the highest amount of protein tested (FIG. 29D). Rather, pre-incubating LNP 98 in ApoE significantly (**p<0.01, ***p<0.001, ****p<0.0001) improved luciferase expression at all amounts of protein tested (FIG. 29E). LNPs were then labeled with the lipophilic fluorescent dye DiD to evaluate intracellular uptake 30 min following treatment; pre-coating LNPs 98 in ApoE significantly (**p<0.01) enhanced LNP uptake in Hep G2 cells (FIG. 29F and FIG. 30A). Together these results suggest that β2-GPI binding to LNP 55 can promote mRNA expression in liver hepatocytes, but only at high amounts of protein. Instead, ApoE binding to the DLin-MC3-DMA LNP significantly improves luciferase expression in Hep G2 cells, consistent with previous results reporting the presence of an ApoE-rich protein corona on liver-tropic LNP formulations.

These experiments were repeated in Jurkat immortalized T cells, a major cellular target in the spleen, and BeWo b30 immortalized trophoblasts, one of the main cell types in the placenta well understood to express β2-GPI on the cell surface. Validating the in vivo results with luciferase mRNA, β2-GPI significantly (**p<0.01, ****p<0.0001) improves luciferase mRNA delivery for LNP 55 in Jurkat and BeWo b30 cells, yet has no effect for LNP 98 (FIG. 29G and FIG. 29J). Interesting, in Jurkat cells, ApoE promotes luciferase expression with LNP 98, but only at the lowest doses of protein, demonstrating a rapid decline in expression at increasing amounts of protein (FIG. 29H). A similar but less drastic effect is observed in the BeWo b30 cells, where ApoE significantly (****p<0.0001) improves LNP 98-mediated luciferase expression at all amounts of protein tested but has no effect for LNP 55 (FIG. 29K). Besides mRNA expression, β2-GPI promotes significantly higher intracellular uptake of DiD-labeled LNPs in both Jurkat (*p<0.05) (FIG. 29J and FIG. 30B) and BeWo b30 (**p<0.01) (FIG. 29L and FIG. 30C) cells than uncoated LNPs.

These results are consistent with previous work suggesting that DLin-MC3-DMA LNPs with their ApoE-rich protein corona mediate delivery to the liver through the abundant expression of low density lipoprotein receptors (LDLR) on hepatocytes. It is proposed herein that the binding of β2-GPI to LNP 55 promotes delivery to and intracellular uptake in BeWo b30 trophoblasts. Physiologically β2-GPI is a plasma protein that binds anionic phospholipids via a highly positively charged amino acid sequence. Throughout placental development, cytotrophoblasts fuse and differentiate to form the syncytiotrophoblast layer; during fusion, trophoblasts have been shown to externalize phosphatidyl serine, an anionic phospholipid, promoting β2-GPI adhesion. Perhaps the ether groups in the ionizable lipid structure in LNP 55 mimic the anionic nature of phosphatidyl serine (FIG. 29C) to promote β2-GPI binding and subsequent placental tropism.

### Example 14: Pre-Eclampsia Increases LNP Delivery to Placental Cells

After observing potent in vivo luciferase mRNA delivery to the placenta with LNP 55, the potential for an mRNA LNP platform to treat pre-eclampsia was examined. Before encapsulating VEGF mRNA, a therapeutically relevant cargo, exploration of the effects of pre-eclampsia on LNP delivery as maternal blood pressure is elevated in this disease state to compensate for impaired placental vascularization was undertaken. An ultra-low dose of LPS (1 μg/kg) was administered to pregnant mice on gestational day E7.5 via intraperitoneal injection to induce an early onset model of pre-eclampsia. Early onset pre-eclampsia is often more severe than late onset pre-eclampsia as it is associated with abnormal placental development and fetal growth restriction.

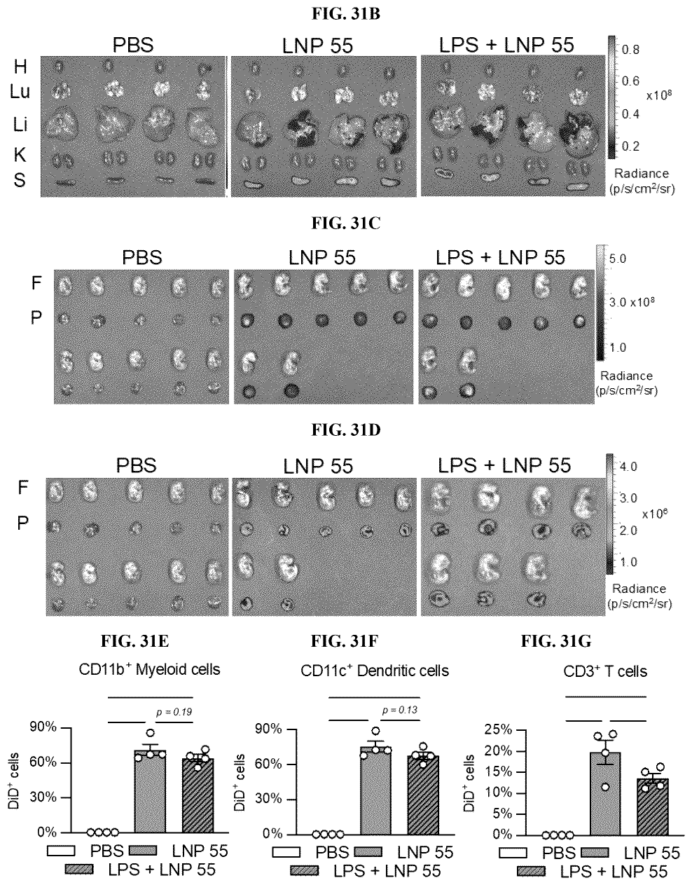

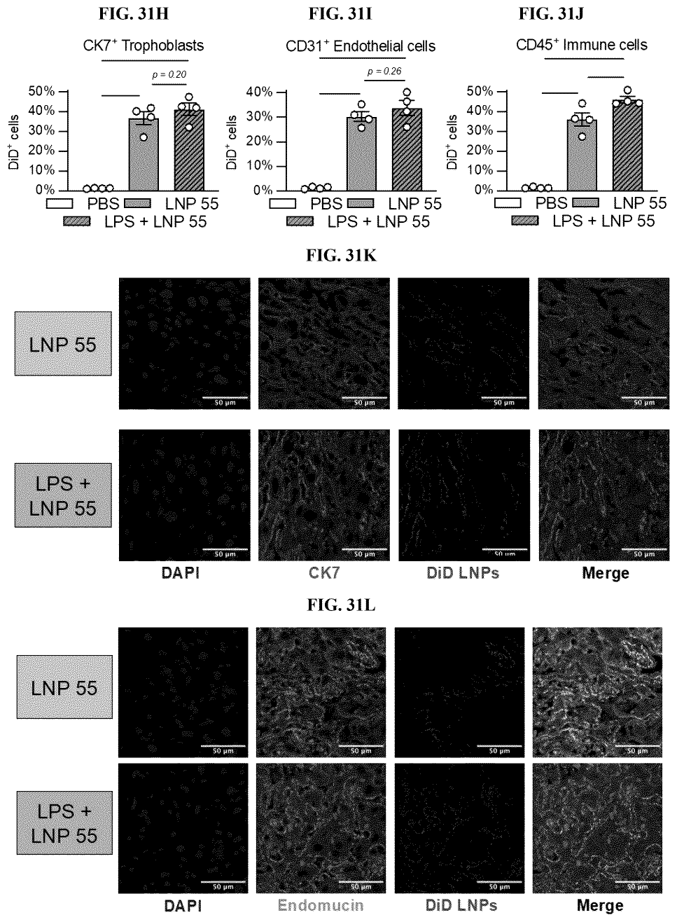

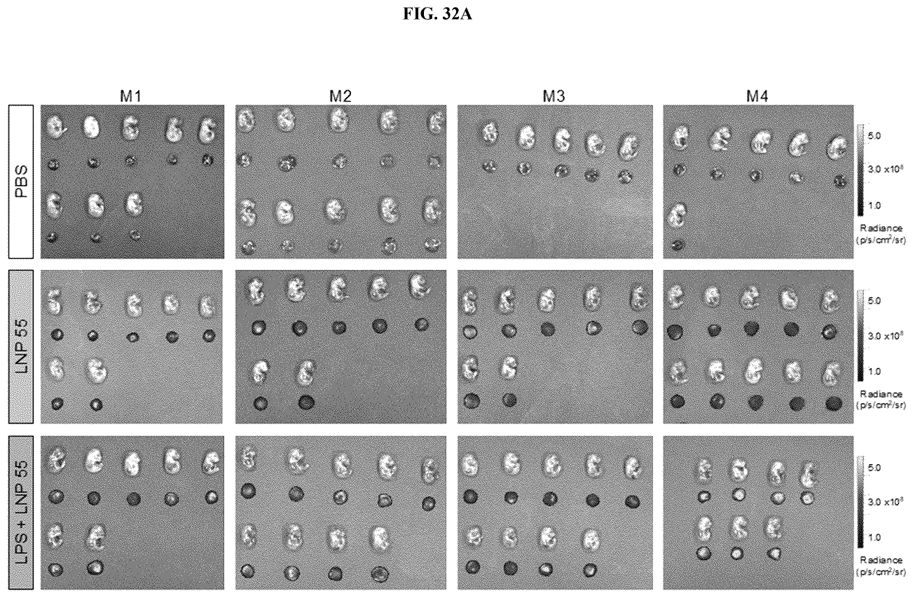

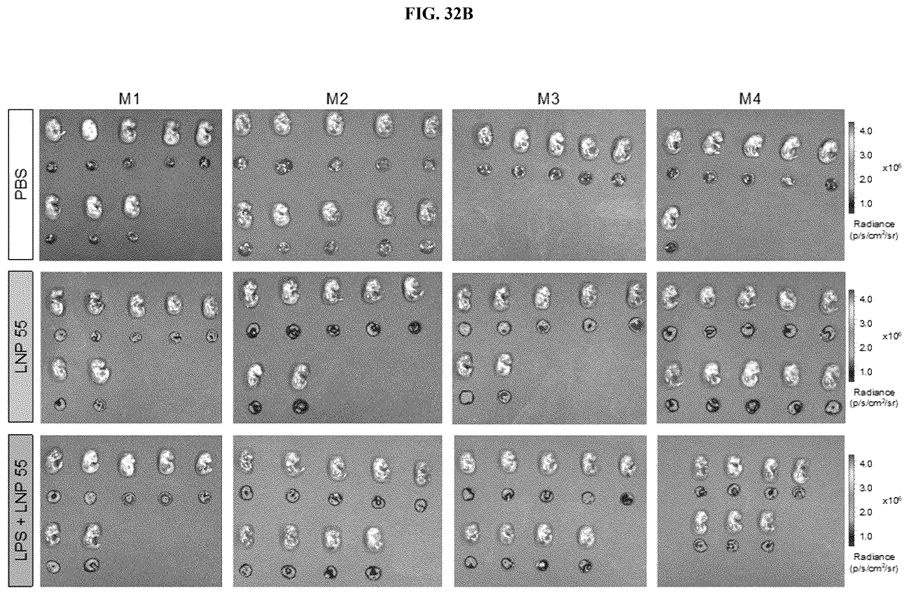

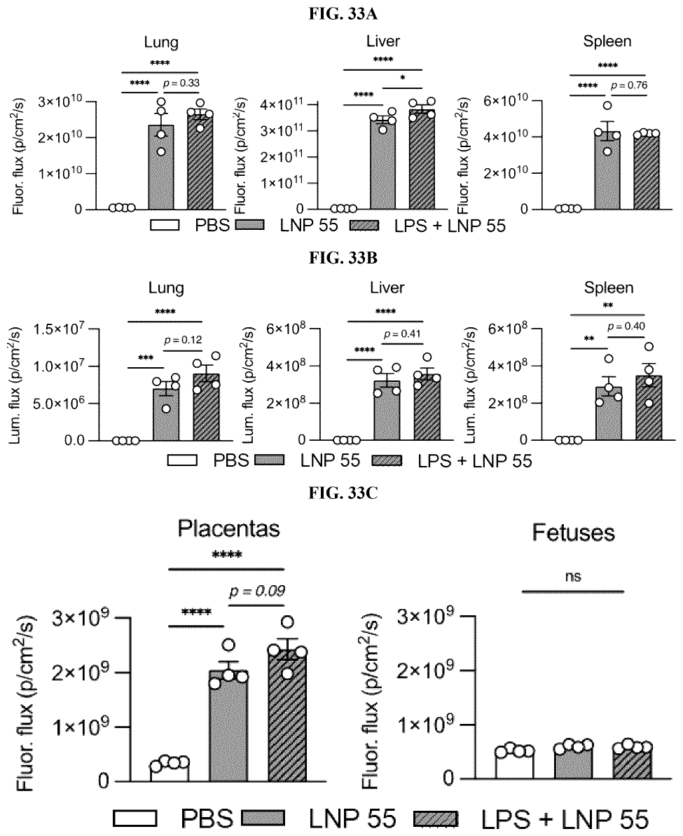

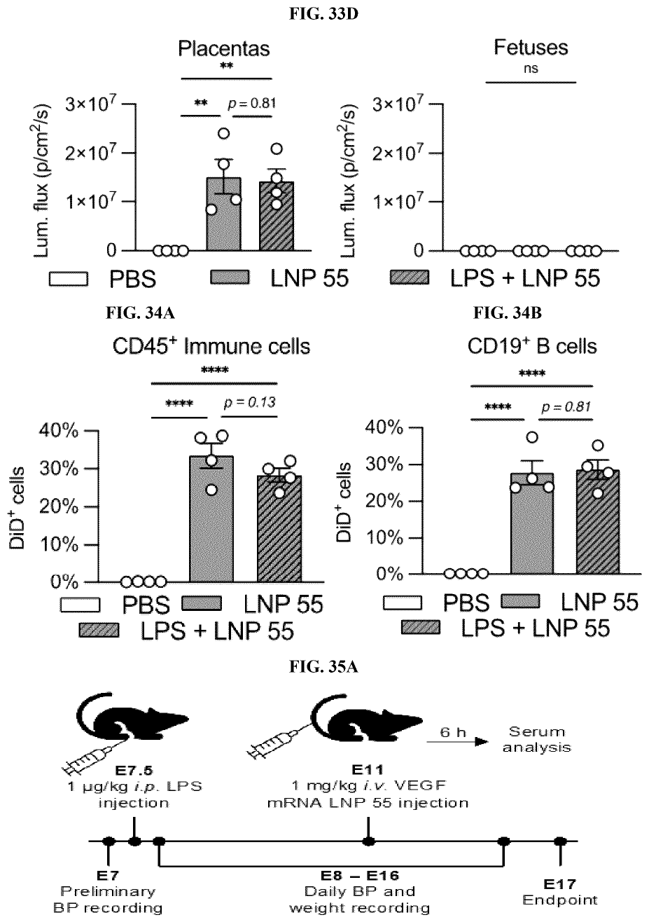

DiD-labeled LNPs encapsulating luciferase mRNA were administered to both healthy and pre-eclamptic mice at a dose of 1 mg of mRNA/kg. 12 h following administration, the maternal organs and placentas and fetuses were dissected and imaged using IVIS (FIGS. 31A-31D and FIGS. 32A-32B). DiD fluorescent flux was used to evaluate LNP biodistribution, while bioluminescent flux was used to evaluate luciferase mRNA expression. Interestingly, IVIS images of DiD fluorescence suggests that LNPs are accumulating in the liver without enabling potent luciferase expression (FIGS. 31A-31B). This could suggest that while the first pass hepatic clearance effect still mediates LNP accumulation in the liver tissue, perhaps reduced ApoE binding on LNP 55 limits intracellular uptake and subsequent protein expression. DiD fluorescent flux is higher (p=0.09) in the placentas and significantly higher (*p<0.05) in the liver from mice with pre-eclampsia than the healthy control mice (FIG. 33A and FIG. 33C). Similarly, luciferase luminescent flux is higher in the lung (p=0.12), liver (p=0.41) and spleen (p=0.40) during pre-eclampsia (FIG. 33B). This increased LNP delivery to the liver and placentas during pre-eclampsia could be explained by changes in blood flow in the disease model compared to healthy pregnant mice.

After assessing whole organ biodistribution, exploration of the cellular LNP delivery to the spleen and placenta was sought, as these organs also mediated potent luciferase expression. Spleens were processed to generate cell suspensions and stained for the cell surface markers CD45, CD3, CD19, CD11b, and CD11c in order to quantify LNP delivery on a single cell level using flow cytometry. An impressive ˜65% of CD11b⁺ myeloid cells and CD11b⁺ CD11c⁺ dendritic cells were DiD⁺ (FIGS. 31E-31F). The percent of DiD⁺ cells was lower in immune cells (p=0.13), myeloid cells (p=0.19), dendritic cells (p=0.13), and T cells (*p<0.05) in the pre-eclampsia group compared to healthy mice (FIGS. 31E-31G and FIG. 34A). These results are encouraging, suggesting the potential for less off-target delivery to the spleen in pre-eclampsia.

Placentas were processed similarly and stained for cell surface markers CD31 and CD45 before fixation and permeabilization to stain for the pan-trophoblast, intracellular marker cytokeratin 7 (CK7). Converse to the results observed in the spleen, pre-eclampsia appeared to improve LNP delivery to placental cells. The percent of DiD⁺ cells was higher in placental trophoblasts (p=0.20), endothelial cells (p=0.26), and immune cells (**p<0.01) in pre-eclamptic mice than healthy controls (FIGS. 31H-31J). Immunofluorescence staining on placental tissue sections was used to visualize delivery of DiD-labeled LNPs in the placental vasculature. Consistent with the flow cytometry results, co-localization of DiD LNPs was observed with regions staining positive for CK7 (i.e., trophoblasts) and endomucin (i.e., endothelial cells) (FIGS. 31K-31L). Similarly, the effects of pre-eclampsia on placental vascularization with smaller, constricted blood vessels in the LPS treatment group compared to the healthy pregnant mice was observed (FIGS. 31K-31L).

### Example 15: VEGF mRNA LNP Rescues Pre-Eclamptic Phenotype

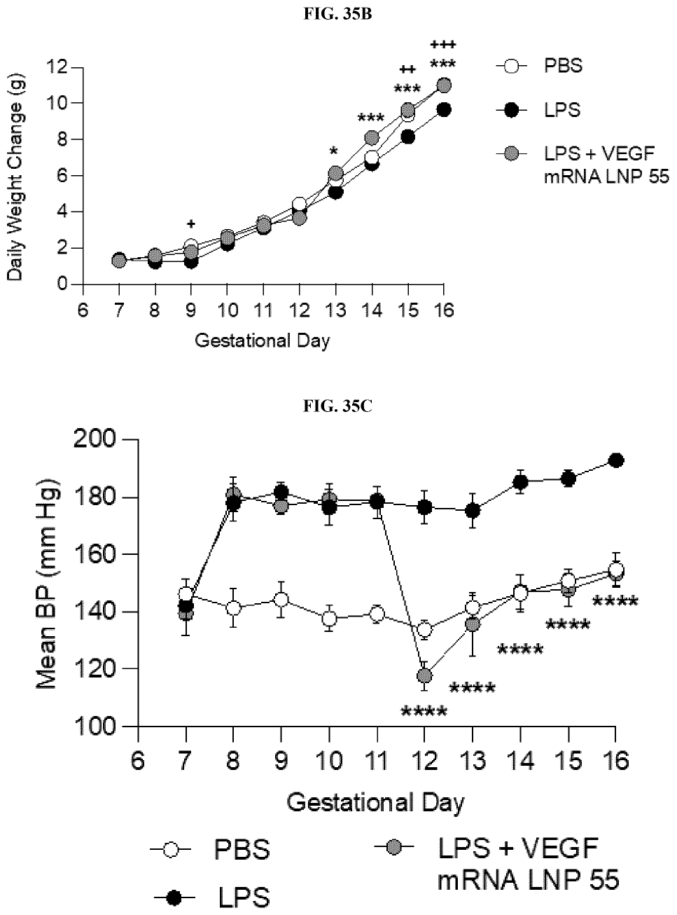

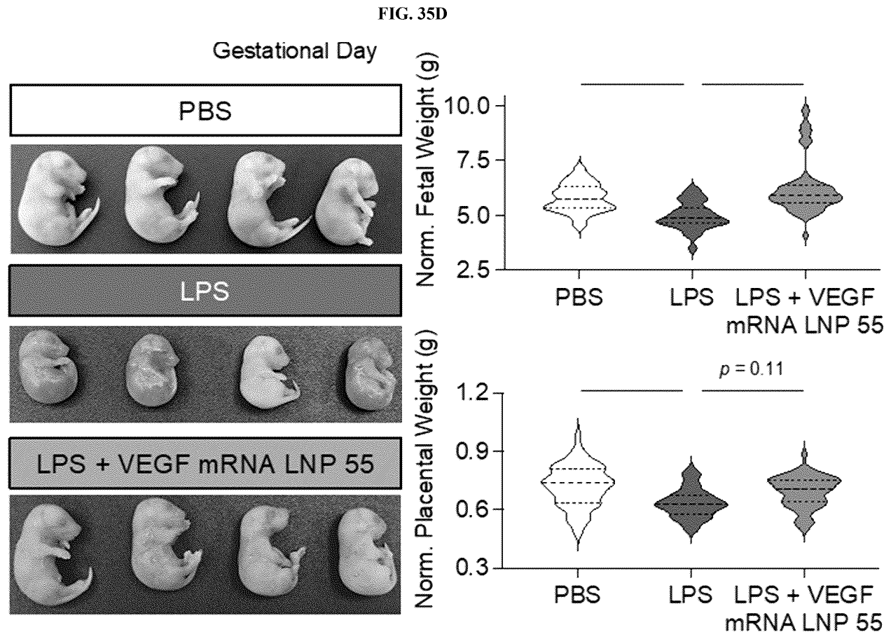

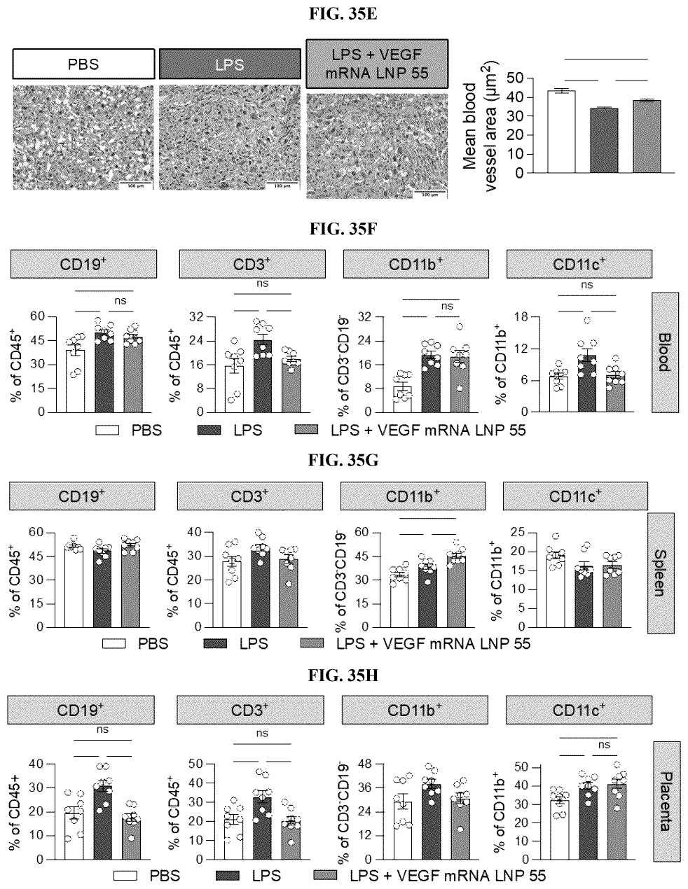

Finally, LNP 55 was used to encapsulate VEGF mRNA as a therapeutic cargo to treat pre-eclampsia during pregnancy. Before inducing pre-eclampsia, mice were trained using a noninvasive tail cuff blood pressure system and preliminary maternal weight was recorded. As above, an ultra-low dose of LPS was administered to pregnant mice on gestational day E7.5 to induce an early-onset model of pre-eclampsia (FIG. 35A). Maternal weight and blood pressure were recorded daily from gestational day E7 to E16 and a single injection of VEGF mRNA LNPs were administered via tail vein injection on gestational day E11. The model was concluded on gestational day E17 before parturition.

Daily changes in maternal weight showed a significant (++p<0.01, +++p<0.001) divergence in maternal weight for healthy (PBS) versus pre-eclamptic (LPS) mice at the end of gestation on days E15 and E16 (FIG. 35B). Pre-eclamptic mice treated with VEGF mRNA LNP 55 had a significantly higher weight than the LPS only treatment group starting on E13, with no significant differences in maternal weight compared to healthy controls (FIG. 35B). Mean blood pressure was used as a primary outcome for this model as elevated blood pressure during pregnancy is one of the key clinical indicators of pre-eclampsia. After LPS administration on E7.5, blood pressure increases by approximately 40 mm Hg and remained elevated compared to healthy mice through the end of gestation, confirming that pre-eclampsia was successfully established (FIG. 35C). Administration of VEGF mRNA LNP 55 rescues maternal blood pressure in pre-eclamptic mice, significantly (****p<0.0001) reducing blood pressure compared to the diseased mice one day after injection through the end of pregnancy (FIG. 35C).

Mice were euthanized before parturition on E17 in order to record fetal and placental weights from each dam. As fetal growth restriction is often associated with early-onset pre-eclampsia, a significant (*p<0.05) decrease in fetal weight for the pre-eclamptic mice compared to healthy controls was observed, while VEGF mRNA LNPs were able to rescue fetal weight (*p<0.05) compared to the diseased mice (FIG. 35D). Similarly, placental weights were significantly (*p<0.05) lower for the LPS treatment group compared to healthy pregnant mice, which was partially rescued (p=0.11) through the administration of VEGF mRNA LNPs (FIG. 35D). This could be explained by the fact that, in this model, the majority of placental development occurs before the administration of LNPs at the equivalent of approximately early second trimester in humans.

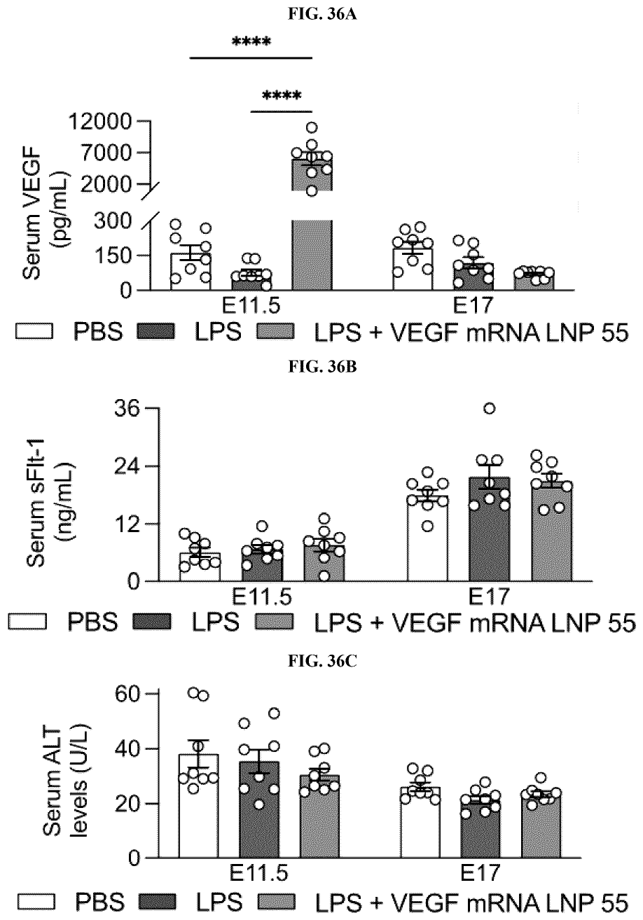

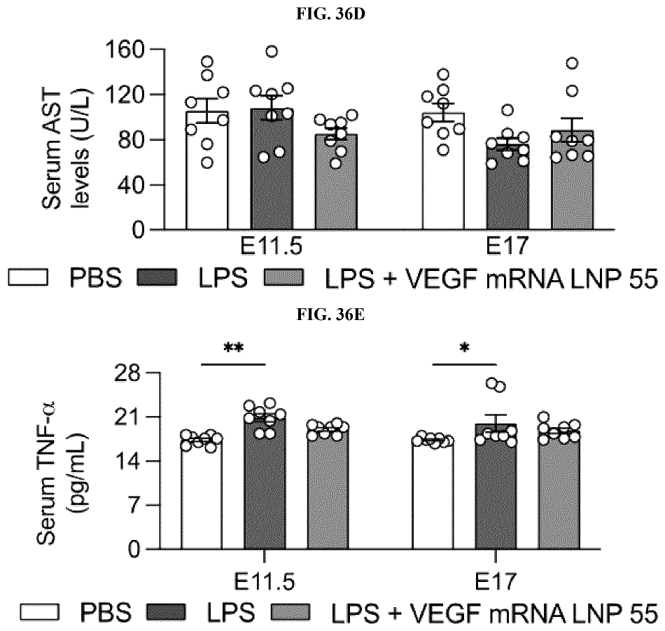

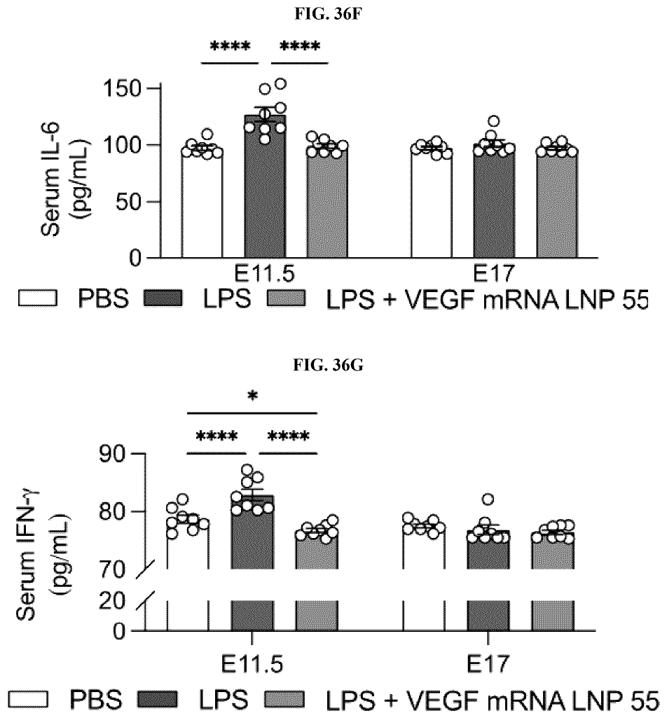

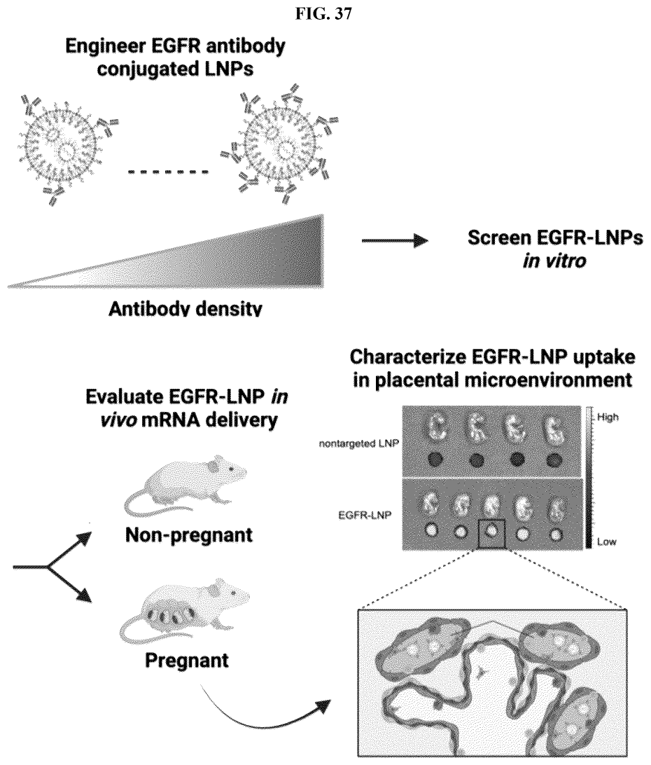

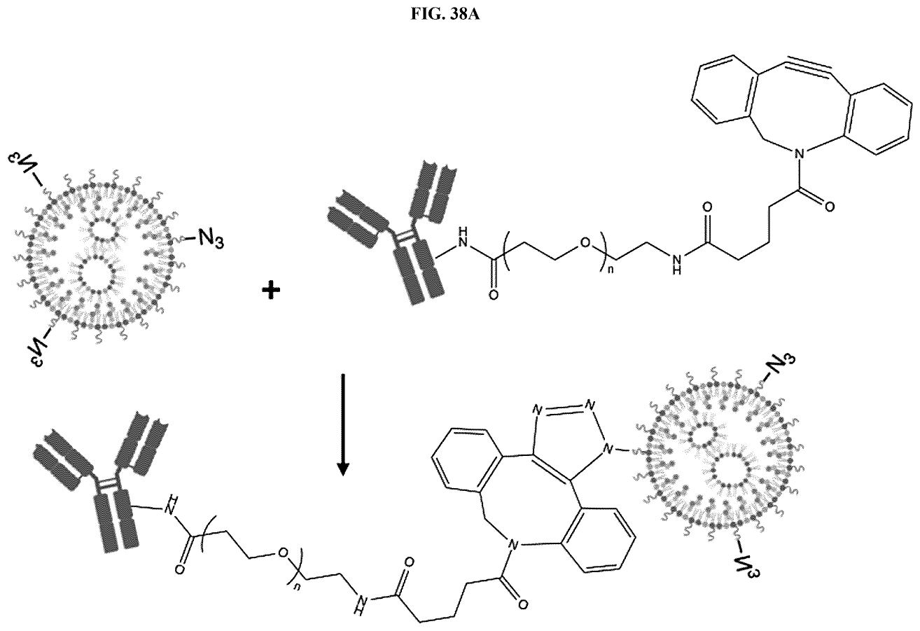

On gestational day E11.5, 6 h following administration of VEGF mRNA LNPs, serum VEGF levels were significantly (****p<0.0001) elevated by over an order of magnitude compared to the healthy and LPS treatment groups, indicating successful expression of VEGF and secretion into circulation (FIG. 36A). By gestational day E17, VEGF levels returned to baseline demonstrating the transient nature of mRNA therapeutics. Levels of sFlt-1, the soluble receptor that binds VEGF in circulation, were observed, therefore limiting placental vasodilation during pre-eclampsia. Though there were no significant differences in the serum levels of sFlt-1 across treatment groups, the mean for the pre-eclampsia group (21.8±2.4 ng/mL) was higher than that of healthy mice (18.0±1.2 ng/mL) at the end of gestation on E17 (FIG. 36B). Serum levels of the liver enzymes alanine transaminase (ALT) and aspartate aminotransferase (AST) are often used to indicate liver toxicity following systemic LNP administration. There were no significant differences in the levels of either liver enzyme, immediately following LNP administration on E11.5 or at the study endpoint on E17 (FIGS. 36C-36D). Similarly, serum cytokine levels are used to assess any LNP-mediated immune response. Serum levels of tumor necrosis factor-alpha (TNF-α) were significantly higher for the pre-eclamptic group on both E11.5 (**p<0.01) and E17 (*p<0.05) compared to healthy mice (FIG. 36E). Interleukin-6 (IL-6) (FIG. 36F) and interferon-gamma (IFN-γ) (FIG. 36G) were significantly elevated in serum on E11.5 for the pre-eclampsia group compared to both healthy mice (****p<0.0001) and those receiving VEGF mRNA LNPs (****p<0.0001). Together, these results suggest that VEGF mRNA LNP administration does not exacerbate the pro-inflammatory phenotype associated with pre-eclampsia, but rather appears to help return levels of pro-inflammatory cytokines towards those of healthy mice, particularly at short timepoints following injection.

Next, hematoxylin and eosin (H&E) staining of placental sections was used to visualize vasculature in the placental labyrinth; sections were imaged and used to quantify blood vessel area with ImageJ (FIG. 35E). Mean blood vessel area was significantly (*p<0.05) decreased in the pre-eclampsia group compared to healthy mice, which was significantly (*p<0.05) improved through the administration of VEGF mRNA LNP 55 (FIG. 35E). From representative images, there also appeared to be increased accumulation of red blood cells in the LPS placenta, leading us to explore differences in the immunophenotype amongst the treatment groups.

### Example 16: VEGF mRNA LNPs Modulate the Pre-Eclampsia Immunophenotype

While there is still active investigation on the exact mechanism, it is well appreciated that the innate immune system plays a critical role in the progression of a healthy pregnancy and that dysfunctional immunophenotypes may play a part in the development of pre-eclampsia. To this end, characterization of the immunophenotype in this model of pre-eclampsia and evaluation of any immunomodulatory effect of the VEGF mRNA LNP therapeutic described herein was sought.

First, immune cell populations in the blood including the percent of CD19⁺ B cells, CD3⁺ T cells, CD11b⁺ myeloid cells, and CD11b⁺CD11c⁺ dendritic cells, were evaluated, which were all significantly elevated in pre-eclampsia compared to healthy mice (FIG. 35F). VEGF mRNA LNP 55 was able to able to significantly (*p<0.05) reduce the percent of both T cells and dendritic cells in the blood, rescuing the phenotype towards those of healthy mice (FIG. 35F). These results are consistent with previous work demonstrating that the ratio of myeloid dendritic cells (CD11c⁺) to plasmacytoid dendritic cells (CD11c⁻) is significantly elevated in peripheral blood samples collected from patients with pre-eclampsia than those from healthy pregnancies.

In the spleen, the percent of CD11b⁺ myeloid cells was significantly elevated in LPS mice (*p<0.05) as well as the VEGF mRNA LNP treatment group (***p<0.001) (FIG. 35G), perhaps suggesting the recruitment of myeloid cells due to the spleen tropism associated with LNP 55. Additionally, the percent of CD4⁺ helper T cells was reduced while the percent of CD8⁺ cytotoxic T cells was significantly elevated for the two LPS treatment groups (FIG. 36B); however, there was no significant difference between the pre-eclampsia only and VEGF mRNA LNP treatment groups, suggesting that LNPs do not exacerbate this immunophenotype. CD4⁺CD25⁺ regulatory T cells were also evaluated, which have previously been shown to be depleted in the peripheral blood of patients with pre-eclampsia compared to healthy pregnancies. No significant changes in the regulatory T cell immunophenotype in the blood or spleen was observed, and the population was too small to detect accurately in the placenta (FIGS. 36A-36G).

Finally, evaluating the immunophenotype in the placenta, the percent of CD19⁺ B cells, CD3⁺ T cells, and CD11b⁺CD11c⁺ dendritic cells were all significantly elevated in pre-eclampsia (FIG. 35H). Administration of VEGF mRNA LNPs then significantly reduced the levels of B (**p<0.01) and T (*p<0.05) cells, with no significant differences compared to healthy mice (FIG. 35H). These results are encouraging as innate B1 cells are abnormally activated in pre-eclampsia and produce angiotensin II type I receptor agonistic autoantibodies; these autoantibodies induce vasoconstriction and promote the secretion of anti-angiogenic factors such as sFlt-1. Together these results suggest that the VEGF mRNA LNP platform described herein has the potent to modulate the immunophenotype in a mouse model of pre-eclampsia.

### Example 17: Design and Characterization of Exemplary EGFR-LNPs

In one aspect, the present disclosure relates to LNPs formulated with the C12-494 ionizable lipid and conjugated EGFR antibodies onto the LNP surface to enact active targeting to the placenta. LNPs were formulated via microfluidic chaotic mixing of an organic lipid phase and an aqueous mRNA phase as previously described, using the standard LNP formulation for mRNA delivery composed with 35% ionizable lipid:16% DOPE:46.5% cholesterol:2.5% PEG molar composition. To facilitate antibody decoration, lipid-anchored PEG-azide was substituted as a fraction of the total PEG at varying ratios, as the addition of azide to the LNP surface allows for conjugation with DBCO-labeled antibodies via SPAAC as demonstrated by previous works.

It has been reported that the density of targeting moieties on the surface of nanoparticles can influence pharmacokinetic behavior. Specifically, studies have demonstrated that intermediate ligand densities may be preferred, with too high of ligand densities saturating cellular receptors. However, the relationship between targeting ligand density and nanoparticle uptake may depend on other factors, such as receptor density in the cellular membrane and receptor spatial orientation.

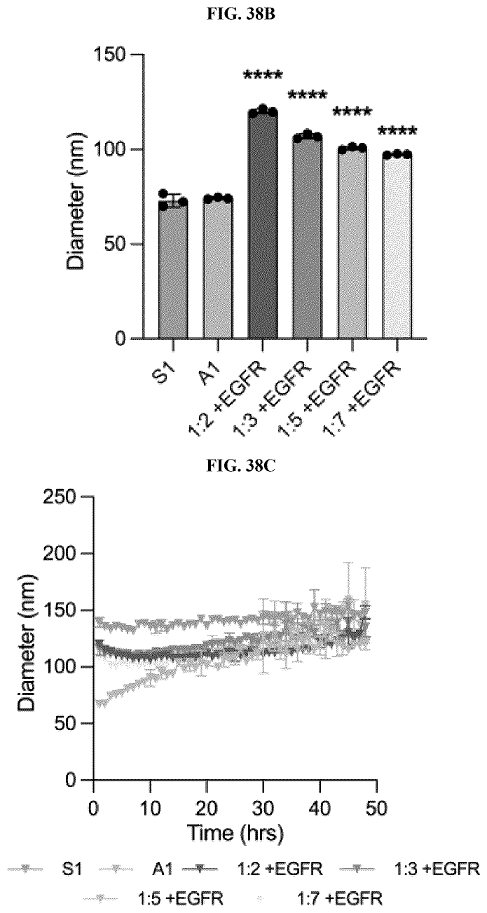

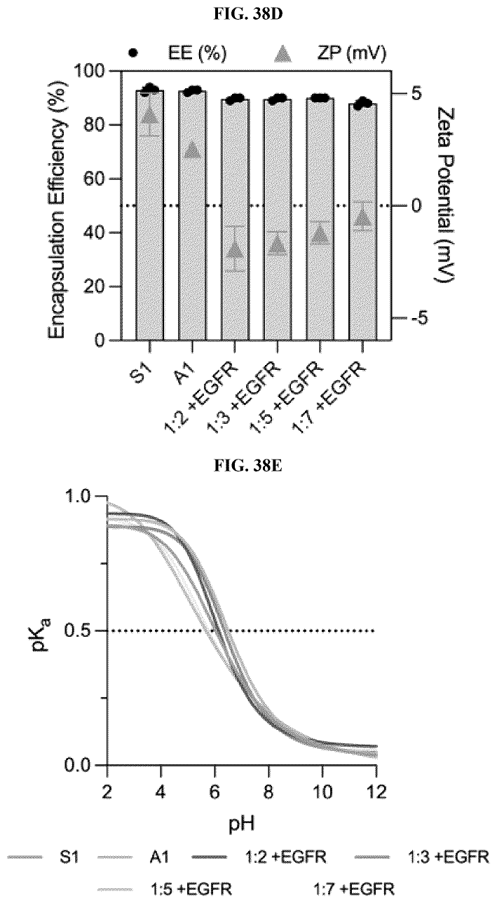

Here, LNPs were generated with four different substitutions of PEG-azide:PEG (1:2, 1:3, 1:5, and 1:7) to test the influence of decreasing densities of antibody decoration. After microfluidic formulation, azide-containing LNPs were incubated overnight with DBCO-labeled EGFR antibodies to generate EGFR-LNPs (FIG. 38A), and unbound antibody was removed via size exclusion chromatography. Throughout this work, EGFR-LNPs were compared against two nontargeted formulations —S1, a standard formulation containing no azide, and A1, an azide control formulation containing a 1:5 substitution of PEG-azide:PEG (Table 9).

TABLE 9
Composition of exemplary LNP formulations
(i.e., A1-A4) and control (S1)*

| | C12- | | | Total | PEG-azide:PEG |
|---|---|---|---|---|---|
| LNP | 494 | DOPE | Chol. | PEG | ratio |
| S1 | 35 | 16 | 46.5 | 2.5 | 0:1 |
| A1 | 35 | 16 | 46.5 | 2.5 | 1:2 |
| A2 | 35 | 16 | 46.5 | 2.5 | 1:3 |
| A3 | 35 | 16 | 46.5 | 2.5 | 1:5 |
| A4 | 35 | 16 | 46.5 | 2.5 | 1:7 |

*Refers to LNP compositions prior to conjugation with an EGFR antibody; Upon conjugation, all LNP nomenclature remains identical, with the exception of A3, which is indicated as “S2” without conjugation, in the experiments described herein.

After processing, LNPs were characterized for size, stability, zeta potential, mRNA encapsulation efficiency, and pKa. Successful antibody conjugation was marked by an increase in LNP size as confirmed via dynamic light scattering (DLS). The addition of PEG-azide alone (referred to herein as “A1” for EGFR-LNP related experiments) did not result in changes in LNP diameter when compared to the standard formulation (referred to herein as “S1” for EGFR-LNP related experiments) and, thus, the observed increases in LNP diameter in EGFR-LNPs were attributed to antibody conjugation. Interestingly, LNP size increased linearly with increasing substitution of PEG-azide and subsequent antibody decoration density (FIG. 38B), speaking to the highly efficient kinetics and selectivity of SPAAC chemistry. To examine whether antibody modification affects LNP stability in solution and storage, LNPs were incubated in aqueous solution for 48 h and sampled each hour via DLS. All LNPs remained stable over a 48-hour time course, with EGFR-LNPs demonstrating no significant aggregation distinguished by large changes in size, when compared to LNPs S1 and A1 (FIG. 38C). The zeta potential of all LNPs remained overall neutral, with the surface charge of EGFR-LNPs characterized by a slight decrease in zeta potential consistent with the weak negative charge carried by immunoglobulins (FIG. 38D). Antibody conjugation and post-processing did not affect mRNA encapsulation efficiencies, and pKa values ranged from 5.7-6.6 (FIG. 38E), with the pKa of EGFR-LNPs remaining comparable to nontargeted formulations.

### Example 18: EGFR-LNPs Enhance mRNA Delivery In Vitro to Trophoblasts

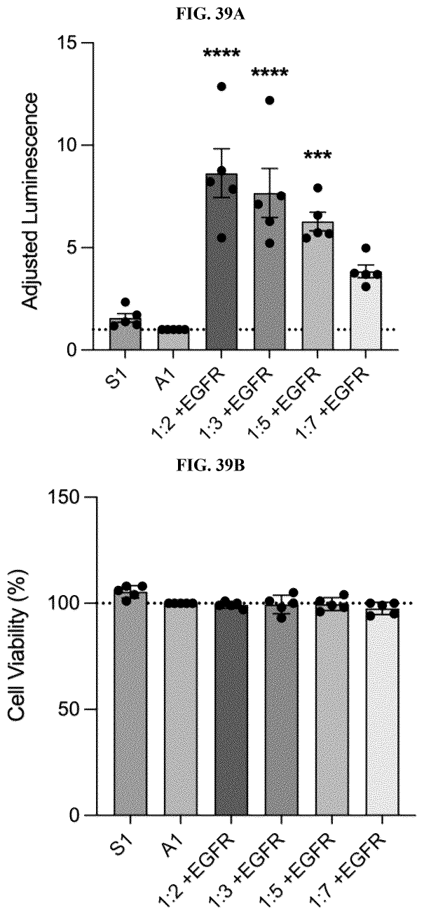

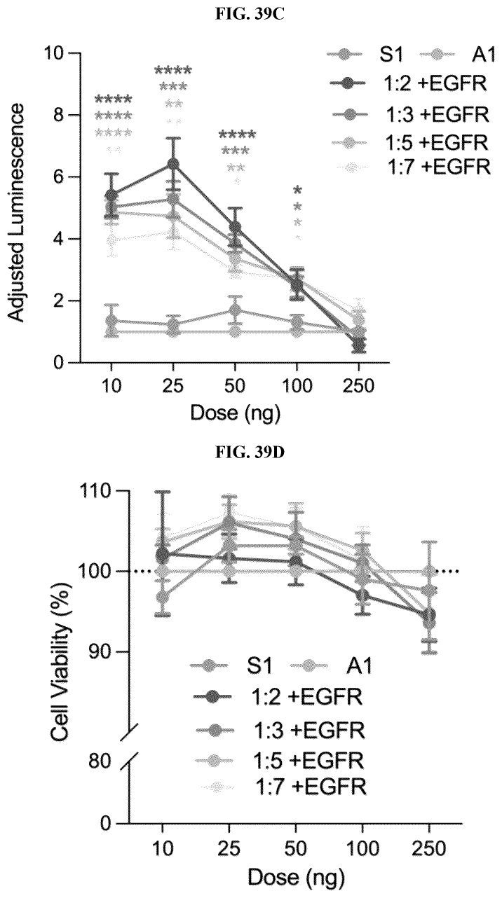

To evaluate whether the engineering of LNPs with EGFR antibodies could influence mRNA delivery in vitro, LNPs were formulated encapsulating luciferase mRNA as a reporter cargo. The EGFR+ human choriocarcinoma JEG-3 cell line, a common in vitro model of human placental trophoblasts, were treated with S1, A1, and EGFR-LNPs at a dose of 50 ng mRNA/50,000 cells. Luciferase expression as a measure of functional mRNA delivery was evaluated 24 h following treatment (FIG. 39A). The A1 formulation did not perform significantly differently than the previously validated placenta-tropic S1 formulation, confirming that the addition of azide alone does not confer active targeting capabilities. Given this finding, the performance of all EGFR-LNPs was normalized to the A1 formulation in all subsequent experiments. Excitingly, three of the EGFR-LNP densities (1:2, 1:3, and 1:5 +EGFR) demonstrated significantly enhanced luciferase delivery compared to AL. Although not statistically significantly different from one another, the highest luciferase expression was observed following treatment with the LNP with the highest antibody density (1:2 +EGFR), with luciferase expression slowly decreasing in a linear fashion across intermediate antibody densities (1:3 +EGFR and 1:5 +EGFR). Luciferase expression following treatment with the 1:7 +EGFR LNP did not statistically differ from the S1 and A1 nontargeted controls. Cell viability was not affected across formulations at a dose of 50 ng mRNA/50,000 cells. (FIG. 39B).

To examine whether the observed antibody targeting effects exhibited dose-dependent behavior, LNPs were screened in a dose response in vitro and evaluated for luciferase expression and cytotoxicity. At doses ranging from 10 ng-100 ng mRNA/50,000 cells, all four EGFR-LNPs demonstrated enhanced luciferase expression compared to A1 (FIG. 39C). Interestingly, in the initial screen at a dose of 50 ng mRNA/50,000 cells, the LNP with the lowest antibody density (1:7 +EGFR) did not demonstrate significantly improved expression compared to A1. However, at the lower doses of 10 and 25 ng mRNA in dose response experiments, enhanced expression was observed. Similarly, the remaining EGFR-LNPs demonstrated the highest enhancement in luciferase expression at doses of 10 ng and 25 ng mRNA, consistent with previous works reporting lower required doses with subsequent higher therapeutic efficacy when using actively targeted nanoparticles compared to nontargeted counterparts. In accordance with the initial screen, the most densely decorated nanoparticle (1:2 +EGFR) appeared to exhibit the highest improvements and the least densely decorated nanoparticle (1:7 +EGFR) appeared to exhibit the smallest improvements in luciferase mRNA delivery compared to A1 across most doses. As the mRNA dose increased, the enhancement in luciferase expression of all EGFR-LNPs decreased, most likely due to a saturation of EGFR receptors and a subsequent decrease in active targeting capabilities. At the dose of 250 ng mRNA/50,000 cells, none of the EGFR-LNPs different significantly from A1. Importantly, no significant cytotoxicity was observed across formulations when compared to A1 (FIG. 39D). As expected, initial signs of toxicity were observed at the highest dose of 250 ng mRNA across all LNP formulations.

### Example 19: Biodistribution of EGFR-LNPs Differs In Vivo in Non-Pregnant and Pregnant Mice

The LNP which demonstrated the greatest improvement in mRNA delivery in vitro was found to be (1:2 +EGFR). However, further studies were performed to determine whether the influence of antibody density in vivo would correlate with in vitro findings. It is well understood that in vitro cell models cannot properly recapitulate the complex nature of biological systems and, thus, in vitro LNP delivery trends are not always predictive of in vivo behavior. LNP ligand decoration may further exacerbate these shortcomings; studies have demonstrated that active targeting moieties are often more efficient in vitro than in vivo. Because three out of four EGFR-LNP formulations demonstrated strong and consistent improvements in luciferase mRNA delivery compared to A1 across most doses, the 1:2, 1:3, and 1:5 +EGFR formulates were selected for further screening in vivo.

Given the current lack of knowledge surrounding changes in nanoparticle pharmacokinetics due to physiologic changes that occur during pregnancy, exploration of the differential biodistribution of the EGFR-LNPs in non-pregnant and pregnant mice was first explored. Non-pregnant and gestational day E16 pregnant mice were tail vein injected with PBS, S1, A1, or EGFR-LNPs at a dose of 0.4 mg mRNA/kg body mass. Mice received an intraperitoneal injection of luciferin 6 h after treatment before undergoing euthanasia. Maternal organs, placentas, and fetuses were removed and imaged for bioluminescence using in vivo imaging system (IVIS).

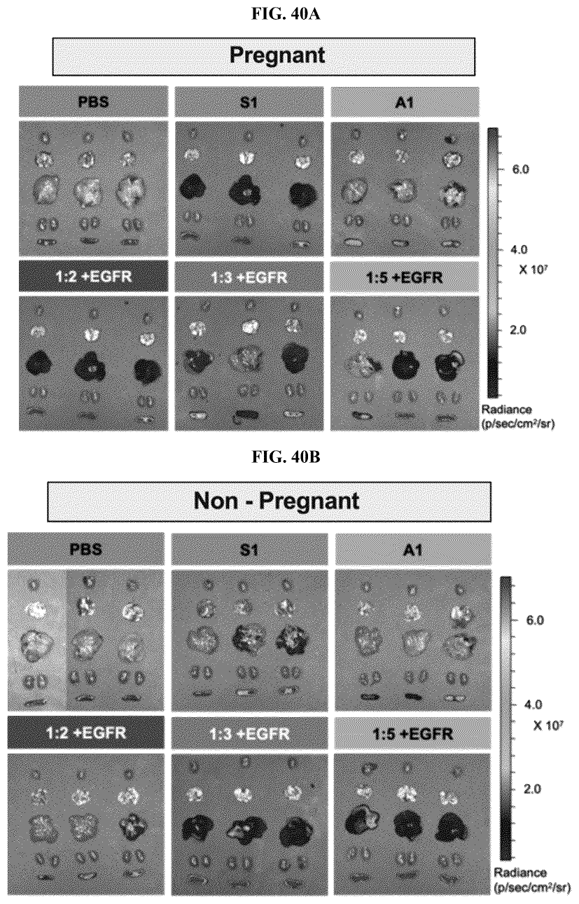

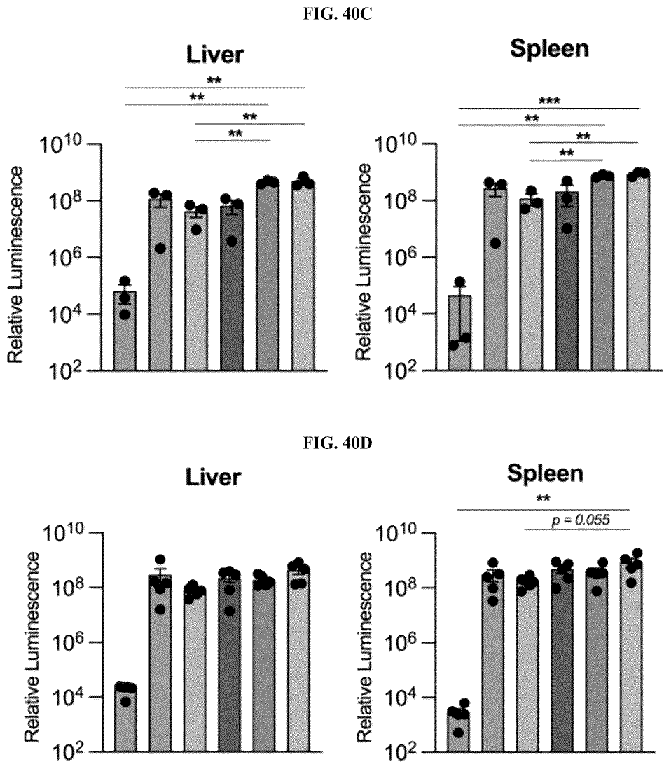

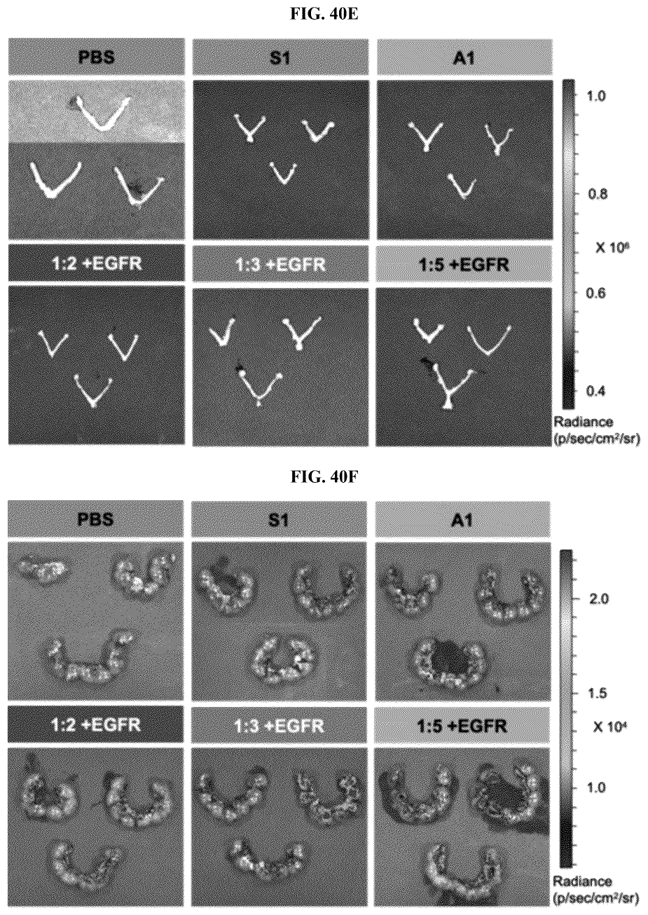

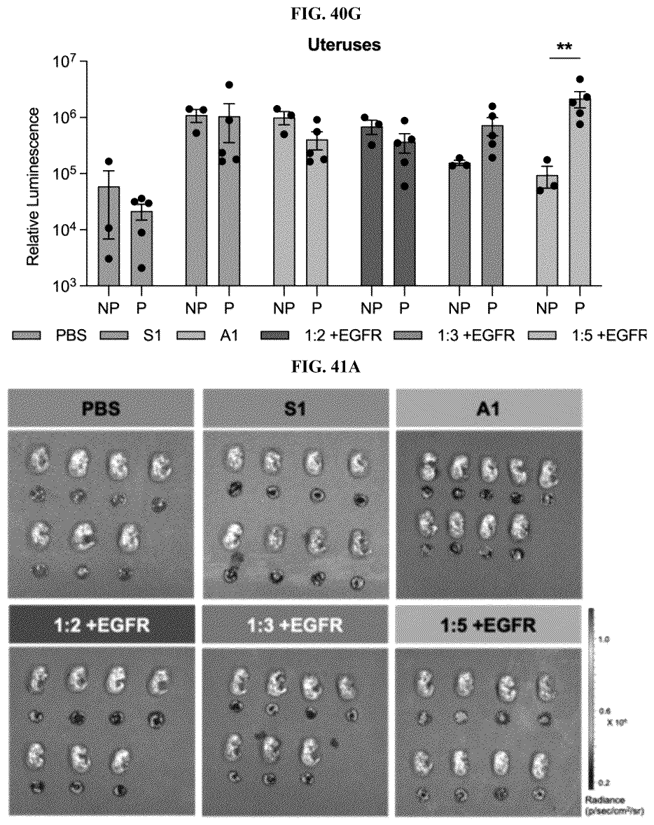

LNPs formulated with the placenta-tropic C12-494 ionizable lipid were found to demonstrate reduced liver delivery and enhanced splenic delivery in addition to the observed placental delivery, likely due to electronegativity conferred by the presence of ester bonds in the C12-494 structure. All LNP formulations described herein resulted in low luminescence in the livers and high luminescence in the spleens of both non-pregnant and pregnant mice (FIGS. 40A-40B). Further, LNP formulations also demonstrated a reduction in the spleen:liver ratio in pregnant mice compared to non-pregnant mice, suggesting that changes in cardiac output during pregnancy likely alter LNP biodistribution kinetics at large, and may shunt LNPs away from maternal organs and towards reproductive organs.

In non-pregnant mice, the 1:3 and 1:5 +EGFR formulations demonstrated enhanced luciferase expression in both the livers and spleens compared to A1, (FIG. 40C) whereas, in pregnant mice, none of the treatment groups differed significantly from A1 (FIG. 40D). Only treatment with 1:5 +EGFR LNPs resulted in higher luciferase expression (p=0.055) in the spleens of pregnant mice compared to treatment with A1 LNPs. In humans, the placenta is the highest non-malignant EGFR-expressing organ; however, in mice, both the placentas and the liver have been shown to have abundant EGFR expression. Thus, in non-pregnant mice, when no placentas are present, EGFR-LNPs showed enhanced, targeted delivery to EGFR-expressing cells in the liver. The reduction in observed liver delivery of EGFR-LNPs in pregnant mice compared to non-pregnant mice is likely then a result of shifted cardiac output, combined with active-targeted to the EGFR-rich placentas.

The enhanced splenic delivery of 1:5 +EGFR LNPs in both non-pregnant and pregnant mice is most likely a result of antibody trafficking to the spleen. It has been reported that the spleen is a site of accumulation of monoclonal antibodies, although the specific mechanisms governing antibody trafficking to and clearance in the spleen have not been fully elucidated. Given its role as a phagocytic blood filter, splenic macrophages and neutrophils can interact with Fc regions on antibodies, leading to their subsequent internalization and destruction. However, this clearance mechanism may be in opposition with immunoglobulin recycling via the neonatal Fc receptor (FcRn), which has been shown to have rich expression in the spleen. One recent study reported that the highest catabolic activity was observed in the spleen compared to all other organs for the clearance of antibodies with FcRn affinity but not for antibodies lacking FcRn affinity. Importantly, 1:5 +EGFR-LNP splenic delivery was more enhanced in non-pregnant mice, which can be explained by the decreased alloimmune responses observed during pregnancy due to the presence of the partially allogenic fetus.

Together, these results suggest that conjugation of LNPs with monoclonal antibodies may encourage extrahepatic delivery to the spleen. Further, the use of antibody targeting may be more advantageous during pregnancy, as reduced alloimmune responses may limit antibody trafficking to the spleen and subsequently enhance targeted delivery to the placenta. These findings highlight the importance of screening therapeutic platforms in pregnant models, as pregnancy can result in not only organ-level changes in biodistribution, but also cellular-level changes in nanoparticle trafficking and uptake.

An interest also existed in exploring LNP delivery to the uteruses in both non-pregnant and pregnant mice (FIGS. 40E-40F). Interestingly, the highest uterine luminescence in non-pregnant mice was observed with control formulations S1 and A1, with 1:3 and 1:5 +EGFR LNPs exhibiting very little delivery. On the contrary, pregnant uteruses showed increased luminescence with 1:3 and 1:5 +EGFR LNPs. These results may be explained by the role of EGFR in the murine uterine stroma, where EGFR signaling regulates uterine development and embryo implantation during pregnancy. Significantly enhanced luciferase expression was observed in the uteruses of pregnant mice following treatment with the 1:5 +EGFR LNP when compared to non-pregnant mice, suggesting that an intermediate antibody density may be optimal for EGFR targeting to the uterus during pregnancy (FIG. 40G). Of note, luciferase expression in all maternal organs following treatment with the 1:2 +EGFR formulation, the lead candidate identified during in vitro screening, did not differ significantly from S1 and A1 mediated luciferase expression in both non-pregnant and pregnant groups. These results reiterate previous findings that in vitro screening is not always predictive of in vivo nanoparticle behavior.

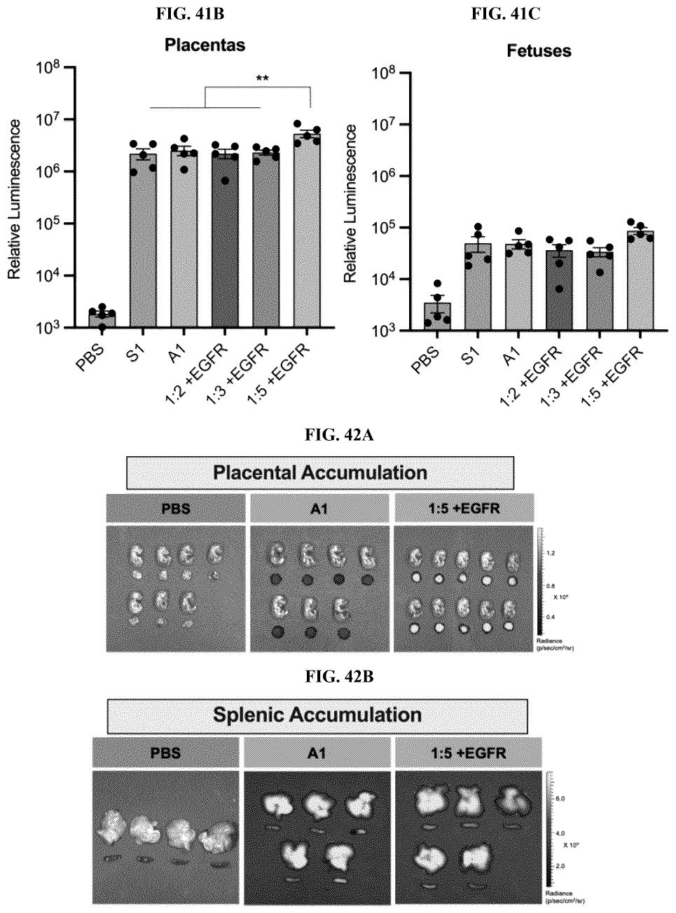

Bioluminescence was next quantified in the placentas and fetuses of pregnant mice to evaluate whether EGFR-LNPs could promote active targeting to EGFR-rich placentas (FIG. 41A). Consistent with the observed uterine delivery in pregnant mice, the 1:5 +EGFR formulation resulted in significantly enhanced (˜2×) luciferase expression in placentas compared not only to A1, but all other treatment groups (FIG. 41B). In contrast with the in vitro findings, EGFR-LNPs with higher antibody densities (1:2 and 1:3 +EGFR) did not result in enhanced placental luciferase expression. These results confirm that densely decorated LNPs may perform best in a dish given the static nature of the cells and widespread availability of receptors; however, intermediate densities of targeting moieties appear to be optimal in vivo, potentially due to reduced steric hindrance effects and subsequent availability of antigen binding sites.

Given that whole antibodies are present on the EGFR-LNPs, it is also possible that the increase in available Fc regions on more densely decorated LNPs (1:2 +EGFR) increases the likelihood of recognition and phagocytosis by macrophages of the mononuclear phagocyte system (MPS), leading to their rapid clearance and reduced targeting capabilities. placental transfer of LNPs into fetal circulation was observed across any treatment group (FIG. 41C). Together, these results demonstrate the potential of EGFR-targeted LNPs in promoting extrahepatic, tissue-specific delivery to the EGFR-rich placenta for use during pregnancy complications and highlight the importance of ligand density in designing LNP platforms capable of active targeting in vivo.

### Example 20: EGFR-LNPs Enhance Uptake in Placental Trophoblasts and Immune Cells

One of the main advantages conferred by actively targeted LNPs is their ability to promote LNP uptake in specific receptor-expressing cells. Given the observed enhancement in placental luciferase expression following treatment with the 1:5 +EGFR LNP, evaluation of EGFR-LNP uptake and accumulation on a cellular level was sought in murine placentas compared to LNP A1. Because enhanced splenic signal was also observed during the biodistribution experiments following treatment with the 1:5 +EGFR LNP in both non-pregnant and pregnant mice, cellular uptake of EGFR-LNPs in murine spleens was also investigated in pregnant mice in order to further elucidate mechanisms behind splenic antibody accumulation. To this end, DiR-labeled LNPs encapsulating mCherry mRNA were formulated and administered to pregnant mice at a dose of 1 mg mRNA/kg body weight. 12 h after injection, mice were euthanized and organs previously demonstrating luminescent flux—the liver, spleen, placentas, and fetuses—were imaged using IVIS with filters paired for DiR and mCherry. Fluorescent flux values were calculated using ROIs.

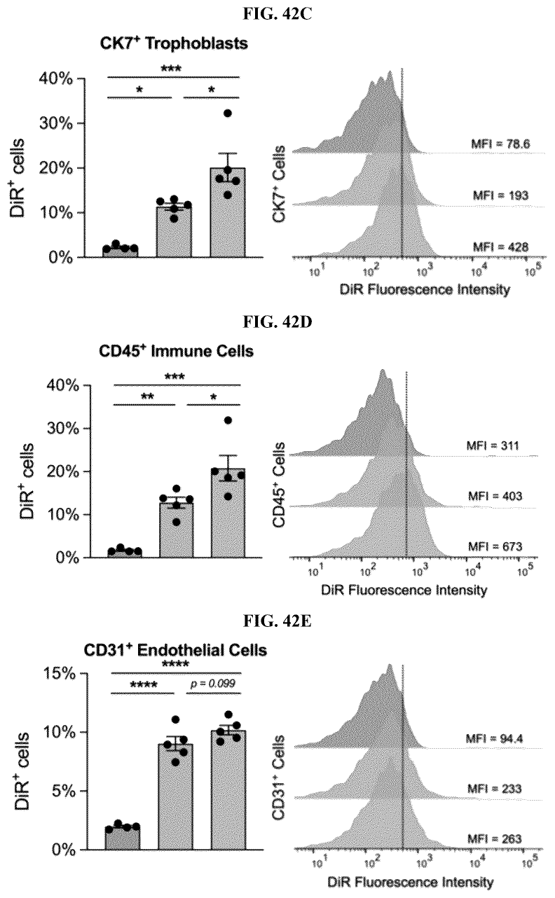

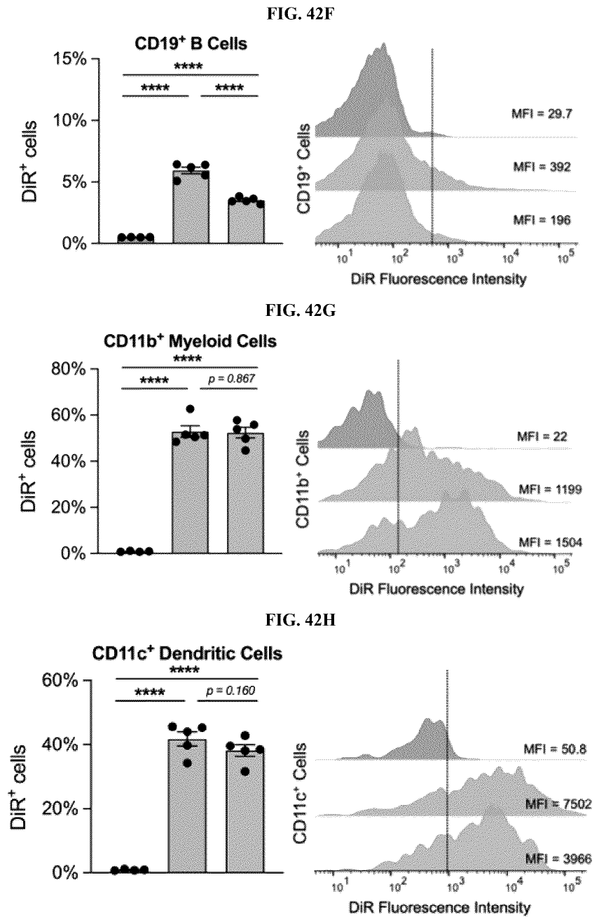

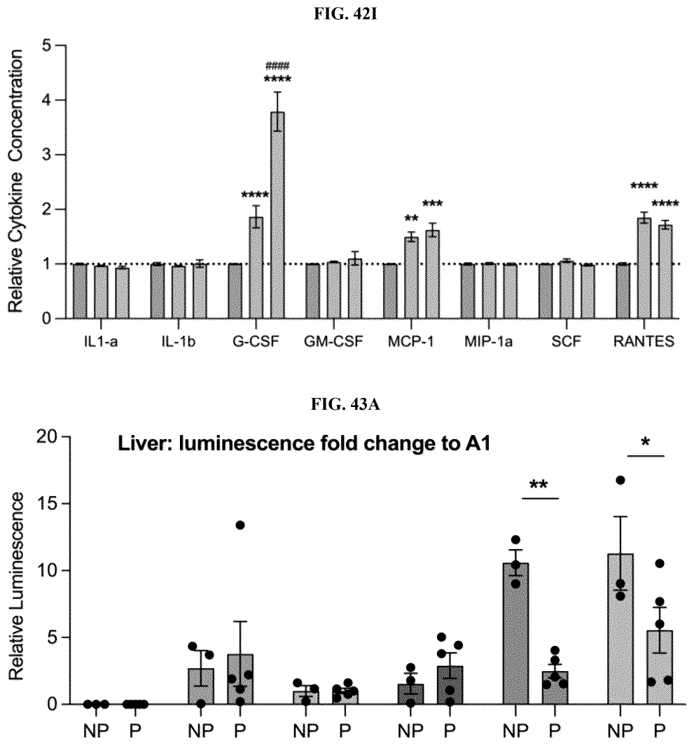

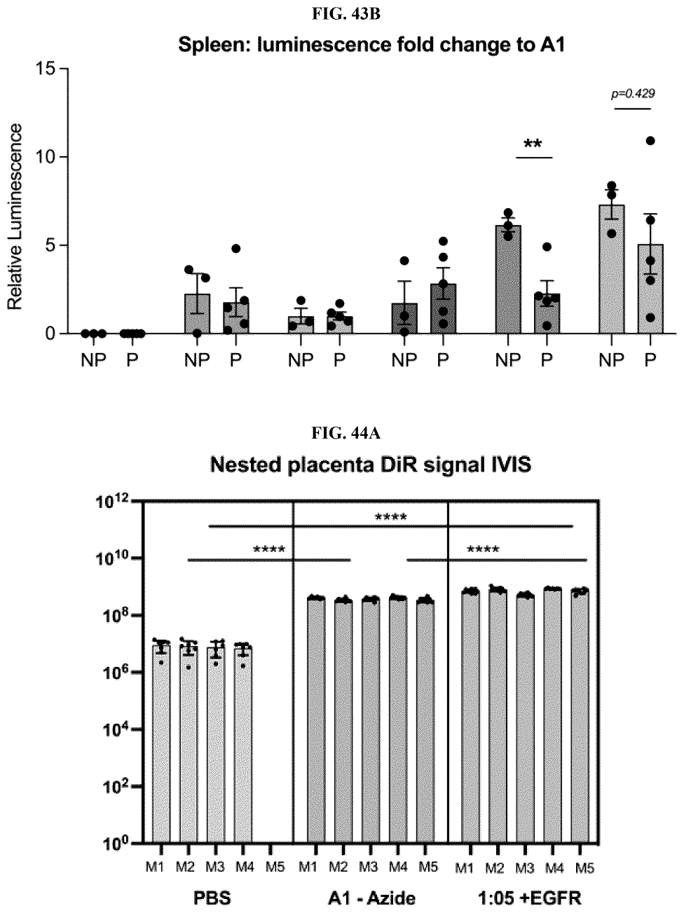

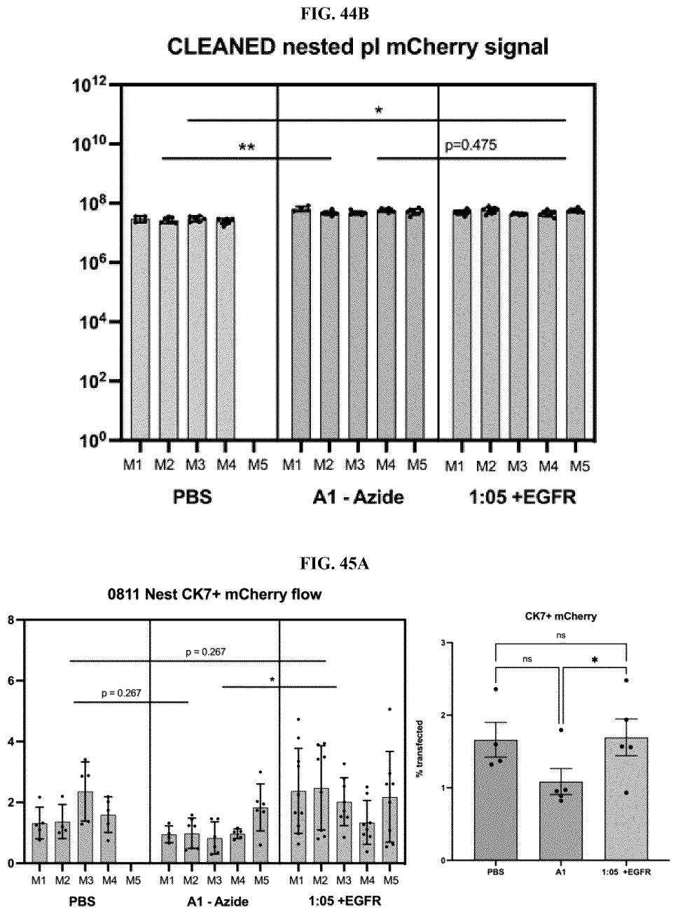

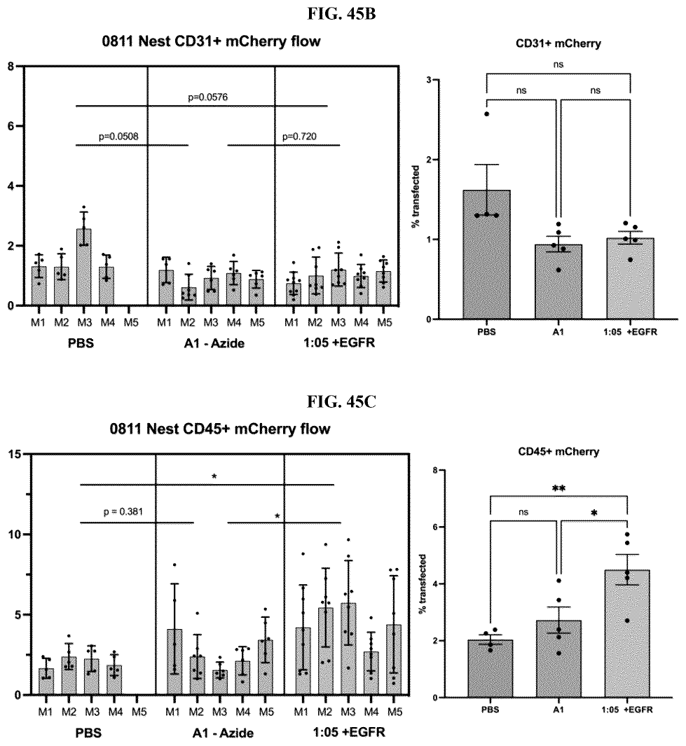

In the spleen, DiR fluorescent flux was significantly higher in the A1 treatment group than the 1:5 +EGFR treatment group (FIG. 42A), however mCherry fluorescent flux was not different between the two groups, suggesting that modification of the base spleen-tropic particle with EGFR antibodies actually shunts accumulation away from the spleen in pregnant mice. These results are in agreement with the observed decrease in luminescent flux in the spleens of pregnant mice in biodistribution experiments when compared to non-pregnant mice. In the placentas, DiR fluorescent flux was significantly higher in the 1:5 +EGFR treatment group than the A1 group, suggesting that EGFR targeting drives increased accumulation in placental cells (FIG. 42B). This is further supported by reduced DiR fluorescent flux observed in the liver in the 1:5 +EGFR treatment group, suggesting that EGFR-LNPs are trafficked to the EGFR rich placentas. mCherry fluorescent flux was not different between the two groups, however both groups demonstrated significantly increased fluorescent signal compared to PBS, confirming that antibody modification does not impair ability to deliver mRNA to placental cells.

In order to quantify LNP uptake on a cellular level, placentas and spleens were processed to generate single cell suspensions and examined for DiR fluorescence via flow cytometry. It has been widely established that fluorescent proteins, such as mCherry and GFP, often require multiple copies in order to detect signal via flow cytometry, and, thus, are not an ideal model for evaluating mRNA delivery in vivo. Consistent with these reports, mCherry positivity rates were found to be low in the spleen and below the limit of detection in the placenta. Given that the biodistribution studies confirmed the ability of EGFR-LNPs to facilitate functional luciferase mRNA expression, evaluation of the bright DiR fluorescent dye here as a metric of EGFR antibody-mediated LNP targeting of and uptake in specific cell types in the placenta and spleen was instead selected. To probe cellular uptake, placentas were stained for the cell surface markers CD45, CD31, and the intracellular marker CK7, while spleens were stained for the cell surface markers CD45, CD3, CD19, CD11b, and CD11c.

Excitingly, treatment with 1:5 +EGFR LNPs doubled the percent of DiR+ CK7+ trophoblasts, confirming that LNPs targeted to EGFR-rich trophoblasts can not only traffic to the placenta, but can increase LNP internalization in designated receptor-expressing cell types. Approximately 10% and 20% of trophoblasts were DiR+ following treatment with A1 and 1:5 +EGFR LNPs respectively (FIG. 42F), demonstrating significantly enhanced LNP extravasation through placental vasculature to adjacent EGFR+ trophoblasts via active targeting. 1:5 +EGFR LNPs also demonstrated significantly enhanced uptake in CD45+ immune cells (20% DiR+) when compared to A1 LNPs (13% DiR+), likely due to the robust presence of immune cells at the maternal-fetal interface (FIG. 42G). Given that placental immune cells are a key mediator in many placental disorders, including preeclampsia, enhanced uptake of EGFR-LNPs specifically in placental immune cells could confer additional benefits for treating these conditions. Around 10% of CD31+ endothelial cells in the placenta were DiR+ regardless of treatment group (FIG. 42H), representative of successful LNP trafficking to the placenta due to changes in cardiac output during pregnancy.

In spleens, around 52% of CD11b+ myeloid cells were DiR+ with no significant differences observed between LNP treatment groups (FIG. 42C). Roughly 42% and 38% of CD11c+ dendritic cells were DiR+ following treatment with A1 and 1:5 +EGFR LNPs respectively (FIG. 42D). These results are consistent with previous works, which have reported LNP accumulation in myeloid and dendritic cells, likely due to LNP opsonization and subsequent phagocytosis by the MPS. Given the increased luciferase expression observed in spleens in the biodistribution experiments following treatment with 1:5 +EGFR LNPs, it was suspected that antibody modification may lead to increased recognition and internalization by splenic macrophages and immune cells. However, DiR positivity in CD11b+ and CD11c+ cells was not significantly different between A1 and 1:5 +EGFR LNP treatment groups, suggesting that the presence of EGFR antibodies at an intermediate density does not exacerbate phagocytic action by splenic immune cells.

Approximately 8% of splenic CD3+ T cells were DiR+ across both LNP treatment groups. Interestingly, treatment with 1:5 +EGFR LNPs resulted in significantly less uptake in CD19+ B cells, with approximately 6% and 3% DiR+ B cells observed following A1 and 1:5 +EGFR LNP treatment respectively (FIG. 42E). As splenic B cells play a vital role in antibody production against foreign antigens, the reduction in B cell accumulation of 1:5 +EGFR LNPs further confirms that the presence of EGFR antibodies on LNPs does not elicit an exacerbated immune response when compared to A1 LNPs. Rather, the reduced uptake by B cells may speak to a potentially enhanced safety profile of EGFR-LNPs during pregnancy due to a diminished maternal immune responsiveness to foreign antibodies and a resultant decrease in antibody trafficking to immune cells in the spleen. Additional work is required to further elucidate differences in antibody-mediated immunoreactivity between non-pregnant and pregnant mice.

To further probe the potential effects of antibody presence in eliciting a systemic immune response, inflammatory cytokine markers were selected and measured the relative concentration of each cytokine in serum from PBS-treated compared to LNP-treated mice. At 12 h, relative levels of the common inflammatory markers interleukin 1-alpha (IL-la), interleukin 1-beta (IL-1P), granulocyte-macrophage colony-stimulating factor (GM-CSF), macrophage inflammatory protein 1-alpha (MIP-la), and stem cell factor (SCF) did not differ between PBS-treated and LNP-treated mice. Levels of monocyte chemoattractant protein-1 (MCP-1) and RANTES were significantly higher in LNP-treated mice compared to PBS-treated mice, however cytokine levels did not statistically differ between LNP treatment groups. Granulocyte colony-stimulating factor (G-CSF) levels were also significantly higher in LNP-treated mice compared to PBS-treated mice, and relative G-CSF levels were also significantly higher in the 1:5 +EGFR treated group compared to A1 treated mice. Increased levels of MCP-1, RANTES, and G-CSF have all previously been reported following administration of mRNA LNPs, as elevation of these cytokines has been implicated in the innate immune response to foreign nucleic acids.

Thus, the relative increase in MCP-1, RANTES, and G-CSF levels compared to PBS-treated mice are likely a reaction to the presence of foreign mRNA, and not an immunogenic response to LNPs. The additional increase in G-CSF levels in the 1:5 +EGFR treated group compared to the A1 treated group suggests a potential increase in neutrophil activity in the presence of antibody-conjugated LNPs, as the primary role of G-CSF is regulation of neutrophil proliferation and trafficking. Neutrophil activation has been previously reported following LNP administration, and is, importantly, a transient response, with G-CSF levels and neutrophil activation returning to baseline levels on the order of 48-72 hours. Taken together, these results confirm that EGFR-LNPs do not elicit an exacerbated inflammatory immune response when compared to other LNP formulations, highlighting the safety of 1:5 +EGFR LNPs for targeted mRNA delivery to the placenta during pregnancy.

### Example 21: Formulation and Characterization of LNPs with Tunable Elasticity

Thus far, limited work has been done to characterize the elasticity of LNPs. As such, it was sought to use AFM to measure the Young's modulus of several LNP formulations and assess if LNP elasticity can be modulated through sterol structure. To formulate LNPs, a lipid phase containing an ionizable lipid, phospholipid, sterol and lipid-PEG were combined in ethanol at distinct molar ratios and chaotically mixed with mRNA in an aqueous phase in a microfluidic device (Table 10).

TABLE 10
Formulation details for LNPs characterized by atomic force microscopy (AFM)

| | Ionizable | Phospho- | | Lipid-PEG | Molar ratio |
|---|---|---|---|---|---|
| Formulation | Lipid (IL) | lipid (P) | Sterol (S) | (PEG) | (IL:P:S:PEG:Biotin) |
| SM-102 | SM-102 | DSPC | Cholesterol | DMG-PEG₂ₖ | 50:10:38.5:1.35:0.15 |
| C12-200 | C12-200 | DOPE | Cholesterol | C14-PEG₂ₖ | 35:16:46.5:2.25:0.25 |
| MC3 Chol | MC3 | DSPC | Cholesterol | DMG-PEG₂ₖ | 50:10:38.5:1.31:0.19 |
| MC3 Camp | MC3 | DSPC | Campesterol | DMG-PEG₂ₖ | 50:10:38.5:1.31:0.19 |
| MC3 Sito | MC3 | DSPC | β-sitosterol | DMG-PEG₂ₖ | 50:10:38.5:1.31:0.19 |
| MC3 Stig | MC3 | DSPC | Stigmastanol | DMG-PEG₂ₖ | 50:10:38.5:1.31:0.19 |
| C12-494 | C12-494 | DOPE | Cholesterol | C14-PEG₂ₖ | 35:16:46.5:2.25:0.25 |
| Chol | | | | | |
| C12-494 | C12-494 | DOPE | Campesterol | C14-PEG₂ₖ | 35:16:46.5:2.25:0.25 |
| Camp | | | | | |
| C12-494 Sito | C12-494 | DOPE | β-sitosterol | C14-PEG₂ₖ | 35:16:46.5:2.25:0.25 |
| C12-494 Stig | C12-494 | DOPE | Stigmastanol | C14-PEG₂ₖ | 35:16:46.5:2.25:0.25 |
| C12-494 | C12-494 | DOPE | Cholesterol | C14-PEG₂ₖ | 35:16:31.5:2.25:0.25 |
| Low Chol | | | | | |
| C12-494 | C12-494 | DOPE | Cholesterol | C14-PEG₂ₖ | 35:16:61.5:2.25:0.25 |
| High Chol | | | | | |

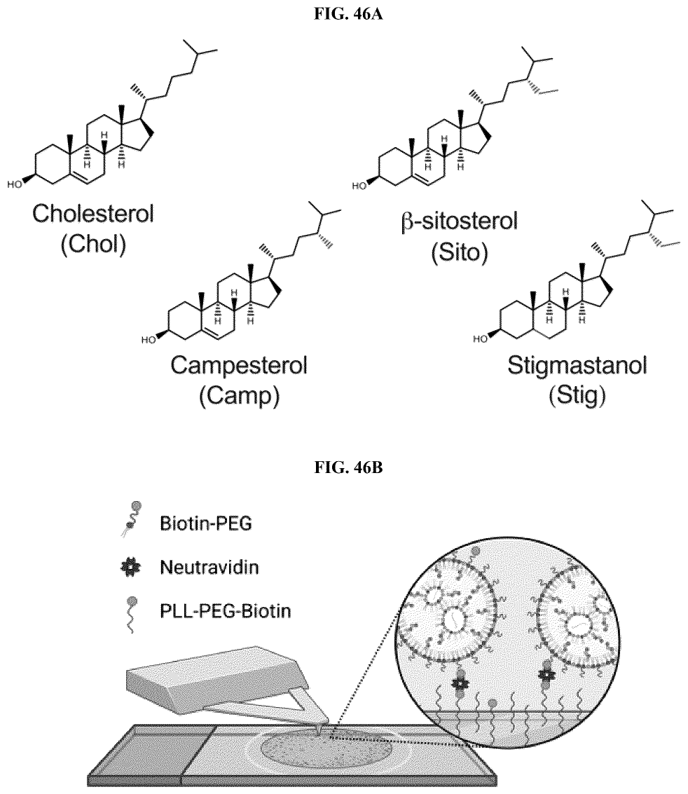

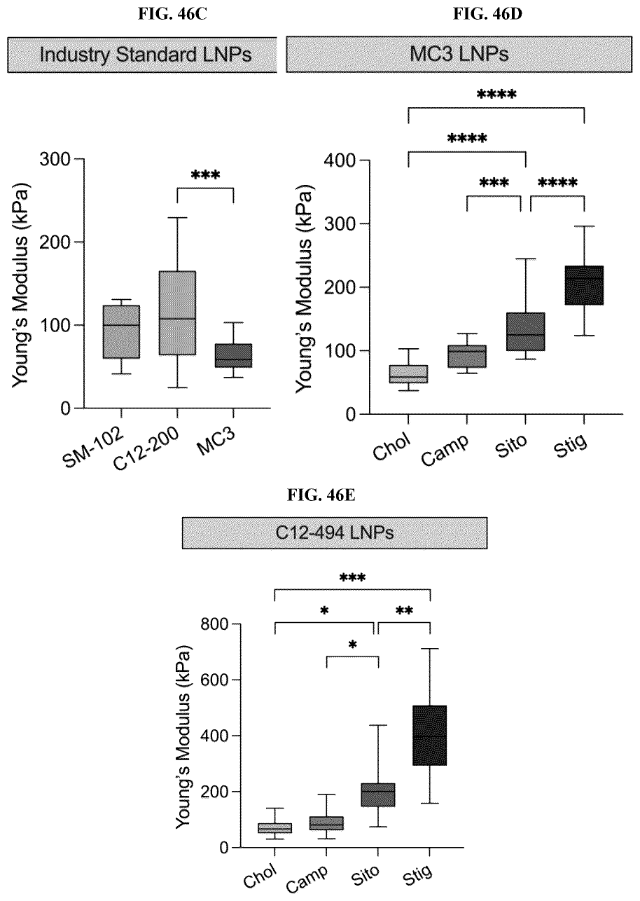

To facilitate LNP immobilization for AFM, a biotin-avidin interaction was utilized as this technique has been employed to immobilize other lipid-based nanoparticle systems for AFM and direct deposition of mRNA LNPs onto glass and mica surfaces have yielded mixed immobilization results. Specifically, biotin-modified LNPs were added to a glass slide coated with biotinylated-PEG bound to neutravidin and immersed in 1×PBS for force-indentation curve collection in liquid (FIG. 46B). Prior to AFM measurements, all LNPs were characterized for size, polydispersity index (PDI) and encapsulation efficiency (Table 11).

TABLE 11
Exemplary characterization data for certain LNPs of the disclosure by AFM

| | Z-average | Polydispersity | Encapsulation | Young's |
|---|---|---|---|---|
| Formulation | Size (nm) | Index | Efficiency (%) | Modulus (kPa) |
| SM-102 | 83.9 ± 2.0 | 0.17 ± 0.10 | 94.0 ± 0.9 | 91.1 ± 32.8 |
| C12-200 | 102.8 ± 0.9 | 0.20 ± 0.04 | 93.3 ± 1.0 | 118.8 ± 61.7 |
| MC3 Chol | 102.3 ± 2.4 | 0.17 ± 0.03 | 87.4 ± 2.4 | 63.6 ± 17.7 |
| MC3 Camp | 97.4 ± 28.0 | 0.33 ± 0.06 | 93.9 ± 2.4 | 93.6 ± 19.2 |
| MC3 Sito | 103.2 ± 1.2 | 0.11 ± 0.01 | 91.6 ± 0.1 | 135.4 ± 45.3 |
| MC3 Stig | 108.6 ± 0.6 | 0.18 ± 0.02 | 91.7 ± 3.5 | 207.9 ± 44.6 |
| C12-494 Chol | 74.8 ± 0.4 | 0.19 ± 0.02 | 95.9 ± 0.6 | 71.0 ± 26.2 |
| C12-494 Camp | 76.1 ± 1.6 | 0.24 ± 0.02 | 94.6 ± 0.2 | 87.3 ± 35.5 |
| C12-494 Sito | 90.9 ± 3.4 | 0.18 ± 0.02 | 94.2 ± 0.6 | 201.7 ± 71.4 |
| C12-494 Stig | 97.2 ± 1.1 | 0.13 ± 0.02 | 94.7 ± 0.1 | 411.4 ± 145.7 |
| C12-494 Low Chol | 79.0 ± 1.4 | 0.13 ± 0.02 | 90.0 ± 3.5 | 61.8 ± 31.1 |
| C12-494 High Chol | 87.7 ± 1.2 | 0.14 ± 0.02 | 90.0 ± 1.5 | 136.2 ± 51.9 |

To begin, AFM was used to measure the Young's modulus of three industry-standard LNP formulations, SM-102, MC3, and C12-200 (FIG. 46C). C12-200 had the highest measured Young's modulus at 118.8±61.7 kPa, followed by SM-102 at 91.1±32.8 kPa, and MC3 at 63.6±17.7 kPa. Next, the Young's modulus of MC3 LNPs formulated with Chol, Camp, Sito or Stig were measured, as these formulations were hypothesized to have different elasticities (FIG. 46D). Formulating MC3 LNPs with Camp increased the modulus to 93.6±19.2 kPa while incorporating Sito doubled the Young's modulus to 135.4±45.3 kPa. MC3 LNPs formulated with Stig had the highest Young's modulus of 207.9±44.6 kPa.

Given the interest in studying the role of LNP elasticity for mRNA delivery to the placenta, it was sought to incorporate these cholesterol analogs into a placenta-tropic LNP formulation. To this end, LNPs were formulated using the previously identified placenta-tropic C12-494 ionizable lipid with cholesterol or a cholesterol analog. Similar to the trends observed for the MC3 formulations, the C12-494 Chol and Camp LNPs had the lowest measured Young's moduli at 71.0±26.2 and 87.3±35.5 kPa, respectively (FIG. 46E). The Sito and Stig LNPs were both significantly stiffer than the Chol LNP with a measured Young's modulus of 201.7±71.4 and 411.4±145.7 kPa, respectively (FIG. 46E). The higher standard deviation observed for the Sito and Stig LNPs may be due to variation in the Young's modulus within each LNP population, where small differences in LNP lamellarity, mRNA loading or size may contribute to differences in elasticity.

The differences in elasticity between LNP formulations may be attributed to the structures of each cholesterol analog. Campesterol, β-sitosterol and stigmastanol all have a C-24 alkyl chain along their tail. Campesterol has an additional ethyl group, β-sitosterol has an additional methyl group and stigmastanol has an additional methyl group and a reduction of the double bond in the cholesterol body. Previous studies have shown that the C-24 alkyl groups induce defects in the ordering of lipid bilayers proportional to the length of the alkyl side chain, suggesting LNPs formulated with cholesterol would have the fewest defects and LNPs formulated with β-sitosterol and stigmastanol would have the most. Additionally, cryo-EM images of MC3 Sito and Stig LNPs show that these LNPs have faceted structures compared to the uniform curvature of cholesterol LNPs. The shift towards a more crystalline, faceted LNP structure when incorporating these cholesterol analogs likely correlates to the increased Young's modulus for each cholesterol analog LNP formulation.

### Example 22: Evaluation of C12-494 LNPs Containing Cholesterol Analogs for In Vitro LNP Uptake and mRNA Transfection in Trophoblasts

Nanoparticle elasticity has been shown to influence nanoparticle uptake into unique cell types. As such, it was sought to investigate the effects of LNP elasticity on both LNP uptake and mRNA transfection in placental trophoblasts using the placenta-tropic C12-494 cholesterol analog LNPs (Table 12).

TABLE 12
Formulation details for LNPs used for in vitro and in vivo screening

| | Ionizable | Phospholipid | | Lipid-PEG | Molar ratio |
|---|---|---|---|---|---|
| Formulation | Lipid (IL) | (P) | Sterol (S) | (PEG) | (IL:P:S:PEG) |
| C12-494 Chol | C12-494 | DOPE | Cholesterol | C14-PEG₂ₖ | 35:16:46.5:2.5 |
| C12-494 Camp | C12-494 | DOPE | Campesterol | C14-PEG₂ₖ | 35:16:46.5:2.5 |
| C12-494 Sito | C12-494 | DOPE | β-sitosterol | C14-PEG₂ₖ | 35:16:46.5:2.5 |
| C12-494 Stig | C12-494 | DOPE | Stigmastanol | C14-PEG₂ₖ | 35:16:46.5:2.5 |
| C12-494 Low | C12-494 | DOPE | Cholesterol | C14-PEG₂ₖ | 35:16:31.5:2.5 |
| Chol | | | | | |
| C12-494 High | C12-494 | DOPE | Cholesterol | C14-PEG₂ₖ | 35:16:61.5:2.5 |
| Chol | | | | | |

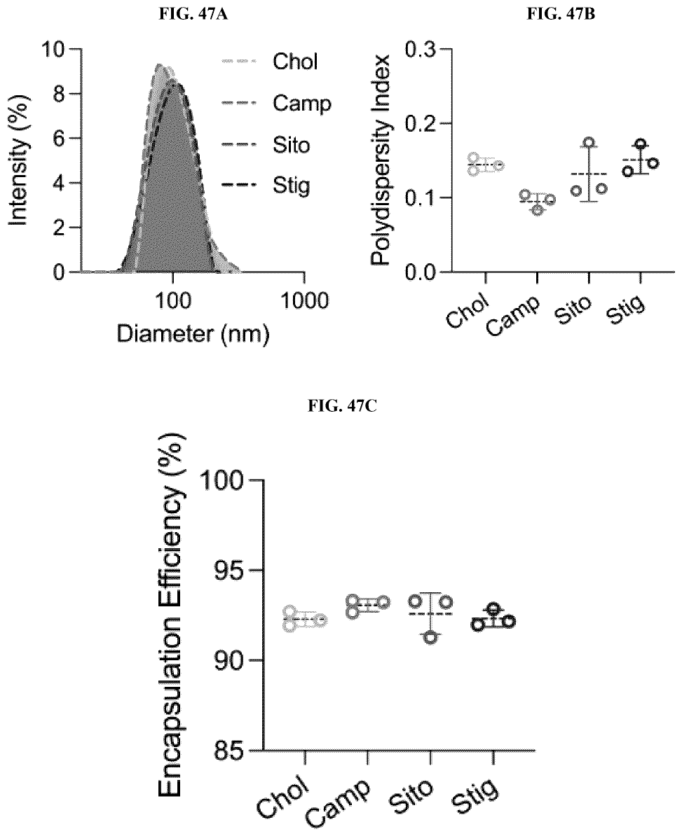

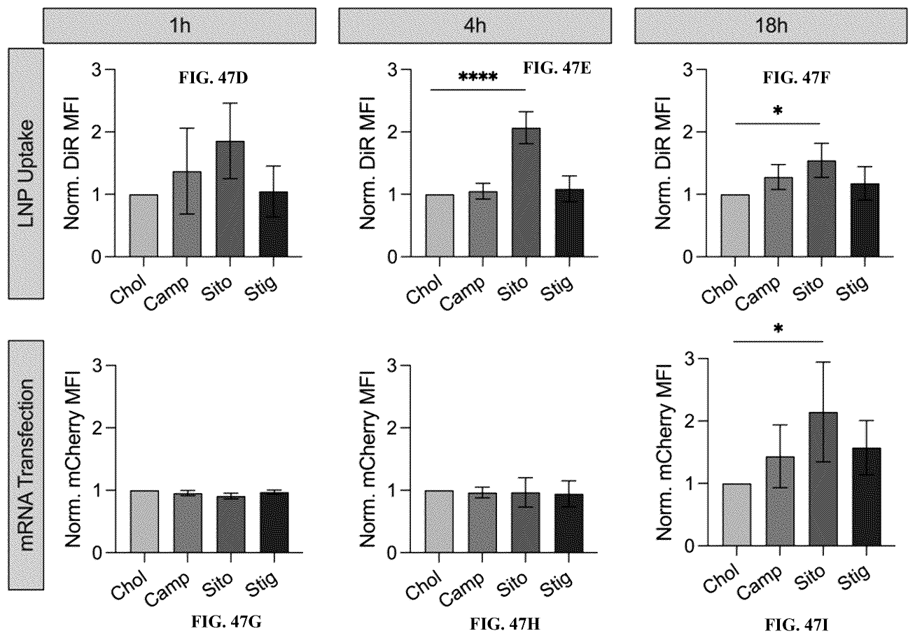

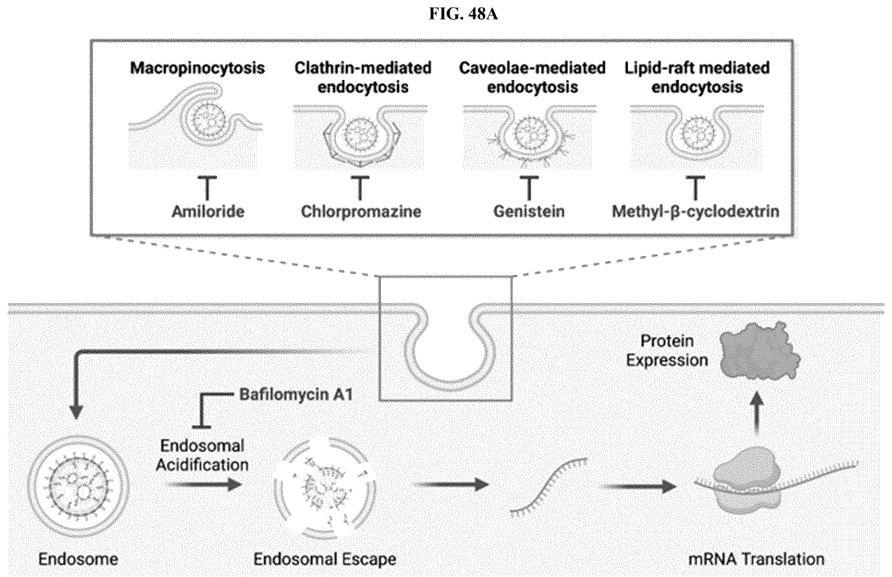

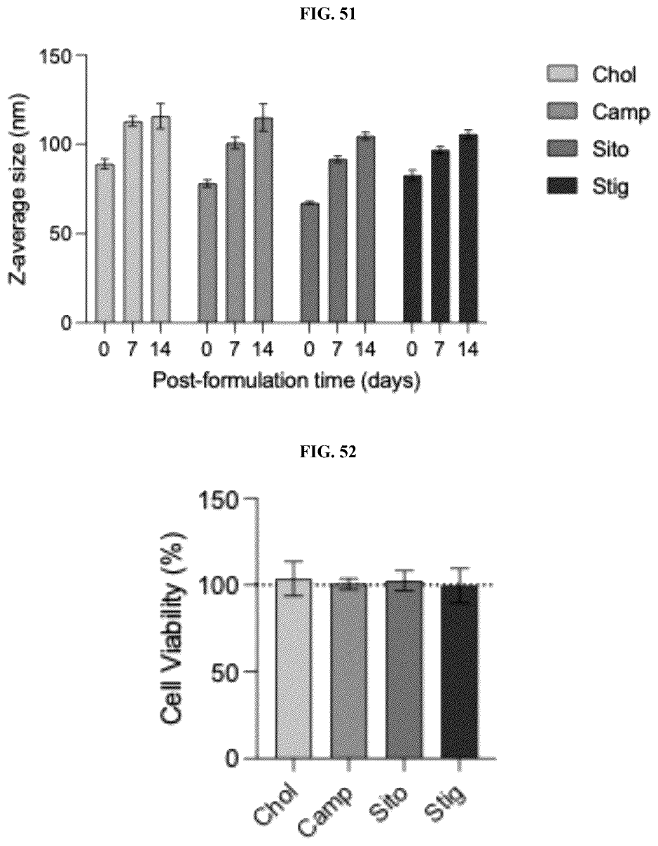

For in vitro screening, LNPs were formulated to encapsulate mCherry mRNA as a reporter cargo and labeled with the lipophilic fluorescent dye DiR to track LNP uptake. LNPs incorporating cholesterol or a cholesterol analog displayed uniform size distributions, low PDIs encapsulation efficiencies greater than 90%, and neutral zeta potentials (FIGS. 47A-47C and Table 13). When stored at 4° C. for two weeks, all LNPs exhibited similar changes in size (FIG. 51).

TABLE 13
Exemplary characterization data for LNPs used for in vitro and in vivo screening

| | Z-average | Polydispersity | Encapsulation | Zeta Potential |
|---|---|---|---|---|
| Formulation | size (nm) | Index | Efficiency (%) | (mV) |
| C12-494 Chol | 101.6 ± 0.3 | 0.14 ± 0.01 | 92.3 ± 0.4 | −7.0 ± 2.1 |
| C12-494 Camp | 91.7 ± 0.4 | 0.09 ± 0.01 | 93.1 ± 0.3 | −8.4 ± 1.9 |
| C12-494 Sito | 93.0 ± 0.6 | 0.13 ± 0.04 | 92.6 ± 1.1 | −7.6 ± 0.7 |
| C12-494 Stig | 92.1 ± 0.8 | 0.15 ± 0.02 | 92.3 ± 0.5 | −7.4 ± 1.8 |
| C12-494 Low Chol | 68.1 ± 1.3 | 0.29 ± 0.03 | 92.4 ± 1.1 | — |
| C12-494 High Chol | 77.0 ± 0.3 | 0.25 ± 0.06 | 93.2 ± 0.7 | — |

To assess both LNP uptake and mRNA transfection, BeWo b30 cells, an immortalized trophoblast cell line, were treated with LNPs. 1, 4, or 18 h after LNP treatment, flow cytometry was used to assess DiR and mCherry signal. These time points were selected as LNP uptake occurs on an earlier time scale than mRNA transfection, allowing us to examine both processes side-by-side. After 1 h of LNP treatment, both the Camp and Sito LNPs demonstrated a slight increase in DiR median fluorescent intensity (MFI) compared to the Chol LNP, suggesting an improvement in LNP uptake. By both 4 h and 18 h, the Sito LNP mediated a significant increase in DiR MFI compared to the Chol LNP (FIGS. 47D-47F). At earlier time points, no differences in mCherry signal were observed across the LNP library but by 18 h, the Sito LNP demonstrated a two-fold increase in mCherry MFI compared to the Chol LNP, confirming its potential to potently deliver mRNA to trophoblasts (FIGS. 47G-47I). Additionally, no cytotoxicity was observed for any of the LNP formulations (FIG. 52). These results indicate that LNP uptake and mRNA delivery to trophoblasts is improved by increasing the Young's modulus of LNPs, but LNPs that are too rigid may encounter challenges with uptake or release of cargo. Thus, an LNP of intermediate elasticity may be preferred as the Sito LNP demonstrated the greatest improvement in LNP uptake and mRNA delivery compared to the Chol LNP.

One limitation to the use of cholesterol analogs to alter LNP elasticity is that differences in mRNA transfection may be a result of the sterol structure, changes in elasticity or a combination of both. To investigate whether the observed differences in in vitro mRNA delivery to trophoblasts held true for other LNPs of varying elasticities, it was sought to alter LNP elasticity by solely changing the amount of cholesterol in the formulation, a strategy employed for many liposomal systems. AFM measurements revealed C12-494 LNPs formulated with a 33% decrease in cholesterol lowered the Young's modulus to 61.8±31.1 kPa while a 33% increase in cholesterol significantly increased the Young's modulus to 136.2±51.9 kPa (FIG. 53A and Table 11). When BeWo b30 cells were treated with either the low cholesterol, Chol or high cholesterol LNPs encapsulating luciferase mRNA, the stiffer, high cholesterol LNPs exhibited a two-fold improvement in luciferase expression compared to the Chol LNP, aligning with the results that LNPs of intermediate rigidity improve mRNA delivery to trophoblasts (FIG. 53B).

### Example 23: Endocytic Pathways Associated with Cholesterol Analog LNP Trafficking in Trophoblasts

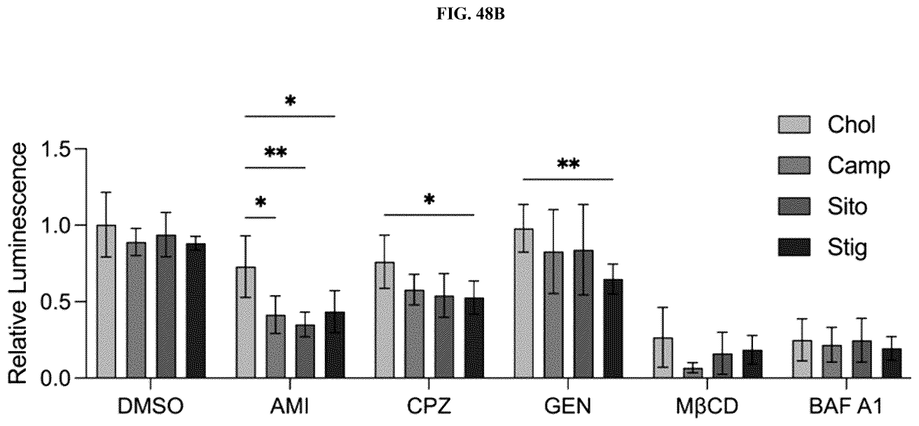

Nanoparticle elasticity has also been shown to influence nanoparticle uptake pathways in cells. While not a uniform trend, stiffer nanoparticles have exhibited preferential uptake through clathrin- and caveolae-mediated endocytosis while softer nanoparticles can be internalized both through non-specific fusion or receptor-independent endocytosis pathways. As such, it was sought to elucidate the underlying pathways governing LNP trafficking in trophoblasts for LNPs of varied elasticity. It was hypothesized that stiffer LNPs may have a greater reliance on clathrin- and caveolae-mediated endocytosis compared to the softer Chol LNPs. To this end, an inhibitor panel was designed, which consisted of five small molecules that inhibit either macropinocytosis (amiloride), clathrin-mediated endocytosis (chlorpromazine), caveolae-mediated endocytosis (genistein), lipid-raft mediated endocytosis (methyl-β-cyclodextrin) or endosomal acidification (bafilomycin A1) (FIG. 48A).

To investigate endocytosis pathways, LNPs were formulated to encapsulate luciferase mRNA and BeWo b30 cells were pre-treated with each inhibitor dissolved in DMSO or DMSO alone to ensure inhibition of mRNA transfection was not a result of DMSO treatment. Across three of the inhibitors tested, namely amiloride, chlorpromazine and genistein, stiffer LNPs exhibited reduced luciferase expression compared to the Chol LNP formulation, suggesting that stiffer LNPs are endocytosed through macropinocytosis, clathrin- and caveolae-mediated endocytosis to a greater degree than softer LNPs (FIG. 48B). Pre-treatment with either methyl-Q-cyclodextrin or bafilomycin A1 substantially reduced mRNA expression for all LNP formulations, demonstrating the importance of lipid-rafts and endosomal acidification for LNP endocytosis into trophoblasts. Additionally, no toxicity was observed for any combination of LNP or inhibitor, ensuring that reduced mRNA expression is a result of endocytosis inhibition rather than inhibitor-associated toxicity (FIG. 54).

### Example 24: Assessment of LNP Biodistribution, mRNA Transfection and Safety of Cholesterol- and Sitosterol-LNPs in Pregnant Mice

Given the improved LNP uptake and mRNA transfection demonstrated by the Sito LNP over the Chol LNP in vitro, it was next assessed if these improvements would be maintained in vivo in pregnant mice. To this end, the Chol and Sito LNPs were formulated with luciferase mRNA, dye labeled with DiR, and intravenously administered to pregnant mice on gestational day E16. A cohort of mice were also treated with PBS. 6 h after treatment, mice were injected with luciferin, euthanized, and the maternal organs, placentas and fetuses were harvested for fluorescent (LNP accumulation) and bioluminescent (mRNA transfection) imaging and quantification by an in vivo imaging system (IVIS) (FIGS. 55A-55B).

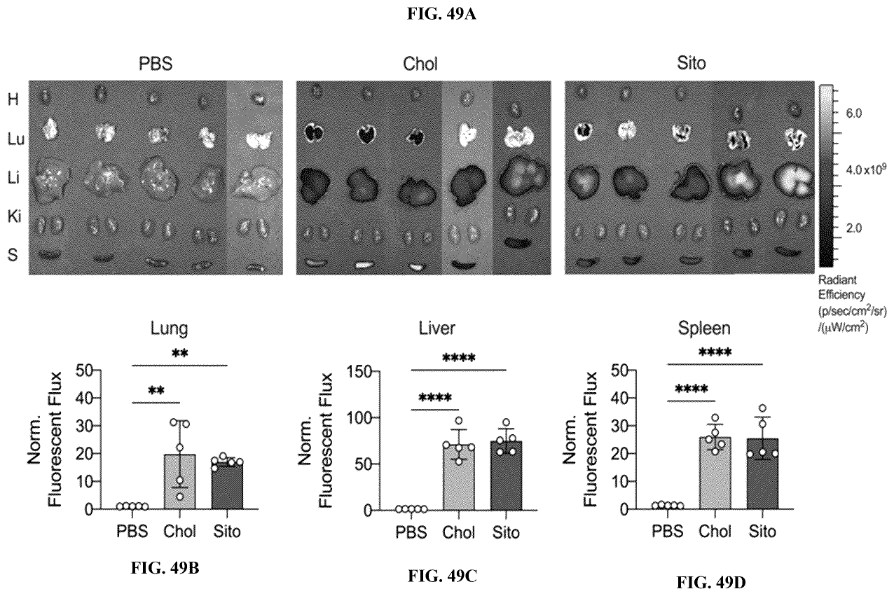

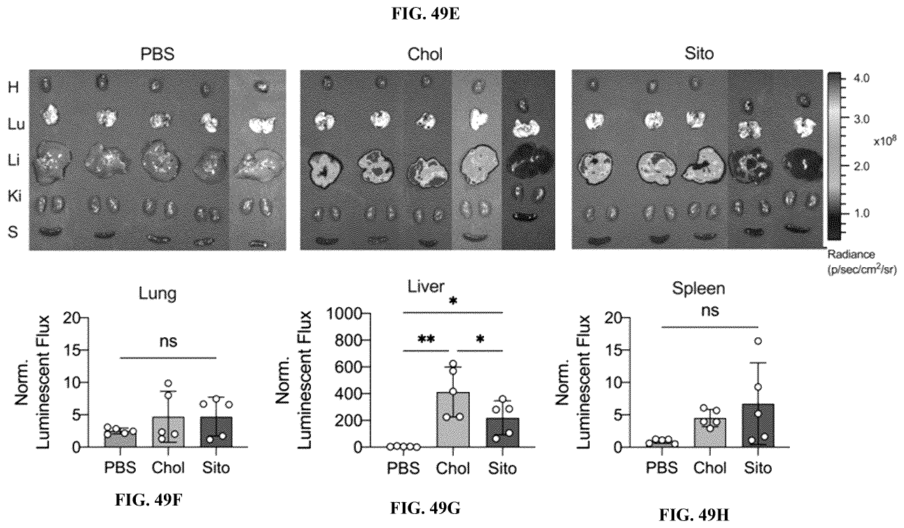

In the maternal organs, fluorescent signal was primarily observed in the lungs, liver and spleen for both the Chol and Sito LNP-treated mice and no differences in LNP biodistribution were observed between either treatment group (FIGS. 49A-49D and FIGS. 56A-56B). However, luciferase mRNA expression was observed primarily in the liver for both treatment groups where mice treated with Sito LNPs had significantly reduced liver mRNA delivery compared to the Chol LNP-treated mice (FIGS. 49E-49H and FIGS. 57A-57B). While not significant, a slight increase in luciferase signal was observed in the spleen for the Sito LNP-treated mice compared to the Chol LNP-treated mice. This trend aligns with previous findings from that found placenta-tropic LNPs also mediate mRNA delivery to the spleen, highlighting their ability to achieve extrahepatic LNP delivery. Finally, no significant differences in luciferase expression were observed between the PBS and LNP-treated mice in the lungs.

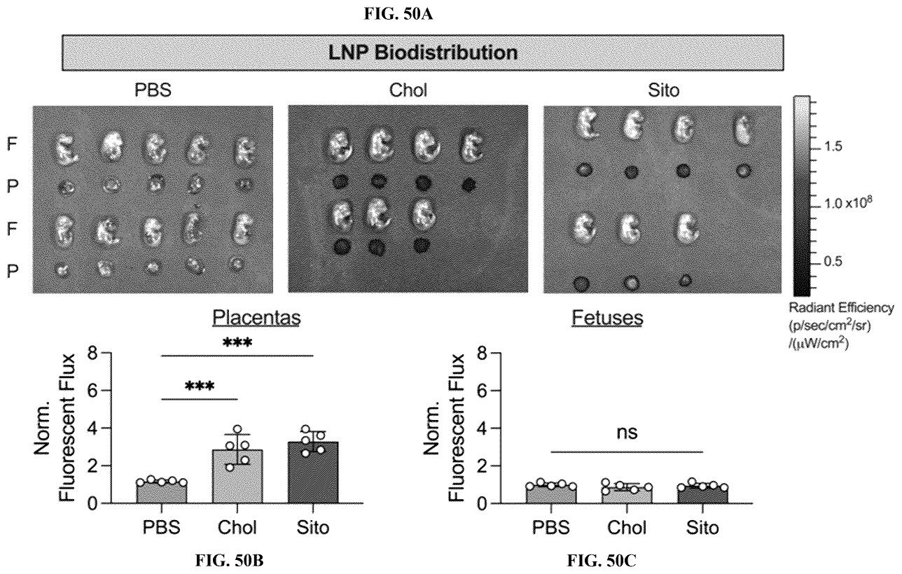

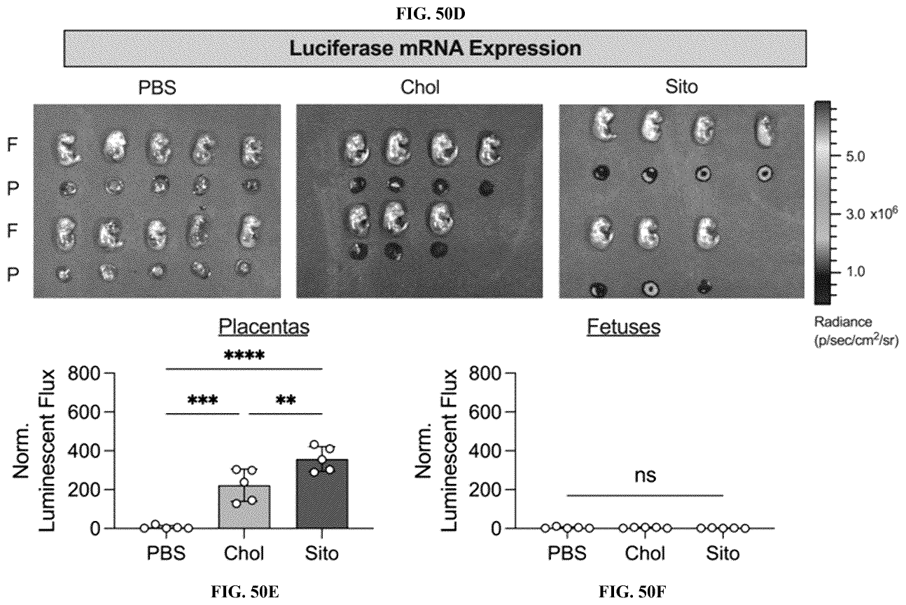

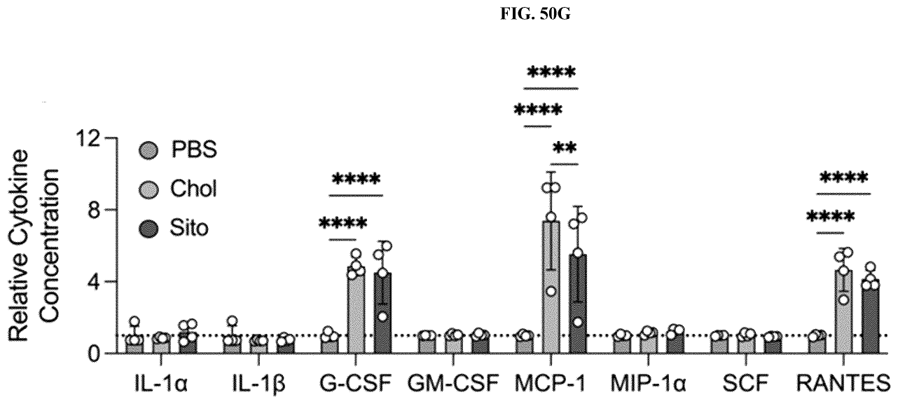

The placentas and fetuses were then imaged and LNP biodistribution and mRNA transfection was assessed for each treatment group. Both the Chol and Sito LNP-treated mice had similar placental LNP accumulation but significantly higher luminescent signal was observed in the placentas for the Sito LNP group compared to the Chol LNP group (FIGS. 50A-50B, FIGS. 50D-50E, and FIGS. 58-59). Consistent with previous findings, no fluorescent or luminescent signal was observed in the fetuses for the LNP-treated groups (FIG. 50C, FIG. 50F, and FIGS. 58-59). Given the decrease in liver and increase in placental luciferase signal for the Sito LNP-treated mice, a liver:placenta (L:P) delivery ratio was also calculated for both the Chol and Sito LNP groups (FIG. 60). The L:P ratio was 3-fold lower for the Sito LNP group compared to the Chol LNP group. While this result is not statistically significant, it suggests that the Sito LNPs are more effective at reducing liver and increasing placental mRNA delivery compared to the Chol LNP.

Finally, it was sought to assess any risks of LNP-associated inflammation between the LNP-treated and PBS-treated mice. To this end, a panel of inflammatory cytokines were selected and the relative concentration of each cytokine in serum for mice treated with PBS, Chol or Sito LNPs was assessed (FIG. 50G). Six (6) h after LNP administration, relative levels of granulocyte colony-stimulating factor (G-CSF) and RANTES were significantly higher for both Chol and Sito LNP-treated mice compared to the PBS-treated mice. Monocyte chemoattractant protein-1 (MCP-1) was elevated for both LNP-treated mice compared to PBS-treated mice, however, significantly lower levels of MCP-1 were observed for the mice treated with Sito LNP compared to Chol LNP. Elevated levels of G-CSF, MCP-1 and RANTES have been previously reported after mRNA LNP administration as these cytokines have been implicated in the innate immune response to foreign nucleic acids. MCP-1 is a key initiator in the inflammation process, recruiting or enhancing the expression of inflammatory factors or cells. The observed decrease in MCP-1 levels with the Sito LNP compared to the Chol LNP, suggests that the Sito LNP may not induce as acute of an inflammatory response compared to the Chol LNP. This is particularly important when developing LNP therapies to treat placental disorders such as pre-eclampsia, where exacerbation of the already pro-inflammatory maternal condition must be avoided.

### Sequence Listing

(EGFR IgG1)
SEQ ID NO: 1
QGFFSSPSTSRTPLLSSLSATSNNSTVACIDR
NGLQSCPIKEDSFLQRYSSDPTGALTEDSIDD
TFLPVPEYINQSVPKRPAGSVQNPVYHNQPLN
PAPSRDPHYQDPHSTAVGNPEYLNTVQPTCVN
STFDSPAHWAQKGSHQISLDNPDYQQDFFPKE
AKPNGIFKGSTAENAEYLRVAPQSSEFIGA

### Enumerated Embodiments

The following exemplary embodiments are provided, the numbering of which is not to be construed as designating levels of importance:

Embodiment 1 provides a lipid nanoparticle (LNP) composition comprising:
(a) at least one ionizable lipid;
(b) at least one helper lipid;
(c) cholesterol and/or a derivative thereof; and
(d) at least one polymer conjugated lipid.

Embodiment 2 provides a lipid nanoparticle (LNP) composition comprising:
(a) at least one ionizable lipid;
(b) at least one helper lipid;
(c) at least one cholesterol lipid and/or a derivative thereof;
(d) at least one polymer conjugated lipid and/or a modified derivative thereof; and
(e) an epidermal growth factor (EGFR) targeting domain, wherein the EGFR targeting domain is covalently conjugated to at least one component of the LNP.

Embodiment 3 provides the LNP of Embodiment 1 or 2, wherein the at least one ionizable lipid comprises an ionizable lipid of Formula (I), or a salt, solvate, stereoisomer, or isotopologue thereof:

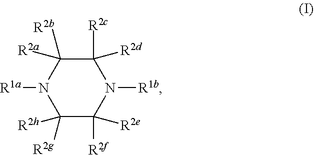

wherein:
R^{1a} and R^{1b} are each independently

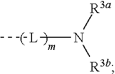

R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₇-C₁₃ aralkyl, optionally substituted C₆-C₁₀ aryl, and optionally substituted C₂-C₁₀ heteroaryl;
each occurrence of R^{3a}, R^{3b}, and R^{3c} is independently selected from the group consisting of H, -(optionally substituted C₁-C₆ alkylenyl)-C(═O)OR⁴, -(optionally substituted C₁-C₆ alkylenyl)-C(═O)N(R⁴)(Rf), -(optionally substituted C₁-C₆ alkylenyl)-C(═O)R⁴, -(optionally substituted C₁-C₆ alkylenyl)-(R⁴), —C(═O)OR⁴, —C(═O)N(R⁴)(R⁵), —C(═O)R⁴, and R⁴,
   wherein no more than one of each occurrence of R^{3a}, R^{3b}, and R³, is H;
R⁴ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁵ is selected from the group consisting of H and optionally substituted C₁-C₆ alkyl;
each occurrence of L is independently selected from the group consisting of -(optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—; and
each occurrence of m is independently an integer selected from the group consisting of 1, 2, 3, and 4.

Embodiment 4 provides the LNP of Embodiment 3, wherein at least one of the following applies:
(a) at least one selected from the group consisting of R^{2a}, R^{2b}, R^{2c}, R^{2d}, R^{2e}, R^{2f}, R^{2g}, and R^{2h} is H;
(b) at least two selected from the group consisting of R^{2a}, R^{2b}, R^{2c}, R^{2d}, R^{2e}, R^{2f}, R^{2g}, and R^{2h} are H;
(c) at least three selected from the group consisting of R^{2a}, R^{2b}, R^{2c}, R^{2d}, R^{2e}, R^{2f}, R^{2g}, and R^{2h} are H;
(d) at least four selected from the group consisting of R^{2a}, R^{2b}, R^{2c}, R^{2d}, R^{2e}, R^{2f}, R^{2g}, and R^{2h} are H;
(e) at least five selected from the group consisting of R^{2a}, R^{2b}, R^{2c}, R^{2d}, R^{2e}, R^{2f}, R^{2g}, and R^{2h} are H;
(f) at least six selected from the group consisting of R^{2a}, R^{2b}, R^{2c}, R^{2d}, R^{2e}, R^{2f}, R^{2g}, and R^{2h} are H;
(g) at least seven selected from the group consisting of R^{2a}, R^{2b}, R^{2C}, R^{2d}, R^{2e}, R^{2f}, R^{2g}, and R^{2h} are H; and
(h) each of R^{2a}, R^{2b}, R^{2c}, R^{2d}, R^{2e}, R^{2f}, R^{2g}, and R^{2h} are H.

Embodiment 5 provides the LNP of Embodiment 4, wherein each occurrence of L is independently selected from the group consisting of —CH₂—, —(CH₂)₂—, —(CH₂)₃—, —(CH₂)₁₀—, —(CH₂)₂O—, —(CH₂)₃O—, —CH₂CH(OR⁵)CH₂—, —(CH₂)₂NR^{3c}—,

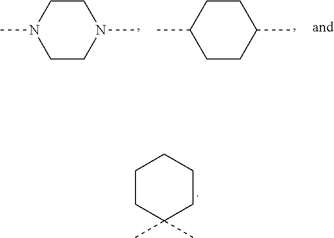

Embodiment 6 provides the LNP of any one of Embodiments 3-5, wherein the ionizable lipid of Formula (I) is selected from the group consisting of:

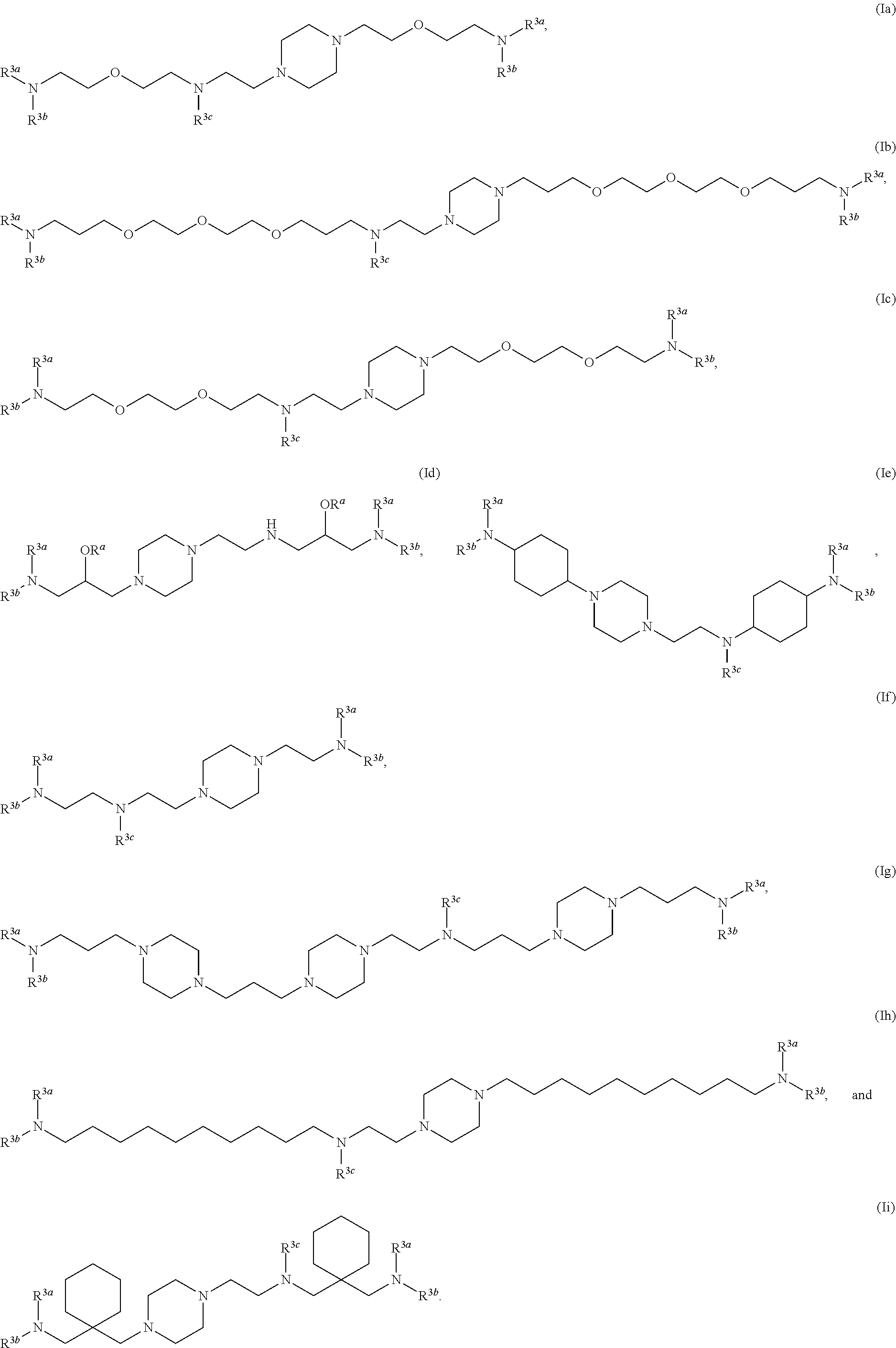

Embodiment 7 provides the LNP of any one of Embodiments 3-6, wherein each occurrence of R^{3a}, R^{3b}, and R^{3c} is independently selected from the group consisting of H, —CH₂CH(OH) (optionally substituted C₁-C₂₈ alkyl), —CH₂CH(OH) (optionally substituted C₂-C₂₈ alkenyl), —CH₂CH₂C(═O)O (optionally substituted C₁-C₂₈ alkyl), and —CH₂CH₂C(═O)NH (optionally substituted C₁-C₂₈ alkyl).

Embodiment 8 provides the LNP of any one of Embodiments 3-7, wherein each occurrence of R^{3a}, R^{3b}, and R^{3c} is independently selected from the group consisting of —CH₂CH(OH)(CH₂)₉CH₃, —CH₂CH(OH)(CH₂)₁₁CH₃, and —CH₂CH(OH)(CH₂)₁₃CH₃.

Embodiment 9 provides the LNP of any one of Embodiments 3-8, wherein each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, optionally substituted heteroalkylenyl, optionally substituted cycloalkylenyl, and optionally substituted heterocycloalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

Embodiment 10 provides the LNP of any one of Embodiments 3-9, wherein the ionizable lipid of Formula (I) is:

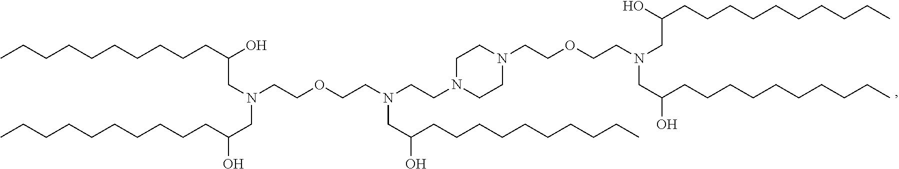 [SMILES: CCCCCCCCCCC(O)CN(CCOCCN(CC(O)CCCCCCCCCC)CC(O)CCCCCCCCCC)CCN1CCN(CCOCCN(CC(O)CCCCCCCCCC)CC(O)CCCCCCCCCC)CC1]

1,1′-((2-(2-(4-(2-((2-(2-(bis(2-hydroxydodecyl)amino)ethoxy)ethyl)(2-hydroxydodecyl)amino)ethyl)piperazin-1-yl)ethoxy)ethyl)azanediyl)bis(dodecan-2-ol) (A4);

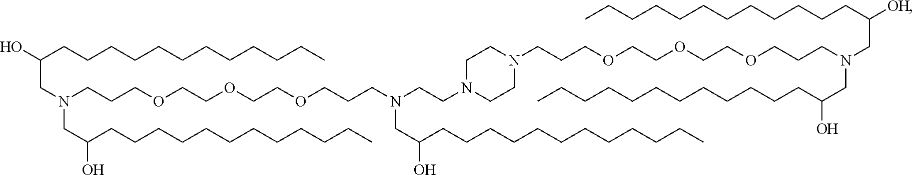 [SMILES: CCCCCCCCCCCCC(O)CN(CCCOCCOCCOCCCN(CC(O)CCCCCCCCCCCC)CC(O)CCCCCCCCCCCC)CCN1CCN(CCCOCCOCCOCCCN(CC(O)CCCCCCCCCCCC)CC(O)CCCCCCCCCCCC)CC1]

15-(2-(4-(16-hydroxy-14-(2-hydroxytetradecyl)-4,7,10-trioxa-14-azaoctacosyl)piperazin-1-yl)ethyl)-29-(2-hydroxytetradecyl)-19,22,25-trioxa-15,29-diazatritetracontane-13,31-diol (B5);

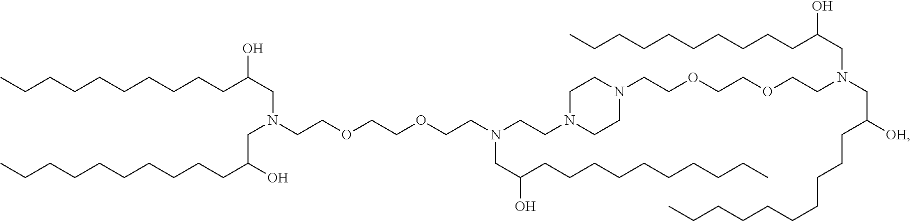 [SMILES: CCCCCCCCCCC(O)CN(CCOCCOCCN(CC(O)CCCCCCCCCC)CC(O)CCCCCCCCCC)CCN1CCN(CCOCCOCCN(CC(O)CCCCCCCCCC)CC(O)CCCCCCCCCC)CC1]

13-(2-(4-(2-(2-(2-(bis(2-hydroxydodecyl)amino)ethoxy)ethoxy)ethyl)piperazin-1-yl)ethyl)-22-(2-hydroxydodecyl)-16,19-dioxa-13,22-diazatetratriacontane-11,24-diol (A2); and

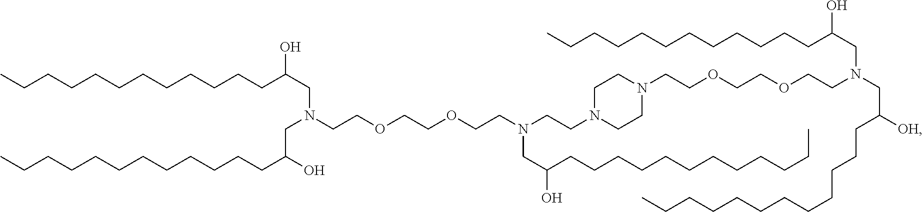 [SMILES: CCCCCCCCCCCCC(O)CN(CCOCCOCCN(CC(O)CCCCCCCCCCCC)CC(O)CCCCCCCCCCCC)CCN1CCN(CCOCCOCCN(CC(O)CCCCCCCCCCCC)CC(O)CCCCCCCCCCCC)CC1]

15-(2-(4-(2-(2-(2-(bis(2-hydroxytetradecyl)amino)ethoxy)ethoxy)ethyl)piperazin-1-yl)ethyl)-24-(2-hydroxytetradecyl)-18,21-dioxa-15,24-diazaoctatriacontane-13,26-diol (B2).

Embodiment 11 provides the LNP of any one of Embodiments 1-10, wherein the at least one ionizable lipid comprises about 10 mol % to about 60 mol % of the LNP.

Embodiment 12 provides the LNP of any one of Embodiments 1-11, wherein the at least one ionizable lipid comprises about 32.4, 35, 49, 51, or about 55 mol % of the LNP.

Embodiment 13 provides the LNP of any one of Embodiments 1-12, wherein the helper lipid comprises at least one selected from the group consisting of dioleoylphosphatidylethanolamine (DOPE) and distearoylphosphatidylcholine (DSPC).

Embodiment 14 provides the LNP of any one of Embodiments 1-13, wherein the at least one helper lipid comprises about 1 to about 50 mol % of the LNP.

Embodiment 15 provides the LNP of any one of Embodiments 1-14, wherein the at least one helper lipid comprises about 14, 16, 22.2, 29, or about 33 mol % of the LNP.

Embodiment 16 provides the LNP of Embodiment 15, wherein the helper lipid is DOPE.

Embodiment 17 provides the LNP of any one of Embodiments 1-16, wherein the cholesterol comprises about 5 to about 70 mol % of the LNP.

Embodiment 18 provides the LNP of any one of Embodiments 1-17, wherein cholesterol comprises about 15, 16, 33, 43.1, or about 46.5 mol % of the LNP.

Embodiment 19 provides the LNP of any one of Embodiments 1-18, wherein the at least one polymer conjugated lipid comprises about 0.1 to about 20.0 mol % of the LNP.

Embodiment 20 provides the LNP of any one of Embodiments 1-19, wherein the at least one polymer conjugated lipid comprises about 1.6, 1.8, 1.9, 2.3, or about 2.5 mol % of the LNP.

Embodiment 21 provides the LNP of any one of Embodiments 1-20, wherein the at least one polymer conjugated lipid comprises C14-PEG2000.

Embodiment 22 provides the LNP of any one of Embodiments 1-21, wherein the LNP has a molar ratio of (a):(b):(c):(d) selected from the group consisting of:
(a) about 30:20:10:1;
(b) about 30:16:8:1;
(c) about 55:15:35:2;
(d) about 35:16:46.5:2.5; and
(e) about 35:24:46.5:2.5.

Embodiment 23 provides the LNP of any one of Embodiments 1-22, wherein the LNP comprises (a):(b):(c):(d) having a molar percentage selected from the group consisting of:
(a) about 49.18:32.79:16.39:1.64;
(b) about 54.55:29.09:14.55:1.82;
(c) about 51.40:14.02:32.71:1.87;
(d) about 35:16:46.5:2.5; and
(e) about 32.4:22.2:43.1:2.3.

Embodiment 24 provides the LNP of any one of Embodiments 1-23, further comprising at least one cargo molecule.

Embodiment 25 provides the LNP of Embodiment 24, wherein the cargo is at least one selected from the group consisting of a nucleic acid, small molecule, protein, therapeutic agent, antibody, and any combinations thereof.

Embodiment 26 provides the LNP of Embodiment 24 or 25, wherein the cargo is a nucleic acid.

Embodiment 27 provides the LNP of Embodiment 25 or 26, wherein the nucleic acid is DNA or RNA.

Embodiment 28 provides the LNP of Embodiment 26 or 27, wherein the nucleic acid is selected from the group consisting of mRNA, cDNA, pDNA, microRNA, siRNA, modified RNA, antagomir, antisense molecule, and any combinations thereof.

Embodiment 29 provides the LNP of any one of Embodiments 24-28, wherein the cargo is at least partially encapsulated in the LNP.

Embodiment 30 provides the LNP of any one of Embodiments 24-29, wherein the cargo is mRNA.

Embodiment 31 provides the LNP of Embodiment 30, wherein the LNP has a weight ratio of ionizable lipid to mRNA ranging from about 5:1 to about 20:1.

Embodiment 32 provides the LNP of Embodiment 30 or 31, wherein the LNP has a weight ratio of ionizable lipid to mRNA of about 10:1.

Embodiment 33 provides the LNP of any one of Embodiments 28-32, wherein the mRNA encodes VEGF.

Embodiment 34 provides the LNP of any one of Embodiments 2-33, wherein the epidermal growth factor (EGFR) targeting domain is covalently conjugated to the at least one polymer conjugated lipid.

Embodiment 35 provides the LNP of Embodiment 34, wherein the targeting domain comprises at least one selected from the group consisting of a polypeptide, a polynucleotide, and a small molecule.

Embodiment 36 provides the LNP of Embodiment 35, wherein the targeting domain comprises a polypeptide, optionally wherein the polypeptide is an antibody, optionally wherein the antibody is EGFR IgG1.

Embodiment 37 provides the LNP of any one of Embodiments 34-36, wherein the at least one polymer conjugated lipid comprises a polyethylene glycol (PEG) conjugated lipid and an EGFR-PEG-conjugated lipid (EGFR-PEG).

Embodiment 38 provides the LNP of Embodiment 37, wherein the EGFR targeting domain is covalently conjugated to the PEG conjugated lipid via a linker comprising a moiety formed by a click reaction, optionally wherein the click reaction is selected from the group consisting of a [3+2] cycloaddition and a [4+2] cycloaddition, optionally wherein the [3+2] cycloaddition is selected from the group consisting of a strain-promoted azide-alkyne cycloaddition (SPAAC), a Cu(I)-catalyzed azide-alkyne cycloaddition (CuAAC), and a strain-promoted alkyne-nitrone cycloaddition (SPANC), and optionally wherein the [4+2] cycloaddition is selected from the group consisting of a Diels-Alder reaction and an alkene/tetrazine inverse-demand Diels-Alder reaction.

Embodiment 39 provides the LNP of Embodiment 38, wherein the moiety comprises a 1,2,3-triazole.

Embodiment 40 provides the LNP of Embodiment 38 or 39, wherein each of the following apply:
(a) the linker has a first terminus which is covalently conjugated to a functional group of a side chain residue or a terminal residue of the polypeptide comprising the epidermal growth factor (EGFR) targeting domain; and
(b) the linker has a second terminus which is covalently conjugated to a terminal hydroxyl of the PEG conjugated lipid.

Embodiment 41 provides the LNP of any one of Embodiments 38-40, wherein the linker is selected from the group consisting of:

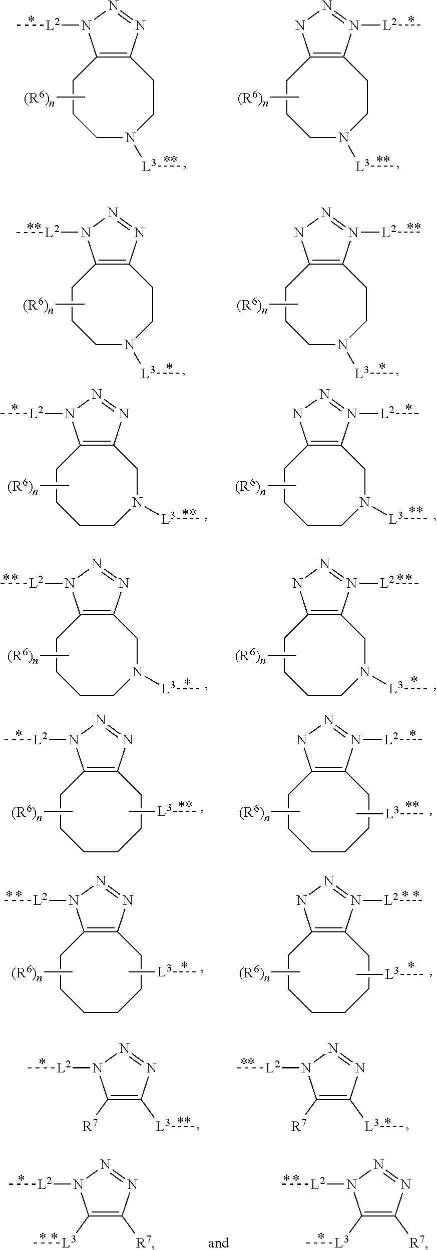

wherein:
L² and L³ are each independently a bond or at least one divalent substituent selected from the group consisting of —C(═O)—, —N(R^{a})—, —O—, —S—, optionally substituted C₁-C₁₂ alkylenyl, optionally substituted C₃-C₁₂ heterocycloalkylenyl, optionally substituted C₂-C₁₂ heteroalkylenyl, and optionally substituted C₂-C₁₂ heterocycloalkylenyl;
R⁶ is selected from the group consisting of optionally substituted C₁-C₆ alkyl, C₂-C₆ heteroalkyl, optionally substituted C₃-C₈ cycloalkyl, optionally substituted phenyl, optionally substituted benzyl, optionally substituted C₂-C₉ heterocyclyl, halogen, OR^{a}, N(R^{a})(R^{b}), SR^{a}, CN, and NO₂,
   wherein two adjacent R⁶ substituents may combine with the atoms to which they are bound to form an optionally substituted phenyl, optionally substituted C₃-C₈ cycloalkyl, or optionally substituted C₂-C₉ heterocyclyl;
each occurrence of R^{a} and R^{b} is independently selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
R⁷ is selected from the group consisting of H, optionally substituted C₁-C₆ alkyl, C₂-C₆ heteroalkyl, optionally substituted C₃-C₈ cycloalkyl, optionally substituted phenyl, optionally substituted benzyl, and optionally substituted C₂-C₉ heterocyclyl;
n is an integer from 0 to 11;
* indicates a bond between the linker and the EGFR targeting domain; and
** indicates a bond between the linker and the polymer conjugated lipid.

Embodiment 42 provides the LNP of Embodiment 41, wherein the linker is

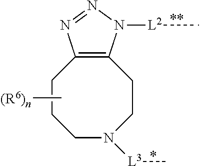

Embodiment 43 provides the LNP of Embodiment 41 or 42, wherein L² and L³ are each independently selected from the group consisting of a bond and —C(═O)—(CH₂)₃—C(═O)NH—(CH₂)₂—(OCH₂CH₂)₁₋₁₀₀—C(═O)—.

Embodiment 44 provides the LNP of any one of Embodiments 41-43, wherein the linker comprises:

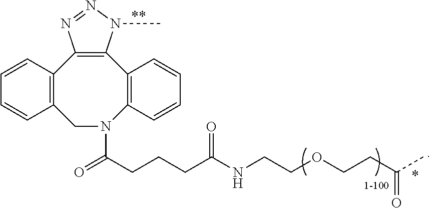

Embodiment 45 provides the LNP of any one of Embodiments 34-44, wherein the at least one polymer conjugated lipid comprises about 0.1 to about 5.0 mol % of the LNP.

Embodiment 46 provides the LNP of any one of Embodiments 34-45, wherein the at least one polymer conjugated lipid comprises about 2.5 mol % of the LNP.

Embodiment 47 provides the LNP of any one of Embodiments 37-46, wherein the EGFR-PEG-conjugated lipid (EGFR-PEG) and the polyethylene glycol (PEG) have a ratio ranging from about 1:1 to about 1:20 (EGFR-PEG:PEG).

Embodiment 48 provides the LNP of any one of Embodiments 37-47, wherein EGFR-PEG-conjugated lipid (EGFR-PEG) and the polyethylene glycol (PEG) have a ratio selected from the group consisting of about 1:2, 1:3, 1:5, and 1:7.

Embodiment 49 provides the LNP of any one of Embodiments 34-48, wherein the LNP has a molar ratio of (a):(b):(c):(d) of about 35:16:46.5:2.5.

Embodiment 50 provides the LNP of any one of Embodiments 1-49, wherein the LNP is selectively delivered to the placenta of a subject.

Embodiment 51 provides a pharmaceutical composition comprising the LNP of any one of Embodiments 1-50 and a pharmaceutically acceptable carrier.

Embodiment 52 provides a method of delivering a cargo to the placenta of a pregnant subject, the method comprising administering to the subject a therapeutically effective amount of at least one lipid nanoparticle (LNP) comprising:
(a) at least one ionizable lipid;
(b) at least one helper lipid;
(c) cholesterol and/or a derivative thereof;
(d) at least one polymer conjugated lipid and/or a modified derivative thereof;
(e) at least one cargo molecule, wherein the at least one cargo molecule is at least partially encapsulated in the LNP;
optionally wherein the LNP further comprises an epidermal growth factor (EGFR) targeting domain, wherein the EGFR targeting domain is covalently conjugated to at least one component of the LNP.

Embodiment 53 provides a method of treating, preventing, and/or ameliorating a placental disease and/or disorder in a subject in need thereof, the method comprising administering to a subject a therapeutically effective amount of at least one lipid nanoparticle comprising:
(a) at least one ionizable lipid;
(b) at least one helper lipid;
(c) cholesterol and/or a modified derivative thereof;
(d) at least one polymer conjugated lipid and/or a modified derivative thereof;
(e) at least one cargo molecule, wherein the at least one cargo molecule is at least partially encapsulated in the LNP;
optionally wherein the LNP further comprises an epidermal growth factor (EGFR) targeting domain, wherein the EGFR targeting domain is covalently conjugated to at least one component of the LNP.

Embodiment 54 provides the method of Embodiment 53, wherein the placental disorder is selected from the group consisting of pre-eclampsia, fetal growth restriction (FGR), intrauterine growth restriction (IUGR), placenta previa, placenta accreta, placenta increta, and placenta percreta.

Embodiment 55 provides the method of any one of Embodiments 52-54, wherein the cargo is at least one selected from the group consisting of a nucleic acid, small molecule, protein, therapeutic agent, antibody, and any combinations thereof.

Embodiment 56 provides the method of any one of Embodiments 52-55, wherein the cargo is a nucleic acid.

Embodiment 57 provides the method of Embodiment 55 or 56, wherein the nucleic acid is DNA or RNA.

Embodiment 58 provides the method of Embodiment 56 or 57, wherein the nucleic acid is selected from the group consisting of mRNA, cDNA, pDNA, microRNA, siRNA, modified RNA, antagomir, antisense molecule, and any combinations thereof.

Embodiment 59 provides the method of any one of Embodiments 52-58, wherein the cargo is mRNA.

Embodiment 60 provides the method of Embodiment 59, wherein the mRNA encodes VEGF.

Embodiment 61 provides the method of any one of Embodiments 52-60, wherein the LNP is administered as a pharmaceutical composition.

Embodiment 62 provides the method of Embodiment 61, wherein the pharmaceutical composition comprises at least one pharmaceutically acceptable carrier.

Embodiment 63 provides the method of any one of Embodiments 52-62, wherein the at least one ionizable lipid comprises an ionizable lipid of Formula (I), or a salt, solvate, stereoisomer, or isotopologue thereof:

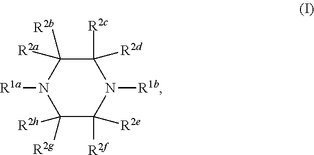

wherein:
R^{1a} and R^{1b} are each independently

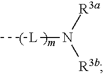

R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₇-C₁₃ aralkyl, optionally substituted C₆-C₁₀ aryl, and optionally substituted C₂-C₁₀ heteroaryl;
each occurrence of R^{3a}, R^{3h}, and R^{3c} is independently selected from the group consisting of H, -(optionally substituted C₁-C₆ alkylenyl)-C(═O)OR⁴, -(optionally substituted C₁-C₆ alkylenyl)-C(═O)N(R⁴)(R⁵), -(optionally substituted C₁-C₆ alkylenyl)-C(═O)R⁴, -(optionally substituted C₁-C₆ alkylenyl)-(R⁴), —C(═O)OR⁴, —C(═O)N(R⁴)(R⁵), —C(═O)R⁴, and R⁴,
   wherein no more than one of each occurrence of R^{3a}, R^{3b}, and R^{3c} is H;
R⁴ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁵ is selected from the group consisting of H and optionally substituted C₁-C₆ alkyl;
each occurrence of L is independently selected from the group consisting of -(optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—; and
each occurrence of m is independently an integer selected from the group consisting of 1, 2, 3, and 4.

Embodiment 64 provides the method of Embodiment 63, wherein at least one of the following applies:
(a) at least one selected from the group consisting of R^{2a}, R^{2e}, R^{2c}, R^{2d}, R^{2e}, R^{2f}, R^{2g}, and R^{2h} is H;
(b) at least two selected from the group consisting of R^{2a}, R^{2h}, R^{2c}, R^{2d}, R^{2e}, R^{2f}, R^{2g}, and R^{2h} are H;
(c) at least three selected from the group consisting of R^{2a}, R^{2b}, R^{2c}, R^{2d}, R^{2e}, R^{2f}, R^{2g}, and R^{2h} are H;
(d) at least four selected from the group consisting of R^{2a}, R^{2b}, R^{2c}, R^{2d}, R^{2e}, R^{2f}, R^{2g}, and R^{2h} are H;
(e) at least five selected from the group consisting of R^{2a}, R^{2b}, R^{2c}, R^{2d}, R^{2e}, R^{2f}, R^{2g}, and R^{2h} are H;
(f) at least six selected from the group consisting of R^{2a}, R^{2b}, R^{2c}, R^{2d}, R^{2e}, R^{2f}, R^{2g}, and R^{2h} are H;
(g) at least seven selected from the group consisting of R^{2a}, R^{2b}, R^{2c}, R^{2d}, R^{2e}, R^{2f}, R^{2g}, and R^{2h} are H; and
(h) each of R^{2a}, R^{2b}, R^{2c}, R^{2d}, R^{2e}, R^{2f}, R^{2g}, and R^{2h} are H.

Embodiment 65 provides the method of Embodiment 63 or 64, wherein each occurrence of L is independently selected from the group consisting of —CH₂—, —(CH₂)₂—, —(CH₂)₃—, —(CH₂)₁₀—, —(CH₂)₂O—, —(CH₂)₃O—, —CH₂CH(OR⁵)CH₂—, —(CH₂)₂NR^{3c}—,

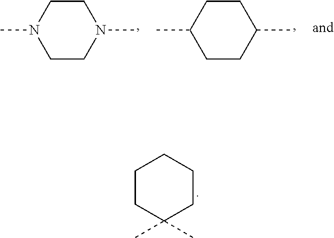

Embodiment 66 provides the method of any one of Embodiments 63-65, wherein the ionizable lipid of Formula (I) is selected from the group consisting of:

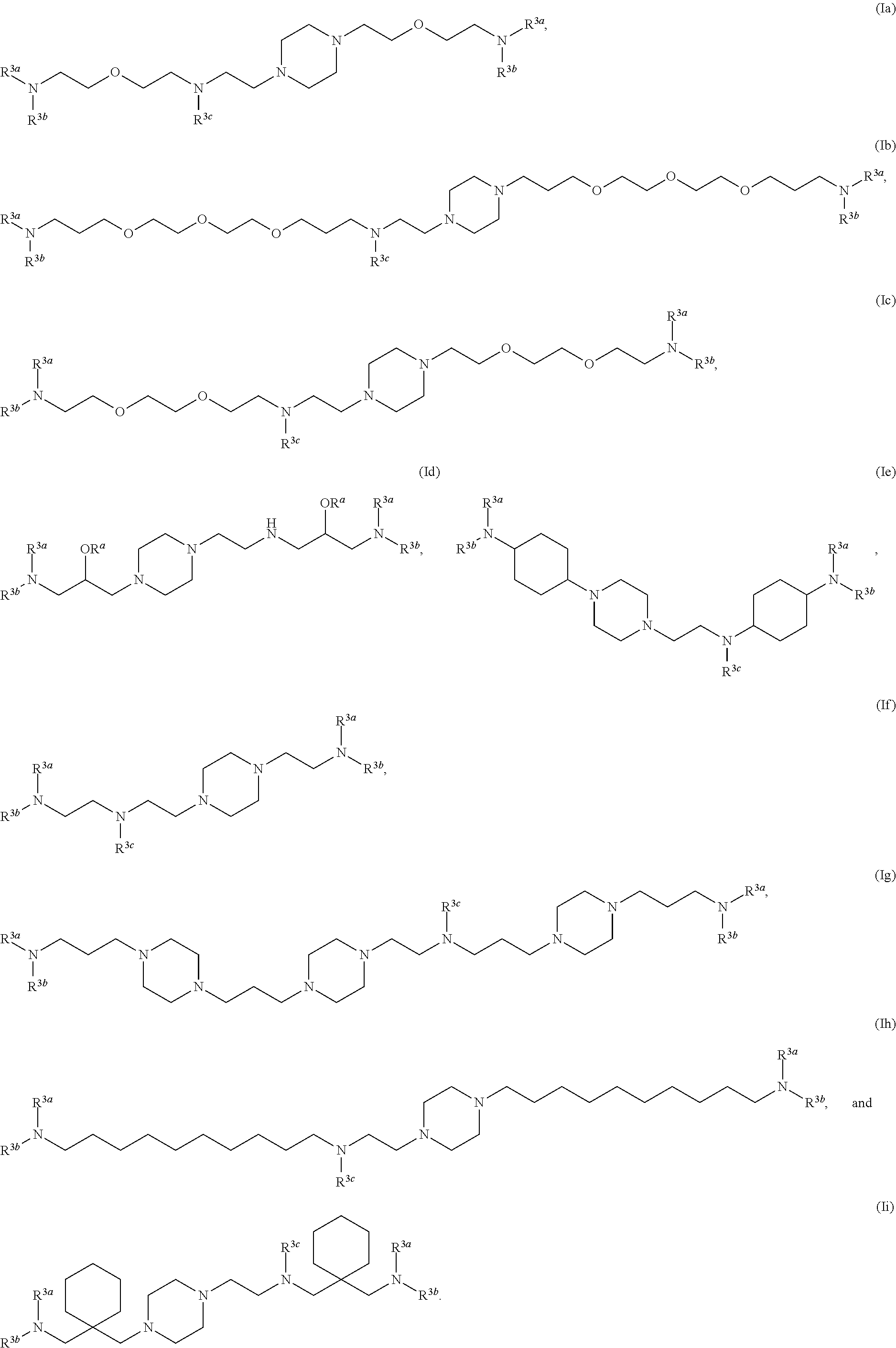

Embodiment 67 provides the method of any one of Embodiments 63-66, wherein each occurrence of R^{3a}, R^{3b}, and R^{3c} is independently selected from the group consisting of H, —CH₂CH(OH) (optionally substituted C₁-C₂₈ alkyl), —CH₂CH(OH) (optionally substituted C₂-C₂₈ alkenyl), —CH₂CH₂C(═O)O (optionally substituted C₁-C₂₈ alkyl), and —CH₂CH₂C(═O)NH (optionally substituted C₁-C₂₈ alkyl).

Embodiment 68 provides the method of any one of Embodiments 63-67, wherein each occurrence of R^{3a}, R^{3b}, and R^{3c} is independently selected from the group consisting of —CH₂CH(OH)(CH₂)₉CH₃, —CH₂CH(OH)(CH₂)₁₁CH₃, and —CH₂CH(OH)(CH₂)₁₃CH₃.

Embodiment 69 provides the method of any one of Embodiments 63-68, wherein each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, optionally substituted heteroalkylenyl, optionally substituted cycloalkylenyl, and optionally substituted heterocycloalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

Embodiment 70 provides the method of any one of Embodiments 63-69, wherein the ionizable lipid of Formula (I) is:

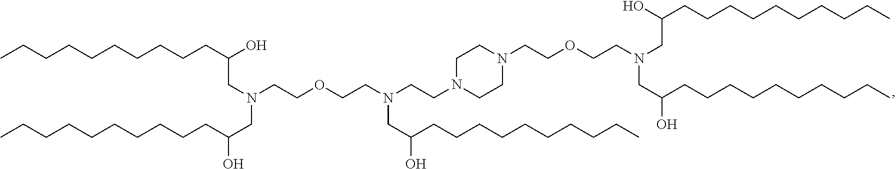 [SMILES: CCCCCCCCCCC(O)CN(CCOCCN(CC(O)CCCCCCCCCC)CC(O)CCCCCCCCCC)CCN1CCN(CCOCCN(CC(O)CCCCCCCCCC)CC(O)CCCCCCCCCC)CC1]

1,1′-((2-(2-(4-(2-((2-(2-(bis(2-hydroxydodecyl)amino)ethoxy)ethyl)(2-hydroxydodecyl)amino)ethyl)piperazin-1-yl)ethoxy)ethyl)azanediyl)bis(dodecan-2-ol) (A4);

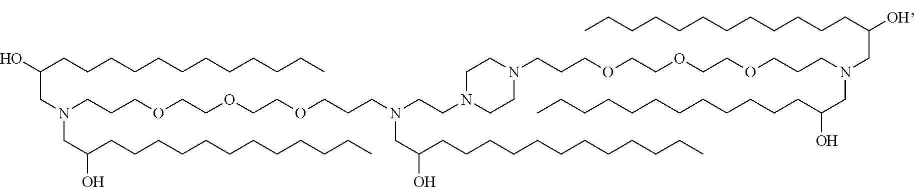 [SMILES: CCCCCCCCCCCCC(O)CN(CCCOCCOCCOCCCN(CC(O)CCCCCCCCCCCC)CC(O)CCCCCCCCCCCC)CCN1CCN(CCCOCCOCCOCCCN(CC(O)CCCCCCCCCCCC)CC(O)CCCCCCCCCCCC)CC1]

15-(2-(4-(16-hydroxy-14-(2-hydroxytetradecyl)-4,7,10-trioxa-14-azaoctacosyl)piperazin-1-yl)ethyl)-29-(2-hydroxytetradecyl)-19,22,25-trioxa-15,29-diazatritetracontane-13,31-diol (B5);

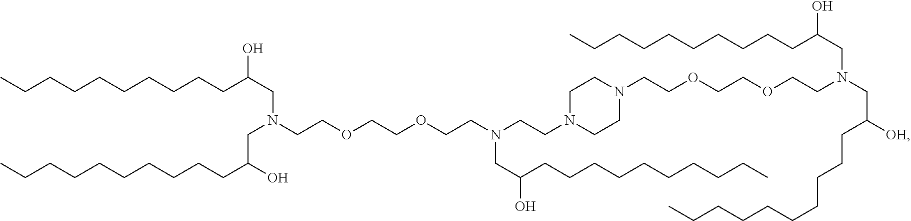 [SMILES: CCCCCCCCCCC(O)CN(CCOCCOCCN(CC(O)CCCCCCCCCC)CC(O)CCCCCCCCCC)CCN1CCN(CCOCCOCCN(CC(O)CCCCCCCCCC)CC(O)CCCCCCCCCC)CC1]

13-(2-(4-(2-(2-(2-(bis(2-hydroxydodecyl)amino)ethoxy)ethoxy)ethyl)piperazin-1-yl)ethyl)-22-(2-hydroxydodecyl)-16,19-dioxa-13,22-diazatetratriacontane-11,24-diol (A2); and

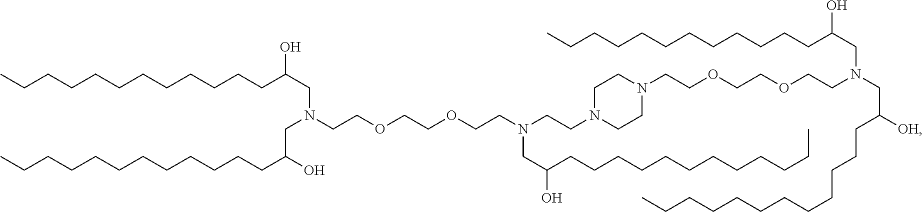 [SMILES: CCCCCCCCCCCCC(O)CN(CCOCCOCCN(CC(O)CCCCCCCCCCCC)CC(O)CCCCCCCCCCCC)CCN1CCN(CCOCCOCCN(CC(O)CCCCCCCCCCCC)CC(O)CCCCCCCCCCCC)CC1]

15-(2-(4-(2-(2-(2-(bis(2-hydroxytetradecyl)amino)ethoxy)ethoxy)ethyl)piperazin-1-yl)ethyl)-24-(2-hydroxytetradecyl)-18,21-dioxa-15,24-diazaoctatriacontane-13,26-diol (B2).

Embodiment 71 provides the method of any one of Embodiments 52-70, wherein the at least one ionizable lipid comprises about 10 mol % to about 60 mol % of the LNP.

Embodiment 72 provides the method of any one of Embodiments 52-71, wherein the at least one ionizable lipid comprises about 32.4, 35, 49, 51, or about 55 mol % of the LNP.

Embodiment 73 provide the method of any one of Embodiments 52-72, wherein the helper lipid comprises at least one selected from the group consisting of dioleoylphosphatidylethanolamine (DOPE) and distearoylphosphatidylcholine (DSPC).

Embodiment 74 provides the method of any one of Embodiments 52-73, wherein the at least one helper lipid comprises about 1 to about 50 mol % of the LNP.

Embodiment 75 provides the method of any one of Embodiments 52-74, wherein the at least one helper lipid comprises about 14, 16, 22.2, 29, or about 33 mol % of the LNP.

Embodiment 76 provides the method of Embodiment 75, wherein the helper lipid is DOPE.

Embodiment 77 provides the method of any one of Embodiments 52-76, wherein the cholesterol comprises about 5 to about 70 mol % of the LNP.

Embodiment 78 provides the method of any one of Embodiments 52-77, wherein cholesterol comprises about 15, 16, 33, or about 46.5 mol % of the LNP.

Embodiment 79 provides the method of any one of Embodiments 52-78, wherein the at least one polymer conjugated lipid comprises about 0.1 to about 20.0 mol % of the LNP.

Embodiment 80 provides the method of any one of Embodiments 52-79, wherein the at least one polymer conjugated lipid comprises about 1.6, 1.8, 1.9, 2.3, or about 2.5 mol % of the LNP.

Embodiment 81 provides the method of any one of Embodiments 52-80, wherein the at least one polymer conjugated lipid comprises C14-PEG2000.

Embodiment 82 provides the method of any one of Embodiments 52-81, wherein the LNP has a molar ratio of (a):(b):(c):(d) selected from the group consisting of:
(a) about 30:20:10:1;
(b) about 30:16:8:1;
(c) about 55:15:35:2;
(d) about 35:16:46.5:2.5;
(e) about 35:24:46.5:2.5.

Embodiment 83 provides the method of any one of Embodiments 52-82, wherein the LNP comprises (a):(b):(c):(d) having a molar percentage selected from the group consisting of:
(a) about 49.18:32.79:16.39:1.64;
(b) about 54.55:29.09:14.55:1.82;
(c) about 51.40:14.02:32.71:1.87;
(d) about 35:16:46.5:2.5; and
(e) about 32.4:22.2:43.1:2.3.

Embodiment 84 provides a method of delivering a cargo to the placenta of a pregnant subject, the method comprising administering to the subject a therapeutically effective amount of at least one lipid nanoparticle (LNP) comprising:
(a) at least one ionizable lipid;
(b) at least one helper or neutral lipid;
(c) cholesterol and/or a derivative thereof;
(d) at least one polymer conjugated lipid and/or a modified derivative thereof;
(e) at least one cargo molecule, wherein the at least one cargo molecule is at least partially encapsulated in the LNP.

Embodiment 85 provides a method of treating, preventing, and/or ameliorating a placental disease and/or disorder in a subject in need thereof, the method comprising administering to a subject a therapeutically effective amount of at least one lipid nanoparticle comprising:
(a) at least one ionizable lipid;
(b) at least one helper or neutral lipid;
(c) cholesterol and/or a modified derivative thereof;
(d) at least one polymer conjugated lipid and/or a modified derivative thereof;
(e) at least one cargo molecule, wherein the at least one cargo molecule is at least partially encapsulated in the LNP.

Embodiment 86 provides the method of Embodiment 84 or 85, wherein the LNP further comprises an epidermal growth factor (EGFR) targeting domain, wherein the EGFR targeting domain is covalently conjugated to at least one component of the LNP.

Embodiment 87 provides the method of Embodiment 85 or 86, wherein the placental disorder is selected from the group consisting of pre-eclampsia, fetal growth restriction (FGR), intrauterine growth restriction (IUGR), placenta previa, placenta accreta, placenta increta, and placenta percreta.

Embodiment 88 provides the method of any one of Embodiments 84-87, wherein the cargo is at least one selected from the group consisting of a nucleic acid, small molecule, protein, therapeutic agent, antibody, and any combinations thereof.

Embodiment 89 provides the method of any one of Embodiments 84-88, wherein the cargo is a nucleic acid.

Embodiment 90 provides the method of any one of Embodiments 84-89, wherein the nucleic acid is DNA or RNA.

Embodiment 91 provides the method of any one of Embodiments 84-90, wherein the nucleic acid is selected from the group consisting of mRNA, cDNA, pDNA, microRNA, siRNA, modified RNA, antagomir, antisense molecule, and any combinations thereof.

Embodiment 92 provides the method of any one of Embodiments 84-91, wherein the cargo is mRNA, optionally wherein the mRNA encodes VEGF.

Embodiment 93 provides the method of any one of Embodiments 84-93, wherein the LNP is administered as a pharmaceutical composition.

Embodiment 94 provides the method of Embodiment 93, wherein the pharmaceutical composition comprises at least one pharmaceutically acceptable carrier.

Embodiment 95 provides the method of any one of Embodiments 84-94, wherein the at least one ionizable lipid comprises an ionizable lipid selected from the group consisting of C12-200, MC3, and a compound of Formula (I), or a salt, solvate, stereoisomer, or isotopologue thereof:

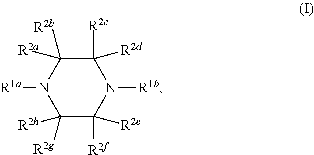

wherein:
R^{1a} and R^{1b} are each independently

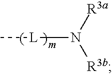

R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₇-C₁₃ aralkyl, optionally substituted C₆-C₁₀ aryl, and optionally substituted C₂-C₁₀ heteroaryl;
each occurrence of R^{3a}, R^{3b}, and R^{3c} is independently selected from the group consisting of H, -(optionally substituted C₁-C₆ alkylenyl)-C(═O)OR⁴, -(optionally substituted C₁-C₆ alkylenyl)-C(═O)N(R⁴)(R⁵), -(optionally substituted C₁-C₆ alkylenyl)-C(═O)R⁴, -(optionally substituted C₁-C₆ alkylenyl)-(R⁴), —C(═O)OR⁴, —C(═O)N(R⁴)(R⁵), —C(═O)R⁴, and R⁴,
wherein no more than one of each occurrence of R^{3a}, R^{3b}, and R^{3c} is H;
R⁴ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁵ is selected from the group consisting of H and optionally substituted C₁-C₆ alkyl;
each occurrence of L is independently selected from the group consisting of -(optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—; and
each occurrence of m is independently an integer selected from the group consisting of 1, 2, 3, and 4.

Embodiment 96 provides the method of Embodiment 95, wherein at least one of the following applies:
(a) at least one selected from the group consisting of R^{2a}, R^{2b}, R^{2c}, R^{2d}, R^{2e}, R^{2f}, R^{2g} and R^{2h} is H;
(b) at least two selected from the group consisting of R^{2a}, R^{2b}, R^{2c}, R^{2d}, R^{2e}, R^{2f}, R^{2g}, and R^{2h} are H;
(c) at least three selected from the group consisting of R^{2a}, R^{2b}, R^{2c}, R^{2d}, R^{2e}, R^{2f}, R^{2g}, and R^{2h} are H;
(d) at least four selected from the group consisting of R^{2a}, R^{2b}, R^{2c}, R^{2d}, R^{2e}, R^{2f}, R^{2g}, and R^{2h} are H;
(e) at least five selected from the group consisting of R^{2a}, R^{2b}, R^{2c}, R^{2d}, R^{2e}, R^{2f}, R^{2g}, and R^{2h} are H;
(f) at least six selected from the group consisting of R^{2a}, R^{2b}, R^{2c}, R^{2d}, R^{2e}, R^{2f}, R^{2g}, and R^{2h} are H;
(g) at least seven selected from the group consisting of R^{2a}, R^{2b}, R^{2c}, R^{2d}, R^{2e}, R^{2f}, R^{2g}, and R^{2h} are H; and
(h) each of R^{2a}, R^{2b}, R^{2c}, R^{2d}, R^{2e}, R^{2f}, R^{2g}, and R^{2h} are H.

Embodiment 97 provides the method of Embodiment 95 or 96, wherein each occurrence of L is independently selected from the group consisting of —CH₂—, —(CH₂)₂—, —(CH₂)₃—, —(CH₂)₁₀—, —(CH₂)₂O—, —(CH₂)₃O—, —CH₂CH(OR⁵)CH₂—, —(CH₂)₂NR^{3c}—,

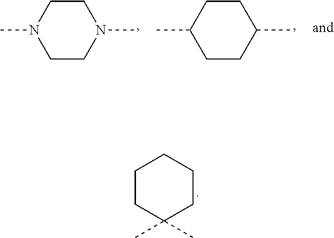

Embodiment 98 provides the method of any one of Embodiments 95-97, wherein the ionizable lipid of Formula (I) is selected from the group consisting of:

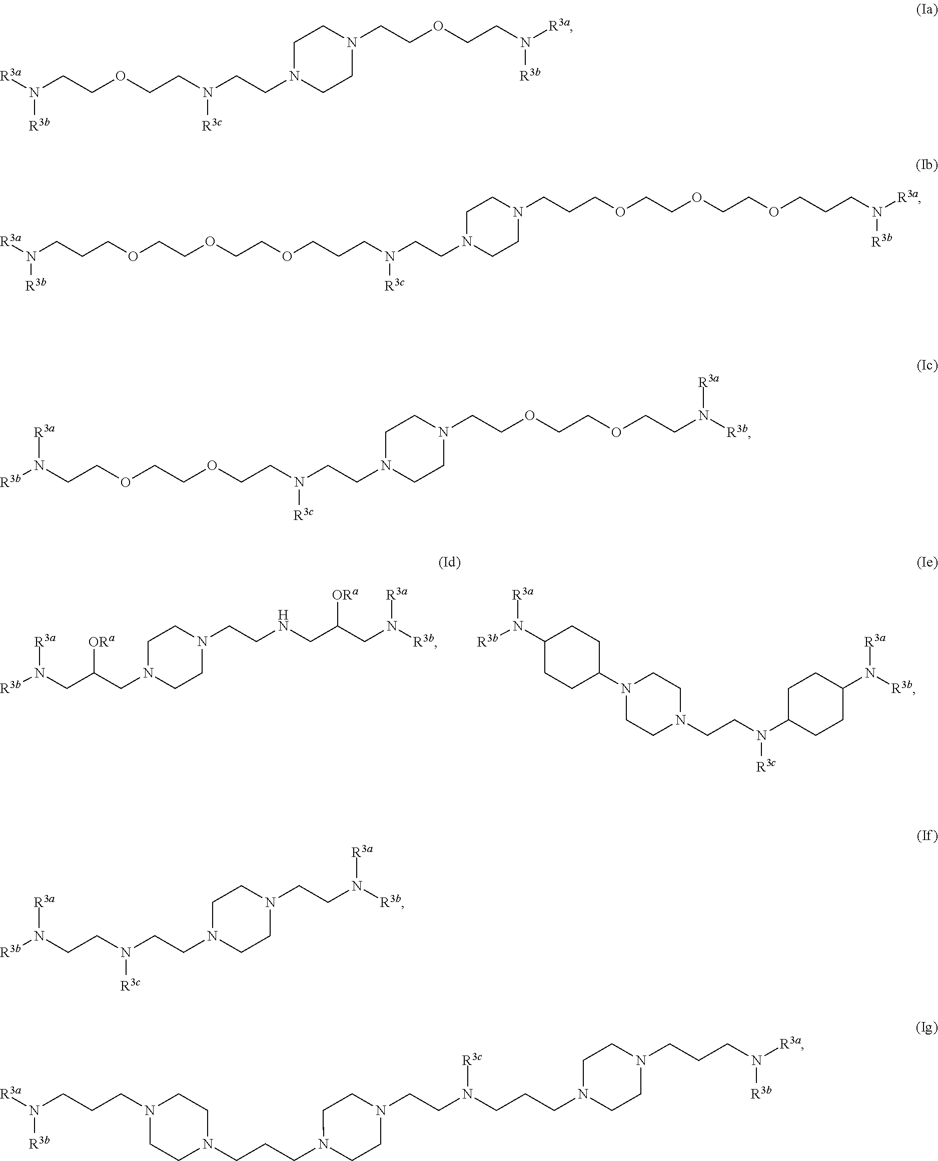

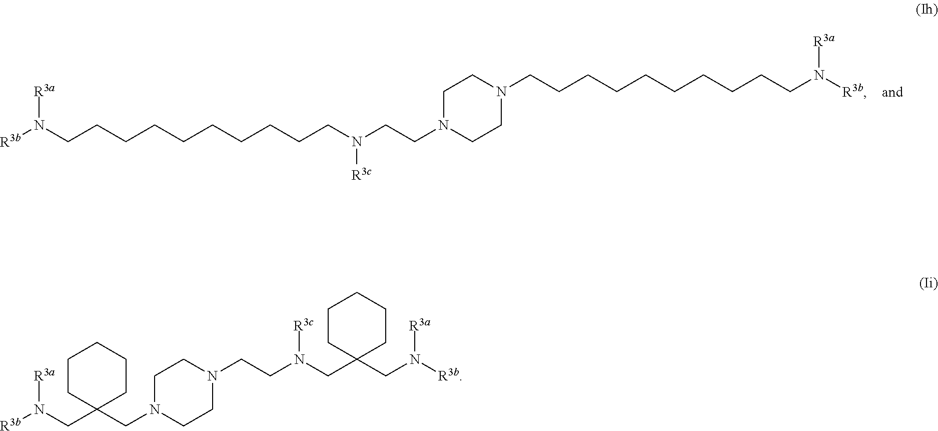

Embodiment 99 provides the method of any one of Embodiments 95-98, wherein each occurrence of R^{3a}, R^{3b}, and R^{3c} is independently selected from the group consisting of H, —CH₂CH(OH) (optionally substituted C₁-C₂₈ alkyl), —CH₂CH(OH) (optionally substituted C₂-C₂₈ alkenyl), —CH₂CH₂C(═O)O (optionally substituted C₁-C₂₈ alkyl), and —CH₂CH₂C(═O)NH (optionally substituted C₁-C₂₈ alkyl).

Embodiment 100 provides the method of any one of Embodiments 95-99, wherein each occurrence of R^{3a}, R^{3b}, and R^{3c} is independently selected from the group consisting of —CH₂CH(OH)(CH₂)₉CH₃, —CH₂CH(OH)(CH₂)₁₁CH₃, and —CH₂CH(OH)(CH₂)₁₃CH₃.

Embodiment 101 provides the method of any one of Embodiments 95-100, wherein each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, optionally substituted heteroalkylenyl, optionally substituted cycloalkylenyl, and optionally substituted heterocycloalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

Embodiment 102 provides the method of any one of Embodiments 95-101, wherein the ionizable lipid of Formula (I) is:

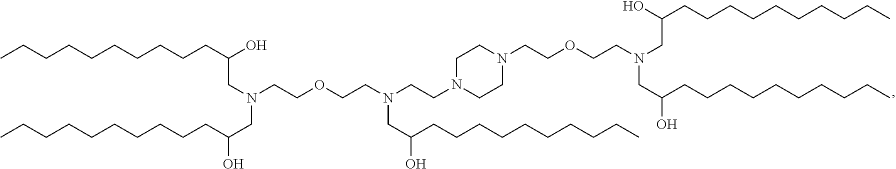 [SMILES: CCCCCCCCCCC(O)CN(CCOCCN(CC(O)CCCCCCCCCC)CC(O)CCCCCCCCCC)CCN1CCN(CCOCCN(CC(O)CCCCCCCCCC)CC(O)CCCCCCCCCC)CC1]

1,1′-((2-(2-(4-(2-((2-(2-(bis(2-hydroxydodecyl)amino)ethoxy)ethyl)(2-hydroxydodecyl)amino)ethyl)piperazin-1-yl)ethoxy)ethyl)azanediyl)bis(dodecan-2-ol) (A4);

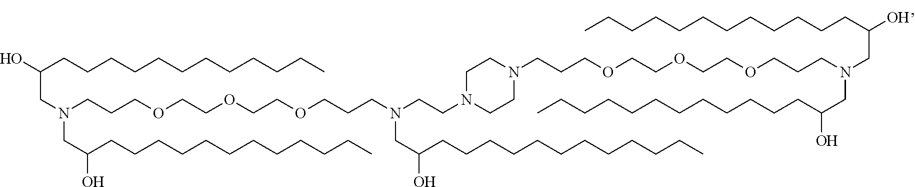 [SMILES: CCCCCCCCCCCCC(O)CN(CCCOCCOCCOCCCN(CC(O)CCCCCCCCCCCC)CC(O)CCCCCCCCCCCC)CCN1CCN(CCCOCCOCCOCCCN(CC(O)CCCCCCCCCCCC)CC(O)CCCCCCCCCCCC)CC1]

15-(2-(4-(16-hydroxy-14-(2-hydroxytetradecyl)-4,7,10-trioxa-14-azaoctacosyl)piperazin-1-yl)ethyl)-29-(2-hydroxytetradecyl)-19,22,25-trioxa-15,29-diazatritetracontane-13,31-diol (B5);

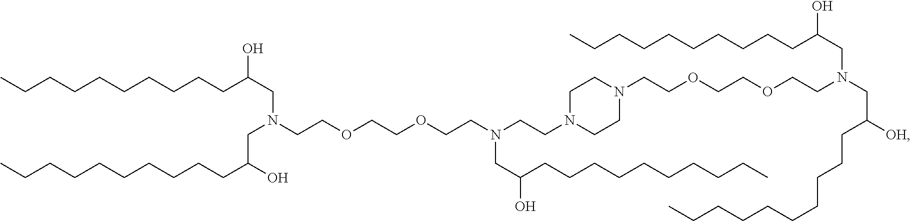 [SMILES: CCCCCCCCCCC(O)CN(CCOCCOCCN(CC(O)CCCCCCCCCC)CC(O)CCCCCCCCCC)CCN1CCN(CCOCCOCCN(CC(O)CCCCCCCCCC)CC(O)CCCCCCCCCC)CC1]

13-(2-(4-(2-(2-(2-(bis(2-hydroxydodecyl)amino)ethoxy)ethoxy)ethyl)piperazin-1-yl)ethyl)-22-(2-hydroxydodecyl)-16,19-dioxa-13,22-diazatetratriacontane-11,24-diol (A2);

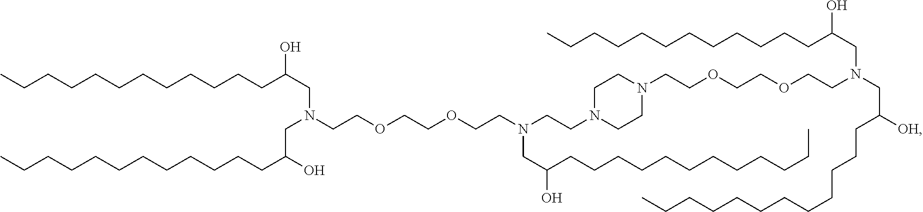 [SMILES: CCCCCCCCCCCCC(O)CN(CCOCCOCCN(CC(O)CCCCCCCCCCCC)CC(O)CCCCCCCCCCCC)CCN1CCN(CCOCCOCCN(CC(O)CCCCCCCCCCCC)CC(O)CCCCCCCCCCCC)CC1]

15-(2-(4-(2-(2-(2-(bis(2-hydroxytetradecyl)amino)ethoxy)ethoxy)ethyl)piperazin-1-yl)ethyl)-24-(2-hydroxytetradecyl)-18,21-dioxa-15,24-diazaoctatriacontane-13,26-diol (B2); and

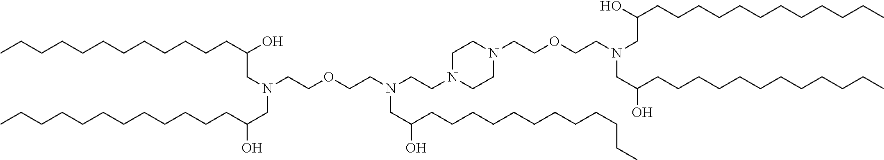 [SMILES: CCCCCCCCCCCCC(O)CN(CCOCCN(CC(O)CCCCCCCCCCCC)CC(O)CCCCCCCCCCCC)CCN1CCN(CCOCCN(CC(O)CCCCCCCCCCCC)CC(O)CCCCCCCCCCCC)CC1]

1,1′-((2-(2-(4-(2-((2-(2-(bis(2-hydroxytetradecyl)amino)ethoxy)ethyl)(2-hydroxytetradecyl)amino)ethyl)piperazin-1-yl)ethoxy)ethyl)azanediyl)bis(tetradecan-2-ol), (C14-494).

Embodiment 103 provides the method of any one of Embodiments 84-102, wherein the at least one ionizable lipid comprises about 10 mol % to about 60 mol % of the LNP.

Embodiment 104 provides the method of any one of Embodiments 84-103, wherein the at least one ionizable lipid comprises about 32.4, 35, 49, 51, or about 55 mol % of the LNP.

Embodiment 105 provides the method of any one of Embodiments 84-104, wherein the helper or neutral lipid comprises at least one selected from the group consisting of dioleoylphosphatidylethanolamine (DOPE) and distearoylphosphatidylcholine (DSPC).

Embodiment 106 provides the method of any one of Embodiments 84-105, wherein the at least one helper or neutral lipid comprises about 1 to about 50 mol % of the LNP.

Embodiment 107 provides the method of any one of Embodiments 84-106, wherein the at least one helper or neutral lipid comprises about 14, 16, 22.2, 29, or about 33 mol % of the LNP.

Embodiment 108 provides the method of any one of Embodiments 84-107, wherein the helper or neutral lipid is DOPE.

Embodiment 109 provides the method of any one of Embodiments 84-108, wherein the cholesterol and/or a derivative thereof comprises about 5 to about 70 mol % of the LNP.

Embodiment 110 provides the method of any one of Embodiments 84-109, wherein the cholesterol and/or a derivative thereof comprises about 15, 16, 31.5, 33, 46.5, or about 61.5 mol % of the LNP.

Embodiment 111 provides the method of any one of Embodiments 84-110, wherein the cholesterol and/or a derivative thereof is selected from the group consisting of cholesterol, 24-α-methyl-cholesterol (campesterol), 24-α-ethyl-cholestanol (stigmastanol), and 24-α-ethyl-cholesterol (β-sitosterol).

Embodiment 112 provides the method of any one of Embodiments 84-111, wherein the at least one polymer conjugated lipid comprises about 0.1 to about 20.0 mol % of the LNP.

Embodiment 113 provides the method of any one of Embodiments 84-112, wherein the at least one polymer conjugated lipid comprises about 1.6, 1.8, 1.9, 2.3, or about 2.5 mol % of the LNP.

Embodiment 114 provides the method of any one of Embodiments 84-113, wherein the at least one polymer conjugated lipid comprises C14-PEG2000 (C14-PEG₂ₖ).

Embodiment 115 provides the method of any one of Embodiments 84-114, wherein the LNP has a molar ratio of (a):(b):(c):(d) selected from the group consisting of:
(a) about 30:20:10:1;
(b) about 30:16:8:1;
(c) about 55:15:35:2;
(d) about 35:16:46.5:2.5;
(e) about 35:24:46.5:2.5; and
(f) about 35:16:61.5:2.5.

Embodiment 116 provides the method of any one of Embodiments 84-115, wherein the LNP comprises (a):(b):(c):(d) having a molar percentage selected from the group consisting of:
(a) about 49.18:32.79:16.39:1.64;
(b) about 54.55:29.09:14.55:1.82;
(c) about 51.40:14.02:32.71:1.87;
(d) about 35:16:46.5:2.5; and
(e) about 32.4:22.2:43.1:2.3.

Embodiment 84 provides a method of delivering a cargo to the placenta of a pregnant subject, the method comprising administering to the subject a therapeutically effective amount of at least one lipid nanoparticle (LNP) comprising:
(a) at least one ionizable lipid;
(b) at least one helper or neutral lipid;
(c) cholesterol and/or a derivative thereof;
(d) at least one polymer conjugated lipid and/or a modified derivative thereof;
(e) at least one cargo molecule, wherein the at least one cargo molecule is at least partially encapsulated in the LNP.

Embodiment 85 provides a method of treating, preventing, and/or ameliorating a placental disease and/or disorder in a subject in need thereof, the method comprising administering to a subject a therapeutically effective amount of at least one lipid nanoparticle comprising:
(a) at least one ionizable lipid;
(b) at least one helper or neutral lipid;
(c) cholesterol and/or a modified derivative thereof;
(d) at least one polymer conjugated lipid and/or a modified derivative thereof;
(e) at least one cargo molecule, wherein the at least one cargo molecule is at least partially encapsulated in the LNP.

Embodiment 86 provides the method of Embodiment 84 or 85, wherein the LNP further comprises an epidermal growth factor (EGFR) targeting domain, wherein the EGFR targeting domain is covalently conjugated to at least one component of the LNP.

Embodiment 87 provides the method of Embodiment 85 or 86, wherein the placental disorder is selected from the group consisting of pre-eclampsia, fetal growth restriction (FGR), intrauterine growth restriction (IUGR), placenta previa, placenta accreta, placenta increta, and placenta percreta.

Embodiment 88 provides the method of any one of Embodiments 84-87, wherein the cargo is at least one selected from the group consisting of a nucleic acid, small molecule, protein, therapeutic agent, antibody, and any combinations thereof.

Embodiment 89 provides the method of any one of Embodiments 84-88, wherein the cargo is a nucleic acid.

Embodiment 90 provides the method of any one of Embodiments 84-89, wherein the nucleic acid is DNA or RNA.

Embodiment 91 provides the method of any one of Embodiments 84-90, wherein the nucleic acid is selected from the group consisting of mRNA, cDNA, pDNA, microRNA, siRNA, modified RNA, antagomir, antisense molecule, and any combinations thereof.

Embodiment 92 provides the method of any one of Embodiments 84-91, wherein the cargo is mRNA, optionally wherein the mRNA encodes VEGF.

Embodiment 93 provides the method of any one of Embodiments 84-93, wherein the LNP is administered as a pharmaceutical composition.

Embodiment 94 provides the method of Embodiment 93, wherein the pharmaceutical composition comprises at least one pharmaceutically acceptable carrier.

Embodiment 95 provides the method of any one of Embodiments 84-94, wherein the at least one ionizable lipid comprises an ionizable lipid selected from the group consisting of C12-200, MC3, and a compound of Formula (I), or a salt, solvate, stereoisomer, or isotopologue thereof:

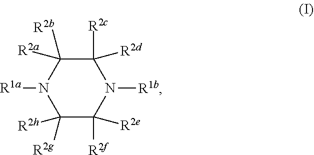

wherein:
R^{1a} and R^{1b} are each independently

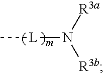

R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₇-C₁₃ aralkyl, optionally substituted C₆-C₁₀ aryl, and optionally substituted C₂-C₁₀ heteroaryl;
each occurrence of R^{3a}, R^{3b}, and R^{3c} is independently selected from the group consisting of H, -(optionally substituted C₁-C₆ alkylenyl)-C(═O)OR⁴, -(optionally substituted C₁-C₆ alkylenyl)-C(═O)N(R⁴)(R⁵), -(optionally substituted C₁-C₆ alkylenyl)-C(═O)R⁴, -(optionally substituted C₁-C₆ alkylenyl)-(R⁴), —C(═O)OR⁴, —C(═O)N(R⁴)(R⁵), —C(═O)R⁴, and R⁴,
   wherein no more than one of each occurrence of R^{3a}, R^{3b}, and R^{3c} is H;
R⁴ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁵ is selected from the group consisting of H and optionally substituted C₁-C₆ alkyl;
each occurrence of L is independently selected from the group consisting of -(optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—; and
each occurrence of m is independently an integer selected from the group consisting of 1, 2, 3, and 4.

Embodiment 96 provides the method of Embodiment 95, wherein at least one of the following applies:
(a) at least one selected from the group consisting of R^{2a}, R^{2b}, R^{2c}, R^{2d}, R^{2e}, R^{2f}, R^{2g}, and R^{2h} is H;
(b) at least two selected from the group consisting of R^{2a}, R^{2b}, R^{2c}, R^{2d}, R^{2e}, R^{2f}, R^{2g}, and R^{2h} are H;
(c) at least three selected from the group consisting of R^{2a}, R^{2b}, R^{2c}, R^{2d}, R^{2e}, R^{2f}, R^{2g}, and R^{2h} are H;
(d) at least four selected from the group consisting of R^{2a}, R^{2b}, R^{2c}, R^{2d}, R^{2e}, R^{2f}, R^{2g}, and R^{2h} are H;
(e) at least five selected from the group consisting of R^{2a}, R^{2b}, R^{2c}, R^{2d}, R^{2e}, R^{2f}, R^{2g}, and R^{2h} are H;
(f) at least six selected from the group consisting of R^{2a}, R^{2b}, R^{2c}, R^{2d}, R^{2e}, R^{2f}, R^{2g}, and R^{2h} are H;
(g) at least seven selected from the group consisting of R^{2a}, R^{2b}, R^{2c}, R^{2d}, R^{2e}, R^{2f}, R^{2g}, and R^{2h} are H; and
(h) each of R^{2a}, R^{2b}, R^{2c}, R^{2d}, R^{2e}, R^{2f}, R^{2g}, and R^{2h} are H.

Embodiment 97 provides the method of Embodiment 95 or 96, wherein each occurrence of L is independently selected from the group consisting of —CH₂—, —(CH₂)₂—, —(CH₂)₃—, —(CH₂)₁₀—, —(CH₂)₂O—, —(CH₂)₃O—, —CH₂CH(OR⁵)CH₂—, —(CH₂)₂NR^{3c}—,

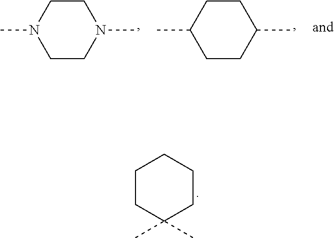

Embodiment 98 provides the method of any one of Embodiments 95-97, wherein the ionizable lipid of Formula (I) is selected from the group consisting of:

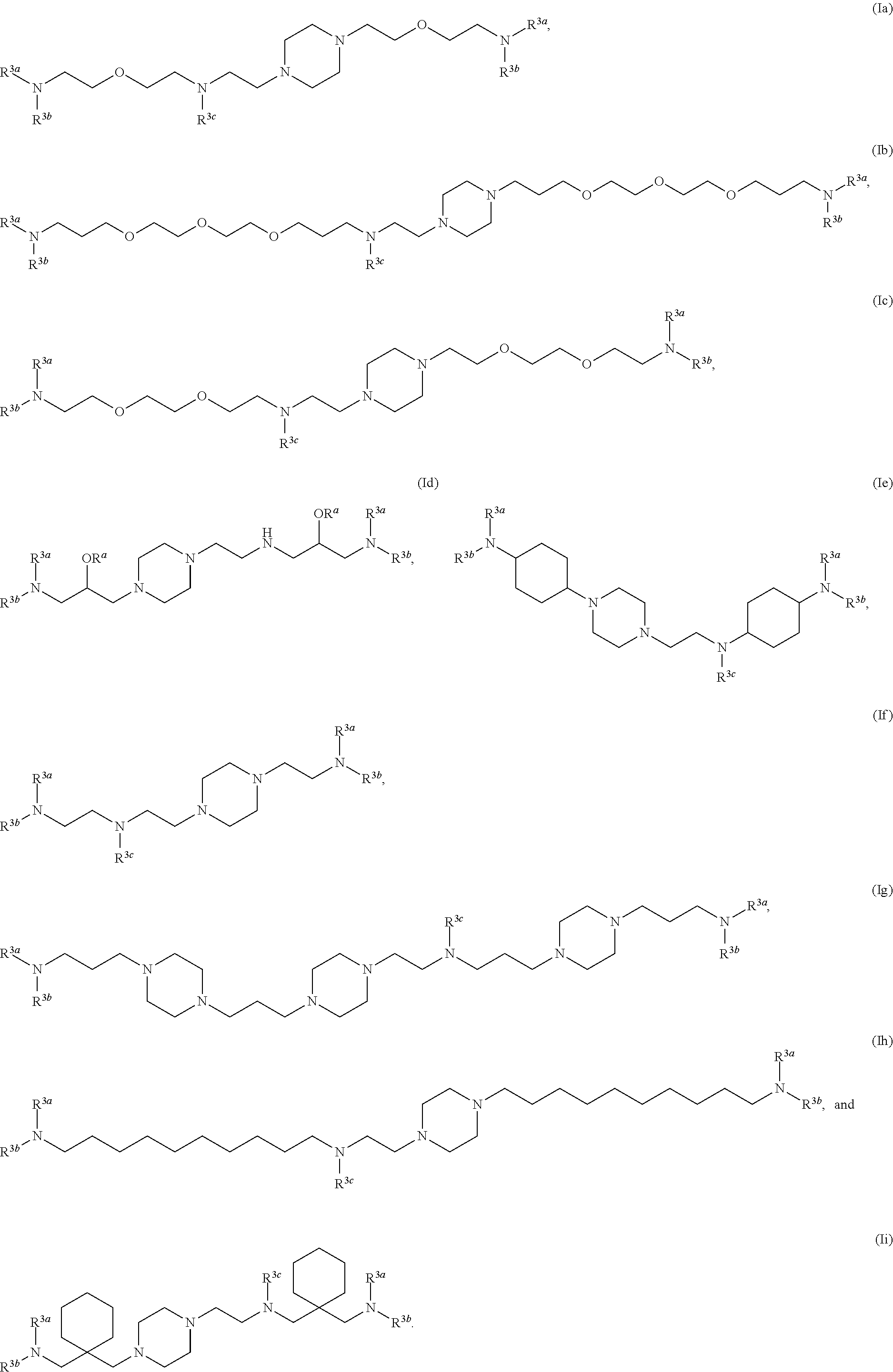

Embodiment 99 provides the method of any one of Embodiments 95-98, wherein each occurrence of R^{3a}, R^{3b}, and R^{3c} is independently selected from the group consisting of H, —CH₂CH(OH) (optionally substituted C₁-C₂₈ alkyl), —CH₂CH(OH) (optionally substituted C₂-C₂₈ alkenyl), —CH₂CH₂C(═O)O (optionally substituted C₁-C₂₈ alkyl), and —CH₂CH₂C(═O)NH (optionally substituted C₁-C₂₈ alkyl).

Embodiment 100 provides the method of any one of Embodiments 95-99, wherein each occurrence of R^{3a}, R^{3b}, and R^{3c} is independently selected from the group consisting of —CH₂CH(OH)(CH₂)₉CH₃, —CH₂CH(OH)(CH₂)₁₁CH₃, and —CH₂CH(OH)(CH₂)₁₃CH₃.

Embodiment 101 provides the method of any one of Embodiments 95-100, wherein each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, optionally substituted heteroalkylenyl, optionally substituted cycloalkylenyl, and optionally substituted heterocycloalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

Embodiment 102 provides the method of any one of Embodiments 95-101, wherein the ionizable lipid of Formula (I is:

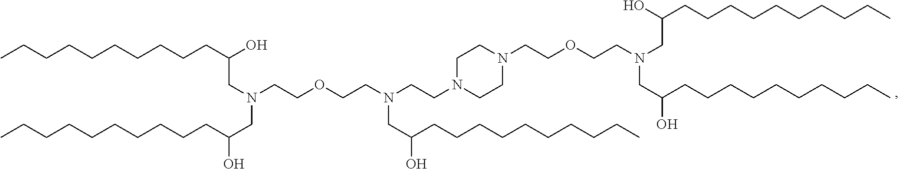 [SMILES: CCCCCCCCCCC(O)CN(CCOCCN(CC(O)CCCCCCCCCC)CC(O)CCCCCCCCCC)CCN1CCN(CCOCCN(CC(O)CCCCCCCCCC)CC(O)CCCCCCCCCC)CC1]

1,1′-((2-(2-(4-(2-((2-(2-(bis(2-hydroxydodecyl)amino)ethoxy)ethyl)(2-hydroxydodecyl)amino)ethyl)piperazin-1-yl)ethoxy)ethyl)azanediyl)bis(dodecan-2-ol) (A4);

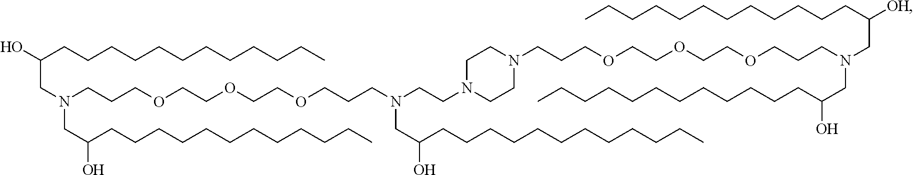 [SMILES: CCCCCCCCCCCCC(O)CN(CCCOCCOCCOCCCN(CC(O)CCCCCCCCCCCC)CC(O)CCCCCCCCCCCC)CCN1CCN(CCCOCCOCCOCCCN(CC(O)CCCCCCCCCCCC)CC(O)CCCCCCCCCCCC)CC1]

15-(2-(4-(16-hydroxy-14-(2-hydroxytetradecyl)-4,7,10-trioxa-14-azaoctacosyl)piperazin-1-yl)ethyl)-29-(2-hydroxytetradecyl)-19,22,25-trioxa-15,29-diazatritetracontane-13,31-diol (B5);

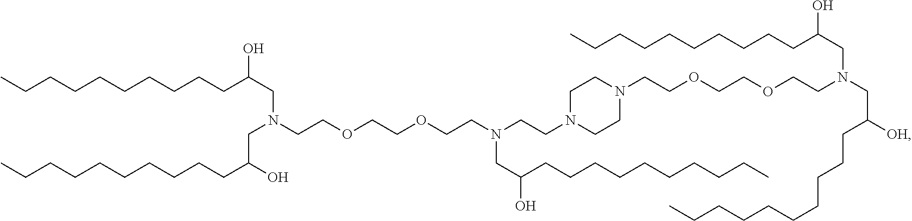 [SMILES: CCCCCCCCCCC(O)CN(CCOCCOCCN(CC(O)CCCCCCCCCC)CC(O)CCCCCCCCCC)CCN1CCN(CCOCCOCCN(CC(O)CCCCCCCCCC)CC(O)CCCCCCCCCC)CC1]

13-(2-(4-(2-(2-(2-(bis(2-hydroxydodecyl)amino)ethoxy)ethoxy)ethyl)piperazin-1-yl)ethyl)-22-(2-hydroxydodecyl)-16,19-dioxa-13,22-diazatetratriacontane-11,24-diol (A2);

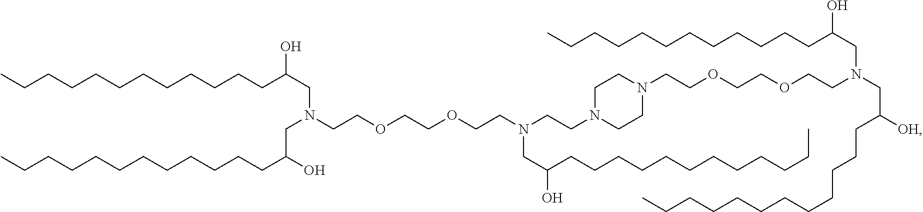 [SMILES: CCCCCCCCCCCCC(O)CN(CCOCCOCCN(CC(O)CCCCCCCCCCCC)CC(O)CCCCCCCCCCCC)CCN1CCN(CCOCCOCCN(CC(O)CCCCCCCCCCCC)CC(O)CCCCCCCCCCCC)CC1]

15-(2-(4-(2-(2-(2-(bis(2-hydroxytetradecyl)amino)ethoxy)ethoxy)ethyl)piperazin-1-yl)ethyl)-24-(2-hydroxytetradecyl)-18,21-dioxa-15,24-diazaoctatriacontane-13,26-diol (B2); and

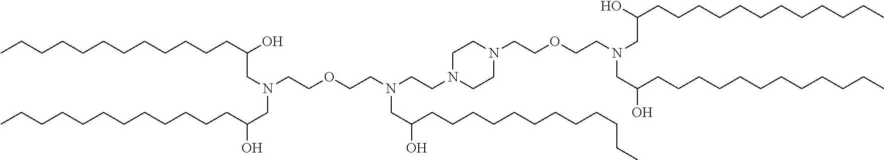 [SMILES: CCCCCCCCCCCCC(O)CN(CCOCCN(CC(O)CCCCCCCCCCCC)CC(O)CCCCCCCCCCCC)CCN1CCN(CCOCCN(CC(O)CCCCCCCCCCCC)CC(O)CCCCCCCCCCCC)CC1]

1,1′-((2-(2-(4-(2-((2-(2-(bis(2-hydroxytetradecyl)amino)ethoxy)ethyl)(2-hydroxytetradecyl)amino)ethyl)piperazin-1-yl)ethoxy)ethyl)azanediyl)bis(tetradecan-2-ol), (C14-494).

Embodiment 103 provides the method of any one of Embodiments 84-102, wherein the at least one ionizable lipid comprises about 10 mol % to about 60 mol % of the LNP.

Embodiment 104 provides the method of any one of Embodiments 84-103, wherein the at least one ionizable lipid comprises about 32.4, 35, 49, 51, or about 55 mol % of the LNP.

Embodiment 105 provides the method of any one of Embodiments 84-104, wherein the helper or neutral lipid comprises at least one selected from the group consisting of dioleoylphosphatidylethanolamine (DOPE) and distearoylphosphatidylcholine (DSPC).

Embodiment 106 provides the method of any one of Embodiments 84-105, wherein the at least one helper or neutral lipid comprises about 1 to about 50 mol % of the LNP.

Embodiment 107 provides the method of any one of Embodiments 84-106, wherein the at least one helper or neutral lipid comprises about 14, 16, 22.2, 29, or about 33 mol % of the LNP.

Embodiment 108 provides the method of any one of Embodiments 84-107, wherein the helper or neutral lipid is DOPE.

Embodiment 109 provides the method of any one of Embodiments 84-108, wherein the cholesterol and/or a derivative thereof comprises about 5 to about 70 mol % of the LNP.

Embodiment 110 provides the method of any one of Embodiments 84-109, wherein the cholesterol and/or a derivative thereof comprises about 15, 16, 31.5, 33, 46.5, or about 61.5 mol % of the LNP.

Embodiment 111 provides the method of any one of Embodiments 84-110, wherein the cholesterol and/or a derivative thereof is selected from the group consisting of cholesterol, 24-α-methyl-cholesterol (campesterol), 24-α-ethyl-cholestanol (stigmastanol), and 24-α-ethyl-cholesterol (β-sitosterol).

Embodiment 112 provides the method of any one of Embodiments 84-111, wherein the at least one polymer conjugated lipid comprises about 0.1 to about 20.0 mol % of the LNP.

Embodiment 113 provides the method of any one of Embodiments 84-112, wherein the at least one polymer conjugated lipid comprises about 1.6, 1.8, 1.9, 2.3, or about 2.5 mol % of the LNP.

Embodiment 114 provides the method of any one of Embodiments 84-113, wherein the at least one polymer conjugated lipid comprises C14-PEG2000 (C14-PEG₂ₖ).

Embodiment 115 provides the method of any one of Embodiments 84-114, wherein the LNP has a molar ratio of (a):(b):(c):(d) selected from the group consisting of:
(a) about 30:20:10:1;
(b) about 30:16:8:1;
(c) about 55:15:35:2;
(d) about 35:16:46.5:2.5;
(e) about 35:24:46.5:2.5; and
(f) about 35:16:61.5:2.5.

Embodiment 116 provides the method of any one of Embodiments 84-115, wherein the LNP comprises (a):(b):(c):(d) having a molar percentage selected from the group consisting of:
(a) about 49.18:32.79:16.39:1.64;
(b) about 54.55:29.09:14.55:1.82;
(c) about 51.40:14.02:32.71:1.87;
(d) about 35:16:46.5:2.5; and
(e) about 32.4:22.2:43.1:2.3.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

What is claimed is:

1. A method of delivering a cargo to the placenta of a pregnant subject, the method comprising administering to the subject a therapeutically effective amount of at least one lipid nanoparticle (LNP) comprising:
(a) at least one ionizable lipid;
(b) at least one helper or neutral lipid;
(c) cholesterol or a derivative thereof;
(d) at least one polymer conjugated lipid or a modified derivative thereof;
(e) at least one cargo molecule, wherein the at least one cargo molecule is at least partially encapsulated in the LNP.

2. A method of treating, preventing, or ameliorating a placental disease or disorder in a subject in need thereof, the method comprising administering to a subject a therapeutically effective amount of at least one lipid nanoparticle comprising:
(a) at least one ionizable lipid;
(b) at least one helper or neutral lipid;
(c) cholesterol and/or a modified derivative thereof;
(d) at least one polymer conjugated lipid or a modified derivative thereof;
(e) at least one cargo molecule, wherein the at least one cargo molecule is at least partially encapsulated in the LNP.

3. The method of claim 1, wherein the LNP further comprises an epidermal growth factor (EGFR) targeting domain, optionally wherein the EGFR targeting domain is covalently conjugated to at least one component of the LNP.

4. The method of claim 2, wherein the placental disorder is selected from the group consisting of pre-eclampsia, fetal growth restriction (FGR), intrauterine growth restriction (IUGR), placenta previa, placenta accreta, placenta increta, and placenta percreta.

5. The method of claim 1, wherein the cargo is at least one selected from the group consisting of a nucleic acid, small molecule, protein, therapeutic agent, antibody, and any combinations thereof.

6. The method of claim 1, wherein the cargo is a nucleic acid, optionally wherein at least one of the following applies:
(a) the nucleic acid is DNA or RNA; and
(b) the nucleic acid is selected from the group consisting of mRNA, cDNA, pDNA, microRNA, siRNA, modified RNA, antagomir, antisense molecule, and any combinations thereof.

7. The method of claim 6, wherein the cargo is mRNA, optionally wherein the mRNA encodes VEGF.

8. The method of claim 1, wherein the LNP is administered as a pharmaceutical composition, optionally wherein the pharmaceutical composition comprises at least one pharmaceutically acceptable carrier.

9. The method of claim 1, wherein the at least one ionizable lipid comprises an ionizable lipid selected from the group consisting of C12-200, MC3, and a compound of Formula (I), or a salt, solvate, stereoisomer, or isotopologue thereof: 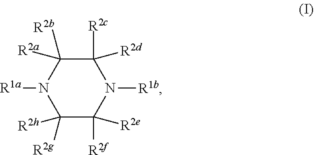
wherein:
R^{1a} and R^{1b} are each independently 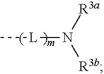
R^{2a}, R^{2b}, R^{2c}, R^{2d}, R^{2e}, R^{2f}, R^{2g}, and R^{2h} are each independently selected from the group consisting of H, optionally substituted C₁-C₁₂ alkyl, optionally substituted C₂-C₁₂ heteroalkyl, optionally substituted C₃-C₈ cycloalkyl, optionally substituted C₂-C₈ heterocycloalkyl, optionally substituted C₂-C₁₂ alkenyl, optionally substituted C₂-C₁₂ alkynyl, optionally substituted C₇-C₁₃ aralkyl, optionally substituted C₆-C₁₀ aryl, and optionally substituted C₂-C₁₀ heteroaryl;
each occurrence of R^{3a}, R^{3b}, and R^{3c} is independently selected from the group consisting of H, -(optionally substituted C₁-C₆ alkylenyl)-C(═O)OR⁴, -(optionally substituted C₁-C₆ alkylenyl)-C(═O)N(R⁴)(R⁵), -(optionally substituted C₁-C₆ alkylenyl)-C(═O) R⁴, -(optionally substituted C₁-C₆ alkylenyl)-(R⁴), —C(═O)OR⁴, —C(═O)N(R⁴)(R⁵), —C(═O)R⁴, and R⁴,
wherein no more than one of each occurrence of R^{3a}, R^{3b}, and R^{3c} is H;
R⁴ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁵ is selected from the group consisting of H and optionally substituted C₁-C₆ alkyl;
each occurrence of L is independently selected from the group consisting of -(optionally substituted C₁-C₁₂ alkylenyl)-X—, -(optionally substituted C₂-C₁₂ alkenylenyl)-X—, -(optionally substituted C₁-C₁₂ alkynylenyl)-X—, -(optionally substituted C₁-C₁₂ heteroalkylenyl)-X—, optionally substituted C₃-C₈ cycloalkylenyl, and optionally substituted C₂-C₈ heterocyloalkylenyl;
each occurrence of X, if present, is independently selected from the group consisting of a bond, —N(R^{3c})—, and —O—; and
each occurrence of m is independently an integer selected from the group consisting of 1, 2, 3, and 4.

10. The method of claim 9, wherein the ionizable lipid of Formula (I) is selected from the group consisting of:

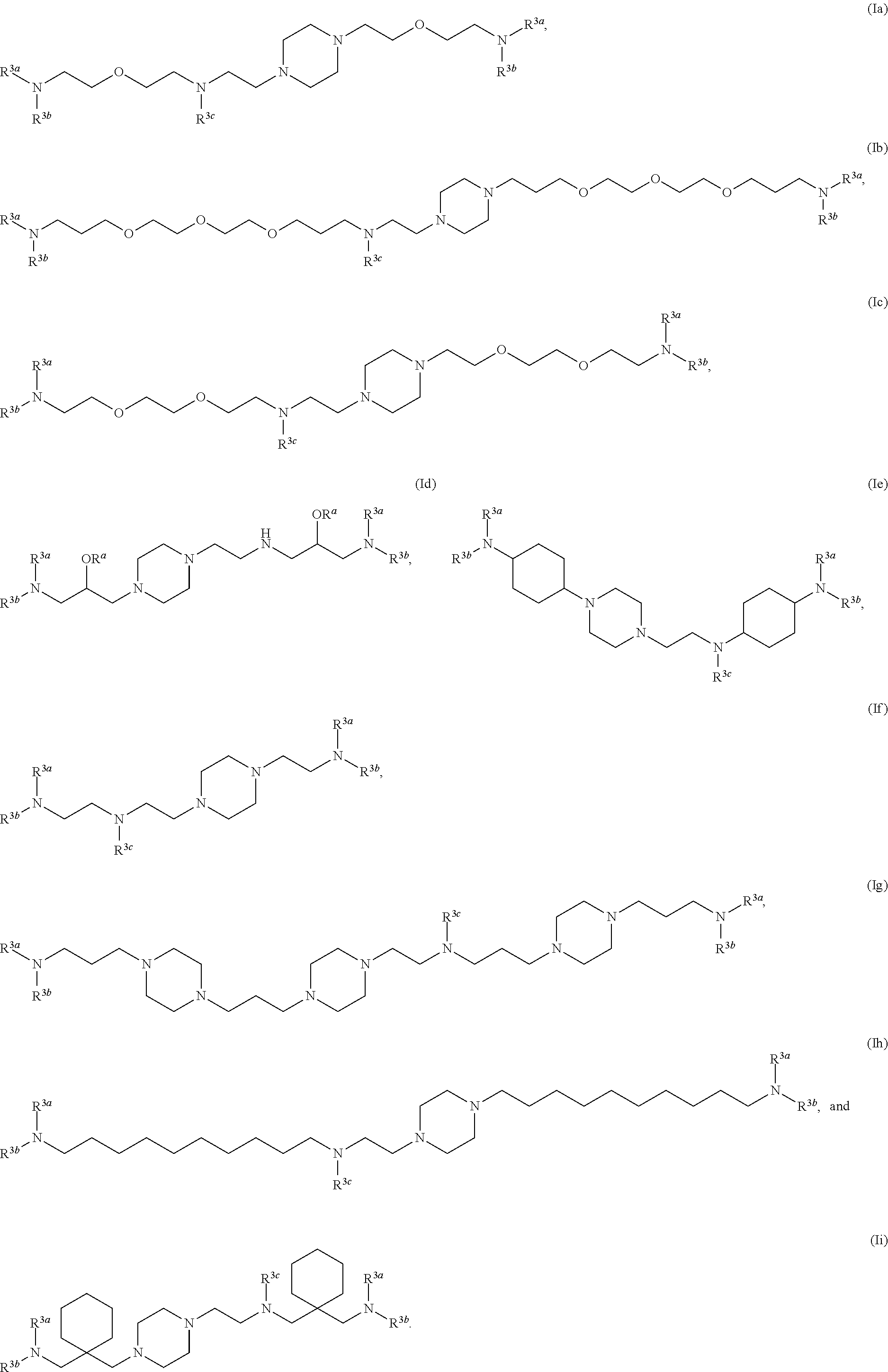

11. The method of claim 12, wherein the ionizable lipid of Formula (I) is: 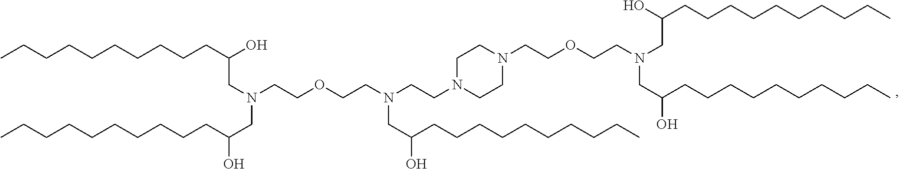 [SMILES: CCCCCCCCCCC(O)CN(CCOCCN(CC(O)CCCCCCCCCC)CC(O)CCCCCCCCCC)CCN1CCN(CCOCCN(CC(O)CCCCCCCCCC)CC(O)CCCCCCCCCC)CC1]
1,1′-((2-(2-(4-(2-((2-(2-(bis(2-hydroxydodecyl)amino)ethoxy)ethyl)(2-hydroxydodecyl)amino)ethyl)piperazin-1-yl)ethoxy)ethyl) azanediyl)bis(dodecan-2-ol) (A4); 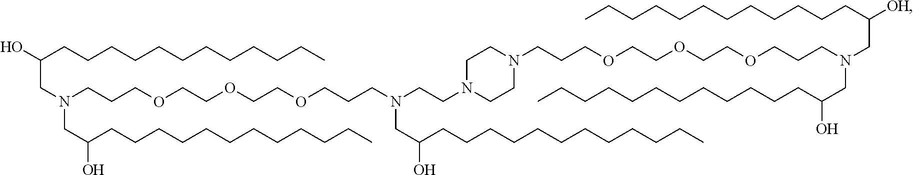 [SMILES: CCCCCCCCCCCCC(O)CN(CCCOCCOCCOCCCN(CC(O)CCCCCCCCCCCC)CC(O)CCCCCCCCCCCC)CCN1CCN(CCCOCCOCCOCCCN(CC(O)CCCCCCCCCCCC)CC(O)CCCCCCCCCCCC)CC1]
15-(2-(4-(16-hydroxy-14-(2-hydroxytetradecyl)-4,7,10-trioxa-14-azaoctacosyl)piperazin-1-yl)ethyl)-29-(2-hydroxytetradecyl)-19,22,25-trioxa-15,29-diazatritetracontane-13,31-diol (B5); 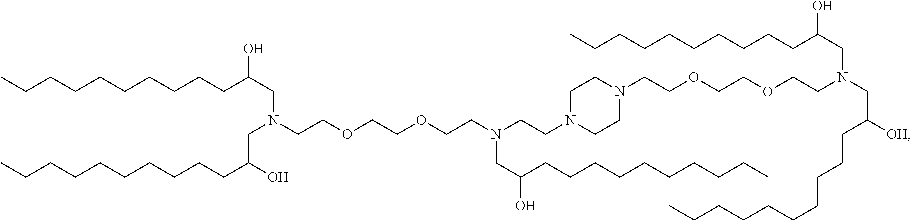 [SMILES: CCCCCCCCCCC(O)CN(CCOCCOCCN(CC(O)CCCCCCCCCC)CC(O)CCCCCCCCCC)CCN1CCN(CCOCCOCCN(CC(O)CCCCCCCCCC)CC(O)CCCCCCCCCC)CC1]
13-(2-(4-(2-(2-(2-(bis(2-hydroxydodecyl)amino)ethoxy)ethoxy)ethyl)piperazin-1-yl)ethyl)-22-(2-hydroxydodecyl)-16,19-dioxa-13,22-diazatetratriacontane-11,24-diol (A2); 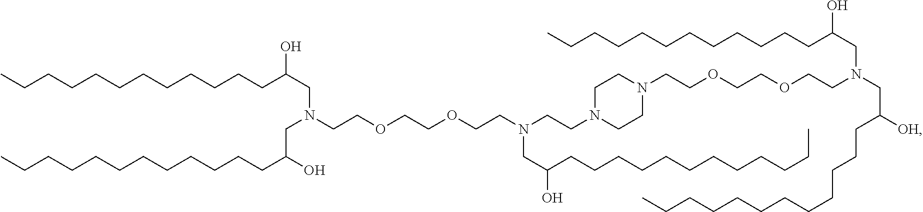 [SMILES: CCCCCCCCCCCCC(O)CN(CCOCCOCCN(CC(O)CCCCCCCCCCCC)CC(O)CCCCCCCCCCCC)CCN1CCN(CCOCCOCCN(CC(O)CCCCCCCCCCCC)CC(O)CCCCCCCCCCCC)CC1]
15-(2-(4-(2-(2-(2-(bis(2-hydroxytetradecyl)amino)ethoxy)ethoxy)ethyl)piperazin-1-yl)ethyl)-24-(2-hydroxytetradecyl)-18,21-dioxa-15,24-diazaoctatriacontane-13,26-diol (B2); and 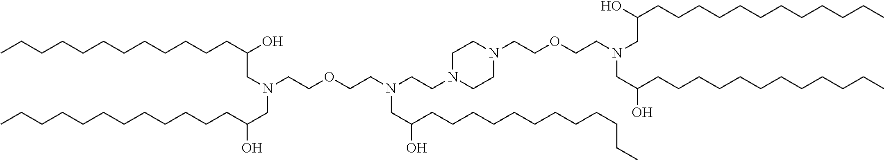 [SMILES: CCCCCCCCCCCCC(O)CN(CCOCCN(CC(O)CCCCCCCCCCCC)CC(O)CCCCCCCCCCCC)CCN1CCN(CCOCCN(CC(O)CCCCCCCCCCCC)CC(O)CCCCCCCCCCCC)CC1]
1,1′-((2-(2-(4-(2-((2-(2-(bis(2-hydroxytetradecyl)amino)ethoxy)ethyl)(2-hydroxytetradecyl)amino)ethyl)piperazin-1-yl)ethoxy)ethyl)azanediyl)bis(tetradecan-2-ol), (C14-494).

12. The method of claim 1, wherein the at least one ionizable lipid comprises about 10 mol % to about 60 mol % of the LNP, optionally wherein the at least one ionizable lipid comprises about 32.4, 35, 49, 51, or about 55 mol % of the LNP.

13. The method of claim 1, wherein the helper or neutral lipid comprises at least one selected from the group consisting of dioleoylphosphatidylethanolamine (DOPE) and distearoylphosphatidylcholine (DSPC).

14. The method of claim 1, wherein the at least one helper or neutral lipid comprises about 1 to about 50 mol % of the LNP, optionally wherein the at least one helper or neutral lipid comprises about 14, 16, 22.2, 29, or about 33 mol % of the LNP.

15. The method of claim 1, wherein the helper or neutral lipid is DOPE.

16. The method of claim 1, wherein the cholesterol or a derivative thereof comprises about 5 to about 70 mol % of the LNP, optionally wherein the cholesterol and/or a derivative thereof comprises about 15, 16, 31.5, 33, 46.5, or about 61.5 mol % of the LNP.

17. The method of claim 1, wherein the cholesterol or a derivative thereof is a sterol, optionally wherein the sterol is selected from the group consisting of cholesterol, 24-α-methyl-cholesterol (campesterol), 24-α-ethyl-cholestanol (stigmastanol), and 24-α-ethyl-cholesterol (β-sitosterol).

18. The method of claim 1, wherein the at least one polymer conjugated lipid comprises about 0.1 to about 20.0 mol % of the LNP.

19. The method of claim 1, wherein the at least one polymer conjugated lipid comprises about 1.6, 1.8, 1.9, 2.3, or about 2.5 mol % of the LNP, optionally wherein the at least one polymer conjugated lipid comprises C14-PEG2000 (C14-PEG₂ₖ).

20. The method of claim 1, wherein the LNP has a molar ratio of (a):(b):(c):(d) selected from the group consisting of:
(a) about 30:20:10:1;
(b) about 30:16:8:1;
(c) about 55:15:35:2;
(d) about 35:16:46.5:2.5;
(e) about 35:24:46.5:2.5;
(f) about 35:16:61.5:2.5;
(g) about 49.18:32.79:16.39:1.64;
(h) about 54.55:29.09:14.55:1.82;
(i) about 51.40:14.02:32.71:1.87; and
(j) about 32.4:22.2:43.1:2.3.


SEQUENCE LISTING (1 sequence)

SEQ ID NO: 1 - protein, 190 aa, synthetic construct
QGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA